(19) United States
(12) Patent Application Publication
(10) Pub. No.: US 2024/0424127 A1
(43) Pub. Date: Dec. 26, 2024

(54) IL-7 POLYPEPTIDES, IMMUNOCYTOKINES COMPRISING SAME, AND USES THEREOF

(71) Applicant: Bright Peak Therapeutics AG, Basel (CH)

(72) Inventors: Vijaya Raghavan PATTABIRAMAN, Volketswil (CH); Bertolt KREFT, Kleinmachnow (DE); Matilde ARÉVALO-RUIZ, Basel (CH); Eric ARMENTANI, Wettingen (CH)

(21) Appl. No.: 18/409,707

(22) Filed: Jan. 10, 2024

Related U.S. Application Data
(60) Provisional application No. 63/438,501, filed on Jan. 11, 2023.

(51) Int. Cl. A61K 47/68; C07K 14/54; C07K 16/28

(52) U.S. Cl. CPC A61K 47/6813; A61K 47/6849; C07K 14/5418; C07K 16/2818; C07K 2317/24

(57) ABSTRACT

The present disclosure relates to IL-7 polypeptides, compositions comprising IL-7 polypeptides, methods of making the same, and methods of using the IL-7 polypeptides for treatment of diseases. In one aspect, the disclosure relates to the treatment of cancer using the IL-7 polypeptides. In some embodiments, the disclosed IL-7 polypeptides comprise at least one modification at an amino acid residue which reduces the affinity of the IL-7 polypeptide to the IL-7 receptor. Also provided herein are synthetic IL-7 polypeptides and methods of manufacture thereof. Further provided herein are immunocytokines comprising immune checkpoint inhibitor molecules, such as anti-PD-1 antibodies, couple to the IL-7 polypeptides provided herein.

This application claims priority to U.S. Provisional Patent Application No. 63/438,501, filed Jan. 11, 2023, which application is incorporated herein by reference in its entirety.

### SEQUENCE LISTING

The instant application contains a Sequence Listing which has been submitted electronically in XML file format and is hereby incorporated by reference in its entirety. Said XML copy, created on Jan. 29, 2024, is named 94917-0112_720201US_SL.xml and is 367,302 bytes in size.

### BACKGROUND

Immunotherapies utilize the immune system of a subject to aid in the treatment of ailments. Immunotherapies can be designed to stimulate or suppress the immune system depending on the nature of the disease being treated. The goal of immunotherapies for the treatment of cancer is to stimulate the immune system so that it recognizes and destroys tumors or other cancerous tissue. One method of stimulating the immune system to attack cancer cells in the body of a subject is cytokine therapy. Cytokines are proteins produced in the body that are important in cell signaling and modulate the immune system. Some cytokine therapy utilizes these properties of cytokines to enhance the immune system of a subject to kill cancer cells.

Interleukin 7 (IL-7) is a non-hematopoietic cell-derived cytokine with a central role in the adaptive immune system. IL-7 promotes lymphocyte development in the thymus and maintains survival of naïve and memory T cell homeostasis in the periphery. IL-7 is secreted by stromal cells in the bond marrow and thymus, and is also produced by keratinocytes, dendritic cells, hepatocytes, neurons, and epithelial cells. IL-7 is not produced by normal lymphocytes. The autocrine production of IL-7 cytokine mediated by T-cell acute lymphoblastic leukemia (T-ALL) can be involved in the oncogenic development of T-ALL. IL-7 is important for the organogenesis of lymph nodes and for the maintenance of activated T cells recruited into the secondary lymphoid organs.

IL-7 is a cytokine important for B and T cell development. The IL-7 cytokine and the hepatocyte growth factor (HGF) form a heterodimer that functions as a pre-pro-B cell growth-stimulating factor. IL-7 is also a cofactor for V(D)J arrangement of the T cell receptor beta (TCRβ) during early T cell development. IL-7 can be produced locally by intestinal epithelial and epithelial goblet cells, and can serve as a regulatory factor for intestinal mucosal lymphocytes. IL-7 stimulates the differentiation of multipotent (pluripotent) hematopoietic stem cells into lymphoid progenitor cells. IL-7 also stimulates proliferation of all cells in the lymphoid lineage, such as B cells, T cells, and NK cells. IL-7 is important for proliferation during certain stages of B-cell maturation, T and NK cell survival, development, and homeostasis.

IL-7 binds to the IL-7 receptor (IL-7R), a heterodimer consisting of IL-7R alpha (IL-7Rα) and common gamma chain receptor (γc). Binding results in a cascade of signals important for T-cell development within the thymus and survival within the periphery, Knockout mice that genetically lack IL-7R exhibit thymic atrophy, arrested T-cell development at the double positive stage, and severe lymphopenia.

### BRIEF SUMMARY

Provided herein in some embodiments are IL-7 polypeptides which comprise modifications, such as modifications which reduce the affinity of the IL-7 polypeptides with the IL-7 receptor or a subunit thereof (e.g., the IL-7 receptor alpha subunit). In some instances, reduced affinity IL-7 polypeptides are better able to preferentially stimulate effector T cells and/or natural killer cells while sparing regulatory T cells, potentially at least partly due to differential expression of the IL-7 receptor on the different T cell subtypes. Such IL-7 polypeptides are potentially useful as immunotherapeutics.

In some embodiments, an IL-7 polypeptide provided herein is synthetic (e.g., prepared from one or more chemically synthesized peptides). In some embodiments, an IL-7 polypeptide which is synthetic comprises one or more of the modifications provided herein which reduce affinity of the IL-7 polypeptide to the IL-7 receptor. The modifications to a synthetic IL-7 polypeptide can be any modification which can be included in a recombinant IL-7 polypeptide. In some instances, an IL-7 polypeptide which is synthetic offers advantages over an IL-7 polypeptide produced by other methods (e.g., recombinantly), such as the ability to easily and site specifically modify the IL-7 polypeptide (e.g., by incorporating modified natural or unnatural amino acids during synthesis). In some embodiments, an IL-7 polypeptide which is synthetic has a similar or substantially identical activity to a corresponding recombinant IL-7 polypeptide (e.g., an IL-7 polypeptide which contains the same functional modifications but without the incorporation of residues necessary for the synthesis (e.g., ligation) of the IL-7 polypeptide). Also provided herein are methods of synthesizing an IL-7 polypeptide.

Further provided herein are immunocytokines comprising the IL-7 polypeptides described herein and immune checkpoint inhibitor molecules (e.g., anti-PD-1 antibodies or antigen binding fragments thereof). In some embodiments, the immunocytokines allow for targeting of IL-7 polypeptides to target tissues (e.g., tumors) and can in some instances provide synergistic therapeutic effects owing to the activity of the immune checkpoint inhibitor molecule and the IL-7 polypeptide on the same molecule and thus simultaneously delivered to a target cell at the same time.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing, of which:

FIG. 1 shows a general synthesis scheme used to produce synthetic IL-7 linear proteins.

FIG. 2A illustrates the mechanism of action of IL-7.

FIG. 2B shows differential expression of CD127 (IL-7R) on a variety of T cell subtypes.

FIGS. 3A-3D shows characterization data for a synthetic IL-7 polypeptide.

FIG. 3A shows characterization data of synthetic IL-7 of SEQ ID. NO: 3 (CMP-036) linear protein, including a) a RP-HPLC trace, showing retention time on the X-axis and absorbance on the Y-axis (top left); and b) an ESI-HRMS trace, with molecular weight on the X-axis and intensity on the Y-axis (top right).

FIG. 3B shows RP-HPLC (top) and MALDI-TOF (bottom) is shown for CMP-036 in the folded state.

FIG. 3C shows a RP-HPLC trace (top, left) and ESI_HRMS (top, right) for CMP-108, synthetic IL-7 attached to a conjugation handle as a linear protein.

FIG. 3D shows an RP-HPLC (top) and ESI-HRMS (bottom) of folded CMP-108.

FIGS. 4A-4D show dose response curves of IL-7 polypeptides and IL-7 immunocytokines for T cell types in terms of pSTAT5%.

FIG. 4A shows the response to doses of recombinant IL-7 (WT) (CMP-035) and synthetic IL-7 (WT) (CMP-036) on STAT5 activation (pSTAT5%) in CD8⁺ memory (top), or CD8⁺ naïve (bottom).

FIG. 4B shows the response to synthetic IL-7 (WT) (CMP-036); pembrolizumab conjugated synthetic IL-7 (WT) DAR=1 (CMP-039); and LZM-009 conjugated synthetic IL-7 (WT) DAR=1 (CMP-041) on pSTAT5% in CD8⁺ memory T cells (top), and CD8⁺ naïve T cells (bottom).

FIGS. 4C and 4D show the effects of synthetic IL-7 polypeptides and conjugates on pSTAT5% in CD8⁺ cells are shown for synthetic IL-7 polypeptides and conjugates: [synthetic IL-7, K81 (L6P), G85N (CMP-111)], [synthetic IL-7, K81 (L6P) (CMP-109)], [synthetic IL-7, G85N (CMP-110)], [synthetic IL-7, WT, conjugated to LZM-009, DAR=1 (CMP-041)], [synthetic IL-7, V15W conjugated to LZM-009, DAR=1 (CMP-116)], and [synthetic IL-7, WT with an N-terminal conjugation handle (CMP-108)], [synthetic IL-7, WT (CMP-039)].

FIG. 4C shows the response to the indicated polypeptides and conjugates in terms of STAT5% activation in a mixed population of CD8+ memory and T_{eff} cell populations.

FIG. 4D shows the response to the indicated polypeptides and conjugates in terms of STAT5% activation in a mixed population of CD8+ naïve and T_{eff} cell populations.

FIG. 4E shows the pSTAT5% response in a mixed population of CD8+ memory and Teff cells synthetic IL-7 polypeptides: CMP-108 is synthetic IL-7 with an N-term conjugation handle; CMP-107 is synthetic IL-7 with V15W; CMP-109 is synthetic IL-7, K81 (L6P); CMP-110 is synthetic IL-7 (G85N); CMP-112 is synthetic IL-7 with K81 (L11P); CMP-113 is synthetic IL-7 with C2A, C92A; CMP-114 (synthetic IL-7 of SEQ ID NO: 3 with N-terminal 30 kDa PEG conjugated); and CMP-115 is synthetic IL-7 with K81 (L2P).

FIG. 4F shows the STAT5% response of CD8 naïve and Teff cells for the synthetic IL-7 polypeptides as described for FIG. 4E.

FIG. 5A shows an illustration of a folded IL-7 polypeptide.

FIG. 5B shows an illustration of a folded IL-7 polypeptide with an N-terminal conjugation handle.

FIG. 5C shows an illustration of a folded IL-7 polypeptide with a PEG group attached to the N-terminal residue.

FIG. 6A shows site-selective modification of anti-PD-1 antibody by AJICAP™ technology to introduce one conjugation handle.

FIG. 6B shows site-selective conjugation reaction of IL-7 cytokine to generate anti-PD-1-IL7 with drug/antibody ratio of 1 (DAR1) or DAR2.

FIG. 7A shows ELISA assay results of dose dependent binding of antibodies to PD-1. Assayed are pembrolizumab and synthetic IL-7/pembrolizumab conjugates with DAR=1 and DAR=2 (CMP-039 and CMP-040, respectively).

FIG. 7B shows ELISA assay for results of dose dependent binding of antibodies to PD-1. Assayed are LZM-009, and synthetic IL-7 (WT) LZM-009 conjugate DAR=1 (CMP-041).

FIG. 7C shows the dose dependent binding of antibodies and conjugates to PD-1. Dose response curves are shown for Pembrolizumab; LZM-009; synthetic IL-7 (V15W) conjugated to LZM-009, DAR=1 (CMP-116); and synthetic IL-7 (WT) conjugated to LZM-009, DAR=1 (CMP-041) in an ELISA assay.

FIG. 8 shows plots measuring ability of the unmodified LZM-009 and synthetic IL-7 conjugates to LZM-009 to interfere with PD1/PDL1 binding. The readout was reflected in transcriptional activity of the NFAT promoter, which is downstream of PD-1 signal transduction. Assayed are LZM-009, LZM-009 conjugated to synthetic IL-7 (WT) (CMP-041) and LZM conjugated to synthetic IL-7 (V15W) (CMP-116).

FIG. 9A shows individual plots of ELISA results for dose dependent binding to human FcRn by LZM-009 and LZM-009 conjugates: synthetic IL-7 (V15W) (CMP-116); or synthetic IL-7 (WT) (CMP-041).

FIG. 9B shows individual plots of ELISA results for dose dependent binding to mouse FcRn by LZM-009 or LZM-009 conjugates: CMP-116 or CMP-041.

FIG. 10A shows changes in relative mouse body weight over a two week period in percentage (y-axis) while weekly dosing of LZM-009 (10 mg/kg) and synthetic IL-7 (WT) (CMP-041) (1, 3, and 10 mg/kg). Arrows indicate dosing days.

FIG. 10B shows relative tumor volume % (y-axis) over a two week period while weckly dosing of LZM-009 (10 mg/kg) and CMP-041 (1, 3, and 10 mg/kg). Arrows indicate dosing days.

FIGS. 11A-11C show changes in T cell stimulation in CD8 naïve, CD4 naïve, CD8 memory, CD4 memory, and CD4 Treg cells upon treatment with IL-2 (FIG. 11A); wild type IL-7 (with his-tag) (CMP-035) (FIG. 11B); and IL-7 with V15W substitution and his-tag (CMP-106) (FIG. 11C). The X-axis shows the concentration (nM) of the agonist, and the Y-axis shows pSTAT5%. The V15W substitution in CMP-106 results in a change of potency in IL-7 from 3 fold to 30 fold for Teff vs Treg cells.

FIGS. 12A-B show pSTAT5 assay results in human pan T cells of S—S bond variants as EC50 (FIG. 12A) and fold change EC50 (FIG. 12B) compared to WT syntein, gated on CD4 memory T cells.

FIG. 13 shows internalization of IL7R after its engagement with wt IL7variant.

FIGS. 14A-O show Bio-Layer Interferometry analysis of IL7 variants with CD127. CMP-035 (FIG. 14A); CMP-035 (FIG. 14A); CMP-108 (FIG. 14B); CMP-106 (FIG. 14C); CMP-107 (FIG. 14D); CMP-109 (FIG. 14E); CMP-110 (FIG. 14F); CMP-111 (FIG. 14G); CMP-108-009 (FIG. 14H); CMP-110-001 (FIG. 14I); CMP-111 (FIG. 14J); CMP-200 (FIG. 14K); CMP-201 (FIG. 14L); CMP-202 (FIG. 14M); CMP-116 (FIG. 140).

### DETAILED DESCRIPTION

### I. IL-7 Polypeptides

Interleukin-7 (IL-7) belongs to the γ_{c}-chain family of cytokines, which also includes IL-2, IL-4, IL-9, IL-15, and IL-21. Like all receptors of the γ_{c}-chain cytokine family, the IL-7 receptor utilizes the common γ_{c}-chain subunit (CD132, also referred to herein as the IL-7 receptor gamma subunit) in conjunction with another subunit specific for IL-7 named IL-7 receptor alpha subunit (IL-7Rα, a.k.a. CD127). Thus, the IL-7 receptor is a heterodimer of the IL-7a subunit and the common γ_{c}-chain.

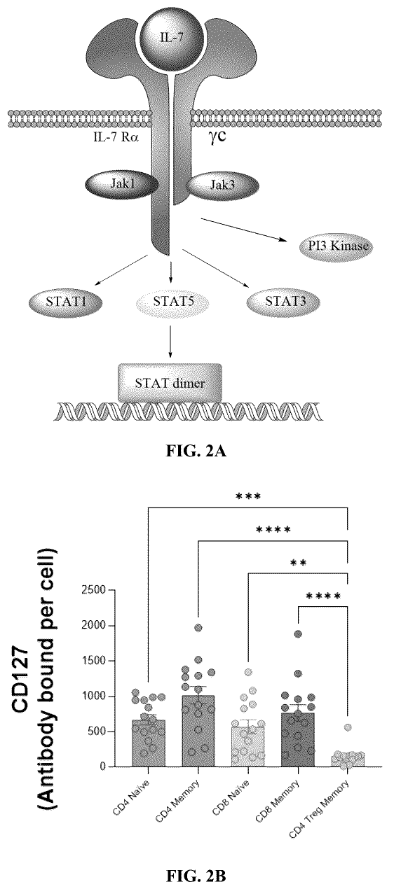

FIG. 2A illustrates the mechanism of action of IL-7. T cell-mediated immunomodulation can be defined as altering the T_{reg}:T_{eff} ratio. Immunosuppression skews the net T_{reg}:T_{eff} ratio towards the ‘tolerogenic’ Treg component, while immunostimulation skews the response toward the ‘proinflammatory’ Teff component. In the treatment of autoimmune diseases, achieving an immunosuppressive state is desirable to prevent ongoing injury by activated T_{eff} cells. In contrast, an innate or induced immunosuppressive state can prevent pathogen-induced disease while allowing for the progression of cancer. Methods of attenuating an existing endogenous immunosuppressive state that prevents effective T cell-mediated immune recognition of cancer cells can be used to modulate T_{reg}:T_{eff} ratios to treat autoimmune diseases and cancers.

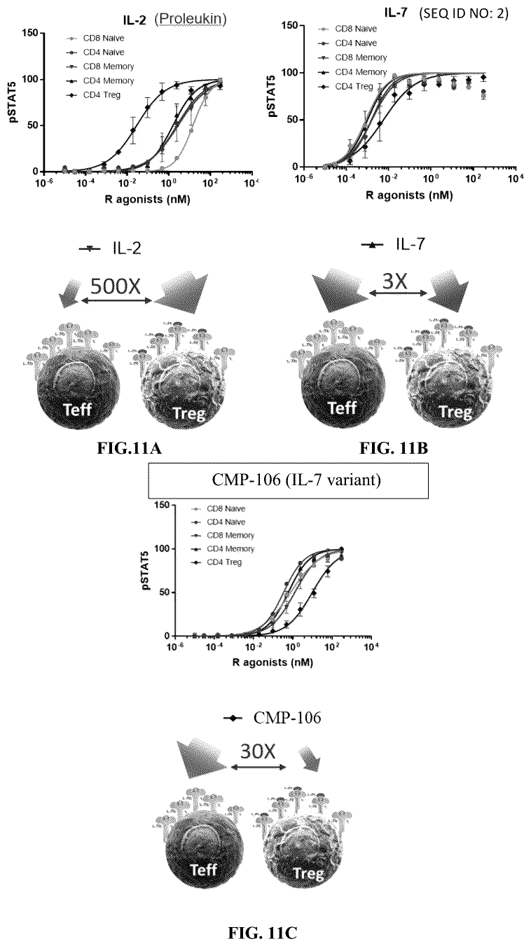

The IL-7Rα/γ heterodimer (IL-7R) is expressed on T cells, pre-B cells, and dendritic cells. Because IL-7R is expressed across immune T cell subtypes, IL-7 can act as a “pan-T cell” cytokine, stimulating numerous effector T (T_{eff}) and regulatory T (T_{reg}) cells (e.g., CD8 Naïve, CD4 Naïve, CD8 memory, CD4 memory, and CD4 T_{reg} cells) with nearly identical potency. This is in contrast to other cytokines such as IL-2, which is known to be a strong stimulator of T_{reg} cells and displays ˜500× more potent stimulation of T_{reg} cells compared to T_{eff} cells (see FIG. 11A). Conversely, IL-7 shows nearly identical potency for T_{eff} and T_{reg} cells, with a modest ˜3× increase in potency for stimulation of T_{eff} cells compared to T_{reg} cells (see FIG. 11B).

Although many T cell subtypes express IL-7R, different subtypes express IL-7R at different levels. In particular, many T_{eff} subtypes express IL-7R at significantly higher levels than T_{reg} cells. This is shown in FIG. 2B, which shows the amount of anti-CD127 antibody bound per cell (Y-axis) across a number of T-cell subtypes (x-axis). Notably, the T_{eff} subtypes (CD4 Naïve, CD4 Memory, CD8 Naïve, and CD8 Memory) all expressed significantly more IL-7R than the T_{reg} subtype (CD4 T_{reg} Memory). As T_{eff} and T_{reg} subtypes express IL-7R at different levels, IL-7 variants with a lesser affinity for IL-7R that still specifically bind to IL-7R may, in some instances, preferentially stimulate T_{eff} cells while leaving T_{reg} cells in an unstimulated state. Thus, IL-7 variants with reduced affinity for IL-7R offer potential as immunotherapies that create a superior immune response in the individual, such as an improved anti-tumor response. By way of non-limiting example, an IL-7 polypeptide provided herein with reduced binding to the IL-7 receptor (due to a V15W substitution as provided herein) results in a 30-fold increase in potency for stimulation of T_{eff} cells compared to T_{reg} cells (see FIG. 11C). Similar effects are observed for other IL-7 polypeptides provided herein (e.g., those containing other substitutions or modifications as provided herein, such as G85N substitutions, polymers attached at residue 81, etc.).

Provided herein in some embodiments are interleukin-7 (IL-7) polypeptides which have a reduced affinity to one or both of the IL-7 receptor α/γ_{c} heterodimer (IL-7R) and/or the IL-7 receptor a subunit. In some embodiments, IL-7 polypeptides provided herein still retain the ability to specifically bind to the IL-7 receptor, which can be useful for purposes such as to maintain the ability to effectuate signaling through IL-7R at physiologically and/or therapeutically relevant concentrations. In some embodiments, IL-7 polypeptides as provided herein display a greater potency for stimulation of effector T (T_{eff}) cell IL-7 signaling compared to regulatory T (T_{reg}) cells.

In some instances, IL-7 polypeptides provided herein comprise modifications at one or more amino acid residues at the binding interface of IL-7R and IL-7. In some embodiments, the IL-7 polypeptide comprises modifications at amino acid residues at the binding interface of IL-7Ra and IL-7. In some embodiments, the IL-7 polypeptide comprises modifications at amino acid residues at the binding interface of the γ_{c}-chain and IL-7.

In some embodiments, IL-7 polypeptides provided herein are synthetic (e.g., prepared from one or more chemically synthesized precursor fragments). Further disclosed herein are methods of preparing an IL-7 polypeptide comprising (a) synthesizing at least two building blocks; (b) ligating the at least two building blocks; and (c) forming a full length IL-7 polypeptide. Also provided herein is a method of treating a cancer, the method comprising administering a therapeutically effective amount of an IL-7 polypeptide of the disclosure.

In one aspect, provided herein, is an IL-7 polypeptide comprising at least one modification to the amino acid sequence as set forth in SEQ ID NO: 1, wherein the at least one modification is a natural amino acid substitution or an additional group covalently attached to a side chain of an amino acid residue of the IL-7 polypeptide, and wherein the IL-7 polypeptide is synthetic.

In one aspect, provided herein, is an IL-7 polypeptide comprising at least one modification to the amino acid sequence as set forth in SEQ ID NO: 3, wherein the IL-7 polypeptide exhibits reduced binding to the IL-7 receptor as compared to an IL-7 polypeptide of SEQ ID NO: 3 without the modification. In some embodiments, the modification is a natural amino acid substitution relative to SEQ ID NO: 3. In some embodiments, the modification is the attachment of a polymer to a side chain of a residue of the IL-7 polypeptide.

In one aspect, provided herein, is an IL-7 polypeptide comprising at least one modification to the amino acid sequence as set forth in SEQ ID NO: 3, wherein the at least one modification is a natural amino acid substitution or an additional group covalently attached to a side chain of an amino acid residue of the IL-7 polypeptide, and wherein the IL-7 polypeptide retains each residue in SEQ ID NO: 3 which is substituted relative to SEQ ID NO: 1.

In one aspect, provided herein, is an IL-7 polypeptide comprising a) an amino acid substitution selected from K7A, S14H, V15W, N36S, N70K, N70Y, S71N, S71R, S71V, T72H, T72N, T72W, D74A, D74G, D74W, D76S, L77E, L77H, L77Q, L77T, L77V, H78A, H78R, H78Y, L79A, L80K, L80Q, L80W, K81Q, K81W, E84F, E84N, E84R, E84W, E84Y, G85A, G85N, G85Q, G85W, I88A, I88D, I88E, I88F, and E114S; and/or b) an additional group covalently attached to a side chain of an amino acid residue of the IL-7 polypeptide selected from residues 2, 7, 11, 14, 15, 18, 34, 47, 70, 71, 72, 74, 77, 78, 79, 80, 81, 84, 85, 88, 92, 129, 141, and 142, wherein residue position numbering is based on SEQ ID NO: 1 as a reference sequence.

In one aspect, provided herein, is an IL-7 polypeptide comprising a modification or set of modifications selected from: a) a G85N substitution; b) a polymer attached to an amino acid residue at position 81 of the IL-7 polypeptide; c) a G85N substitution and a polymer attached to an amino acid residue at position 81 of the IL-7 polypeptide; and d) a V15W substitution.

### Modifications to IL-7 Polypeptides

The present disclosure relates to IL-7 polypeptides useful as therapeutic agents. IL-7 polypeptides provided herein can be used as immunotherapies or as parts of immunotherapy regimens. Such IL-7 polypeptides in some instances display binding characteristics for the IL-7 receptor (IL-7R) that differ from wild-type IL-7.

In some embodiments, the binding affinity of the IL-7 polypeptide to the IL-7 receptor or a subunit thereof is modified due to a modification at one or more amino acid residues of the IL-7 polypeptide. In some embodiments, the IL-7 polypeptide comprises modifications at an amino acid residue that is implicated in binding of IL-7 to one or both of the IL-7 receptor or the IL-7 receptor alpha subunit. In some embodiments, the IL-7 polypeptide comprises a modification at an amino acid residue that interacts with IL-7 receptor alpha subunit. In some embodiments, the IL-7 polypeptide comprises a modification at an amino acid residue which interacts with the IL-7 receptor. In some embodiments, the IL-7 polypeptide comprises a modification at a residue that interacts with the gamma chain of the IL-7 receptor.

In some embodiments, an IL-7 polypeptide described herein comprises one or more modifications at one or more amino acid residues. In some embodiments, the residue position numbering of the IL-7 polypeptide is based on SEQ ID NO: 1 as a reference sequence. SEQ ID NO: 1 represents a wild-type human IL-7 polypeptide.

Modifications to the polypeptides described herein encompass substitutions, addition of various functionalities, deletion of amino acids, addition of amino acids, or any other alteration of the wild-type version of the protein or protein fragment. Functionalities which may be added to polypeptides include polymers, linking groups, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides. In some embodiments, functionalities are incorporated site-specifically into the polypeptides.

In some embodiments, the IL-7 polypeptides described herein contain 1, 2, 3, 4, 5, 6, 7, 8, 9, 10, 11, 12, 13, 14, 15, 16, 17, 18, 19, or 20 modified amino acid residues.

In some embodiments, the IL-7 polypeptide comprises an amino acid sequence having at least 80%, 85%, 90%, 91%, 92%, 93%, 94%, 95%, 96%, 97%, 98%, 99%, or 100% sequence identity to the sequence set forth in SEQ ID NO: 1. In some embodiments wherein the IL-7 polypeptide has an identical sequence to SEQ ID NO: 1, the IL-7 polypeptide comprises groups attached to the natural amino acid residues of SEQ ID NO: 1 (e.g., a polymer attached at residue K81).

In some embodiments, the IL-7 polypeptide comprises 1 substitution to 15 substitutions. In some embodiments, the IL-7 polypeptide comprises 1 substitution to 3 substitutions, 1 substitution to 5 substitutions, 1 substitution to 7 substitutions, 1 substitution to 9 substitutions, 1 substitution to 11 substitutions, 1 substitution to 13 substitutions, 1 substitution to 15 substitutions, 3 substitutions to 5 substitutions, 3 substitutions to 7 substitutions, 3 substitutions to 9 substitutions, 3 substitutions to 11 substitutions, 3 substitutions to 13 substitutions, 3 substitutions to 15 substitutions, 5 substitutions to 7 substitutions, 5 substitutions to 9 substitutions, 5 substitutions to 11 substitutions, 5 substitutions to 13 substitutions, 5 substitutions to 15 substitutions, 7 substitutions to 9 substitutions, 7 substitutions to 11 substitutions, 7 substitutions to 13 substitutions, 7 substitutions to 15 substitutions, 9 substitutions to 11 substitutions, 9 substitutions to 13 substitutions, 9 substitutions to 15 substitutions, 11 substitutions to 13 substitutions, 11 substitutions to 15 substitutions, or 13 substitutions to 15 substitutions. In some embodiments, the IL-7 polypeptide comprises 1 substitution, 3 substitutions, 5 substitutions, 7 substitutions, 9 substitutions, 11 substitutions, 13 substitutions, or 15 substitutions. In some embodiments, the IL-7 polypeptide comprises at least 1 substitution, 3 substitutions, 5 substitutions, 7 substitutions, 9 substitutions, 11 substitutions, or 13 substitutions. In some embodiments, the IL-7 polypeptide comprises at most 3 substitutions, 5 substitutions, 7 substitutions, 9 substitutions, 11 substitutions, 13 substitutions, or 15 substitutions.

In some embodiments, the IL-7 polypeptide described herein comprises at least 1, at least 2, at least 3, at least 4, at least 5, at least 6, at least 7, at least 8, or at least 9 natural amino acid substitutions, wherein the natural amino acid substitutions are relative to SEQ ID NO: 1. In some embodiments, the IL-7 polypeptide comprises 1 to 9 natural amino acid substitutions. In some embodiments, the IL-7 polypeptide comprises 1 or 2 natural amino acid substitutions, 1 to 3 natural amino acid substitutions, 1 to 4 natural amino acid substitutions, 1 to 5 natural amino acid substitutions, 1 to 6 natural amino acid substitutions, 1 to 7 natural amino acid substitutions, 1 to 8 natural amino acid substitutions, 2 to 3 natural amino acid substitutions, 2 to 4 natural amino acid substitutions, 2 to 5 natural amino acid substitutions, 2 to 6 natural amino acid substitutions, 2 to 7 natural amino acid substitutions, 2 to 8 natural amino acid substitutions, 2 to 9 natural amino acid substitutions, 3 or 4 natural amino acid substitutions, 3 to 5 natural amino acid substitutions, 3 to 6 natural amino acid substitutions, 3 to 7 natural amino acid substitutions, 3 to 9 natural amino acid substitutions, 4 or 5 natural amino acid substitutions, 4 to 6 natural amino acid substitutions, 4 to 7 amino acid substitutions, 4 to 9 natural amino acid substitutions, 5 or 6 natural amino acid substitutions, 5 to 7 amino acid substitutions, 5 to 9 natural amino acid substitutions, 6 or 7 natural amino acid substitutions, 6 to 9 natural amino acid substitutions, or 7 to 9 natural amino acid substitutions. In some embodiments, the IL-7 polypeptide comprises 3 natural amino acid substitutions, 4 natural amino acid substitutions, 5 amino acid substitutions, 6 natural amino acid substitutions, 7 natural amino acid substitutions, or 9 natural amino acid substitutions. In some embodiments, the IL-7 polypeptide comprises at most 4 natural amino acid substitutions, 5 natural amino acid substitutions, 6 natural amino acid substitutions, 7 natural amino acid substitutions, or 9 natural amino acid substitutions. In some embodiments, the IL-7 polypeptide comprises no natural amino acid substitutions. In some embodiments, the IL-7 polypeptide comprises no natural amino acid substitutions and a polymer attached to a side chain of a residue of the IL-7 polypeptide (e.g., at residue K81 of the IL-7 polypeptide). In some embodiments, the IL-7 polypeptide comprises one or more natural amino acid substitutions and a polymer attached to a side chain of a residue of the IL-7 polypeptide (e.g., at residue K81 of the IL-7 polypeptide). In some embodiments, the IL-7 polypeptide described herein further comprises up to 10 unnatural amino acid substitutions. In some embodiments, the IL-7 polypeptide comprises 1, 2, 3, 4, 5, 6, 7, 8, 9 or 10 unnatural amino acid substitutions. In some embodiments, the IL-7 polypeptide further comprises unnatural amino acid substitutions at residues M17, M27, N36, M54, M69, D76, E114, and/or M147. In some embodiments, the unnatural amino acid residues substituted for the methionines are each independently norleucine or O-methyl-homoserine. In some embodiments, the IL-7 comprises homoserine (Hse) 36, Hse 76, and Hse 114. In some embodiments, the IL-7 polypeptide further comprises M17Nle, M27Nle, N36Hse, M54Nle, M69Nle, D76Hse, E114Hsc, and/or M147Nlc.

In some embodiments, a modification is at a position which binds to the IL-7Rα. Substitutions in residues of IL-7 which may affect binding or interact with IL-7Rα include those listed in Table 1, or other substitutions at the residue positions indicated in Table 1. In some embodiments, the modification results in a disruption of the interaction between the IL-7 polypeptide and IL-7Rα. In some embodiments, the modification is one which makes the residue larger, thus creating steric hindrance between IL-7 and IL-7Rα.

In some embodiments, a modification is a position which binds to the common gamma chain (e.g., residue W142). In some embodiments, this modification results in decreased binding between the IL-7 polypeptide and the gamma chain, thereby disrupting binding between the IL-7 polypeptide and the IL-7R heterodimer. In some embodiments, the modification results in increased binding between the IL-7 polypeptide and the gamma chain, thereby biasing the IL-7 polypeptide in favor of the gamma chain and weakening the affinity for the IL-7R heterodimer.

In some embodiments, the IL-7 polypeptides comprise at least one modification in the range of amino acid residues 1-152, based on the sequence of mature human IL-7 (SEQ ID NO: 1). In some embodiments, the at least one modification is in the range of amino acid residues 2-147. In some embodiments, the at least one modification is in the range of amino acid residues 15-147. In some embodiments, the at least one modification is at amino acid residue 1. In some embodiments, the at least one modification is at amino acid residue 2. In some embodiments, the at least one modification is at amino acid residue 7. In some embodiments, the at least one modification is at amino acid residue 11. In some embodiments, the at least one modification is at amino acid residue 14. In some embodiments, the at least one modification is at amino acid residue 15. In some embodiments, the at least one modification is at amino acid residue 17. In some embodiments, the at least one modification is at amino acid residue 18. In some embodiments, the at least one modification is at amino acid residue 27. In some embodiments, the at least one modification is at amino acid residue 34. In some embodiments, the at least one modification is at amino acid residue 36. In some embodiments, the at least one modification is at amino acid residue 47. In some embodiments, the at least one modification is at amino acid residue 54. In some embodiments, the at least one modification is at amino acid residue 69. In some embodiments, the at least one modification is at amino acid residue 70. In some embodiments, the at least one modification is at amino acid residue 71. In some embodiments, the at least one modification is at amino acid residue 74. In some embodiments, the at least one modification is at amino acid residue 76. In some embodiments, the at least one modification is at amino acid residue 77. In some embodiments, the at least one modification is at amino acid residue 78. In some embodiments, the at least one modification is at amino acid residue 79. In some embodiments, the at least one modification is at amino acid residue 80. In some embodiments, the at least one modification is at amino acid residue 81. In some embodiments, the at least one modification is at amino acid residue 84. In some embodiments, the at least one modification is at amino acid residue 85. In some embodiments, the at least one modification is at amino acid residue 88. In some embodiments, the at least one modification is at amino acid residue 92. In some embodiments, the at least one modification is at amino acid residue 114. In some embodiments, the at least one modification is at amino acid residue 129. In some embodiments, the at least one modification is at amino acid residue 141. In some embodiments, the at least one modification is at amino acid residue 142. In some embodiments, the at least one modification is at amino acid residue 147. In some embodiments, the at least one modification is at the N-terminal residue.

In some embodiments, the IL-7 polypeptide comprises an amino acid substitutions selected from C2S, C2A, K7A, Q11F, S14H, V15A, V15W, V18A, C34S, C34A, N36S, C47S, C47A, N70K, N70Y, S71N, S71R, S71V, T72H, T72N, T72W, D74A, D74G, D74N, D74Q, D74W, D76S, L77A, L77D, L77E, L77H, L77Q, L77T, L77V, H78A, H78R, H78Y, L79A, L80K, L80Q, L80W, K81A, K81E, K81M, K81Q, K81W, E84F, E84N, E84R, E84W, E84Y, G85A, G85N, G85Q, G85W, I88A, I88D, I88E, I88F, I88R, I88T, C92S, C92A, E114S, C129S, C129A, C141S, and C141A, and combinations thereof.

In some embodiments, the IL-7 polypeptide comprises an amino acid substitution selected from K7A, Q11F, S14H, V15W, N36S, N70K, N70Y, S71N, S71R, S71V, T72H, T72N, T72W, D74A, D74G, D74W, D76S, L77E, L77H, L77Q, L77T, L77V, H78A, H78R, H78Y, L79A, L80K, L80Q, L80W, K81Q, K81W, E84F, E84N, E84R, E84W, E84Y, G85A, G85N, G85Q, G85W, 188A, 188D, 188E, 188F, and E114S, and combinations thereof. In some embodiments, the IL-7 polypeptide comprises K7A. In some embodiments, the IL-7 polypeptide comprises Q11F. In some embodiments, the IL-7 polypeptide comprises S14H. In some embodiments, the IL-7 polypeptide comprises V15W. In some embodiments, the IL-7 polypeptide comprises N36S. In some embodiments, the IL-7 polypeptide comprises N70K. In some embodiments, the IL-7 polypeptide comprises N70Y. In some embodiments, the IL-7 polypeptide comprises S71N. In some embodiments, the IL-7 polypeptide comprises S71R. In some embodiments, the IL-7 polypeptide comprises S71V. In some embodiments, the IL-7 polypeptide comprises T72H. In some embodiments, the IL-7 polypeptide comprises T72N. In some embodiments, the IL-7 polypeptide comprises T72W. In some embodiments, the IL-7 polypeptide comprises D74A. In some embodiments, the IL-7 polypeptide comprises D74G. In some embodiments, the IL-7 polypeptide comprises D74W. In some embodiments, the IL-7 polypeptide comprises D76S. In some embodiments, the IL-7 polypeptide comprises L77E. In some embodiments, the IL-7 polypeptide comprises L77H. In some embodiments, the IL-7 polypeptide comprises L77Q. In some embodiments, the IL-7 polypeptide comprises L77T. In some embodiments, the IL-7 polypeptide comprises L77V. In some embodiments, the IL-7 polypeptide comprises H78A. In some embodiments, the IL-7 polypeptide comprises H78R. In some embodiments, the IL-7 polypeptide comprises H78Y. In some embodiments, the IL-7 polypeptide comprises L79A. In some embodiments, the IL-7 polypeptide comprises L80K. In some embodiments, the IL-7 polypeptide comprises L80Q. In some embodiments, the IL-7 polypeptide comprises L80W. In some embodiments, the IL-7 polypeptide comprises K81Q. In some embodiments, the IL-7 polypeptide comprises K81W. In some embodiments, the IL-7 polypeptide comprises E84F. In some embodiments, the IL-7 polypeptide comprises E84N. In some embodiments, the IL-7 polypeptide comprises E84R. In some embodiments, the IL-7 polypeptide comprises E84W. In some embodiments, the IL-7 polypeptide comprises E84Y. In some embodiments, the IL-7 polypeptide comprises G85A. In some embodiments, the IL-7 polypeptide comprises G85N. In some embodiments, the IL-7 polypeptide comprises G85Q. In some embodiments, the IL-7 polypeptide comprises G85W. In some embodiments, the IL-7 polypeptide comprises 188A. In some embodiments, the IL-7 polypeptide comprises 188D. In some embodiments, the IL-7 polypeptide comprises 188E. In some embodiments, the IL-7 polypeptide comprises 188F. In some embodiments, the IL-7 polypeptide comprises E114S.

In some embodiments, the IL-7 polypeptide comprises an amino acid substitution at residue V15, wherein residue position numbering is based on SEQ ID NO: 1 as a reference sequence. In some embodiments, the amino acid substitution at residue V15 is for an amino acid with a bulkier side chain than valine (e.g., leucine, isoleucine, norleucine, methionine, O-methyl-homoserine, histidine, phenylalanine, tyrosine, or tryptophan).

In some embodiments, the amino acid substitution at residue V15 comprises a substitution with an aromatic amino acid. In some embodiments, the substitution with an aromatic amino acid is for a natural amino acid (e.g., tryptophan, phenylalanine, or tyrosine). In some embodiments, the IL-7 polypeptide comprises a V15W, V15F, or V15Y substitution. In some embodiments, the IL-7 polypeptide comprises a V15W substitution. In some embodiments, the amino acid substitution at residue V15 is for a derivative of an aromatic amino acid, such as an aromatic amino acid with a lengthened/shortened methylene chain (e.g., a tryptophan having 0, 2, 3, 4, 5, or more methylene groups between the amino acid α carbon and the indole group) or a substituent on the aromatic ring (e.g., alkylated or halogenated on the indole).

In some embodiments, the IL-7 polypeptide comprises an amino acid modification at residue K81, wherein residue position numbering is based on SEQ ID NO: 1 as a reference sequence. In some embodiments, the amino acid modification is an additional group attached to a side chain of the amino acid at residue 81. In some embodiments, the amino acid modification is a polymer or small molecule attached to the lysine residue side chain. In some embodiments, the modified lysine residue comprises a PEG2 attached to lysine 81 side chain amine (referred to as K81 (L2P)). In some embodiments, the modified lysine residue comprises a PEG6 group attached to the lysine 81 side chain amine (referred to as K81 (L6P)). In some embodiments the modified lysine residue comprises a PEG11 group attached to the lysine 81 side chain amine (referred to as K81 (L11P)). In some embodiments the modified lysine residue comprises an adamantine group attached to the lysine 81 side chain amine (referred to as K81 (ADA)).

In some embodiments, the IL-7 polypeptide comprises a lysine with a polymer attached, such as a residue K81 of the IL-7 polypeptide. In some embodiments, the lysine with a polymer attached has a structure

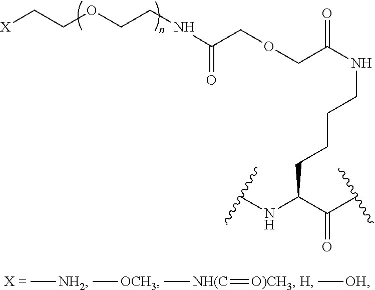

wherein n is an integer from 1-30. The structure L6P provided herein as the above structure wherein n is 7 and X is —NH₂ (see synthesis of CMP-109 in the Examples, which uses Structure 8 provided therein as the building block to form L6P).

In some embodiments, the IL-7 polypeptide comprises a lysine with a polymer attached, such as a residue K81 of the IL-7 polypeptide. In some embodiments, the lysine with a polymer attached has a structure

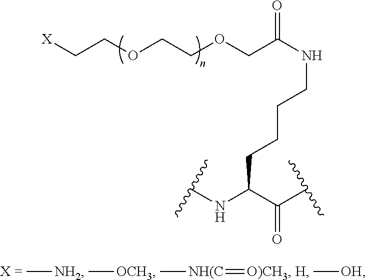

wherein n is an integer from 1-30. In some embodiments, the structure L2P provided herein has the above structure wherein n is 2 and X is —OCH₃ (as in CMP-115). The structure L11P provided herein has the above structure wherein n is 10 and X is —OCH₃ (as in CMP-112).

In some embodiments, the IL-7 polypeptide comprises an amino acid substitution at residue G85, wherein residue position numbering is based on SEQ ID NO: 1 as a reference sequence. In some embodiments, the substitution at is selected from G85A, G85E, G85K, G85N, G85Q, and G85W. In some embodiments, the substitution is a G85N substitution.

In some embodiments, the IL-7 polypeptide comprises both a modification at K81 (e.g., a polymer attached to the side chain of the lysine) and a G85 substitution as provided herein. In some embodiments, the IL-7 polypeptide comprises a modification selected from V15W substitution, a polymer attached at residue 81, a G85N substitution, and any combination thereof. In some embodiments, the IL-7 polypeptide comprises a polymer attached at residue 81 and a G85N substitution. In some embodiments, the IL-7 comprises a V15W and a polymer attached at residue 81. In some embodiments, the IL-7 comprises a V15W and a G85N substitution.

In some embodiments, the IL-7 polypeptide comprises an amino acid substitution at a residue set forth in Table 1 or a substitution set forth in Table 1. The substitutions set forth in Table 1 can be in addition to the other modifications provided herein (e.g., substitutions or modifications at residue V15 (e.g., V15W), G85 (e.g., G85N), K81 (e.g., attachment of polymer), or any modifications of a synthetic IL-7). In some instances, the substitutions listed in Table 1 can affect binding of the IL-7 polypeptide with the IL-7 receptor complex (e.g., reduce binding of the IL-7 polypeptide with the IL-7 receptor complex).

TABLE 1
Residues and Substitutions in IL-7

| WT IL-7 Residue | Substitution |
|---|---|
| C2 | S |
| K10 | I, M, C |
| Q11 | E, R |
| Y12 | F |
| E13 | A, K |
| S14 | T |
| V15 | A, K |
| M17 | L |
| V18 | A, K |
| S19 | Q |
| D21 | A, K |
| Q22 | A, D, E, K |
| D25 | A, K |
| I30 | H |
| C34 | S |
| L35 | N, Q |
| C47 | S |
| D48 | N |
| N50 | D |
| E52 | Q |
| M69 | Q, S |
| N70 | D, Q, V |
| T72 | A, V |
| D74 | E, N |
| L77 | A, K |
| K81 | A, E, M, R |
| E84 | A, K, Q |
| G85 | E, K |
| 188 | K, T, R |
| N91 | A, D, Q |
| C92 | S |
| N116 | A, D, Q |
| L128 | Q, R |
| C129 | S |
| Q136 | A, K |
| E137 | Q |
| K139 | A, E |
| C141 | S |
| W142 | A, D, F, H, I, M, P, Q, R, |
| | S, Y |
| N143 | D, K |
| M147 | A |

In some embodiments, the IL-7 polypeptide comprises an amino acid substitution at residue W142. In some embodiments, the IL-7 polypeptide comprises a W142A, W142C, W142F, W142G, W142H, W142I, W142L, W142M, W142V, or W142Y substitution. In some embodiments, the IL-7 polypeptide comprises a W142H, W142A, W142I, or W142V substitution. In some embodiments, the IL-7 polypeptide comprises a W142H substitution. In some embodiments, the IL-7 polypeptide comprises a W142A substitution. In some embodiments, the IL-7 polypeptide comprises a W142I substitution. In some embodiments, the IL-7 polypeptide comprises a W142V substitution. The substitution at residue W142 can be in addition to any other modifications provided herein (e.g., V15W substitutions, polymers attached at residue 81, and/or G85N substitutions, or in addition to modifications for a synthetic IL-7 polypeptide), or can be the only modification included in an IL-7 polypeptide.

In some embodiments, the IL-7 polypeptide comprises substitutions of one or more cysteine residues of SEQ ID NO: 1. In some embodiments, the mutations of one or more cysteine residues disrupts disulfide formation compared to wild type IL-7. Wild type IL-7 contains 3 disulfide bridges between residues C47/C141, C34/C129, and C2/C92. In some embodiments, onc, two, or all three disulfide bonds are disrupted due to substitutions of one or both cysteines from each disrupted pair. In some embodiments, the IL-7 polypeptide comprises an amino acid substitution at residues C2, C34, C47, C92, C129, C141, or any combination thereof. In some embodiments, the IL-7 polypeptide comprises amino acid substitutions at residues C2 and C92; C34 and C129; and/or C47 and C147. In some embodiments, each substitution at a cysteine residue is for an alanine or a serine residue. In some embodiments, each substitution at a cysteine residue is for a serine residue. The substitution of the one or more cysteine residues can be in addition to any other modifications provided herein (e.g., V15W substitutions, polymers attached at residue 81, and/or G85N substitutions, or in addition to modifications for a synthetic IL-7 polypeptide).

In some embodiments, the IL-7 polypeptide comprises M17Nle, M27Nle, N36Hsc, M54Nlc, M69Nle, D76Hse, E114Hsc, and M147Nle, wherein residue position numbering of the IL-7 polypeptide is based on SEQ ID NO: 1 as a reference sequence. In some embodiments, the IL-7 polypeptide comprises V15W, M17Nlc, M27Nle, N36Hse, M54Nle, M69Nle, D76Hsc, E114Hsc, and M147Nlc, wherein residue position numbering of the IL-7 polypeptide is based on SEQ ID NO: 1 as a reference sequence. In some embodiments, the IL-7 polypeptide comprises M17Nlc, M27Nle, N36Hse, M54Nle, M69Nlc, D76Hsc, K81 (with a polymer attached), E114Hsc, and M147Nle, wherein residue position numbering of the IL-7 polypeptide is based on SEQ ID NO: 1 as a reference sequence. In some embodiments, the IL-7 polypeptide comprises M17Nle, M27Nle, N36Hse, M54Nle, M69Nlc, D76Hsc, G85N, E114Hse, and M147Nle, wherein residue position numbering of the IL-7 polypeptide is based on SEQ ID NO: 1 as a reference sequence. In some embodiments, the IL-7 polypeptide comprises V15W, M17Nle, M27Nle, N36Hsc, M54Nlc, M69Nle, D76Hsc, K81 (with a polymer attached), E114Hsc, and M147Nle, wherein residue position numbering of the IL-7 polypeptide is based on SEQ ID NO: 1 as a reference sequence. In some embodiments, the IL-7 polypeptide comprises V15W, M17Nle, M27Nle, N36Hse, M54Nle, M69Nlc, D76Hsc, G85N, E114Hsc, and M147Nle, wherein residue position numbering of the IL-7 polypeptide is based on SEQ ID NO: 1 as a reference sequence. In some embodiments, the IL-7 polypeptide comprises M17Nle, M27Nle, N36Hse, M54Nle, M69Nle, D76Hse, K81 (with a polymer attached), G85N, E114Hsc, and M147Nle, wherein residue position numbering of the IL-7 polypeptide is based on SEQ ID NO: 1 as a reference sequence. In some embodiments, the IL-7 polypeptide comprises V15W, M17Nle, M27Nle, N36Hse, M54Nle, M69Nle, D76Hsc, K81 (with a polymer attached), G85N, E114Hsc, and M147Nle, wherein residue position numbering of the IL-7 polypeptide is based on SEQ ID NO: 1 as a reference sequence.

In some embodiments, the IL-7 polypeptide comprises M17Nle, M27Nlc, N36Hse, C47S, M54Nle, M69Nle, D76Hse, E114Hse, C141S, and M147Nle, wherein residue position numbering of the IL-7 polypeptide is based on SEQ ID NO: 1 as a reference sequence. In some embodiments, the IL-7 polypeptide comprises M17Nle, M27Nle, C34S, N36Hse, M54Nle, M69Nle, D76Hse, E114Hsc, C129S, and M147Nle, wherein residue position numbering of the IL-7 polypeptide is based on SEQ ID NO: 1 as a reference sequence. In some embodiments, the IL-7 polypeptide comprises C2S, M17Nle, M27Nle, N36Hse, M54Nlc, M69Nlc, D76Hse, C92S, E114Hsc, and M147Nlc, wherein residue position numbering of the IL-7 polypeptide is based on SEQ ID NO: 1 as a reference sequence. In some embodiments, the IL-7 polypeptide comprises M17Nle, M27Nlc, C34S, N36Hse, C47S, M54Nle, M69Nle, D76Hsc, E114Hsc, C129S, C141S, and M147Nlc, wherein residue position numbering of the IL-7 polypeptide is based on SEQ ID NO: 1 as a reference sequence. In some embodiments, the IL-7 polypeptide comprises C2S, M17Nlc, M27Nlc, N36Hsc, C47S, M54Nle, M69Nlc, D76Hsc, C92S, E114Hsc, C141S, and M147Nle, wherein residue position numbering of the IL-7 polypeptide is based on SEQ ID NO: 1 as a reference sequence. In some embodiments, the IL-7 polypeptide comprises C2S, M17Nle, M27Nle, C34S, N36Hse, M54Nle, M69Nle, D76Hsc, C92S, E114Hsc, C129S, and M147Nlc, wherein residue position numbering of the IL-7 polypeptide is based on SEQ ID NO: 1 as a reference sequence. In some embodiments, the IL-7 polypeptide comprises C2S, M17Nle, M27Nlc, C34S, N36Hsc, C47S M54Nle, M69Nlc, D76Hse, C92S, E114Hse, C129S, C141S, and M147Nle, wherein residue position numbering of the IL-7 polypeptide is based on SEQ ID NO: 1 as a reference sequence.

In some embodiments, the IL-7 polypeptide comprises V15W, C47S, and C141S, wherein residue position numbering of the IL-7 polypeptide is based on SEQ ID NO: 1 as a reference sequence. In some embodiments, the IL-7 polypeptide comprises C47S, K81 with a polymer attached, and C141S, wherein residue position numbering of the IL-7 polypeptide is based on SEQ ID NO: 1 as a reference sequence. In some embodiments, the IL-7 polypeptide comprises C47S, G85N, and C141S, wherein residue position numbering of the IL-7 polypeptide is based on SEQ ID NO: 1 as a reference sequence. In some embodiments, the IL-7 polypeptide comprises C47S, K81 with a polymer attached, G85N, and C141S, wherein residue position numbering of the IL-7 polypeptide is based on SEQ ID NO: 1 as a reference sequence. In some embodiments, the IL-7 polypeptide comprises M17Nle, M27Nle, N36Hsc, C47S, M54Nle, M69Nlc, D76Hse, K81 with a polymer attached, E114Hse, C141S, and M147Nle, wherein residue position numbering of the IL-7 polypeptide is based on SEQ ID NO: 1 as a reference sequence. In some embodiments, the IL-7 polypeptide comprises M17Nle, M27Nle, N36Hsc, C47S, M54Nle, M69Nle, D76Hsc, G85N, E114Hsc, C141S, and M147Nle, wherein residue position numbering of the IL-7 polypeptide is based on SEQ ID NO: 1 as a reference sequence. In some embodiments, the IL-7 polypeptide comprises M17Nle, M27Nle, N36Hsc, C47S, M54Nle, M69Nle, D76Hsc, K81 with a polymer attached, G85N, E114Hsc, C141S, and M147Nle, wherein residue position numbering of the IL-7 polypeptide is based on SEQ ID NO: 1 as a reference sequence.

In some embodiments, the IL-7 polypeptide comprises V15W, C34S, and C129S, wherein residue position numbering of the IL-7 polypeptide is based on SEQ ID NO: 1 as a reference sequence. In some embodiments, the IL-7 polypeptide comprises C34S, K81 with a polymer attached, and C129S, wherein residue position numbering of the IL-7 polypeptide is based on SEQ ID NO: 1 as a reference sequence. In some embodiments, the IL-7 polypeptide comprises C34S, G85N, and C129S, wherein residue position numbering of the IL-7 polypeptide is based on SEQ ID NO: 1 as a reference sequence. In some embodiments, the IL-7 polypeptide comprises C34S, K81 with a polymer attached, G85N, and C129S, wherein residue position numbering of the IL-7 polypeptide is based on SEQ ID NO: 1 as a reference sequence. In some embodiments, the IL-7 polypeptide comprises V15W, C34S, K81 with a polymer attached, G85N, and C129S, wherein residue position numbering of the IL-7 polypeptide is based on SEQ ID NO: 1 as a reference sequence. In some embodiments, the IL-7 polypeptide comprises M17Nle, M27Nlc, C34S, N36Hsc, M54Nlc, M69Nlc, D76Hsc, K81 with a polymer attached, E114Hsc, C129S, and M147Nle, wherein residue position numbering of the IL-7 polypeptide is based on SEQ ID NO: 1 as a reference sequence. In some embodiments, the IL-7 polypeptide comprises M17Nle, M27Nlc, C34S, N36Hsc, M54Nlc, M69Nlc, D76Hsc, G85N, E114Hsc, C129S, and M147Nle, wherein residue position numbering of the IL-7 polypeptide is based on SEQ ID NO: 1 as a reference sequence. In some embodiments, the IL-7 polypeptide comprises M17Nle, M27Nle, C34S, N36Hsc, M54Nle, M69Nlc, D76Hse, K81 with a polymer attached, G85N, E114Hse, C129S, and M147Nle, wherein residue position numbering of the IL-7 polypeptide is based on SEQ ID NO: 1 as a reference sequence.

In some embodiments, the IL-7 polypeptide comprises C2S, V15W, and C92S, wherein residue position numbering of the IL-7 polypeptide is based on SEQ ID NO: 1 as a reference sequence. In some embodiments, the IL-7 polypeptide comprises C2S, K81 with a polymer attached, and C92S, wherein residue position numbering of the IL-7 polypeptide is based on SEQ ID NO: 1 as a reference sequence. In some embodiments, the IL-7 polypeptide comprises C2S, G85N, and C92S, wherein residue position numbering of the IL-7 polypeptide is based on SEQ ID NO: 1 as a reference sequence. In some embodiments, the IL-7 polypeptide comprises C2S, K81 with a polymer attached, G85N, and C92S, wherein residue position numbering of the IL-7 polypeptide is based on SEQ ID NO: 1 as a reference sequence. In some embodiments, the IL-7 polypeptide comprises C2S, V15W, K81 with a polymer attached, G85N, and C92S, wherein residue position numbering of the IL-7 polypeptide is based on SEQ ID NO: 1 as a reference sequence. In some embodiments, the IL-7 polypeptide comprises C2S, M17Nle, M27Nle, N36Hsc, M54Nlc, M69Nlc, D76Hsc, K81 with a polymer attached, C92S, E114Hsc, and M147Nle, wherein residue position numbering of the IL-7 polypeptide is based on SEQ ID NO: 1 as a reference sequence. In some embodiments, the IL-7 polypeptide comprises C2S, M17Nle, M27Nle, N36Hse, M54Nle, M69Nle, D76Hse, G85N, C92S, E114Hse, and M147Nle, wherein residue position numbering of the IL-7 polypeptide is based on SEQ ID NO: 1 as a reference sequence. In some embodiments, the IL-7 polypeptide comprises C2S, M17Nle, M27Nle, N36Hse, M54Nle, M69Nle, D76Hse, K81 with a polymer attached, G85N, C92S, E114Hse, and M147Nle, wherein residue position numbering of the IL-7 polypeptide is based on SEQ ID NO: 1 as a reference sequence.

In some embodiments, the IL-7 polypeptide comprises V15W, C34S, C47S, C129S, and C141S, wherein residue position numbering of the IL-7 polypeptide is based on SEQ ID NO: 1 as a reference sequence. In some embodiments, the IL-7 polypeptide comprises C34S, C47S, K81 with a polymer attached, C129S, and C141S, wherein residue position numbering of the IL-7 polypeptide is based on SEQ ID NO: 1 as a reference sequence. In some embodiments, the IL-7 polypeptide comprises C34S, C47S, G85N, C129S, and C141S, wherein residue position numbering of the IL-7 polypeptide is based on SEQ ID NO: 1 as a reference sequence. In some embodiments, the IL-7 polypeptide comprises M17Nle, M27Nle, C34S, N36Hse, C47S, M54Nle, D76Hse, K81 with a polymer attached, E114Hse, C129S, C141S, and M147Nle, wherein residue position numbering of the IL-7 polypeptide is based on SEQ ID NO: 1 as a reference sequence. In some embodiments, the IL-7 polypeptide comprises M17Nle, M27Nle, C34S, N36Hse, C47S, M54Nle, D76Hse, G85N, E114Hse, C129S, C141S, and M147Nle, wherein residue position numbering of the IL-7 polypeptide is based on SEQ ID NO: 1 as a reference sequence. In some embodiments, the IL-7 polypeptide comprises M17Nle, M27Nle, C34S, N36Hse, C47S, M54Nle, D76Hse, K81 with a polymer attached, G85N, E114Hse, C129S, C141S, and M147Nle, wherein residue position numbering of the IL-7 polypeptide is based on SEQ ID NO: 1 as a reference sequence.

In some embodiments, the IL-7 polypeptide comprises C2S, V15W, C47S, C92S, and C141S, wherein residue position numbering of the IL-7 polypeptide is based on SEQ ID NO: 1 as a reference sequence. In some embodiments, the IL-7 polypeptide comprises C2S, C47S, K81 with a polymer attached, C92S, and C141S, wherein residue position numbering of the IL-7 polypeptide is based on SEQ ID NO: 1 as a reference sequence. In some embodiments, the IL-7 polypeptide comprises C2S, C47S, G85N, C92S, and C141S, wherein residue position numbering of the IL-7 polypeptide is based on SEQ ID NO: 1 as a reference sequence. In some embodiments, the IL-7 polypeptide comprises C2S, C47S, K81 with a polymer attached, G85N, C92S, and C141S, wherein residue position numbering of the IL-7 polypeptide is based on SEQ ID NO: 1 as a reference sequence. In some embodiments, the IL-7 polypeptide comprises C2S, M17Nlc, M27Nlc, N36Hsc, C47S, M54Nlc, M69Nlc, D76Hse, K81 with a polymer attached, C92S, E114Hsc, C141S, and M147Nle, wherein residue position numbering of the IL-7 polypeptide is based on SEQ ID NO: 1 as a reference sequence. In some embodiments, the IL-7 polypeptide comprises C2S, M17Nlc, M27Nle, N36Hsc, C47S, M54Nle, M69Nlc, D76Hsc, G85N, C92S, E114Hsc, C141S, and M147Nle, wherein residue position numbering of the IL-7 polypeptide is based on SEQ ID NO: 1 as a reference sequence. In some embodiments, the IL-7 polypeptide comprises C2S, M17Nle, M27Nle, N36Hsc, C47S, M54Nle, M69Nle, D76Hse, K81 with a polymer attached, G85N, C92S, E114Hsc, C141S, and M147Nle, wherein residue position numbering of the IL-7 polypeptide is based on SEQ ID NO: 1 as a reference sequence.

In some embodiments, the IL-7 polypeptide comprises C2S, V15W, C34S, C92S, and C129S, wherein residue position numbering of the IL-7 polypeptide is based on SEQ ID NO: 1 as a reference sequence. In some embodiments, the IL-7 polypeptide comprises C2S, C34S, K81 with a polymer attached C92S, and C129S, wherein residue position numbering of the IL-7 polypeptide is based on SEQ ID NO: 1 as a reference sequence. In some embodiments, the IL-7 polypeptide comprises C2S, C34S, K81 with a polymer attached, G85N, C92S, and C129S, wherein residue position numbering of the IL-7 polypeptide is based on SEQ ID NO: 1 as a reference sequence. In some embodiments, the IL-7 polypeptide comprises C2S, C34S, G85N, C92S, and C129S, wherein residue position numbering of the IL-7 polypeptide is based on SEQ ID NO: 1 as a reference sequence. In some embodiments, the IL-7 polypeptide comprises C2S, M17Nle, M27Nle C34S, N36Hsc, M54Nle, M69Nlc, D76Hse, K81 with a polymer attached, C92S, E114Hsc, C129S, and M147Nle, wherein residue position numbering of the IL-7 polypeptide is based on SEQ ID NO: 1 as a reference sequence. In some embodiments, the IL-7 polypeptide comprises C2S, M17Nle, M27Nle C34S, N36Hsc, M54Nle, M69Nlc, D76Hse, G85N, C92S, E114Hsc, C129S, and M147Nle, wherein residue position numbering of the IL-7 polypeptide is based on SEQ ID NO: 1 as a reference sequence. In some embodiments, the IL-7 polypeptide comprises C2S, M17Nle, M27Nle C34S, N36Hsc, M54Nle, M69Nle, D76Hsc, K81 with a polymer attached, G85N, C92S, E114Hsc, C129S, and M147Nle, wherein residue position numbering of the IL-7 polypeptide is based on SEQ ID NO: 1 as a reference sequence.

In some embodiments, the IL-7 polypeptide comprises C2S, V15W, C34S, C47S, C92S, C129S, and C141S wherein residue position numbering of the IL-7 polypeptide is based on SEQ ID NO: 1 as a reference sequence. In some embodiments, the IL-7 polypeptide comprises C2S, C34S, C47S, C92S, K81 with a polymer attached, C92S, C129S, and C141S wherein residue position numbering of the IL-7 polypeptide is based on SEQ ID NO: 1 as a reference sequence. In some embodiments, the IL-7 polypeptide comprises C2S, C34S, C47S, K81 with a polymer attached, G85N, C92S, C129S, and C141S wherein residue position numbering of the IL-7 polypeptide is based on SEQ ID NO: 1 as a reference sequence. In some embodiments, the IL-7 polypeptide comprises C2S, C34S, C47S, G85N, C92S, C129S, and C141S wherein residue position numbering of the IL-7 polypeptide is based on SEQ ID NO: 1 as a reference sequence. In some embodiments, the IL-7 polypeptide comprises C2S, M17Nlc, M27Nlc C34S, N36Hsc, C47S, M54Nle, M69Nle, D76Hse, K81 with a polymer attached, C92S, E114Hsc, C129S, C141S, and M147Nle, wherein residue position numbering of the IL-7 polypeptide is based on SEQ ID NO: 1 as a reference sequence. In some embodiments, the IL-7 polypeptide comprises C2S, M17Nlc, M27Nlc C34S, N36Hsc, C47S, M54Nle, M69Nle, D76Hsc, G85N, C92S, E114Hsc, C129S, C141S, and M147Nle, wherein residue position numbering of the IL-7 polypeptide is based on SEQ ID NO: 1 as a reference sequence. In some embodiments, the IL-7 polypeptide comprises C2S, M17Nle, M27Nle C34S, N36Hsc, C47S, M54Nle, M69Nle, D76Hsc, K81 with a polymer attached, G85N, C92S, E114Hsc, C129S, C141S, and M147Nle, wherein residue position numbering of the IL-7 polypeptide is based on SEQ ID NO: 1 as a reference sequence.

In some embodiments, the IL-7 polypeptide comprises an amino acid substitution at a residue listed in Table 2 or an amino acid substitution listed in Table 2. In some embodiments, the substitutions listed in Table 2 can affect binding of the IL-7 polypeptide to the IL-7 receptor complex (or a subunit thereof), or can have another or no effect on the IL-7 polypeptide. The substitutions in Table 2 can be in addition to any other modifications provided herein (e.g., V15W substitutions, polymers attached at residue 81, and/or G85N substitutions, or in addition to modifications for a synthetic IL-7 polypeptide).

TABLE 2
Additional Exemplary IL-7 Substitutions:

| WT IL-7 | |
|---|---|
| Residue | Substitution |
| C2 | S |
| K10 | I, M, C |
| Q11 | E, R |
| Y12 | F |
| E13 | A, K |
| S14 | T |
| V15 | A, K |
| M17 | L |
| V18 | A, K |
| S19 | Q |
| D21 | A, K |
| Q22 | A, D, E, K |
| L24 | D |
| D25 | A, K |
| K28 | N |
| I30 | H, N, S, T |
| S32 | T |
| C34 | S |
| L35 | N, Q, S, T |
| E38 | S, T |
| F39 | P, S, T |
| F42 | S, T |
| C47 | S |
| D48 | N |
| N50 | D |
| E52 | Q, S, T |
| M54 | A, N |
| F57 | K, N |
| R58 | D |
| A60 | S |
| R61 | R |
| L63 | A, P, V |
| K68 | D |
| M69 | D, Q, S |
| N70 | D, Q, V |
| T72 | A, V |
| D74 | E, N, Q |
| L77 | A, D, K |
| K81 | A, E, M, R |
| V82 | N |
| E84 | A, K, Q, S, T |
| G85 | E, K, M |
| I88 | K, T, R |
| N91 | A, D, Q |
| C92 | S |
| V96 | G |
| K97 | N |
| R99 | S, T |
| A102 | N |
| L104 | N, S, T, V |
| E106 | S, T |
| N116 | A, D, Q |
| K120 | A |
| L128 | A, P, Q, R, S, T, V |
| C129 | S |
| L135 | A |
| Q136 | A, K |
| E137 | Q |
| K139 | A, E |
| C141 | S |
| W142 | A, D, F, H, I, M, P, Q, R, |
| | S, Y |
| N143 | D, K |
| I145 | N |
| M147 | A, K, M, S, T |
| T149 | S |

In some embodiments, the IL-7 polypeptide comprises amino acid substitutions potentially effecting glycosylation of the IL-7 polypeptide relative to wild type (e.g., those described in U.S. Pat. No. 7,708,985). In some embodiments, the IL-7 polypeptide comprises a substitution selected from K28N, I30S, I30T, I30N, S32T, T49S, N70A, N70D, N70Q, D74N, K81E, K81R, V82N, E84S, E84Q, N91A, N91D, N91Q, L104N, E106S, E106T, N116A, N116D, N116Q, I145N, M147N, M147S, and M147T. In some embodiments, the IL-7 polypeptide comprises a set of amino acid substitutions selected from: K28N and I30S; K28N and I30T; I30N and S32T; T49S and M147N; N70A and N91A and N116A; N70D, N91D and N116D; N70Q, N91Q and N116Q; D74N and K81E; D74N and K81R; D74N and E84Q; V82N and E84S; L104N and E106S; L104N and E106T; I145N and M147S; and I145N and M147T. Such substitutions can be in addition to any other modifications provided herein (e.g., V15W substitutions, polymers attached at residue 81, and/or G85N substitutions, or in addition to modifications for a synthetic IL-7 polypeptide).

In some embodiments, the IL-7 polypeptide comprises a truncation of amino acids or an extension peptide relative to the amino acid sequence set forth in SEQ ID NO: 1. In some embodiments, the IL-7 polypeptide comprises a truncation of the amino acid sequence set forth in SEQ ID NO: 1. In some embodiments, the truncation is a deletion of one or more residues starting from the N-terminal residue set forth in SEQ ID NO: 1. In some embodiments, the truncation is a deletion of one or more residues starting from the C-terminal residue set forth in SEQ ID NO: 1. In some embodiments, the truncation is of one, two, three, four, five, six, seven, eight, nine, ten, or more amino acids. Truncations or extensions can be in addition to any other modifications provided herein (e.g., V15W substitutions, polymers attached at residue 81, and/or G85N substitutions, or in addition to modifications for a synthetic IL-7 polypeptide).

In some embodiments, the IL-7 polypeptide comprises an extension peptide relative to the amino acid sequence set forth in SEQ ID NO: 1. The extension peptide comprises one or more amino acid residues appended to a residue corresponding to the N-terminal residue of SEQ ID NO: 1 or a residue corresponding to the C-terminal residue of SEQ ID NO: 1. In some embodiments, the extension peptide is at one or both of the N-terminus or the C-terminus of the IL-7 polypeptide. In some embodiments, the extension peptide extends the amino acid sequence of the IL-7 polypeptide by one, two, three, four, five, six, seven, eight, nine, ten, or more amino acids. In some embodiments, the extension peptide comprises an additional protein (e.g., as in a fusion protein fused at the N- or C-terminus of the IL-7 polypeptide, optionally through a linking peptide).

In some embodiments, the IL-7 polypeptide comprises a C-terminal extension peptide. In some embodiments, the C-terminal extension peptide comprises a histidine tag. In some instances, the histidine tag is used for purification of the IL-7 polypeptide after recombinant expression. In some embodiments, the C-terminal extension peptide comprises a linker peptide sequence between the poly-histidine portion of the extension peptide and the residue corresponding to the C-terminal residue of SEQ ID NO: 1.

In some embodiments, the IL-7 polypeptide comprises an N-terminal extension peptide. In some embodiments, the N-terminal extension peptide extends the amino acid sequence of the IL-7 polypeptide by one, two, three, four, five, six, seven, eight, nine, ten, or more amino acids. Exemplary N-terminal extension peptides of IL-7 polypeptides can be found, for example, in U.S. Pat. No. 10,208,099. In some embodiments, the N-terminal extension peptide consists of amino acid residues selected from glycine and methionine. In some embodiments, the N-terminal extension peptide is selected from methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine.

In some embodiments, the IL-7 polypeptide herein comprises a polypeptide sequence having at least about 80%, at least about 85%, at least about 90%, at least about 95%, or identical to any one of SEQ ID NOs: 3-12 or 50-56. In some embodiments, the polypeptide sequence is at least about 95%, at least about 96%, at least about 97%, at least about 99%, or 100% identical to any one of SEQ ID NOs: 3-12 or 50-56. In some embodiments, the IL-7 polypeptide herein comprises a polypeptide sequence having at least about 95%, at least about 96%, at least about 97%, at least about 99%, or 100% identical to any one of sequences listed Table 10.

In some embodiments, the IL-7 polypeptide sequence comprises a polypeptide sequence that is at least about 80% identical to SEQ ID NO: 3. In some embodiments, the IL-7 polypeptide sequence comprises a polypeptide sequence that is at least about 90% identical to SEQ ID NO: 3. In some embodiments, the IL-7 polypeptide sequence comprises a polypeptide sequence that is at least about 95% identical to SEQ ID NO: 3. In some embodiments, the IL-7 polypeptide sequence comprises a polypeptide sequence that is at least about 96% identical to SEQ ID NO: 3. In some embodiments, the IL-7 polypeptide sequence comprises a polypeptide sequence that is at least about 97% identical to SEQ ID NO: 3. In some embodiments, the IL-7 polypeptide sequence comprises a polypeptide sequence that is at least about 98% identical to SEQ ID NO: 3. In some embodiments, the IL-7 polypeptide sequence comprises a polypeptide sequence that is at least about 99% identical to SEQ ID NO: 3. In some embodiments, the IL-7 polypeptide sequence comprises a polypeptide sequence that is 100% identical to SEQ ID NO: 3, wherein the IL-7 polypeptide comprises a polymer attached to a side chain of an amino acid residue of the IL-7 polypeptide (e.g., at K81). In some embodiments, the IL-7 polypeptide retains each of the substitutions present in SEQ ID NO: 3 relative to SEQ ID NO: 1.

In some embodiments, the IL-7 polypeptide sequence comprises a polypeptide sequence that is at least about 80% identical to SEQ ID NO: 5. In some embodiments, the IL-7 polypeptide sequence comprises a polypeptide sequence that is at least about 90% identical to SEQ ID NO: 5. In some embodiments, the IL-7 polypeptide sequence comprises a polypeptide sequence that is at least about 95% identical to SEQ ID NO: 5. In some embodiments, the IL-7 polypeptide sequence comprises a polypeptide sequence that is at least about 96% identical to SEQ ID NO: 5. In some embodiments, the IL-7 polypeptide sequence comprises a polypeptide sequence that is at least about 97% identical to SEQ ID NO: 5. In some embodiments, the IL-7 polypeptide sequence comprises a polypeptide sequence that is at least about 98% identical to SEQ ID NO: 5. In some embodiments, the IL-7 polypeptide sequence comprises a polypeptide sequence that is at least about 99% identical to SEQ ID NO: 5. In some embodiments, the IL-7 polypeptide sequence comprises a polypeptide sequence that is 100% identical to SEQ ID NO: 5. In some embodiments, the IL-7 polypeptide retains each of the substitutions present in SEQ ID NO: 5 relative to SEQ ID NO: 1.

In some embodiments, the IL-7 polypeptide sequence comprises a polypeptide sequence that is at least about 80% identical to SEQ ID NO: 8. In some embodiments, the IL-7 polypeptide sequence comprises a polypeptide sequence that is at least about 90% identical to SEQ ID NO: 8. In some embodiments, the IL-7 polypeptide sequence comprises a polypeptide sequence that is at least about 95% identical to SEQ ID NO: 8. In some embodiments, the IL-7 polypeptide sequence comprises a polypeptide sequence that is at least about 96% identical to SEQ ID NO: 8. In some embodiments, the IL-7 polypeptide sequence comprises a polypeptide sequence that is at least about 97% identical to SEQ ID NO: 8. In some embodiments, the IL-7 polypeptide sequence comprises a polypeptide sequence that is at least about 98% identical to SEQ ID NO: 8. In some embodiments, the IL-7 polypeptide sequence comprises a polypeptide sequence that is at least about 99% identical to SEQ ID NO: 8. In some embodiments, the IL-7 polypeptide sequence comprises a polypeptide sequence that is 100% identical to SEQ ID NO: 8. In some embodiments, the IL-7 polypeptide retains each of the substitutions present in SEQ ID NO: 8 relative to SEQ ID NO: 1.

In some embodiments, the IL-7 polypeptide sequence comprises a polypeptide sequence that is at least about 80% identical to SEQ ID NO: 6. In some embodiments, the IL-7 polypeptide sequence comprises a polypeptide sequence that is at least about 90% identical to SEQ ID NO: 6. In some embodiments, the IL-7 polypeptide sequence comprises a polypeptide sequence that is at least about 95% identical to SEQ ID NO: 6. In some embodiments, the IL-7 polypeptide sequence comprises a polypeptide sequence that is at least about 96% identical to SEQ ID NO: 6. In some embodiments, the IL-7 polypeptide sequence comprises a polypeptide sequence that is at least about 97% identical to SEQ ID NO: 6. In some embodiments, the IL-7 polypeptide sequence comprises a polypeptide sequence that is at least about 98% identical to SEQ ID NO: 6. In some embodiments, the IL-7 polypeptide sequence comprises a polypeptide sequence that is at least about 99% identical to SEQ ID NO: 6. In some embodiments, the IL-7 polypeptide sequence comprises a polypeptide sequence that is 100% identical to SEQ ID NO: 6. In some embodiments, the IL-7 polypeptide retains each of the substitutions present in SEQ ID NO: 6 relative to SEQ ID NO: 1.

In some embodiments, the IL-7 polypeptide sequence comprises a polypeptide sequence that is at least about 80% identical to SEQ ID NO: 7. In some embodiments, the IL-7 polypeptide sequence comprises a polypeptide sequence that is at least about 90% identical to SEQ ID NO: 7. In some embodiments, the IL-7 polypeptide sequence comprises a polypeptide sequence that is at least about 95% identical to SEQ ID NO: 7. In some embodiments, the IL-7 polypeptide sequence comprises a polypeptide sequence that is at least about 96% identical to SEQ ID NO: 7. In some embodiments, the IL-7 polypeptide sequence comprises a polypeptide sequence that is at least about 97% identical to SEQ ID NO: 7. In some embodiments, the IL-7 polypeptide sequence comprises a polypeptide sequence that is at least about 98% identical to SEQ ID NO: 7. In some embodiments, the IL-7 polypeptide sequence comprises a polypeptide sequence that is at least about 99% identical to SEQ ID NO: 7. In some embodiments, the IL-7 polypeptide sequence comprises a polypeptide sequence that is 100% identical to SEQ ID NO: 7. In some embodiments, the IL-7 polypeptide retains each of the substitutions present in SEQ ID NO: 7 relative to SEQ ID NO: 1.

In some embodiments, the IL-7 polypeptide sequence comprises a polypeptide sequence that is at least about 80% identical to SEQ ID NO: 51. In some embodiments, the IL-7 polypeptide sequence comprises a polypeptide sequence that is at least about 90% identical to SEQ ID NO: 51. In some embodiments, the IL-7 polypeptide sequence comprises a polypeptide sequence that is at least about 95% identical to SEQ ID NO: 51. In some embodiments, the IL-7 polypeptide sequence comprises a polypeptide sequence that is at least about 96% identical to SEQ ID NO: 51. In some embodiments, the IL-7 polypeptide sequence comprises a polypeptide sequence that is at least about 97% identical to SEQ ID NO: 51. In some embodiments, the IL-7 polypeptide sequence comprises a polypeptide sequence that is at least about 98% identical to SEQ ID NO: 51. In some embodiments, the IL-7 polypeptide sequence comprises a polypeptide sequence that is at least about 99% identical to SEQ ID NO: 51. In some embodiments, the IL-7 polypeptide sequence comprises a polypeptide sequence that is at 100% identical to SEQ ID NO: 51. In some embodiments, the IL-7 polypeptide retains each of the substitutions present in SEQ ID NO: 51 relative to SEQ ID NO: 1.

In some embodiments, the IL-7 polypeptide sequence comprises a polypeptide sequence that is at least about 80% identical to SEQ ID NO: 50. In some embodiments, the IL-7 polypeptide sequence comprises a polypeptide sequence that is at least about 90% identical to SEQ ID NO: 50. In some embodiments, the IL-7 polypeptide sequence comprises a polypeptide sequence that is at least about 95% identical to SEQ ID NO: 50. In some embodiments, the IL-7 polypeptide sequence comprises a polypeptide sequence that is at least about 96% identical to SEQ ID NO: 50. In some embodiments, the IL-7 polypeptide sequence comprises a polypeptide sequence that is at least about 97% identical to SEQ ID NO: 50. In some embodiments, the IL-7 polypeptide sequence comprises a polypeptide sequence that is at least about 98% identical to SEQ ID NO: 50. In some embodiments, the IL-7 polypeptide sequence comprises a polypeptide sequence that is at least about 99% identical to SEQ ID NO: 51. In some embodiments, the IL-7 polypeptide sequence comprises a polypeptide sequence that is 100% identical to SEQ ID NO: 50. In some embodiments, the IL-7 polypeptide retains each of the substitutions present in SEQ ID NO: 50 relative to SEQ ID NO: 1.

### Attachment of Additional Groups (e.g., Polymers, Other Polypeptides) to IL-7 Polypeptides

In some embodiments, the IL-7 polypeptides provided herein comprise additional groups attached to the IL-7 polypeptides. Additional groups can included without limitation polymers (e.g., water soluble polymers such as poly(ethylene glycol) (PEG)), additional polypeptides (e.g., antibodies or antigen binding fragments thereof, such as in immunocytokines provided herein (e.g., PD-1 antibodies or antigen binding fragments thereof), Fc domains, etc.), small molecules (e.g., non-polymeric steric blocking groups, such as adamantane groups), conjugation handles, nanoparticles (e.g., metal nanoparticles), or any other desired additional group. In some embodiments, the additional groups are added at specified or desired residues (e.g., in a site specific manner). Such additional groups can be attached to an IL-7 polypeptide having any other modifications provided herein (e.g., V15W substitutions, G85N substitutions, substitutions provided in Table 1 or 2, or in addition to modifications for a synthetic IL-7 polypeptide).

The IL-7 polypeptides described herein can contain one or more additional groups. The IL-7 polypeptide to which the one or more additional groups are be attached can be any of the IL-7 polypeptides provided herein. In some embodiments, the addition of additional groups to certain amino acid residues can disrupt the binding interaction of the IL-7 polypeptide with the IL-7 receptor complex, the IL-7 receptor alpha subunit, or both. In some embodiments, the addition of additional groups to certain amino acid residues has little or no effect on the binding interaction of the IL-7 polypeptide with the IL-7 receptor complex, the IL-7 receptor alpha subunit, or both.

In some embodiments, the additional groups are attached to the IL-7 polypeptide through a conjugation reaction (e.g., by a reaction with a conjugation handle attached to the IL-7 polypeptide and a complementary conjugation handle attached to the additional group). In some embodiments, the conjugation reaction takes place through a conjugation handle incorporated into the IL-7 in a site specific manner (e.g., by incorporating an unnatural amino acid comprising a conjugation handle into the IL-7 polypeptide during preparation (e.g., synthesis or recombinant expression), or by site specifically attaching it to the IL-7 polypeptide in another manner). In some embodiments, the additional groups are attached to the IL-7 polypeptide directly during preparation of the IL-7 polypeptide (e.g., by incorporating an amino acid with a polymer during synthesis of the IL-7 polypeptide, or by expression of the IL-7 polypeptide linked with an additional polypeptide as a fusion protein).

In some embodiments, IL-7 polypeptides provided herein comprise modified N-terminal residues. In some embodiments, the modified N-terminal residue is modified such that another group can be attached to the N-terminal residue, such as a poly(ethylene glycol) group or an additional polypeptide. Such modified N-terminal residues may comprise conjugation handles to assist in the addition of the additional group. In some embodiments, the N-terminal residue comprises a modification of the N-terminal amino group of the N-terminal residue. In some embodiments, the N-terminal amino group of the N-terminal residue is modified to be attached to a conjugation handle. An exemplary, non-limiting embodiment of one such modification attached to the N-terminal amino group of the N-terminal residue is shown below in Structure 1B:

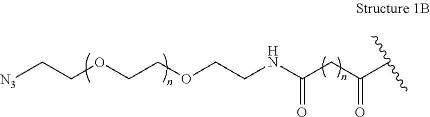

wherein each n is independently an integer from 1-30. In Structure 1B, the wavy line attached to the nitrogen signifies the point of attachment of the N-terminal amine to the backbone of the N-terminal residue. In some embodiments, the N-terminal modification of Structure 1B comprises 2-10 ethylene glycol units (leftmost n) and 3 methylene linkers between the di-carbonyl moiety (rightmost n). Alternatively, the azide functionality of Structure 1B may be replaced with another suitable conjugation handle (e.g., an alkyne functionality, such as a DBCO group). The modification of an N-terminal residue of the IL-7 polypeptide can be in addition to any of the other modifications provided herein (e.g., V15W substitutions, polymers attached at residue 81, G85N substitutions, substitutions provided in Table 1 or 2, or in addition to modifications for a synthetic IL-7 polypeptide).

In some embodiments, the modified N-terminal residue of the IL-7 polypeptide comprises a substitution for another amino acid which comprises a conjugation handle. In some embodiments, the substitution for another amino acid which comprises a conjugation handle is a substitution for an unnatural amino acid comprising a conjugation handle. In some embodiments, the substitution for another amino acid which comprises a conjugation handle is for a natural amino acid which has been modified to incorporate a conjugation handle (e.g., attachment of a conjugation handle to a lysine, aspartate, glutamate, cysteine, serine, threonine, tyrosine, or other suitable natural amino acid). In some embodiments, the residues of the IL-7 polypeptides are substituted with modified lysine residues. In some embodiments, the modified lysine residues comprise an amino, azide, allyl, ester, and/or amide functional groups. In some embodiments, substitution of the N-terminal residue of the IL-7 polypeptide with another amino acid comprising a conjugation handle is a substitution with an amino acid having a structure built from precursors Structure 2B, Structure 3B, Structure 4B, or Structure 5B (e.g., comprises the relevant side chains):

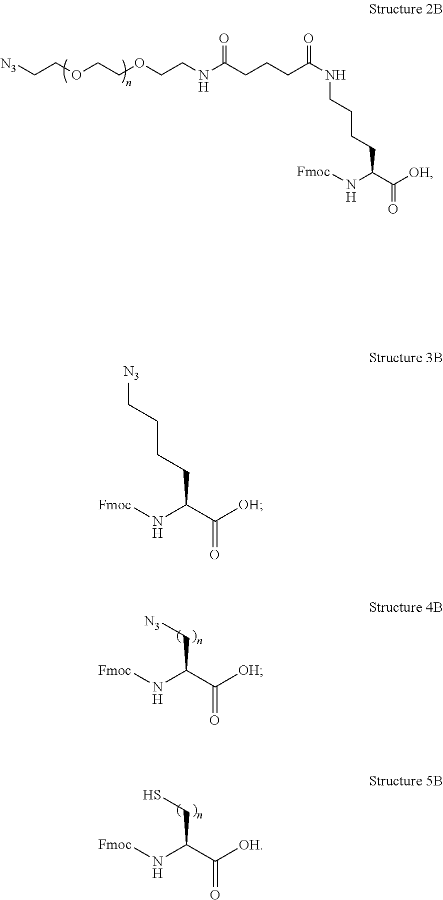

In some embodiments, any of Structure 1B-4B can be substituted for the N-terminal residue of the IL-7 polypeptide to allow conjugation of an additional group. Thus, in embodiments wherein the IL-7 polypeptide comprises a modification to the N-terminal residue, it is contemplated that the modification (e.g., a poly(ethylene glycol) group added to the residue) may be added to the side chain of the residue rather than the N-terminal amine. In some embodiments, any of structures 1B-4B can be substituted for a different residue of the IL-7 polypeptide (e.g., any of residues 2-152 using SEQ ID NO: 1 as a reference sequence) to allow for conjugation at a different site of the IL-7 polypeptide. In some embodiments, the azide functionality may also be replaced with another suitable conjugation handle (e.g., an alkyne such as a DBCO group, or any other conjugation handle as provided herein). In some embodiments, an amino acid comprising a conjugation handle is incorporated at a location other than the N-terminus (e.g., residue K81).

In some embodiments, the IL-7 polypeptide comprises a polymer covalently attached at any amino acid residue of the IL-7 polypeptide. In some embodiments, the IL-7 polypeptide comprises a polymer linked to the N-terminal residue. In some embodiments, the polymer is attached to the N-terminal amine of the N-terminal residue. In some embodiments, the polymer is attached to the side chain of the N-terminal residue. In some embodiments, the N-terminal residue is substituted for a different amino acid than the N-terminal residue of SEQ ID NO: 1. In some embodiments, the N-terminal residue of the IL-7 polypeptide is the first remaining residue of after an N-terminal truncation (e.g., the IL-7 polypeptide comprises an N-terminal truncation of 1, 2, 3, 4, 6, 7, 8, 9, 10, or more residues of SEQ ID NO: 1). In such cases, the N-terminal residue of the IL-7 polypeptide is C2, D3, I4, E5, G6, K7, D8, G9, K10, Q11, etc, or a residue substituted at the corresponding residue, depending on the number of amino acid residues truncated. In some embodiments, the N-terminal residue is the final residue of an N-terminal extension peptide attached to the sequence set forth in SEQ ID NO: 1. For example, if the IL-7 polypeptide comprises an N-terminal extension of the sequence MG-, then M is the N-terminal residue.

The conjugation handles provided herein can be any suitable reactive group capable of reacting with a complementary reactive group (e.g., any of the conjugation handles described herein, such as below or in the “Table of Conjugation Handles” provided herein). In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof).

In some embodiments, the conjugation handle comprises a reactive group selected from azide, alkyne, tetrazine, halide, sulfhydryl, disulfide, maleimide, activated ester, alkene, aldehyde, ketone, imine, hydrazine, acyltrifluoroborate, hydroxylamine, phosphine, trans-cyclooctene, and hydrazide. In some embodiments, the conjugation handle and complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; US10266502B2; and US20190204330A1, each of which is incorporated by reference in its entirety.

In some embodiments, a group attached to the IL-7 polypeptide (e.g., a polymer moiety or an additional polypeptide) comprises a conjugation handle (e.g., for attaching the group to the IL-7 polypeptide) or a reaction product of a conjugation handle with a complementary conjugation handle (e.g., after the group has been attached to the IL-7 polypeptide). In some embodiments, the reaction product of the conjugation handle with the complementary conjugation handle results from a KAT ligation (reaction of potassium acyltrifluoroborate with hydroxylamine), a Staudinger ligation (reaction of an azide with a phosphine), a tetrazine cycloaddition (reaction of a tetrazine with a trans-cyclooctene), or a Huisgen cycloaddition (reaction of an alkyne with an azide). In some embodiments, the group attached to the IL-7 polypeptide (e.g., the polymer or the additional polypeptide) will comprise a reaction product of a conjugation handle with a complementary conjugation handle (or a portion thereof) which was used to attach the group to the IL-7 polypeptide.

In some embodiments, the IL-7 polypeptide comprises at least one polymer attached to the IL-7 polypeptide. In some embodiments, the polymer is attached to the N-terminal residue of the IL-7 polypeptide (e.g., using a modification discussed supra to attach the polymer). In some embodiments, the polymer attached to the N-terminal residue of the IL-7 polypeptide is that of Structure 1B. In some embodiments, Structure 1B is used to attach a larger polymer to the IL-7 polypeptide (or another suitable structure is used to attach a polymer to the IL-7 polypeptide).

In some embodiments, the polymer comprises a water soluble polymer. In some embodiments, the polymer comprises poly(alkylene oxide), polysaccharide, poly(vinyl pyrrolidone), poly(vinyl alcohol), polyoxazoline, poly(acryloylmorpholine), or a combination thereof. In some embodiments, the polymer comprises poly(alkylene oxide). In some embodiments, the polymer comprises polyethylene glycol or polypropylene glycol, or a combination thereof. In some embodiments, the polymer comprises polyethylene glycol.

In some embodiments, the polymer is linear. In some embodiments, the polymer is branched. In some embodiments, the water-soluble polymer is linear or branched. In some embodiments, the polymer is branched and comprises multiple polyethylene glycol chains. In some embodiments, the polymer comprises from 1 to 10 polyethylene glycol chains. In some embodiments, the polymer comprises 1 polyethylene glycol chains to 10 polyethylene glycol chains. In some embodiments, the polymer comprises 1 polyethylene glycol chains to 2 polyethylene glycol chains, 1 polyethylene glycol chains to 4 polyethylene glycol chains, 1 polyethylene glycol chains to 6 polyethylene glycol chains, 1 polyethylene glycol chains to 10 polyethylene glycol chains, 2 polyethylene glycol chains to 4 polyethylene glycol chains, 2 polyethylene glycol chains to 6 polyethylene glycol chains, 2 polyethylene glycol chains to 10 polyethylene glycol chains, 4 polyethylene glycol chains to 6 polyethylene glycol chains, 4 polyethylene glycol chains to 10 polyethylene glycol chains, or 6 polyethylene glycol chains to 10 polyethylene glycol chains.

In some embodiments, the polymer has a molecular weight of at least about 0.1 kDa, 0.5 kDa, 1 kDa, 2 kDa, 3 kDa, 5 kDa, 10 kDa, 15 kDa, 20 kDa, 30 kDa. In some embodiments, the polymer has a molecular weight of at most about 50 kDa, 40 kDa, 30 kDa, 20 kDa, 15 kDa, 10 kDa, 5 kDa, 3 kDa, 2 kDa, or 1 kDa. In some embodiments, the polymer has a molecular weight of from about 0.1 kDa to about 50 kDa, about 0.1 kDa to about 40 kDa, about 0.1 kDa to about 30 kDa, about 0.1 kDa to about 20 kDa, about 0.1 kDa to about 15 kDa, about 0.1 kDa to about 10 kDa, about 0.1 kDa to about 5 kDa, about 0.1 kDa to about 3 kDa, about 0.1 kDa to about 2 kDa, about 0.1 kDa to about 1 kDa, 0.5 kDa to about 50 kDa, about 0.5 kDa to about 40 kDa, about 0.5 kDa to about 30 kDa, about 0.5 kDa to about 20 kDa, about 0.5 kDa to about 15 kDa, about 0.5 kDa to about 10 kDa, about 0.5 kDa to about 5 kDa, about 0.5 kDa to about 3 kDa, about 0.5 kDa to about 2 kDa, about 0.5 kDa to about 1 kDa, about 1 kDa to about 50 kDa, about 1 kDa to about 30 kDa, about 1 kDa to about 15 kDa, about 1 kDa to about 10 kDa, about 1 kDa to about 5 kDa about 1 kDa to about 3 kDa, about 1 kDa to about 2 kDa, about 3 kDa to about 50 kDa, about 3 kDa to about 30 kDa, about 3 kDa to about 15 kDa, about 3 kDa to about 10 kDa, about 5 kDa to about 50 kDa, about 5 kDa to about 30 kDa, about 5 kDa to about 20 kDa, about 10 kDa to about 50 kDa, or about 10 kDa to about 30 kDa.

In some embodiments, the polymer is an end-capped polymer. In some embodiments, the polymer is an end-capped polyethylene glycol. In some embodiments, the polymer is end-capped with a functional group selected from amine, alkoxy (e.g., methoxy, ethoxy, propoxy, etc.), hydroxyl, amide (e.g., —NH(C═O)(C₁-C₄ alkyl)), carboxylate, and ester (e.g., methyl ester, ethyl ester, etc.). In some embodiments, the polymer as an amine end-capped PEG. In some embodiments, the polymer is an acetyl end-capped PEG. In some embodiments, the polymer is a methoxy end-capped PEG.

In some embodiments, the IL-7 polypeptide attached to one or more polymers can retain binding to IL-7Rα and exhibit an increased half-life (t_{1/2}) (e.g., plasma or serum half-life). In some embodiments, the IL-7 polypeptide attached to one or more polymers can have decreased binding (e.g., only slightly decreased binding) to IL-7Rα and exhibit an increased half-life (t_{1/2}). In some embodiments, the IL-7 polypeptide attached to one or more polymer moieties can retain binding to the IL-7Rα/γ heterodimer and exhibit an increased half-life (t_{1/2}). In some embodiments, the IL-7 polypeptide attached to one or more polymer moieties can have decreased binding (e.g., only slightly decreased binding) to the IL-7Rα/γ heterodimer and exhibit an increased half-life (t_{1/2}).

The half-life extending polymers may be of any size, including up to about 6 kDa, up to about 25 kDa, up to about 50 kDa, or up to about 100 kDa. In some embodiments, the half-life extending polymers are PEG polymers. In some embodiments, the half-life extending polymer has an average molecular weight of from about 1,000 Da to about 20,000 Da, for example, PEG 1000, PEG 1450, PEG 1500, PEG 4000, PEG 4600, and PEG 8000, or of from about 1,000 Da to about 100,000 Da.

In some embodiments, the IL-7 polypeptide conjugated to a polymer can retain binding to the IL-7 receptor or the IL-7 receptor alpha subunit as compared to the IL-7 polypeptide without the polymer (e.g., the presence of the polymer has a minimal effect on binding to IL-7 receptor alpha subunit). In some embodiments, the IL-7 polypeptide conjugated to the polymer can retain binding to the IL-7Rα/γ heterodimer as compared to the IL-7 polypeptide without the polymer. In some embodiments, the IL-7 polypeptide conjugated to the polymer moieties can retain pSTAT5 induction of one or more T cells compared to the IL-7 polypeptide without the polymer.

In some embodiments, the IL-7 polypeptide conjugated to the polymer can exhibit reduced binding to the IL-7 receptor or the IL-7 receptor alpha subunit as compared to the IL-7 polypeptide without the polymer. In some embodiments, the IL-7 polypeptide conjugated to one or more polymer moieties can have reduced binding to the IL-7Rα/γ heterodimer as compared to the IL-7 polypeptide without the polymer. In some embodiments, an IL-7 polypeptide conjugated to the polymer can exhibit decreased pSTAT5 induction of one or more T cells compared to the IL-7 polypeptide without the polymer.

In some embodiments, the IL-7 polypeptide comprises a polymer or small molecule (e.g., adamantane) attached to a side chain of an amino acid residue of the IL-7 polypeptide. In some embodiments, the polymer or small molecule is attached a side chain of an amino acid reside of the IL-7 polypeptide at an amino acid residue which interacts with or is near to a residue which interacts with the IL-7 receptor or a subunit thereof (e.g., the IL-7 receptor alpha subunit). In some embodiments, the polymer or small molecule attached to the side chain of the amino acid residue of the IL-7 polypeptide reduces binding of the IL-7 polypeptide to the IL-7 receptor or a subunit thereof. In some embodiments, the polymer or small molecule attached to the side chain of the amino acid residue of the IL-7 polypeptide reduces the ability of the IL-7 polypeptide to signal through the IL-7 receptor. The polymer attached to the side chain of the amino acid residue of the IL-7 polypeptide can be in addition to another polymer attached to the IL-7 polypeptide (e.g., a polymer attached to the N-terminal amine as provided herein). The polymer attached to a side chain of an IL-7 polypeptide can be in addition to any of the other modifications provided herein (e.g., V15W substitutions, polymers attached at residue 81, G85N substitutions, substitutions provided in Table 1 or 2, or in addition to modifications for a synthetic IL-7 polypeptide).

In some embodiments, the IL-7 polypeptide comprises a polymer or small molecule (e.g., adamantane) attached at a residue of the IL-7 polypeptide selected from residues 2, 7, 11, 14, 15, 18, 34, 47, 70, 71, 72, 74, 77, 78, 79, 80, 81, 84, 85, 88, 92, 129, 141, and 142. In some embodiments, the polymer is attached at a residue of the IL-7 polypeptide selected from residues 11, 14, 15, 18, 22, 72, 74, 77, 80, 81, 84, 85, 88, 89, and 142. In some embodiments, the polymer is attached at a residue of the IL-7 polypeptide selected from residues 15, 81, 85, and 142. In some embodiments, the polymer is attached at residue 15 of the IL-7 polypeptide. In some embodiments, the polymer is attached at residue 81 of the IL-7 polypeptide. In some embodiments, the polymer is attached at residue 85 of the IL-7 polypeptide. In some embodiments, the polymer is attached at residue 142 of the IL-7 polypeptide. In some embodiments, a small molecule (e.g., adamantane (e.g., as a 1-adamantanecarboxyl group) or other bulky hydrocarbon group) is attached at one of the residues to which a polymer can be attached (e.g., instead of the polymer).

In some embodiments, the residue to which the polymer is attached at the side chain is a natural amino acid residue. In some embodiments, the residue to which the polymer is covalently attached at the side chain is selected from cysteine, aspartate, asparagine, glutamate, glutamine, serine, threonine, lysine, and tyrosine. In some embodiments, the residue to which the polymer is covalently attached at the side chain is selected from asparagine, aspartic acid, cysteine, glutamic acid, glutamine, lysine, and tyrosine. In some embodiments, the polymer is covalently attached at the side chain of a cysteine. In some embodiments, the polymer is covalently attached at the side chain of a lysine. In some embodiments, the polymer is covalently attached at the side chain of a glutamine. In some embodiments, the polymer is covalently attached at the side chain of a glutamate. In some embodiments, the polymer is covalently attached at the side chain of an asparagine. In some embodiments, the polymer is covalently attached at the side chain of an aspartate. In some embodiments, the residue to which the polymer is attached at the side chain is a tyrosine. In some embodiments, the residue to which the polymer is attached is the natural amino acid in that position in SEQ ID NO: 1 (e.g., K81).

In some embodiments, the polymer attached to the side chain is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative or modified version of any naturally occurring amino acid, or any unnatural amino acid (e.g., an amino acid containing a desired reactive group, such as a CLICK chemistry reagent such as an azide, alkyne, etc.). In some embodiments, the polymer is covalently attached site-specifically to a natural amino acid.

In some embodiments, the polymer attached to a side chain is attached at an unnatural amino acid residue. In some embodiments, the unnatural amino acid residue comprises a conjugation handle. In some embodiments, the conjugation handle facilitates the addition of the polymer to the IL-7 polypeptide. The conjugation handle can be any of the conjugation handles provided herein. In some embodiments, the polymer is covalently attached site-specifically to the unnatural amino acid. Non-limiting examples of amino acid residues comprising conjugation handles can be found, for example, in PCT Pub. Nos. WO2015/054658, WO2014/036492, and WO2021/133839, WO2006/069246, and WO2007/079130, each of which is incorporated by reference as if set forth in its entirety. In some embodiments, the polymer is attached to an unnatural amino acid residue without use of a conjugation handle.

In some embodiments, the polymer attached to a side chain of a residue of the IL-7 polypeptide is covalently attached at residue 81. In some embodiments, the polymer is covalently attached at residue K81E, K81D, K81Q, K81C, K81N, or K81Y. In some embodiments, the polymer is covalently attached at residue K81. In some embodiments, the polymer is covalently attached to an unnatural amino acid at residue 81.

In some embodiments, the polymer attached to the chain of a residue of the IL-7 polypeptide is attached to the lysine at residue 81 of the IL-7 polypeptide. In some embodiments, the polymer is attached to the lysing through a bond formed with the side chain amino group of the lysine. In some embodiments, the bond form with the side chain amino group of the lysine is an amide, a carbamate, a carbamide, or an amino-alkyl bond (e.g., by reductive amination of a ketone or aldehyde group with the side chain amino group of the lysine). In some embodiments, the bond formed with the side chain amino group of the lysine is an amide bond formed with a carboxyl group attached to the polymer.

In some embodiments, the polymer attached to the lysine at residue K81 of the IL-7 polypeptide comprises a structure

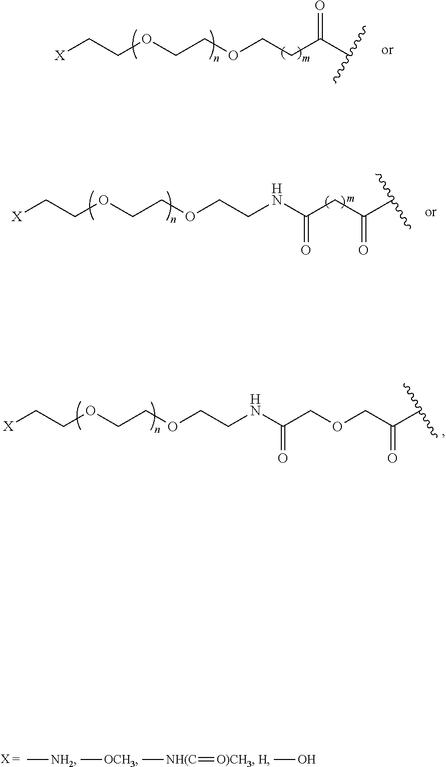

wherein n is an integer from 1-30 and m is an integer from 1-6. In some embodiments, n is an integer from 1-20, 1-15, 1-10, 1-6, 2-30, 2-20, 2-15, 2-10, or 2-6. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 2. In some embodiments, n is 4. In some embodiments, n is 6. In some embodiments, n is 11. In some embodiments, m is 1, 2, 3, or 4. In some embodiments, m is 3. In some embodiments, m is 1. In some embodiments, X is NH₂. In some embodiments, X is —OCH₃.

In some embodiments, polymer attached to the side chain of the residue of the IL-7 polypeptide comprises a water soluble polymer. In some embodiments, the polymer comprises poly(alkylene oxide), polysaccharide, poly(vinyl pyrrolidone), poly(vinyl alcohol), polyoxazoline, poly(acryloylmorpholine), or a combination thereof. In some embodiments, the polymer comprises poly(alkylene oxide). In some embodiments, the polymer comprises polyethylene glycol or polypropylene glycol, or a combination thereof. In some embodiments, the polymer comprises polyethylene glycol.

In some embodiments, the polymer attached to the side chain of the residue of the IL-7 polypeptide (e.g., at K81) has a molecular weight of from about 0.1 kDa to about 5 kDa. In some embodiments, the polymer has a molecule weight of from about 0.1 kDa to about 5 kDa, from about 0.1 kDa to about 4 kDa, from about 0.1 kDa to about 3 kDa, from about 0.1 kDa to about 2 kDa, from about 0.1 kDa to about 1 kDa, from about 0.1 kDa to about 0.5 kDa, from about 0.1 kDa to about 0.4 kDa, from about 0.1 kDa to about 0.3 kDa, about 0.2 kDa to about 5 kDa, from about 0.2 kDa to about 4 kDa, from about 0.2 kDa to about 3 kDa, from about 0.2 kDa to about 2 kDa, from about 0.2 kDa to about 1 kDa, from about 0.2 kDa to about 0.5 kDa, from about 0.2 kDa to about 0.4 kDa, or from about 0.2 kDa to about 0.3 kDa. In some embodiments, the polymer has a molecular weight of at most 5 kDa, at most 4 kDa, at most 3 kDa, at most 2 kDa, at most 3 kDa, at most 2 kDa, at most 1 kDa, at most 0.9 kDa, at most 0.8 kDa, at most 0.7 kDa, at most 0.6 kDa, at most 0.5 kDa, at most 0.4 kDa, or at most 0.3 kDa.

In some embodiments, the polymer attached to the side chain of the amino acid residue of the IL-7 polypeptide (e.g., at K81) comprises polyethylene glycol. In some embodiments, the polymer comprises from 2 to 30 ethylene glycol units. In some embodiments, the polymer comprises 2 ethylene glycol units. In some embodiments, the polymer comprises 4 ethylene glycol units. In some embodiments, the polymer comprises 6 ethylene glycol units. In some embodiments, the polymer comprises 8 ethylene glycol units. In some embodiments, the polymer comprises 11 ethylene glycol units. In some embodiments, the polymer comprises from 1-20, 1-15, 1-10, 1-6, 2-30, 2-20, 2-15, 2-10, or 2-6 ethylene glycol units.

In some embodiments, the polymer attached to the side chain of the amino acid residue of the IL-7 polypeptide (e.g., at K81) is an end-capped polymer. In some embodiments, the polymer is an end-capped polyethylene glycol (PEG). In some embodiments, the polymer is end-capped with a functional group selected from amine, alkoxy (e.g., methoxy, ethoxy, propoxy, etc.), hydroxyl, amide (e.g., —NH(C═O)(C₁-C₄ alkyl)), carboxylate, and ester (e.g., methyl ester, ethyl ester, etc.). In some embodiments, the polymer as an amine end-capped PEG. In some embodiments, the polymer is an acetyl end-capped PEG (e.g., capped with —NH(C═O)CH₃). In some embodiments, the polymer is a methoxy end-capped PEG.

In some embodiments, a herein described IL-7 polypeptide comprises multiple polymers covalently attached thereon (e.g., two of the polymers provided herein, such as a polymer attached to the N-terminus and a polymer attached to a side chain of an amino acid residue, such as residue 81). In some embodiments, the described IL-7 polypeptide comprises 1, 2, 3, 4, 5, 6, 7, 8, 9, 10, or more polymers covalently attached to the IL-7 polypeptide.

In some embodiments, the IL-7 polypeptide comprises from 1 to 10 covalently attached water-soluble polymers. In some embodiments, the IL-7 polypeptide comprises 1 to 10 covalently attached water-soluble polymers. In some embodiments, the IL-7 polypeptide comprises 1 or 2 covalently attached water-soluble polymers, 1 to 3 covalently attached water-soluble polymers, 1 to 4 covalently attached water-soluble polymers, 1 to 6 covalently attached water-soluble polymers, 1 to 8 covalently attached water-soluble polymers, 1 to 10 covalently attached water-soluble polymers, 2 or 3 covalently attached water-soluble polymers, 2 to 4 covalently attached water-soluble polymers, 2 to 6 covalently attached water-soluble polymers, 2 to 8 covalently attached water-soluble polymers, 2 to 10 covalently attached water-soluble polymers, 3 or 4 covalently attached water-soluble polymers, 3 to 6 covalently attached water-soluble polymers, 3 to 8 covalently attached water-soluble polymers, 3 to 10 covalently attached water-soluble polymers, 4 to 6 covalently attached water-soluble polymers, 4 to 8 covalently attached water-soluble polymers, 4 to 10 covalently attached water-soluble polymers, 6 to 8 covalently attached water-soluble polymers, 6 to 10 covalently attached water-soluble polymers, or 8 to 10 covalently attached water-soluble polymers.

In some embodiments, an IL-7 polypeptide described herein comprises a first polymer covalently attached to the N-terminus of the IL-7 polypeptide. In some embodiments, the IL-7 polypeptide comprises a second polymer covalently attached thereto. In some embodiments, the second polymer is covalently attached to a side chain of an amino acid residue of the IL-7 polypeptide (e.g., any of the polymers provided herein attached at any of the side chains provided herein (e.g., the side chain of residue K81)).

In some embodiments, the IL-7 polypeptide is conjugated with an additional polypeptide. The conjugation can take a variety of different forms which link the additional polypeptide with the IL-7 polypeptide. In some embodiments, the IL-7 polypeptide is conjugated with the additional polypeptide as a fusion protein, through a covalent chemical linking group, or through a non-covalent means (e.g., biotin/avidin or streptavidin). In some embodiments, the IL-7 polypeptide and the additional polypeptide form a fusion polypeptide. In some embodiments, the IL-7 polypeptide and the additional polypeptide are conjugated together with a chemical linker. In some embodiments, the additional polypeptide comprises an antibody or binding fragment thereof. In some embodiments, the antibody comprises a humanized antibody, a murine antibody, a chimeric antibody, a bispecific antibody, any fragment thereof, or any combination thereof. In some embodiments, the antibody is a monoclonal antibody or any fragment thereof. In some embodiments, the IL-7 polypeptide is conjugated to a half-life extension polypeptide (e.g., albumin). In some embodiments, the IL-7 polypeptide is conjugated to the additional polypeptide (e.g., the antibody or antigen binding fragment thereof) through the N-terminus of the IL-7 polypeptide (e.g., using a modified N-terminus provided herein, such as an N-terminus modified with Structure 1B). In some embodiments, the IL-7 polypeptide is conjugated to the additional polypeptide through a polymer attached to the IL-7 polypeptide (e.g., a polymer attached to N-terminus of the IL-7 polypeptide). Conjugation with an additional polypeptide can be to an IL-7 polypeptide having any of the other modifications provided herein (e.g., V15W substitutions, polymers attached at residue 81, G85N substitutions, substitutions provided in Table 1 or 2, or in addition to modifications for a synthetic IL-7 polypeptide).

In some embodiments, the IL-7 polypeptides described herein contain a tether group which links the IL-7 polypeptide to the additional moiety (e.g., the polymer, the additional polypeptide, etc.). In some embodiments, the tether groups comprises —CH₂—, —CH₂CH₂—, or —CH₂CH₂CH₂—. In some embodiments, the tether group comprises the product of a biorthogonal reaction (e.g., biocompatible and selective reactions). In some embodiments, the bioorthogonal reaction is a Cu(I)-catalyzed or “copper-free” alkyne-azide triazole-forming reaction, the Staudinger ligation, inverse-electron-demand Diels-Alder (IEDDA) reaction, alkyne-nitrone cycloaddition chemistry, or a metal-mediated process such as olefin metathesis and Suzuki-Miyaura or Sonogashira cross-coupling.

In some embodiments, the tether group comprises a structure of Formula (X)

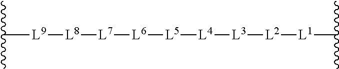

wherein each of L¹, L², L³, L⁴, L⁵, L⁶, L⁷, L⁸, and L⁹ is independently —O—, —NR^{L}—, —(C₁-C₆ alkylene) NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —C(═O)(C₁-C₆ alkenylene)S—, —S(C₁-C₆ alkylene)C(═O)— —S(═O)—, —(═O)₂—, —C(═O)—, —S(C₁-C₆ alkylene)C(═O)—, —C(═O)(C₁-C₆ alkylene)-, —C(═O)(C₁-C₆ alkylene)C(═O)—, —C(═O)O—, —OC(═O)—, —OC(═O)O—, —C(═O) NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}, —(C₁-C₆ alkyl)(CH₂—CH₂—O)_{qa}—, —(C₁-C₆ alkyl)(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene)
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

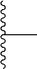

is a point of attachment to the IL-7 polypeptide or the additional group.

In some embodiments, the IL-7 polypeptide comprises a tether group shown in Table 3. In Table 3, each

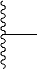

is a point of attachment to either the IL-7 polypeptide (e.g., an amino group of the IL-7 polypeptide) or to the additional group.

TABLE 3
Exemplary tether group strctures for IL-7 polypeptides

| Tether | |
|---|---|
| Group | |
| Identifier | Tether Group Structure |
| Formula A | 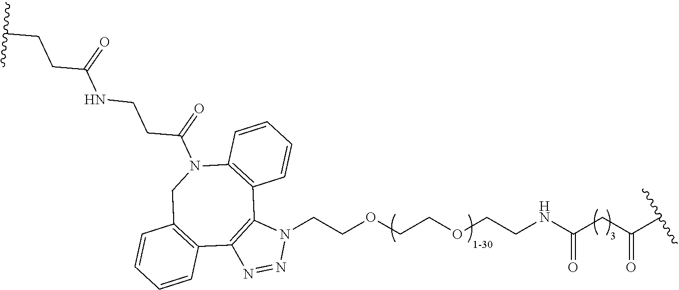 |
| Formula B | 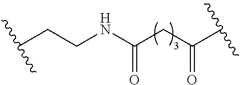 |
| Formula C | 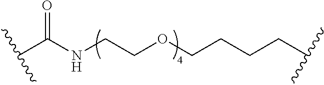 |
| Formula D | 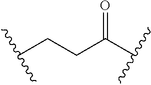 [SMILES: *CCC(*)=O] |
| Formula E | 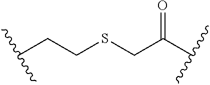 [SMILES: *CCSCC(*)=O] |
| Formula F | 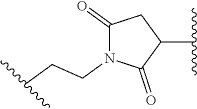 [SMILES: *CCN1C(=O)CC(*)C1=O] |
| Formula G | 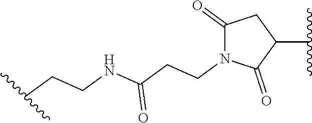 [SMILES: *CCNC(=O)CCN1C(=O)CC(*)C1=O] |

### Synthetic IL-7 Polypeptides

In some embodiments, the IL-7 polypeptide is synthetic. In some embodiments, the IL-7 polypeptide is prepared from one or more chemically synthesized peptides. Any IL-7 polypeptide provided herein may further comprise any of the modifications of a synthetic IL-7 polypeptide provided herein. Similarly, any synthetic IL-7 polypeptide provided herein can comprise any of the modifications for IL-7 polypeptides described above (e.g., V15W substitutions, polymers attached at residue 81, G85N substitutions, substitutions provided in Table 1 or 2, or in addition to modifications for a synthetic IL-7 polypeptide).

In one aspect provided herein is a synthetic IL-7 polypeptide comprising at least one modification to the amino acid sequence as set forth in SEQ ID NO: 1, wherein the at least one modification is a natural amino acid substitution or an additional group covalently attached to a side chain of an amino acid residue of the IL-7 polypeptide. In some embodiments, the natural amino acid substitution is any one of those provided herein (e.g., any of the natural amino acid substitutions provided in Table 1 or Table 2). In some embodiments, the at least one modification is at residue selected from C2, K7, Q11, S14, V15, V18, C34, N36, C47, N70, S71, T72, D74, D76, L77, H78, L79, L80, K81, E84, G85, 188, C92, E114, C129, and C141. In some embodiments, the modification comprises an amino acid substitution selected from C2S, C2A, K7A, Q11F, S14H, V15A, V15W, V18A, C34S, C34A, N36S, C47S, C47A, N70K, N70Y, S71N, S71R, S71V, T72H, T72N, T72W, D74A, D74G, D74N, D74Q, D74W, D76S, L77A, L77D, L77E, L77H, L77Q, L77T, L77V, H78A, H78R, H78Y, L79A, L80K, L80Q, L80W, K81A, K81E, K81M, K81Q, K81W, E84F, E84N, E84R, E84W, E84Y, G85A, G85N, G85Q, G85W, I88A, I88D, I88E, I88F, I88R, I88T, C92S, C92A, E114S, C129S, C129A, C141S, and C141A. In some embodiments, the amino acid substitution selected from K7A, S14H, V15W, D36S, N70K, N70Y, S71N, S71R, S71V, T72H, T72N, T72W, D74A, D74G, D74W, D76S, L77E, L77H, L77Q, L77T, L77V, H78A, H78R, H78Y, L79A, L80K, L80Q, L80W, K81Q, K81W, E84F, E84N, E84R, E84W, E84Y, G85A, G85N, G85Q, G85W, I88A, I88D, I88E, I88F, and E114S. In some embodiments, the modification comprises the additional group covalently attached to the side chain of the amino acid residue, wherein the amino acid residue is at a position selected from residues 2, 7, 11, 14, 15, 18, 34, 47, 70, 71, 72, 74, 77, 78, 79, 80, 81, 84, 85, 88, 92, 129, 141, and 142.

In some embodiments, the IL-7 polypeptide comprises one or more homoserine residues. The homoserine residue can be at any position as provided herein for a synthetic IL-7 polypeptide. In some embodiments, the IL-7 polypeptide comprises a homoserine residue at a position selected from the region of residues 26-46, residues 66-86, and residues 104-124. In some embodiments, the IL-7 polypeptide comprises a homoserine residue at a position selected from the region of residues 31-41, residues 71-81, and residues 109-119. In some embodiments, the IL-7 polypeptide comprises a homoserine at each of the three regions. In some embodiments, the IL-7 polypeptide comprises a homoserine substitution at each of residues 36, 76, and 114.

In some embodiments, the IL-7 polypeptide comprises a homoserine (Hse) residue located in any one of amino acid residues 31-41. In some embodiments, the IL-7 polypeptide comprises a Hse residue located in any one of amino acid residues 71-81. In some embodiments, the IL-7 polypeptide comprises a Hse residue located in any one of amino acid residues 109-119. In some embodiments, the IL-7 polypeptide comprises 1, 2, 3, or more Hse residues. In some embodiments, the IL-7 polypeptide comprises Hse36, Hse76, Hse114, or a combination thereof. In some embodiments, the IL-7 polypeptide comprises Hse36, Hse76, and Hse114. In some embodiments, the IL-7 polypeptide comprises at least two amino acid substitutions, wherein the at least two amino acid substitutions are selected from (a) a homoserine (Hse) residue located in any one of amino acid residues 31-41; (b) a homoserine residue located in any one of amino acid residues 71-81; and (c) a homoserine residue located in any one of amino acid residues 109-119. In some embodiments, the IL-7 polypeptide comprises Hse36 and Hse76. In some embodiments, the IL-7 polypeptide comprises Hse36 and Hse114. In some embodiments, the IL-7 polypeptide comprises Hse76 and Hse114. In some embodiments, the IL-7 polypeptide comprises Hse36. In some embodiments, the IL-7 polypeptide comprises Hse76. In some embodiments, the IL-7 polypeptide comprises Hse114.

In some embodiments, the IL-7 polypeptide comprises a homoserine (Hse) residue at one or more positions within the synthetic polypeptide. In some embodiments, the polypeptide comprises a homoserine residue at a position selected from the region of residues 26-46, residues 66-86, and residues 104-124, wherein residue position numbering of the IL-7 polypeptide is based on SEQ ID NO: 1 as a reference sequence. In some embodiments, the IL-7 comprises homoserine residues at positions selected from the region of residues 29-42, residues 69-83, and residues 107-124 of the IL-7 polypeptide. In some embodiments, the IL-7 comprises homoserine residues at positions selected from the region of residues 31-41, residues 71-81, and residues 109-119 of the IL-7 polypeptide. In some embodiments, the IL-7 comprises homoserine residues at positions selected from the region of residues 33-39, residues 73-79, and residues 111-117 of the IL-7 polypeptide. In some embodiments, the IL-7 comprises homoserine residues at positions selected from the region of residues 34-38, residues 74-78, and residues 112-116 of the IL-7 polypeptide. In some embodiments, the IL-7 polypeptide comprises a homoserine in one, two, or three of the regions provided herein.

In some embodiments, the IL-7 polypeptide comprises a Hse residue in one or more of the regions of residues 31-41, residues 71-81, and residues 109-119, wherein residue position numbering of the IL-7 polypeptide is based on SEQ ID NO: 1 as a reference sequence. In some embodiments, the IL-7 polypeptide comprises a Hse residue in one or more of the regions of residues 31-41, residues 71-81, and residues 109-119. In some embodiments, the IL-7 polypeptide comprises a Hse residue in two of the regions of residues 31-41, residues 71-81, and residues 109-119. In some embodiments, the IL-7 polypeptide comprises a Hse residue in two of the regions of residues 31-41, residues 71-81, and residues 109-119. In some embodiments, the IL-7 polypeptide comprises a Hse residue in each the regions of residues 31-41, residues 71-81, and residues 109-119.

In some embodiments, one or more methionine residues of the IL-7 polypeptide is substituted. The methionine substitutions can be any of the substitutions provided herein for an IL-7 polypeptide. In some embodiments, the IL-7 polypeptide comprises one or more substitutions at a residue which is a methionine in SEQ ID NO: 1. In some embodiments, one or more methionine residues are substituted for a methionine isostere. In some embodiments, one or more methionine residues are substituted for norleucine or O-methyl-homoserine. In some embodiments, the IL-7 polypeptide comprises a norleucine substitution at M17, M27, M54, M69, M147, or any combination thereof. In some embodiments, the IL-7 polypeptide comprises a norleucine substitution one, two, three, four, or five of M17, M27, M54, M69, or M147. In some embodiments, the IL-7 polypeptide comprises a norleucine substitution at M17, M27, M54, M69, and M147.

In some embodiments, the IL-7 polypeptide comprises 1, 2, 3, 4, 5, or more norleucine (Nle) residues. In some embodiments, the IL-7 polypeptide comprises an Nle residue located in any one of residues 12-22. In some embodiments, the IL-7 polypeptide comprises one or more Nle residues located in any one of amino acid residues 22-32. In some embodiments, the IL-7 polypeptide comprises a Nle residue located in any one of amino acid residues 49-59. In some embodiments, the IL-7 polypeptide comprises a Nle residue located in any one of amino acid residues 64-74. In some embodiments, the IL-7 polypeptide comprises a Nle residue located in any one of amino acid residues 142-152. In some embodiments, the IL-7 polypeptide comprises five Nle substitutions. In some embodiments, the IL-7 polypeptide comprises Nle17, Nle27, Nle54, Nle69, and Nle147.

In some embodiments, the IL-7 polypeptide comprises one or more amino acid substitutions selected from: (a) a homoserine residue located at any one of residues 31-41; (b) a homoserine residue located at any one of residues 71-81; (c) a homoserine residue located at any one of residues 109-119; (d) a norleucine or O-methyl-homoserine residue located at any one of residues 12-22; (c) a norleucine or O-methyl-homoserine residue located at any one of residues 22-32; (f) a norleucine or O-methyl-homoserine residue located at any one of residues 49-59; (g) a norleucine or O-methyl-homoserine residue located at any one of residues 64-74; and (h) a norleucine or O-methyl-homoserine residue located at any one of residues 142-152; wherein residue position numbering of the IL-7 polypeptide is based on SEQ ID NO: 1 as a reference sequence. In some embodiments, the IL-7 polypeptide comprises one homoserine at each of (a)-(c). In some embodiments, the IL-7 polypeptide comprises 1, 2, 3, 4, 5, 6, 7, or 8 of the amino acid substitutions of (a)-(h).

In some embodiments, the IL-7 polypeptide comprises one or more amino acid substitutions selected from: (a) an O-methyl-homoserine residue located at any one of residues 12-22, (b) an O-methyl-homoserine residue located at any one of residues 22-32 (c) a homoserine residue located at any one of residues 31-41; (d) an O-methyl-homoserine residue located at any one of residues 49-59, (c) an O-methyl-homoserine residue located at any one of residues 64-74. (f) a homoserine residue located at any one of residues 71-81; (g) a homoserine residue located at any one of residues 109-119; and (h) an O-methyl-homoserine residue located at any one of residues 142-152, wherein residue position numbering of the IL-7 polypeptide is based on SEQ ID NO: 1 as a reference sequence. In some embodiments, the IL-7 polypeptide comprises 1, 2, 3, 4, 5, 6, 7, or 8 of the amino acid substitutions of (a)-(h).

In some embodiments, the IL-7 polypeptide comprises one or more amino acid substitutions selected from: (a) a homoserine residue located at any one of residues 31-41; (b) a homoserine residue located at any one of residues 71-81; (c) a homoserine residue located at any one of residues 109-119; (d) a norleucine residue located at any one of residues 12-22; (e) a norleucine residue located at any one of residues 22-32; (f) a norleucine residue located at any one of residues 49-59; (g) a norleucine residue located at any one of residues 64-74; and (h) a norleucine residue located at any one of residues 142-152; wherein residue position numbering of the IL-7 polypeptide is based on SEQ ID NO: 1 as a reference sequence. In some embodiments, the IL-7 polypeptide comprises 1, 2, 3, 4, 5, 6, 7, or 8 of the amino acid substitutions of (a)-(h).

In some embodiments, the IL-7 polypeptide comprises an amino acid substitution of at least one methionine residue in SEQ ID NO: 1. In some embodiments, the amino acid substitution of at least one methionine residue comprises a substitution at M17, M27, M54, M69, or M147. In some embodiments, the IL-7 polypeptide comprises substitutions of one, two, three, or four methionine residues. In some embodiments, the IL-7 polypeptide comprises substitutions of at least two methionine residues. In some embodiments, the IL-7 polypeptide comprises substitutions of at least three methionine residues. In some embodiments, the IL-7 polypeptide comprises substitutions of at least four methionine residues. In some embodiments, the IL-7 polypeptide comprises substitutions of all five methionine residues.

In some embodiments, one or more methionine residues in the IL-7 polypeptide of SEQ ID NO: 1 are substituted for residues that do not contain sulfur atoms. In some embodiments, one or more methionine residues are each independently substituted for a methionine isostere. In some embodiments, one or more methionine residues are each independently substituted for norleucine (Nle) or O-methyl-homoserine (Omh). In some embodiments, at least one methionine residue is substituted for a Nle or Omh residue. In some embodiments, one methionine residue is substituted for Nle on Omh residue. In some embodiments, two methionine residues are each independently substituted for Nle or Omh residues. In some embodiments, three methionine residues are each independently substituted for Nle or Omh residues. In some embodiments, four methionine residues are each independently substituted for Nle or Omh residues. In some embodiments, each methionine is independently substituted for a Nle or Omh residue.

In some embodiments, the IL-7 peptide comprises an amino acid substitution with norleucine. In some embodiments, the IL-7 peptide comprises an amino acid substitution with norleucine at positions Met 17, Met 27, Met 54, Met 69 or Met 147. In some embodiments, the IL-7 polypeptide comprises one or more amino acid substitutions selected from norleucine (Nle) 17, O-methyl-homoserine (Omh) 17, Nle 27, Omh 27, homoserine (Hsc) 36, Nle54, Omh54, Nle69, Omh69, Hse76, Hse114, Nle147, and Omh147. In some embodiments, each methionine is substituted with Nle or Omh.

In some embodiments, at least one methionine residue is substituted for a Nle residue. In some embodiments, one methionine residue is substituted for Nle residue. In some embodiments, two methionine residues are substituted for Nle residues. In some embodiments, three methionine residues are substituted for Nle residues. In some embodiments, four methionine residues are substituted for Nle residues. In some embodiments, each methionine substitution is for Nle residues.

In some embodiments, the IL-7 peptide comprises an amino acid substitution with O-methyl-L-homoserine. In some embodiments, the IL-7 peptide comprises an amino acid substitution with O-methyl-L-homoserine at positions Met 17, Met 27, Met 54, Met 69, or Met 147. In some embodiments, the IL-7 polypeptide comprises one or more amino acid substitutions selected from norleucine (Nle) 17, O-methyl-homoserine (Omh) 17, Nle27, Omh27, homoserine (Hsc) 36, Nle54, Omh54, Nle69, Omh69, Hse76, Hse114, Nle147, and Omh147.

In some embodiments, the IL-7 polypeptide is prepared from one or more chemically synthesized peptides. In some embodiments, the IL-7 polypeptide is synthesized from one or more chemically synthesized precursor fragments. In some embodiments, the IL-7 polypeptide is prepared from one or more chemically synthesized precursor fragments that are ligated together to produce the full-length IL-7 polypeptide. In some embodiments, the IL-7 polypeptide (e.g., one prepared synthetically) as provided herein is incorporated into an immunocytokine (e.g., is attached via a linker) with a polypeptide which binds specifically to an immune checkpoint inhibitor molecule (e.g., an anti-PD-1 antibody or antigen binding fragment thereof). In some embodiments, the IL-7 polypeptide as provided herein is attached via a linker moiety to an additional group, such as a polymer or an antibody or antigen binding fragment thereof. In some embodiments, chemically synthesized peptides or precursor fragments are produced by solid phase peptide synthesis.

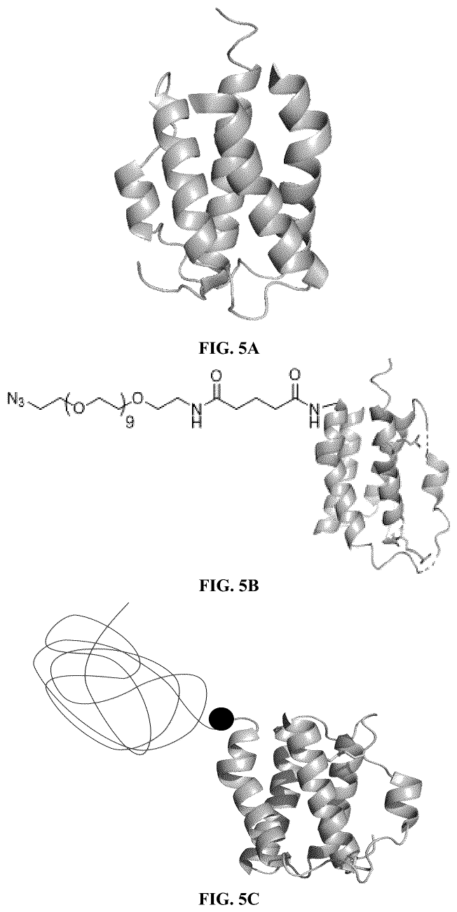

In some embodiments, the IL-7 polypeptide exhibits a similar or substantially identical activity to a corresponding recombinant IL-7 (e.g., an IL-7 having the same functional modifications to the structure or sequence of the IL-7 polypeptide but lacks homoserine, norleucine, and O-methyl-homoserine residues as provided herein). In some embodiments, the IL-7 polypeptide adopts a tertiary structure similar or substantially identical to that of wild type IL-7 (e.g., the conformation shown in FIG. 5A, which shows a 3D representation of a properly folded IL-7).

In some embodiments, the IL-7 polypeptide is prepared from one or more chemically synthesized fragments. In some embodiments, the IL-7 polypeptide is prepared from 1, 2, 3, 4, 5, 6, 7, 8, or more chemically synthesized fragments. In some embodiments, the IL-7 polypeptide is prepared from 4 chemically synthesized fragments. In some embodiments, the IL-7 polypeptide is prepared from 4 or 5 chemically synthesized fragments.

In some embodiments, the IL-7 polypeptide comprises an amino acid sequence at least 80%, 85%, 90%, 95%, 99%, or 100% identical to SEQ ID NO: 3. In some embodiments, the IL-7 polypeptide comprises an amino acid sequence of SEQ ID NO: 3. In some embodiments, the IL-7 polypeptide consists of an amino acid sequence at least 80%, 85%, 90%, 95%, or 99%, or 100% identical to the sequence of SEQ ID NO: 3.

In some embodiments, the IL-7 polypeptide comprises an amino acid sequence at least about 75% identical to that of SEQ ID NO: 3. In some embodiments, the IL-7 polypeptide comprises an amino acid sequence at least about 80% identical to that of SEQ ID NO: 3. In some embodiments, the IL-7 polypeptide comprises an amino acid sequence at least about 85% identical to that of SEQ ID NO: 3. In some embodiments, the IL-7 polypeptide comprises an amino acid sequence at least about 90% identical to that of SEQ ID NO: 3. In some embodiments, the IL-7 polypeptide comprises an amino acid sequence at least about 95% identical to that of SEQ ID NO: 3. In some embodiments, the IL-7 polypeptide comprises an amino acid sequence at least about 96% identical to that of SEQ ID NO: 3. In some embodiments, the IL-7 polypeptide comprises an amino acid sequence at least about 97% identical to that of SEQ ID NO: 3. In some embodiments, the IL-7 polypeptide comprises an amino acid sequence at least about 98% identical to that of SEQ ID NO: 3. In some embodiments, the IL-7 polypeptide comprises an amino acid sequence at least about 99% identical to that of SEQ ID NO: 3. In some embodiments, the IL-7 polypeptide comprises an amino acid sequence identical to that of SEQ ID NO: 3.

In some embodiments, the IL-7 polypeptide comprises an amino acid sequence at least 80%, 85%, 90%, 95%, 99%, or 100% identical to any one of SEQ ID NOs: 4-12. In some embodiments, the IL-7 polypeptide comprises an amino acid sequence of SEQ ID NO: 4. In some embodiments, the IL-7 polypeptide comprises an amino acid sequence of SEQ ID NO: 5. In some embodiments, the IL-7 polypeptide comprises an amino acid sequence of SEQ ID NO: 6. In some embodiments, the IL-7 polypeptide comprises an amino acid sequence of SEQ ID NO: 7. In some embodiments, the IL-7 polypeptide comprises an amino acid sequence of SEQ ID NO: 8. In some embodiments, the IL-7 polypeptide comprises an amino acid sequence of SEQ ID NO: 9. In some embodiments, the IL-7 polypeptide comprises an amino acid sequence of SEQ ID NO: 10. In some embodiments, the IL-7 polypeptide comprises an amino acid sequence of SEQ ID NO: 11. In some embodiments, the IL-7 polypeptide comprises an amino acid sequence of SEQ ID NO: 12. In some embodiments, the IL-7 polypeptide consists of an amino acid sequence at least 80%, 85%, 90%, 95%, 99%, or 100% identical to the sequence of SEQ ID NO: 4-12.

In some embodiments, the synthetic IL-7 polypeptide comprises an amino acid sequence at least 80%, 85%, 90%, or 95% identical to SEQ ID NO: 1.

### Activity of IL-7 Polypeptides

In some embodiments, the binding affinity between the IL-7 polypeptide and the IL-7Rα/γ_{c} heterodimer is the same as or lower than the binding affinity between a wild-type IL-7 and the IL-7Rα/γ_{c} heterodimer.

In some embodiments, the binding affinity between the IL-7 polypeptide and IL-7 receptor alpha subunit (IL-7Rα) is the same as or lower than the binding affinity between a wild-type IL-7 and IL-7Rα.

In some embodiments, IL-7 polypeptides described herein have decreased affinity for the IL-7 alpha subunit (IL-7Rα) compared to wild type IL-7. In some embodiments, IL-7 polypeptides described herein have decreased affinity for the IL-7Rα/γ_{c} heterodimer (IL-7R) compared to wild type IL-7. In some instances, IL-7 polypeptides provided herein have decreased affinity for both IL-7R and IL-7Rαcompared to wild type IL-7. In some embodiments, IL-7 polypeptides provided herein retain specific binding to IL-7R and IL-7Rα, but have a decreased affinity as compared to wild type IL-7.

### Binding Affinity

In some embodiments, the affinity of an IL-7 polypeptide to IL-7Rα and/or IL-7Rα/γ_{c} heterodimer is measured by a dissociation constant (K_{D}). As used herein, the phrase “the K_{D} of the IL-7 polypeptide/IL-7Rα” means the dissociation constant of the binding interaction of the IL-7 polypeptide and IL-7Rα. The phrase “the K_{D} of the IL-7 polypeptide/IL-7Rα/γ.” means the dissociation constant of the binding interaction of the IL-7 polypeptide and the IL-7Rα/γ_{c} heterodimer.

In some embodiments, the IL-7 polypeptide binds to IL-7Rα. In some embodiments, the IL-7 polypeptide has a K_{D} that is greater than the K_{D} of WT IL-7. In some embodiments, the IL-7 polypeptide binds to IL-7Rα with a K_{D} of less than about 50 nM. In some embodiments, the IL-7 polypeptide binds to IL-7Rα with a K_{D} of less than about 10 nM. In some embodiments, the IL-7 polypeptide binds to an IL-7Rα/γ_{c} heterodimer. In some embodiments, the IL-7 polypeptide binds to the IL-7Rα/γ_{c} heterodimer with a K_{D} of less than about 10 nM. In some embodiments, the IL-7 polypeptide binds to the IL-7Rα/γ_{c} heterodimer with a K_{D} of less than about 2 nM.

In some embodiments, the K_{D} of the IL-7 polypeptide/IL-7Rα is less than 1000 nM, less than 750 nM, less than 500 nM, less than 450, less than 400 nM, less than 350 nM, less than 300 nM, less than 250 nM, less than 200 nM, less than 150 nM, less than 140 nM, less than 130 nM, less than 125 nM, less than 120 nM, less than 100 nM. In some embodiments, the K_{D} of the IL-7 polypeptide/IL-7Rα is less than 150 nM, less than 50 nM, less than 25 nM, or less than 10 nM. In some embodiments, the K_{D} of the IL-7 polypeptide/IL-7Rα is less than 50 nM. In some embodiments, the K_{D} of the IL-7 polypeptide/IL-7Rα is less than 10 nM.

In some embodiments, the K_{D} of the IL-7 polypeptide/IL-7Rα/γ_{c} heterodimer is less than 1000 nM, less than 750 nM, less than 500 nM, less than 450, less than 400 nM, less than 350 nM, less than 300 nM, less than 250 nM, less than 200 nM, less than 150 nM, less than 140 nM, less than 130 nM, less than 125 nM, less than 120 nM, less than 100 nM. In some embodiments, the K_{D} of the IL-7 polypeptide/IL-7Rα/γ_{c} heterodimer is less than 150 nM, less than 50 nM, less than 25 nM, less than 10 nM, less than 5 nM, or less than 2 nM. In some embodiments, the K_{D} of the IL-7 polypeptide/IL-7Rα/γ_{c} heterodimer is less than 50 nM. In some embodiments, the K_{D} of the IL-7 polypeptide/IL-7Rα/γ_{c} heterodimer is less than 10 nM. In some embodiments, the K_{D} of the IL-7 polypeptide/IL-7Rα/γ_{c} heterodimer is less than 5 nM.

In some embodiments, the K_{D} of an IL-7 polypeptide/IL-7Rα is substantially the same as the K_{D} of wild-type IL-7/IL-7Rα. In some embodiments, the K_{D} of the IL-7 polypeptide/IL-7Rα is greater than the K_{D} of wild-type IL-7/IL-7Rα. In some embodiments, the K_{D} of the IL-7 polypeptide/IL-7Rα is at most 10%, at most 20%, at most 30%, at most 40%, at most 50%, at most 60%, at most 70%, at most 80%, at most 85%, or at most 90% greater than the K_{D} of wild-type IL-7/IL-7Rα. In some embodiments, the K_{D} of the IL-7 polypeptide/IL-7Rα is at least 20% greater than the K_{D} of wild-type IL-7/IL-7Rα. In some embodiments, the K_{D} of the IL-7 polypeptide/IL-7Rα is at least 25% greater than the K_{D} of wild-type IL-7/IL-7Rα.

In some embodiments, the K_{D} of the IL-7 polypeptide/IL-7Rα is at least 100%, at least 200%, at least 300%, at least 400%, at least 500%, or at least 600% greater than the K_{D} of wild-type IL-7/IL-7Rα. In some embodiments, the K_{D} of the IL-7 polypeptide/IL-7Rα is at least 500% greater than the K_{D} of wild-type IL-7/IL-7Rα. In some embodiments, the K_{D} of the IL-7 polypeptide/IL-7Rα is about 500% greater than the K_{D} of wild-type IL-7/IL-7Rα.

In some embodiments, the K_{D} of the IL-7 polypeptide/IL-7Rα/γ_{c} heterodimer is at least 100%, at least 200%, at least 300%, at least 400%, at least 500% greater than the K_{D} of wild-type IL-7/IL-7Rα/γ_{c} heterodimer. In some embodiments, the K_{D} of the IL-7 polypeptide/IL-7Rα/γ_{c} heterodimer is at least 350% greater than the K_{D} of wild-type IL-7/IL-7Rα/γ_{c} heterodimer.

### T-Cell Signaling

In some embodiments, the IL-7 polypeptide as provided herein displays a modified ability to stimulate one or more T-cell subtypes as compared to WT IL-7. In some embodiments, the modified ability to stimulate the one or more T-cell subtypes stems from a modified ability of the IL-7 polypeptide to bind to the IL-7 receptor (IL-7R) or at least one subunit of the IL-7R, such as the alpha subunit (IL-7Rα) or the IL-7Rα/γ_{c} heterodimer complex. In some embodiments, the IL-7 polypeptide can retain binding to IL-7Rα. In some embodiments, the IL-7 polypeptide can exhibit increased binding to IL-7Rα. In some embodiments, the IL-7 polypeptide can exhibit reduced binding to IL-7Rα. In some embodiments, the IL-7 polypeptide can retain binding to the IL-7Rα/γ_{c} heterodimer. In some embodiments, the IL-7 polypeptide can have increased binding to the IL-7Rα/γ_{c} heterodimer. In some embodiments, the IL-7 polypeptide can have reduced binding to the IL-7Rα/γ_{c} heterodimer. In some embodiments, the one or more T-cell subtype stimulated by the IL-7 polypeptide is a CD4 T_{reg} cell, a CD8 Naïve T cell, a CD8 Memory cell, a CD4 Naïve cell, or a CD4 Memory cell. In some embodiments, the one or more T-cell subtype stimulated by the IL-7 polypeptide is a CD4 T_{reg} cell.

In some embodiments, IL-7 engagement with the IL-7Rα/γ_{c} heterodimer correlates with STAT5 phosphorylation. In some embodiments, IL-7 engagement with the IL-7Rα/γ_{c} heterodimer leads to JAK1 and JAK3 kinase activation. In some embodiments JAK kinase activation correlates with STAT5 phosphorylation (pSTAT5) and modification of transcription regulated by pSTAT5.

In some embodiments, the IL-7 can retain pSTAT5 induction compared to wild type IL-7 in one or more T-cell subtypes. In some embodiments, the IL-7 polypeptide can exhibit increased pSTAT5 induction compared to wild type IL-7 in one or more T-cell subtypes. In some embodiments, the IL-7 polypeptide can exhibit decreased pSTAT5 induction compared to wild type IL-7 in one or more T-cell subtypes. In some embodiments, the one or more T-cell subtype is a CD4 T_{reg} cell, a CD8 Naïve T cell, a CD8 Memory cell, a CD4 Naïve cell, or a CD4 Memory cell. In some embodiments, the one or more T-cell subtype is a CD4 T_{reg} cell.

In some embodiments, the IL-7 polypeptide displays an enhanced selectivity for stimulation of T-effector (T_{eff}) cells over T-regulatory (T_{reg}) cells as compared to a wild type IL-7 polypeptide of SEQ ID NO: 1. In some embodiments, the selectivity is determined by comparing a ratio of EC₅₀ values for stimulation of one or more T_{eff} cells (e.g., one or more of a CD8 Naïve T cell, a CD8 Memory cell, a CD4 Naïve cell, or a CD4 Memory cell) versus stimulation of one or more T_{reg} cells (e.g., a CD4 T_{reg} cell) as compared to the ratio for a WT IL-7. In some embodiments, the ratio reflects stimulation of a CD8 Naïve T cell compared to stimulation of a CD4 T_{reg} cell. In some embodiments, the ratio reflects stimulation of a CD8 Memory cell compared to stimulation of a CD4 T_{reg} cell. In some embodiments, the ratio reflects stimulation of a CD4 Naïve cell compared to stimulation of a CD4 T_{reg} cell. In some embodiments, the ratio reflects stimulation of a CD4 Memory cell compared to stimulation of a CD4 T_{reg} cell.

In some embodiments, the ratio of stimulation of the one or more T_{eff} cells over the stimulation of a T_{reg} cell by the IL-7 polypeptide is at least about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200% greater compared to the same ratio for a WT IL-7 polypeptide. In some embodiments, the ratio of stimulation of the one or more T_{eff} cells over the stimulation of a T_{reg} cell by the IL-7 polypeptide is at least about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, or about 30-fold greater compared to the same ratio for a WT IL-7 polypeptide.

In some embodiments, the IL-7 polypeptide can increase stimulation of T_{eff} compared to stimulation of T_{reg} cells. In some embodiments, an IL-7 polypeptide can increase stimulation of T_{eff} by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200% compared to stimulation of T_{reg} cells. In some embodiments, the IL-7 polypeptide can increase stimulation of T_{eff} by about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, or about 30-fold compared to stimulation of T_{reg} cells. In some embodiments, the IL-7 polypeptide can increase stimulation of T_{eff} by about 3-fold compared to stimulation of T_{reg} cells. In some embodiments, the IL-7 polypeptide can increase stimulation of T_{eff} by about 10-fold compared to stimulation of T_{reg} cells. In some embodiments, the IL-7 polypeptide can increase stimulation of T_{eff} by about 30-fold compared to stimulation of T_{reg} cells. In some embodiments, the comparison is made at the same concentration of the IL-7 polypeptide. In some embodiments, the increased stimulation of the T-cells is measured in an in vitro T-cell stimulation assay (e.g., pSTAT5 phosphorylation assay). In some embodiments, the increased stimulation of the T-cells is measured in an in vivo assay.

In some embodiments, the IL-7 polypeptide can increase stimulation of T_{eff} compared to WT IL-7. In some embodiments, the IL-7 polypeptide can increase stimulation of T_{eff} compared to WT IL-7 by about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold. In some embodiments, the IL-7 polypeptide can increase stimulation of T_{eff} compared to WT IL-7 by about 5-fold. In some embodiments, the IL-7 polypeptide can increase stimulation of T_{eff} compared to WT IL-7 by about 10-fold. In some embodiments, the comparison is made between the IL-7 polypeptide and the WT IL-7 at the same concentration. In some embodiments, the increased stimulation of the T-cells is measured in an in vitro T-cell stimulation assay (e.g., pSTAT5 phosphorylation assay). In some embodiments, the increased stimulation of the T-cells is measured in an in vivo assay.

In some embodiments, the IL-7 polypeptide displays a greater half maximal effective concentration (EC50) value of pSTAT5 induction of at least one primary human T cell as compared to an EC50 value of an IL-7 polypeptide of SEQ ID NO: 1, wherein the EC50 value of pSTAT5 induction is measured by a pSTAT5 phosphorylation assay. In some embodiments, the IL-7 polypeptide of SEQ ID NO: 1 is a wild type IL-7.

In some embodiments, the at least one primary human T cell is a CD4 Treg cell. In some embodiments, the EC50 value of the modified the IL-7 polypeptide of pSTAT5 induction of a CD4 Treg cell is at least about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold higher than the EC50 value of pSTAT5 induction of a CD8 Naïve T cell, a CD8 Memory cell, a CD4 Naïve cell, or a CD4 Memory cell. In some embodiments, the EC50 value of the modified the IL-7 polypeptide of pSTAT5 induction of a CD4 Treg cell is at least 5-fold higher than the EC50 value of pSTAT5 induction of a CD8 Naïve T cell, a CD8 Memory cell, a CD4 Naïve cell, or a CD4 Memory cell. In some embodiments, the EC50 value of the modified the IL-7 polypeptide of pSTAT5 induction of a CD4 Treg cell is at least 10-fold higher than the EC50 value of pSTAT5 induction of a CD8 Naïve T cell, a CD8 Memory cell, a CD4 Naïve cell, or a CD4 Memory cell. In some embodiments, the EC50 value of the modified the IL-7 polypeptide of pSTAT5 induction of a CD4 Treg cell is at least 30-fold higher than the EC50 value of pSTAT5 induction of a CD8 Naïve T cell, a CD8 Memory cell, a CD4 Naïve cell, or a CD4 Memory cell. In some embodiments, the EC50 value of the modified the IL-7 polypeptide of pSTAT5 induction of a CD4 Treg cell is higher than each of a CD8 Naïve T cell a CD8 Memory cell, a CD4 Naïve cell, and a CD4 Memory cell. In some embodiments, the EC50 value of the modified the IL-7 polypeptide of pSTAT5 induction of a CD4 Treg cell is higher than a CD8 Naïve T cell. In some embodiments, the EC50 value of the modified the IL-7 polypeptide of pSTAT5 induction of a CD4 Treg cell is higher than a CD8 Memory cell. In some embodiments, the EC50 value of the modified the IL-7 polypeptide of pSTAT5 induction of a CD4 Treg cell is higher than a CD4 Naïve cell. In some embodiments, the EC50 value of the modified the IL-7 polypeptide of pSTAT5 induction of a CD4 Treg cell is higher than a CD4 Memory cell.

### Preparation of Synthetic IL-7 Polypeptides

Also provided herein is a method synthesizing an IL-7 polypeptide, such as any of the IL-7 polypeptides provided herein. In some cases, the IL-7 polypeptide is synthesized chemically rather than recombinantly expressed. In some instances, several fragment peptide precursors of the IL-7 polypeptide are prepared and subsequently ligated together using a suitable ligation methodology (e.g., alpha-keto acid hydroxylamine (KAHA) ligation). In some cases, after ligation, the resulting IL-7 polypeptide is folded to produce an IL-7 polypeptide having a secondary and tertiary structure substantially identical to that of a recombinant or wild type IL-7 polypeptide.

In one aspect, described herein, is a method of making a synthetic IL-7 polypeptide. In some embodiments, the IL-7 polypeptide is synthesized by ligating together precursor peptide fragments of the full length IL-7 polypeptide. After ligation of the separate peptides to form a linear, full length IL-7 polypeptide (or desired variant), the linear peptide is desirably folded to a conformation which is substantially identical to wild type IL-7, or to a substantially identical conformation of IL-7 performed by recombinant means.

In one aspect, provided herein, is a method of making a synthetic IL-7 polypeptide, comprising: a) providing two or more fragments of the IL-7 polypeptide; and b) ligating the fragments. In some embodiments, the IL-7 polypeptide comprises at least one modification to the amino acid sequence as set forth in SEQ ID NO: 1, wherein the at least one modification is a natural amino acid substitution or an additional group covalently attached to a side chain of an amino acid residue of the IL-7 polypeptide.

In one aspect, provided herein, is a method of making a synthetic IL-7 polypeptide, comprising: a) providing two or more fragments of the IL-7 polypeptide; b) ligating the fragments; and c) folding the ligated fragments.

In some embodiments, the method comprises providing two or more fragments. In some embodiments, the two or more fragments are precursor peptides containing a subset of the amino acids of the full length polypeptide. In some embodiments, the two or more fragments are precursor peptides that have been synthesized chemically. In some embodiments, at least one, two, or all of the fragments are chemically synthesized precursor peptides.

In some embodiments, providing two or more fragments comprises synthesizing the two or more fragments. In some embodiments, the two or more fragments of the IL-7 polypeptide are synthesized by solid phase peptide synthesis. In some embodiments, the two or more fragments of the IL-7 polypeptide are synthesized on an automated peptide synthesizer. In some embodiments, the two or more fragments are synthesized containing protecting groups which are removed at a desired stage of making the full length IL-7 polypeptide. In some embodiments, protecting groups are removed prior to ligating the fragments. In some embodiments, protecting groups are removed after folding the ligated fragments. In some embodiments, certain protecting groups are removed at one stage (e.g., prior to ligating the fragments) and certain other protecting groups are removed at a second stage (e.g., after folding the ligated fragments).

In some embodiments, the IL-7 polypeptide is ligated from 2, 3, 4, 5, 6, 7, 8, 9, 10, or more fragments. In some embodiments, the modified peptide is ligated from 2 fragments. In some embodiments, the IL-7 polypeptide is ligated from 3 fragments. In some embodiments, the IL-7 polypeptide is ligated from 4 fragments. In some embodiments, the IL-7 polypeptide is ligated from 2 to 10 fragments. In some embodiments, the IL-7 polypeptide is ligated from 2 to 4 fragments.

In some embodiments, the two or more fragments comprise an N-terminal fragment, a C-terminal fragment, and optionally one or more interior fragments. In some embodiments, the N-terminal fragment comprises the N-terminus of the IL-7 polypeptide. In some embodiments, the C-terminal fragment comprises the C-terminus of the IL-7 polypeptide. In some embodiments, the two or more fragments comprise 0, 1, 2, 3, 4, 5, 6, 7, or 8 interior fragments. In some embodiments, the two or more fragments comprise 0 interior fragments. In some embodiments, the two or more fragments comprise 2 interior fragments. In some embodiments, the two or more fragments comprise 1 interior fragment.

When assembling a synthetic IL-7 polypeptide using one or more interior fragments, the fragments should be ligated together such that the desired final amino acid sequence is achieved. While there is an order in which the two or more fragments must be finally assembled in the final product, the fragments themselves may be assembled in any desired order. For example, for an IL-7 polypeptide made of 4 fragments (an N-terminal fragment, a first interior fragment, a second interior fragment, and a C-terminal fragment), the 4 fragments can be ligated in any order. For example, in some embodiments, the N-terminal fragment is ligated first to the first interior fragment, and the second interior fragment is ligated to the C-terminal fragment. Then, in some embodiments, the ligated N-terminal fragment/first interior fragment is then then ligated to the second interior fragment/C-terminal fragment previously ligated to form the full length IL-7 polypeptide. Alternatively, the fragments could be assembled in a different order, such as the N-terminal fragment ligated to the first interior fragment, followed by a ligation of the ligated N-terminal/first interior fragment to the second interior fragment, followed by a subsequent ligation of the C-terminal fragment. While the fragments can be assembled in any desired order, considerations of compatible protecting group strategies should be considered.

Generally, the two or more fragments each comprise the necessary reactive groups in order to allow ligation of each fragment to the other fragments. The required reactive group depends on the ligation chemistry being used for the ligation. In embodiments wherein the synthetic polypeptide is ligated using alpha-keto acid hydroxylamine (KAHA) ligation, each fragment must have one or, in the case of an interior fragment, both of a) an alpha-keto amino acid; and b) a hydroxylamine containing moiety or a suitable precursor (e.g., a cyclized hydroxylamine such as 5-oxaproline). Where other ligation methods are used, different reactive group pairs are necessary (e.g., native chemical ligation (C-terminal thioester/N-terminal cysteine); potassium acyltrifluoroborate (KAT) ligation (C-terminal potassium acyltrifluoroborate/N-terminal hydroxylamine); bis(2-sulfanylethyl)amido (SEA) ligation (C-terminal bis(2-sulfanylethyl)amide/N-terminal cysteine); serine/threonine ligation (C-terminal salicylaldehyde ester/N-terminal serine or threonine); Staudinger ligation (C-terminal ester/N-terminal azide); etc.).

In some embodiments, each of the N-terminal fragment and any interior fragments of the IL-7 comprise the required C-terminal reactive group for the desired ligation at or attached to the C-terminal residue. In some embodiments, each of the N-terminal fragment and any of the interior fragments (e.g., the first interior fragment and the second interior fragment) comprise an alpha-keto amino acid as the C-terminal residue of each fragment. In some embodiments, the alpha-keto amino acid is the alpha-keto version of the natural amino acid at the corresponding position in SEQ ID NO: 1 in the full length IL-7 polypeptide. In some embodiments, the alpha-keto amino acid is an amino acid substitution (e.g., has a different side chain compared to the natural amino acid) relative to the corresponding position in SEQ ID NO: 1. Where the synthetic IL-7 is made from more than two peptides, each of the N-terminal fragment and any interior fragments may comprise different alpha-keto amino acids. In some embodiments, the alpha-keto acid comprises a side chain of a natural or unnatural amino acid. In some embodiments, each alpha-keto amino acid is an aliphatic or aromatic amino acid. In some embodiments, each alpha-keto amino acid is independently selected from alpha-keto-phenylalanine, alpha-keto-tyrosine, alpha-keto-tryptophan, alpha-keto alanine, alpha-keto-beta-alanine, alpha-keto-proline, alpha-keto-valine, alpha-keto-leucine, alpha-keto-isoleucine, alpha-keto-norleucine, alpha-keto-methionine, alpha-keto-serine, alpha-keto-threonine, and alpha-keto-O-methylhomoserine, or any derivative thereof. In some embodiments, each alpha-keto amino acid is independently selected from alpha-keto-phenylalanine, alpha-keto-tyrosine, alpha-keto-valine, alpha-keto-leucine, alpha-keto-isoleucine, alpha-keto-norleucine, and alpha-keto-O-methylhomoserine, or any derivative thereof. In some embodiments, each alpha-keto amino acid is independently selected from alpha-keto-phenylalanine, alpha-keto-tyrosine, alpha-keto-valine, alpha-keto-leucine, alpha-keto-isoleucine, alpha-keto-norleucine, and alpha-keto-O-methylhomoserine.

In some embodiments, each of the C-terminal fragment and any interior fragments of the IL-7 comprise the required N-terminal reactive group for the desired ligation at or attached to the N-terminal residue. In some embodiments, each of the C-terminal fragment and any of the interior fragments (e.g., the first interior fragment and the second interior fragment) comprise a residue having a hydroxylamine or a cyclic hydroxylamine functionality as the N-terminal residue of each fragment. Where the synthetic IL-7 is made from more than two peptides, each of the C-terminal fragment and any interior fragments may comprise different residues having a hydroxylamine or a cyclic hydroxylamine functionality as the N-terminal residue. In some embodiments, each residue having a hydroxylamine or a cyclic hydroxylamine functionality is independently 5-oxaproline or 1,2-oxazetidine-3-carboxylic acid. In some embodiments, each residue having a hydroxylamine or a cyclic hydroxylamine functionality is 5-oxaproline.

In some embodiments, providing two or more fragments of the synthetic IL-7 polypeptide comprises providing four fragments. In some embodiments, the four fragments comprise an N-terminal fragment, a first interior fragment, a second interior fragment, and a C-terminal fragment. In some embodiments, the four fragments are arranged in the following order from N-terminus to C-terminus of the full length IL-7 polypeptide: the N-terminal fragment, the first interior fragment, the second interior fragment, and the C-terminal fragment. In some embodiments, the N-terminal fragment, the first interior fragment, the second interior fragment, and the C-terminal fragment are arranged from the N-terminus to the C-terminus, respectively, in the IL-7 polypeptide.

In some embodiments, the two or more fragments of the IL-7 polypeptide comprise an N-terminal fragment. In some embodiments, the N-terminal fragment comprises residues which correspond to amino acids 1-46, amino acids 1-41, amino acids 1-39, amino acids 1-37, amino acids 1-36, amino acids 1-35, amino acids 1-34, amino acids 1-33, or amino acids 1-31 of the IL-7 polypeptide. In some embodiments, the IL-7 polypeptide comprises a truncation of residues from the N-terminus of the amino acid sequence as set forth in SEQ ID NO: 1. In such cases, the N-terminal fragment will contain fewer of the relevant amino acids (e.g., 1, 2, 3, 4, 5, 6, 7, 8, 9, 10, or more amino acids removed). Thus, in some embodiments, the N-terminal fragment comprises residues which correspond to amino acids 2-35, 3-35, 4-35, 5-35, 6-35, 7-35, 8-35, 9-35, or 10-35, or any other relevant N-terminal truncation.

In some embodiments, ligating the fragments forms a bond between two amino acid residues at a position in one or more regions. In some embodiments, ligating the fragments forms a bond between two amino acid residues at a position in one, two, or three of the regions. In some embodiments, ligating the fragments forms a bond between two amino acid residues at positions in each of the three regions. In some embodiments, the regions are selected from residues 26-46, residues 66-86, and residues 104-124, wherein residue position numbering of the IL-7 polypeptide is based on SEQ ID NO: 1 as a reference sequence. In some embodiments, the regions are selected from residues 31-41, residues 71-81, and residues 109-119. In some embodiments, the regions are selected from residues 33-39, residues 73-79, and residues 111-117. In some embodiments, the regions are selected from residues 35-37, residues 75-77, and residues 113-115. In some embodiments, ligating the fragments forms a bond between residues 35/36, residues 75/76, residues 113/114, or any combination thereof. In some embodiments, ligating the fragments forms a bond between two or more sets of residues selected from residues 35/36, residues 75/76, and residues 113/114. In some embodiments, ligating the fragments forms a bond between each of residues 35/36, residues 75/76, and residues 113/114.

In some embodiments, the N-terminal fragment comprises residues which correspond to amino acids 1-35 of the IL-7 polypeptide, wherein residue position numbering of the IL-7 polypeptide is based on SEQ ID NO: 1 as a reference sequence. In some embodiments, the N-terminal fragment comprises an N-terminal extension as compared to the sequence of SEQ ID NO: 1. In some embodiments, the N-terminal fragment comprises an N-terminal truncation as compared to the sequence of SEQ ID NO: 1. In some embodiments, the N-terminal fragment comprises an amino acid sequence having at least 60% sequence identity with the amino acid sequence as set forth in SEQ ID NO: 101. In some embodiments, the N-terminal fragment comprises an amino acid sequence having at least 70% sequence identity with the amino acid sequence as set forth in SEQ ID NO: 101. In some embodiments, the N-terminal fragment comprises an amino acid sequence having at least 75% sequence identity with the amino acid sequence as set forth in SEQ ID NO: 101. In some embodiments, the N-terminal fragment comprises an amino acid sequence having at least 80% sequence identity with the amino acid sequence as set forth in SEQ ID NO: 101. In some embodiments, the N-terminal fragment comprises an amino acid sequence having at least 85% sequence identity with the amino acid sequence as set forth in SEQ ID NO: 101. In some embodiments, the N-terminal fragment comprises an amino acid sequence having at least 90% sequence identity with the amino acid sequence as set forth in SEQ ID NO: 101. In some embodiments, the N-terminal fragment comprises an amino acid sequence having at least 95% sequence identity with the amino acid sequence as set forth in SEQ ID NO: 101. In some embodiments, the N-terminal fragment comprises an amino acid sequence having a sequence as set forth in any one of SEQ ID NOs: 101-105.

In some embodiments, the first interior fragment comprises residues which correspond to amino acids 36-75 of the IL-7 polypeptide, wherein residue position numbering of the IL-7 polypeptide is based on SEQ ID NO: 1 as a reference sequence. In some embodiments, the first interior fragment comprises an amino acid sequence having at least 60% sequence identity with the amino acid sequence as set forth in SEQ ID NO: 106. In some embodiments, the first interior fragment comprises an amino acid sequence having at least 70% sequence identity with the amino acid sequence as set forth in SEQ ID NO: 106. In some embodiments, the first interior fragment comprises an amino acid sequence having at least 75% sequence identity with the amino acid sequence as set forth in SEQ ID NO: 106. In some embodiments, the first interior fragment comprises an amino acid sequence having at least 80% sequence identity with the amino acid sequence as set forth in SEQ ID NO: 106. In some embodiments, the first interior fragment comprises an amino acid sequence having at least 85% sequence identity with the amino acid sequence as set forth in SEQ ID NO: 106. In some embodiments, the first interior fragment comprises an amino acid sequence having at least 90% sequence identity with the amino acid sequence as set forth in SEQ ID NO: 106. In some embodiments, the first interior fragment comprises an amino acid sequence having at least 95% sequence identity with the amino acid sequence as set forth in SEQ ID NO: 106. In some embodiments, the first interior fragment comprises an amino acid sequence having a sequence as set forth in any one of SEQ ID NOs: 106-108.

In some embodiments, the second interior fragment comprises residues which correspond to amino acids 76-113 of the IL-7 polypeptide, wherein residue position numbering of the IL-7 polypeptide is based on SEQ ID NO: 1 as a reference sequence. In some embodiments, the second interior fragment comprises an amino acid sequence having at least 60% sequence identity with the amino acid sequence as set forth in SEQ ID NO: 109. In some embodiments, the second interior fragment comprises an amino acid sequence having at least 70% sequence identity with the amino acid sequence as set forth in SEQ ID NO: 109. In some embodiments, the second interior fragment comprises an amino acid sequence having at least 75% sequence identity with the amino acid sequence as set forth in SEQ ID NO: 109. In some embodiments, the second interior fragment comprises an amino acid sequence having at least 80% sequence identity with the amino acid sequence as set forth in SEQ ID NO: 109. In some embodiments, the second interior fragment comprises an amino acid sequence having at least 85% sequence identity with the amino acid sequence as set forth in SEQ ID NO: 109. In some embodiments, the second interior fragment comprises an amino acid sequence having at least 90% sequence identity with the amino acid sequence as set forth in SEQ ID NO: 109. In some embodiments, the second interior fragment comprises an amino acid sequence having at least 95% sequence identity with the amino acid sequence as set forth in SEQ ID NO: 109. In some embodiments, the second interior fragment comprises an amino acid sequence having a sequence as set forth in any one of SEQ ID NOs: 109-115.

In some embodiments, the C-terminal fragment comprises residues which correspond to amino acids 114-152 of the IL-7 polypeptide, wherein residue position numbering of the IL-7 polypeptide is based on SEQ ID NO: 1 as a reference sequence. In some embodiments, the C-terminal fragment comprises a C-terminal extension as compared to the sequence of SEQ ID NO: 1. In some embodiments, the C-terminal fragment comprises a C-terminal truncation as compared to the sequence of SEQ ID NO: 1. In some embodiments, the C-terminal fragment comprises an amino acid sequence having at least 60% sequence identity with the amino acid sequence as set forth in SEQ ID NO: 116. In some embodiments, the C-terminal fragment comprises an amino acid sequence having at least 70% sequence identity with the amino acid sequence as set forth in SEQ ID NO: 116. In some embodiments, the C-terminal fragment comprises an amino acid sequence having at least 75% sequence identity with the amino acid sequence as set forth in SEQ ID NO: 116. In some embodiments, the C-terminal fragment comprises an amino acid sequence having at least 80% sequence identity with the amino acid sequence as set forth in SEQ ID NO: 116. In some embodiments, the C-terminal fragment comprises an amino acid sequence having at least 85% sequence identity with the amino acid sequence as set forth in SEQ ID NO: 116. In some embodiments, the C-terminal fragment comprises an amino acid sequence having at least 90% sequence identity with the amino acid sequence as set forth in SEQ ID NO: 116. In some embodiments, the C-terminal fragment comprises an amino acid sequence having at least 95% sequence identity with the amino acid sequence as set forth in SEQ ID NO: 116. In some embodiments, the C-terminal fragment comprises an amino acid sequence having a sequence as set forth in any one of SEQ ID NOs: 116-119.

Table 4A below shows exemplary peptides which can be sued to synthesize and IL-7 polypeptide as described herein.

TABLE 4A
Exemplary Precursor Peptides of IL-7 Polypeptides

| SEQ ID | | |
|---|---|---|
| NO | Description | Sequence |
| 101 | IL-7 | DCDIEGKDGK QYESVLXVSI |
| | 1-35 (N-terminal | DQLLDSXKEI GSNC(Akl) |
| | fragment) | |
| | M17Nle, M27Nle | |
| 102 | IL-7 | DCDIEGKDGK QYESVLMVSI |
| | 1-35 (N-terminal | DQLLDSMKEI GSNC(Akl) |
| | fragment) | |
| 103 | IL-7 | DCDIEGKDGK QYESWLXVSI |
| | 1-35 (N-terminal | DQLLDSXKEI GSNC(Akl) |
| | fragment) | |
| | V15W, M17Nle, | |
| | M27Nle | |
| 104 | IL-7 | DSDIEGKDGK QYESVLXVSI |
| | 1-35 (N-terminal | DQLLDSXKEI GSNC(Akl) |
| | fragment) | |
| | C2S, M17Nle, M27Nle | |
| 105 | IL-7 | DCDIEGKDGK QYESVLXVSI |
| | 1-35 (N-terminal | DQLLDSXKEI GSNS(Akl) |
| | fragment) | |
| | M17Nle, M27Nle, | |
| | C34S | |
| 106 | IL-7 | (Opr)NEFN FFKRHICDAN |
| | 36-75 (First interior | KEGXFLFRAA RKLRQFLKXN |
| | fragment) | STGD(Akf) |
| | M54Nle, M69Nle | |
| 107 | IL-7 | (Opr)NEFN FFKRHICDAN |
| | 36-75 (First interior | KEGMFLFRAA RKLRQFLKMN |
| | fragment) | STGD(Akf) |
| 108 | IL-7 | (Opr)NEFN FFKRHISDAN |
| | 36-75 (First interior | KEGXFLFRAA RKLRQFLKXN |
| | fragment) | STGD(Akf) |
| | C47S, M54Nle, | |
| | M69Nle | |
| 109 | IL-7 | (Opr)LHLL KVSEGTTILL |
| | 76-113 (Second | NCTGQVKGRK PAALGEAQPT |
| | interior fragment) | KS(Akl) |
| 110 | IL-7 | (Opr)LHLL OVSEGTTILL |
| | 76-113 (Second | NCTGQVKGRK PAALGEAQPT |
| | interior fragment) | KS(Akl) |
| | K81(L6P) | |
| 111 | IL-7 | (Opr)LHLL OVSENTTILL |
| | 76-113 (Second | NCTGQVKGRK PAALGEAQPT |
| | interior fragment) | KS(Akl) |
| | K81(L6P), G85N | |
| 112 | IL-7 | (Opr)LHLL BVSEGTTILL |
| | 76-113 (Second | NCTGQVKGRK PAALGEAQPT |
| | interior fragment) | KS(Akl) |
| | K81(L2P) | |
| 113 | IL-7 | (Opr)LHLL JVSEGTTILL |
| | 76-113 (Second | NCTGQVKGRK PAALGEAQPT |
| | interior fragment) | KS(Akl) |
| | K81(L11P) | |
| 114 | IL-7 | (Opr)LHLL KVSENTTILL |
| | 76-113 (Second | NCTGQVKGRK PAALGEAQPT |
| | interior fragment) | KS(Akl) |
| | G85N | |
| 115 | IL-7 | (Opr)LHLL KVSEGTTILL |
| | 76-113 (Second | NSTGQVKGRK PAALGEAQPT |
| | interior fragment) | KS(Akl) |
| | C92S | |
| 116 | IL-7 | (Opr)ENKSLK EQKKLNDLCF |
| | C-terminal fragment | LKRLLQEIKT CWNKILXGTK EH |
| | (114-152) | |
| | M147Nle | |
| 117 | IL-7 | (Opr)ENKSLK EQKKLNDLCF |
| | C-terminal fragment | LKRLLQEIKT CWNKILMGTK EH |
| | (114-152) | |
| 118 | IL-7 | (Opr)ENKSLK EQKKLNDLSF |
| | C-terminal fragment | LKRLLQEIKT CWNKILXGTK EH |
| | (114-152) | |
| | C129S, M147Nle | |
| 119 | IL-7 | (Opr)ENKSLK EQKKLNDLCF |
| | C-terminal fragment | LKRLLQEIKT SWNKILXGTK EH |
| | (114-152) | |
| | C141S, M147Nle | |

In the table above, X is Nle (norleucine), Z is Hse (homoserine), Opr is 5-oxaproline, Akf is alpha-keto-phenylalanine, Akl is alpha-keto-leucine, O is lysine + PEG6-NH₂ (L6P), B is lysine + PEG2-NH₂ (L2P), J is lysine + PEG11-OCH₃ (L11P).

In some embodiments, the two or more fragments of the IL-7 polypeptide are ligated together. In some embodiments, three or more fragments of the IL-7 polypeptide are ligated in a sequential fashion. In some embodiments, three or more fragments of the IL-7 polypeptide are ligated in a one-pot reaction.

In some embodiments, at least one of the fragments of the IL-7 polypeptide comprises a conjugation handle. In some embodiments, the conjugation handle is incorporated into a specific desired residue during the synthesis of the fragments. In some embodiments, the conjugation handle is incorporated in its protected form, and the protecting group is removed at a later stage to reveal the active conjugation handle (e.g., an aldehyde conjugation handle can be protected during synthesis and ligation as an acetal functional group). The conjugation handle can be incorporated into any desired residue of the final IL-7 polypeptide by incorporating the residue with the conjugation handle during synthesis of the relevant fragment. The conjugation handle can be incorporated as an unnatural amino acid (e.g., azidolysine) or as a modified natural amino acid (e.g., an acylated lysine residue with an azide functionality attached through a tether group). The conjugation handle can also be incorporated as an attachment to the C-terminus or the N-terminus of the IL-7 polypeptide (e.g., Structure 1 provided herein).

In some embodiments, the method further comprises removing protecting groups from the fragments or the ligated fragments. In some embodiments, protecting groups are removed from the fragments before the folding step. In some embodiments, cysteine protecting groups are removed prior to the folding step. In some embodiment, the cysteine protecting groups are acetamidomethyl (Acm) groups. In some embodiments, the cysteine protecting groups are removed after rearrangement of the ligated fragments.

In some embodiments, the method further comprises rearranging the ligated fragments. In some embodiments, rearranging the ligated fragments involves rearranging one or more depsipeptide bonds of the linear IL-7 polypeptide. In some embodiments, the one or more depsipeptide bonds are rearranged to form one or more amide bonds. In some embodiments, the depsipeptide bonds are formed as a result of the ligation of the fragments. In some embodiments, the depsipeptide bonds are between the hydroxyl moiety of a serine or homoserine residue and an amino acid adjacent to the serine or homoserine residue. In some embodiments, the depsipeptide bonds are between the hydroxyl moiety of a homoserine residue and an amino acid adjacent to the homoserine residue. In some embodiments, rearranging the ligated fragments occurs after each of the fragments have been ligated.

In some embodiments, ligated fragments are folded. In some embodiments, folding comprises forming one or more disulfide bonds within the IL-7 polypeptide. In some embodiments, the ligated fragments are subjected to a folding process. In some embodiments, the ligated fragments are folding using methods well known in the art.

In some embodiments, the ligated polypeptide or the folded polypeptide are further modified by attaching one or more polymers thereto. In some embodiments, the method further comprises attaching a water-soluble polymer to the folded, ligated fragments. In some embodiments, the ligated polypeptide or the folded polypeptide are further modified by PEGylation. In some embodiments, the ligated polypeptide or the folded polypeptide are further modified by attaching one or more additional polypeptides thereto (e.g., an antibody or antigen binding fragment thereof, such as one specific for PD-1 as in an immunocytokine provided herein).

### Host Cells

In one aspect, described herein is a host cell expressing an IL-7 polypeptide provided herein.

In one aspect, described herein is a method of producing an IL-7 polypeptide herein, wherein the method comprises expressing the IL-7 polypeptide in a host cell.

In some embodiments, the host cell is a prokaryotic cell or a eukaryotic cell. In some embodiments, the host cell is a mammalian cell, an avian cell, a fungal cell, or an insect cell. In some embodiments, the host cell is a CHO cell, a COS cell, or a yeast cell.

### Exemplary IL-7 Polypeptides

Exemplary IL-7 Peptides of the instant disclosure are disclosed in Table 4B below

TABLE 4B
Sequences (SEQ ID NOS) of IL-7 Polypeptides

| Composit | SEQ ID | Sequence | |
|---|---|---|---|
| ion No. | NO: | modifications* | Sequence |
| | 1 | Human Native IL-7 | DCDIEGKDGK QYESVLMVSI |
| | | | DQLLDSMKEI GSNCLNNEFN |
| | | | FFKRHICDAN KEGMFLFRAA |
| | | | RKLRQFLKMN STGDFDLHLL |
| | | | KVSEGTTILL NCTGQVKGRK |
| | | | PAALGEAQPT KSLEENKSLK |
| | | | EQKKLNDLCF LKRLLQEIKT |
| | | | CWNKILMGTK EH |
| CMP-035 | 2 | Human Native IL-7 + | RKLRQFLKMN STGDFDLHLL |
| | | His-tag | DCDIEGKDGK QYESVLMVSI |
| | | | DQLLDSMKEI GSNCLNNEFN |
| | | | FFKRHICDAN KEGMFLFRAA |
| | | | KVSEGTTILL NCTGQVKGRK |
| | | | PAALGEAQPT KSLEENKSLK |
| | | | EQKKLNDLCF LKRLLQEIKT |
| | | | CWNKILMGTK EHVDHHHHHH |
| CMP-036 | 3 | M17Nle, M27Nle, | DCDIEGKDGK QYESVLXVSI |
| (No N- | | N36Hse, M54Nle, | DOLLDSXKEI GSNCLZNEFN |
| terminal | | M69Nle, D76Hse, | FFKRHICDAN KEGXFLFRAA |
| conjugation | | E114Hse, M147Nle | RKLRQFLKXN STGDFZLHLL |
| handle)/ | | | KVSEGTTILL NCTGQVKGRK |
| CMP-108 | | | PAALGEAQPT KSLZENKSLK |
| (includes | | | EQKKLNDLCF LKRLLQEIKT |
| glutaryl- | | | CWNKILXGTK EH |
| PEG9- | | | |
| azide at | | | |
| N- | | | |
| terminus) | | | |
| | 4 | N36Hse, D76Hse, | DCDIEGKDGK QYESVLMVSI |
| | | E114Hse | DQLLDSMKEI GSNCLZNEFN |
| | | | FFKRHICDAN KEGMFLFRAA |
| | | | RKLRQFLKMN STGDFZLHLL |
| | | | KVSEGTTILL NCTGQVKGRK |
| | | | PAALGEAQPT KSLZENKSLK |
| | | | EQKKLNDLCF LKRLLQEIKT |
| | | | CWNKILMGTK EH |
| CMP-107 | 5 | V15W, M17Nle, | DCDIEGKDGK QYESWLXVSI |
| (includes | | M27Nle, N36Hse, | DQLLDSXKEI GSNCLZNEFN |
| glutaryl- | | M54Nle, M69Nle, | FFKRHICDAN KEGXFLFRAA |
| PEG₉- | | D76Hse, E114Hse, | RKLRQFLKXN STGDFZLHLL |
| azide at | | M147Nle | KVSEGTTILL NCTGQVKGRK |
| N- | | | PAALGEAQPT KSLZENKSLK |
| terminus) | | | EQKKLNDLCF LKRLLQEIKT |
| | | | CWNKILXGTK EH |
| CMP-109 | 6 | M17Nle, M27Nle, | DCDIEGKDGK QYESVLXVSI |
| (includes | | N36Hse, M54Nle, | DQLLDSXKEI GSNCLZNEFN |
| glutaryl- | | M69Nle, D76Hse, | FFKRHICDAN KEGXFLFRAA |
| PEG₉- | | K81(L6P), E114Hse, | RKLRQFLKXN STGDFZLHLL |
| azide at | | M147Nle | OVSEGTTILL NCTGQVKGRK |
| N- | | | PAALGEAQPT KSLZENKSLK |
| terminus) | | | EQKKLNDLCF LKRLLQEIKT |
| | | | CWNKILXGTK EH |
| CMP-110 | 7 | M17Nle, M27Nle, | DCDIEGKDGK QYESVLXVSI |
| (includes | | N36Hse, M54Nle, | DQLLDSXKEI GSNCLZNEFN |
| glutaryl- | | M69Nle, D76Hse, | FFKRHICDAN KEGXFLFRAA |
| PEG₉- | | G85N, E114Hse, | RKLRQFLKXN STGDFZLHLL |
| azide at | | M147Nle | KVSENTTILL NCTGQVKGRK |
| N- | | | PAALGEAQPT KSLZENKSLK |
| terminus) | | | EQKKLNDLCF LKRLLQEIKT |
| | | | CWNKILXGTK EH |
| CMP-111 | 8 | M17Nle, M27Nle, | DCDIEGKDGK QYESVLXVSI |
| (includes | | N36Hse, M54Nle, | DQLLDSXKEI GSNCLZNEFN |
| glutaryl- | | M69Nle, D76Hse, | FFKRHICDAN KEGXFLFRAA |
| PEG₉- | | K81(L6P), G85N, | RKLRQFLKXN STGDFZLHLL |
| azide at | | E114Hse, M147Nle | OVSENTTILL NCTGQVKGRK |
| N- | | | PAALGEAQPT KSLZENKSLK |
| terminus) | | | EQKKLNDLCF LKRLLQEIKT |
| | | | CWNKILXGTK EH |
| CMP-112 | 9 | M17Nle, M27Nle, | DCDIEGKDGK QYESVLXVSI |
| (includes | | N36Hse, M54Nle, | DQLLDSXKEI GSNCLZNEFN |
| glutaryl- | | M69Nle, D76Hse, | FFKRHICDAN KEGXFLFRAA |
| PEG₉- | | K81(L11P), E114Hse, | RKLRQFLKXN STGDFZLHLL |
| azide at | | M147Nle | JVSEGTTILL NCTGQVKGRK |
| N- | | | PAALGEAQPT KSLZENKSLK |
| terminus) | | | EQKKLNDLCF LKRLLQEIKT |
| | | | CWNKILXGTK EH |
| CMP-113 | 10 | C2A, M17Nle, | DADIEGKDGK QYESVLXVSI |
| (includes | | M27Nle, N36Hse, | DQLLDSXKEI GSNCLZNEFN |
| glutaryl- | | M54Nle, M69Nle, | FFKRHICDAN KEGXFLFRAA |
| PEG₉- | | D76Hse, C92A, | RKLRQFLKXN STGDFZLHLL |
| azide at | | E114Hse, M147Nle | KVSEGTTILL NATGQVKGRK |
| N- | | | PAALGEAQPT KSLZENKSLK |
| terminus) | | | EQKKLNDLCF LKRLLQEIKT |
| | | | CWNKILXGTK EH |
| CMP-114 | 11 (same | M17Nle, M27Nle, | DCDIEGKDGK QYESVLXVSI |
| (CMP-108 | base | N36Hse, M54Nle, | DQLLDSXKEI GSNCLZNEFN |
| conugated | sequence | M69Nle, D76Hse, | FFKRHICDAN KEGXFLFRAA |
| to 30 kDa | as SEQ | E114Hse, M147Nle | RKLRQFLKXN STGDFZLHLL |
| DBCO | ID NO: 3) | | KVSEGTTILL NCTGQVKGRK |
| PEG) | | | PAALGEAQPT KSLZENKSLK |
| | | | EQKKLNDLCF LKRLLQEIKT |
| | | | CWNKILXGTK EH |
| CMP-115 | 12 | M17Nle, M27Nle, | DCDIEGKDGK QYESVLXVSI |
| (includes | | N36Hse, M54Nle, | DQLLDSXKEI GSNCLZNEFN |
| glutaryl- | | M69Nle, D76Hse, | FFKRHICDAN KEGXFLFRAA |
| PEG9- | | K81(L2P), E114Hse, | RKLRQFLKXN STGDFZLHLL |
| azide at | | M147Nle | BVSEGTTILL NCTGQVKGRK |
| N- | | | PAALGEAQPT KSLZENKSLK |
| terminus) | | | EQKKLNDLCF LKRLLQEIKT |
| | | | CWNKILXGTK EH |
| | 50 | V15W | DCDIEGKDGK QYESWLMVSI |
| | | | DQLLDSMKEI GSNCLNNEFN |
| | | | FFKRHICDAN KEGMFLFRAA |
| | | | RKLRQFLKMN STGDFDLHLL |
| | | | KVSEGTTILL NCTGQVKGRK |
| | | | PAALGEAQPT KSLEENKSLK |
| | | | EQKKLNDLCF LKRLLQEIKT |
| | | | CWNKILMGTK EH |
| CMP-106 | 51 | V15W, | DCDIEGKDGK QYESWLMVSI |
| | | His-tag | DQLLDSMKEI GSNCLNNEFN |
| | | | FFKRHICDAN KEGMFLFRAA |
| | | | RKLRQFLKMN STGDFDLHLL |
| | | | KVSEGTTILL NCTGQVKGRK |
| | | | PAALGEAQPT KSLEENKSLK |
| | | | EQKKLNDLCF LKRLLQEIKT |
| | | | CWNKILMGTK EHVDHHHHHH |
| | 52 | K81L6P, G85N, | DCDIEGKDGK QYESVLMVSI |
| | | | DQLLDSMKEI GSNCLNNEFN |
| | | | FFKRHICDAN KEGMFLFRAA |
| | | | RKLRQFLKMN STGDFDLHLL |
| | | | OVSENTTILL NCTGQVKGRK |
| | | | PAALGEAQPT KSLEENKSLK |
| | | | EQKKLNDLCF LKRLLQEIKT |
| | | | CWNKILMGTK EH |
| | 53 | V15W, G85N, | DCDIEGKDGK QYESWLMVSI |
| | | | DQLLDSMKEI GSNCLNNEFN |
| | | | FFKRHICDAN KEGMFLFRAA |
| | | | RKLRQFLKMN STGDFDLHLL |
| | | | KVSENTTILL NCTGQVKGRK |
| | | | PAALGEAQPT KSLEENKSLK |
| | | | EQKKLNDLCF LKRLLQEIKT |
| | | | CWNKILMGTK EH |
| | 54 | V15W, K81(L6P), | DCDIEGKDGK QYESWLMVSI |
| | | G85N | DQLLDSMKEI GSNCLNNEFN |
| | | | FFKRHICDAN KEGMFLFRAA |
| | | | RKLRQFLKMN STGDFDLHLL |
| | | | OVSENTTILL NCTGQVKGRK |
| | | | PAALGEAQPT KSLEENKSLK |
| | | | EQKKLNDLCF LKRLLQEIKT |
| | | | CWNKILMGTK EH |
| | 55 | G85N | DCDIEGKDGK QYESVLMVSI |
| | | | DQLLDSMKEI GSNCLNNEFN |
| | | | FFKRHICDAN KEGMFLFRAA |
| | | | RKLRQFLKMN STGDFDLHLL |
| | | | KVSENTTILL NCTGQVKGRK |
| | | | PAALGEAQPT KSLEENKSLK |
| | | | EQKKLNDLCF LKRLLQEIKT |
| | | | CWNKILMGTK EH |
| CMP-117 | 56 | Q11F, His-tag | DCDIEGKDGK FYESVLMVSI |
| | | | DQLLDSMKEI GSNCLNNEFN |
| | | | FFKRHICDAN KEGMFLFRAA |
| | | | RKLRQFLKMN STGDFDLHLL |
| | | | KVSEGTTILL NCTGQVKGRK |
| | | | PAALGEAQPT KSLEENKSLK |
| | | | EQKKLNDLCF LKRLLQEIKT |
| | | | CWNKILMGTK EHVDHHHHHH |
| | 57 | Q11F | DCDIEGKDGK FYESVLMVSI |
| | | | DQLLDSMKEI GSNCLNNEFN |
| | | | FFKRHICDAN KEGMFLFRAA |
| | | | RKLRQFLKMN STGDFDLHLL |
| | | | KVSEGTTILL NCTGQVKGRK |
| | | | PAALGEAQPT KSLEENKSLK |
| | | | EQKKLNDLCF LKRLLQEIKT |
| | | | CWNKILMGTK EH |

In Table 4B above, X is Nle (norleucine), Z is Hse (homoserine), O is lysine + PEG6-NH₂ (L6P),
B is lysine + PEG2-NH₂ (L2P), J is lysine + PEG11-OCH₃ (L11P).

In some instances in Table 4B above, the description of the composition number indicates that the composition “includes glutaryl-PEG9-azide at N-terminus.” For the indicated compositions, the CMP number thus refers to an IL-7 polypeptide having the indicated sequence with the N-terminal amine modified to include a glutaryl-PEG9-azide functionality shown below

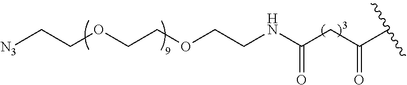

For sake of clarity, it is not intended the the indicated SEQ ID NO associated with each CMP number contains the glutaryl-PEG9-azide functionality, though reference to the CMP number will include this functionality where it is indicated to be present in Table 4B. Where the CMP number indicates the composition does include the indicated functionality, versions of the IL-7 polypeptide of the corresponding SEQ ID NO without the functionality attached are also contemplated to be within the scope of the disclosure herein.

### II. Immune Checkpoint Inhibitor Molecule and IL-7 Immunocytokines

In some instances, the IL-7 polypeptides described herein are incorporated into immunocytokines by linking them with immune checkpoint inhibitor molecules, (e.g., as anti-PD-1 antibodies or antigen binding fragments thereof). In some embodiments, the immunocytokines comprise an IL-7 polypeptide (e.g., any of the IL-7 polypeptides provided herein), an immune checkpoint inhibitor molecule (e.g., an antibody or antigen binding fragment which binds to an immune checkpoint antigen, such as PD-1), and linker connecting to the IL-7 polypeptide and to the immune checkpoint inhibitor molecule.

Immunocytokines provide the advantage of specific delivery of immune stimulatory cytokines to cells demonstrating markers of exhaustion or inhibition (e.g. the PD-1 protein). Systemic admission of cytokines can cause non-targeted tissues to experience deleterious off-target effects. Another potential advantage of immunocytokines is that such conjugates may have demonstrated synergistic effects.

In one aspect, provided herein, is an immunocytokine comprising an immune checkpoint inhibitor molecule (e.g., an anti-PD-1 antibody or antigen binding fragment thereof) linked to an IL-7 polypeptide provided herein.

In one aspect, provided herein, is an immunocytokine comprising an immune checkpoint inhibitor molecule (e.g., an anti-PD-1 antibody or antigen binding fragment thereof) linked to an IL-7 polypeptide provided herein having reduced affinity to the IL-7 receptor compared to an IL-7 polypeptide of SEQ ID NO: 1.

In one aspect, provided herein, is an immunocytokine comprising an immune checkpoint inhibitor molecule (e.g., an anti-PD-1 antibody or antigen binding fragment thereof) linked to an IL-7 polypeptide provided herein having reduced affinity to the IL-7 receptor compared to an IL-7 polypeptide of SEQ ID NO: 3.

In one aspect, provided herein, is an immunocytokine comprising an immune checkpoint inhibitor molecule (e.g., an anti-PD-1 antibody or antigen binding fragment thereof), an IL-7 polypeptide as provided herein, and a linker, wherein the linker comprises points of attachment to both the IL-7 polypeptide and the immune checkpoint inhibitor molecule.

### Immune Checkpoint Inhibitor Molecules

The immunocytokines provided herein comprise an immune checkpoint inhibitor molecule. In some embodiments, the immune checkpoint inhibitor molecule specifically binds to at least one immune checkpoint molecule. In some embodiments, the immune checkpoint molecule is an inhibitory immune checkpoint molecule. In some embodiments, an inhibitory immune checkpoint molecule is an immune system regulator implicated in the deactivation or lowering of an immune response. In some embodiments, an inhibitory immune checkpoint molecule has an effect on an immune response when it binds to its complementary checkpoint molecule.

In some embodiments, the immune checkpoint molecule which is bound by the immune checkpoint inhibitor molecule (e.g., an antibody or antigen binding fragment thereof) of the activatable immunocytokine is adenosine A2A receptor (A2AR), adenosine A2B receptor (A2BR), B7-H3, B7-H4, B and T lymphocyte Attenuater (BTLA), Cytotoxic T-Lymphocyte-Associated protein 4 (CTLA-4), Indolcamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform2 (NOX2), Programmed cell death protein 1 (PD-1), Programmed death ligand 1 (PD-L1), Programmed death ligand 2 (PD-L2), T-cell immunoreceptor with Ig and ITIM domains (TIGIT), T-cell immunoglobulin domain and Mucin domain 3 (TIM-3), V-domain Ig suppressor of T cell activation (VISTA), Sialic acid-binding immunoglobulin-type lectin 7 (SIGLEC7), Sialic acid-binding immunoglobulin-type lectin 9 (SIGLEC9), or any combination thereof. In some embodiments, the immune checkpoint molecule which is bound the immune checkpoint inhibitor molecule is PD-1, PD-L1, PD-L2, or any combination thereof. In some embodiments, the immune checkpoint molecule which is bound the immune checkpoint inhibitor molecule is PD-1, PD-L1, or both. In some embodiments, the immune checkpoint molecule which is bound the immune checkpoint inhibitor molecule is PD-1.

The immunocytokines provided herein utilize linkers to attach the immune checkpoint inhibitor molecule (e.g., an antibody or antigen binding fragment thereof, such as one which binds to PD-1) to the IL-7 polypeptide. In some embodiments, the linkers are attached to each of immune checkpoint inhibitor molecule and the IL-7 polypeptide at specific residues or a specific subset of residues. In some embodiments, the linker of an immunocytokine is attached to each moiety in a site-selective manner, such that a population of the conjugate is substantially uniform. This can be accomplished in a variety of ways as provided herein, including a) by site-selectively adding reagents for a conjugation reaction to a moiety to be conjugated, or b) synthesizing or otherwise preparing a moiety to be conjugated with a desired reagent for a conjugation reaction, or c) a combination of these two approaches. Using these approaches, the sites of attachment (such as specific amino acid residues) of the linker to each moiety can be selected with precision. Additionally, these approaches allow a variety of linkers to be employed for the composition which are not limited to amino acid residues as is required for fusion proteins.

In some embodiments, the immune checkpoint inhibitor molecule which is part of an immunocytokine provided herein is a polypeptide which binds specifically to the immune checkpoint molecule (e.g., PD-1). In some embodiments, the polypeptide is an antibody or antigen binding fragment thereof (e.g., an anti-PD-1 antibody or antigen binding fragment thereof.

In some embodiments, an immune checkpoint inhibitor molecule (e.g., an anti-PD-1 antibody or antigen binding fragment thereof) of the disclosure specifically binds to the immune checkpoint molecule (e.g., PD-1). An immune checkpoint inhibitor molecule selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the antibody, or antigen binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences. Binding of the immune checkpoint inhibitor molecule to the immune checkpoint molecule can block interaction of the immune checkpoint molecule with its ligand. For example, an anti-PD-1 polypeptide or an anti-PD-1 antigen binding fragment of the disclosure can block interaction of PD-1 with a ligand (e.g., PD-L1).

As used herein, the term “antibody” refers to an immunoglobulin (Ig), polypeptide, or a protein having a binding domain which is, or is homologous to, an antigen binding domain. The term further includes “antigen binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen binding fragment comprises an isolated antibody or antigen binding fragment, a purified antibody or antigen binding fragment, a recombinant antibody or antigen binding fragment, a modified antibody or antigen binding fragment, or a synthetic antibody or antigen binding fragment.

Antibodies and antigen binding fragments herein can be partly or wholly synthetically produced. An antibody or antigen binding fragment can be a polypeptide or protein having a binding domain which can be, or can be homologous to, an antigen binding domain. In one instance, an antibody or an antigen binding fragment can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. An Ig or portion thereof can, in some cases, be a human Ig. In some instances, a C_{H}3 domain can be from an immunoglobulin. In some cases, a chain or a part of an antibody or antigen binding fragment, a modified antibody or antigen binding fragment, or a binding agent can be from an Ig. In such cases, an Ig can be IgG, an IgA, an IgD, an IgE, or an IgM, or is derived therefrom. In cases where the Ig is an IgG, it can be a subtype of IgG, wherein subtypes of IgG can include IgG1, an IgG2a, an IgG2b, an IgG3, or an IgG4. In some cases, a C_{H}3 domain can be from an immunoglobulin selected from the group consisting of an IgG, an IgA, an IgD, an IgE, and an IgM, or derived therefrom. In some embodiments, an antibody or antigen binding fragment described herein comprises an IgG or is derived therefrom. In some instances, an antibody or antigen binding fragment comprises an IgG1 or is derived therefrom. In some instances, an antibody or antigen binding fragment comprises an IgG4 or is derived therefrom. In some embodiments, an antibody or antigen binding fragment described herein comprises an IgM, is derived therefrom, or is a monomeric form of IgM. In some embodiments, an antibody or antigen binding fragment described herein comprises an IgE or is derived therefrom. In some embodiments, an antibody or antigen binding fragment described herein comprises an IgD or is derived therefrom. In some embodiments, an antibody or antigen binding fragment described herein comprises an IgA or is derived therefrom.

The “light chains” of antibodies (immunoglobulins) from any vertebrate species can be assigned to one of two clearly distinct types, called kappa (“K” or “K”) or lambda (“2”), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (e.g., Kabat et al., Sequences of Proteins of Immunological Interest, (5th Ed., 1991, National Institutes of Health, Bethesda Md. (1991), pages 647-669; hereafter “Kabat”); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen binding site of antibodies (see, Kabat).

The terms “hypervariable region” and “CDR” when used herein, refer to the amino acid residues of an antibody which are responsible for antigen binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk (J. Mol. Biol., 196:901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), and light chain third complementarity determining region (VL CDR3).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is generally defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, C_{H}2 and C_{H}3.

“Antibodies” useful in the present disclosure encompass, but are not limited to, monoclonal antibodies, polyclonal antibodies, chimeric antibodies, bispecific antibodies, multispecific antibodies, heteroconjugate antibodies, humanized antibodies, human antibodies, grafted antibodies, deimmunized antibodies, mutants thereof, fusions thereof, immunoconjugates thereof, antigen binding fragments thereof, and/or any other modified configuration of the immunoglobulin molecule that comprises an antigen recognition site of the required specificity, including glycosylation variants of antibodies, amino acid sequence variants of antibodies, and covalently modified antibodies.

In some instances, an antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTopc™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Alternatively, the human antibody may be prepared by immortalizing human B lymphocytes that produce an antibody directed against a target antigen (such B lymphocytes may be recovered from an individual or may have been immunized in vitro).

Any of the antibodies herein can be bispecific. Bispecific antibodies are antibodies that have binding specificities for at least two different antigens and can be prepared using the antibodies disclosed herein. Traditionally, the recombinant production of bispecific antibodies was based on the coexpression of two immunoglobulin heavy chain-light chain pairs, with the two heavy chains having different specificities. Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH2 and CH3 regions. The first heavy chain constant region (CH1), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc. Chimeric or hybrid antibodies also may be prepared in vitro using suitable methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Provided herein are antibodies and antigen binding fragments thereof, modified antibodies and antigen binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

Antigen binding fragments of any of the antibodies herein are also contemplated. The terms “antigen binding portion of an antibody,” “antigen binding fragment,” “antigen binding domain,” “antibody fragment,” or a “functional fragment of an antibody” are used interchangeably herein to refer to one or more fragments of an antibody that retain the ability to specifically bind to an antigen. Representative antigen binding fragments include, but are not limited to, a Fab, a Fab′, a F(ab′)₂, a bispecific F(ab′)₂, a trispecific F(ab′)₂, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′; two different disulfide-stabilized Fv antibody fragments connected by flexible linker peptides).

Heteroconjugate polypeptides comprising two covalently joined antibodies or antigen binding fragments of antibodies are also within the scope of the disclosure. Suitable linkers may be used to multimerize binding agents. Non-limiting examples of linking peptides include, but are not limited to, (GS)ₙ (SEQ ID NO: 351), (GGS)ₙ (SEQ ID NO: 352), (GGGS)ₙ (SEQ ID NO: 353), (GGSG)ₙ (SEQ ID NO: 354), or (GGSGG)ₙ (SEQ ID NO: 355), (GGGGS)ₙ (SEQ ID NO: 356), wherein n is 1, 2, 3, 4, 5, 6, 7, 8, 9, or 10. For example, a linking peptide can be (GGGGS)₃ (SEQ ID NO: 357) or (GGGGS)₄ (SEQ ID NO: 358). In some embodiments, a linking peptide bridges approximately 3.5 nm between the carboxy terminus of one variable region and the amino terminus of the other variable region. Linkers of other sequences have been designed and used. Linkers can in turn be modified for additional functions, such as attachment of drugs or attachment to solid supports.

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as K_{D}. The binding affinity (K_{D}) of an antibody or antigen binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

Also provided herein are affinity matured antibodies. The following methods may be used for adjusting the affinity of an antibody and for characterizing a CDR. One way of characterizing a CDR of an antibody and/or altering (such as improving) the binding affinity of a polypeptide, such as an antibody, is termed “library scanning mutagenesis.” Generally, library scanning mutagenesis works as follows. One or more amino acid position in the CDR is replaced with two or more (such as 3, 4, 5, 6, 7, 8, 9, 10, 11, 12, 13, 14, 15, 16, 17, 18, 19, or 20) amino acids. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, for example, about 20-80 clones (depending on the complexity of the library), from each library can be screened for binding specificity or affinity to the target polypeptide (or other binding target), and candidates with increased, the same, decreased, or no binding are identified. Binding affinity may be determined using Biacore surface plasmon resonance analysis, which detects differences in binding affinity of about 2-fold or greater.

In some instances, an antibody or antigen binding fragment is bispecific or multispecific and can specifically bind to more than one antigen. In some cases, such a bispecific or multispecific antibody or antigen binding fragment can specifically bind to 2 or more different antigens. In some cases, a bispecific antibody or antigen binding fragment can be a bivalent antibody or antigen binding fragment. In some cases, a multi specific antibody or antigen binding fragment can be a bivalent antibody or antigen binding fragment, a trivalent antibody or antigen binding fragment, or a quatravalent antibody or antigen binding fragment.

An antibody or antigen binding fragment described herein can be isolated, purified, recombinant, or synthetic.

The antibodies described herein may be made by any suitable method. Antibodies can often be produced in large quantities, particularly when utilizing high level expression vectors.

### Anti-PD-1 Immune Checkpoint Inhibitor Molecules

In some preferred embodiments, the immune checkpoint inhibitor molecule of an immunocytokine as provided herein is one which binds to PD-1. Programmed cell death protein 1 (also known as PD-1 and CD279), is a cell surface receptor that plays an role in down-regulating the immune system and promoting self-tolerance by suppressing T cell inflammatory activity. PD-1 is an immune cell inhibitory molecule that is expressed on activated B cells, T cells, and myeloid cells. PD-1 represents an immune checkpoint and guards against autoimmunity via a dual mechanism of promoting apoptosis (programmed cell death) in antigen-specific T-cells in lymph nodes while reducing apoptosis in regulatory T cells. PD-1 is a member of the CD28/CTLA-4/ICOS costimulatory receptor family that delivers negative signals that affect T and B cell immunity. PD-1 is monomeric both in solution as well as on cell surface, in contrast to CTLA-4 and other family members that are all disulfide-linked homodimers. Signaling through the PD-1 inhibitory receptor upon binding its ligand, PD-L1, suppresses immune responses against autoantigens and tumors and plays a role in the maintenance of peripheral immune tolerance. The interaction between PD-1 and PD-L1 results in a decrease in tumor infiltrating lymphocytes, a decrease in T cell receptor mediated proliferation, and immune evasion by the cancerous cells. A non-limiting, exemplary, human PD-1 amino acid sequence is

(SEQ ID NO: 359)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

In some embodiments, an immunocytokine of the instant disclosure comprises a polypeptide which is specific for PD-1. In some embodiments, the immunocytokine comprises an anti-PD-1 antibody or antigen binding fragment thereof. Such immunocytokines as provided herein are in some embodiments effective for simultaneously delivering the IL-7 polypeptide and the polypeptide which selectively binds to PD-1 to a target cell (e.g., a target T cell).

In some embodiments, the IL-7 polypeptide of the immunocytokine is delivered to the target cell or the target tissue (e.g., a tumor microenvironment) at the same time as the immune checkpoint inhibitor molecule which is specific for PD-1 (e.g., anti-PD-1 antibody or antigen binding fragment thereof). In some embodiments, simultaneous delivery of both agents to the same cell has numerous potential benefits, including a) potentially improved IL-7 polypeptide selectivity for cells in a target vicinity (e.g., higher selectivity for cells in a tumor microenvironment owing to targeting by the anti-PD-1 antibody or antigen binding fragment thereof), b) potentially enhanced therapeutic potential of the IL-7 polypeptide owing to higher local concentration due to targeting of the IL-7 polypeptide to target cells by the anti-PD-1 polypeptide, and c) potentially synergistic activity owing to the dual activities of IL-7 receptor binding and blocking of PD-1 interaction with PD-L1.

In some embodiments, an anti-PD1 antibody or an anti-PD1 antigen binding fragment of the disclosure comprises a combination of a heavy chain variable region (VH) and a light chain variable region (VL) described herein. In some embodiments, an anti-PD1 antibody or an anti-PD1 antigen binding fragment of the disclosure comprises a combination of complementarity determining regions (VH CDR1, VH CDR2, VH CDR3, VL CDR1, VL CDR2, and VL CDR3) described herein. In one embodiment, an anti-PD-1 antibody or an anti-PD-1 antigen binding fragment of the disclosure comprises a Tislelizumab, Baizcan, 0KVO411B3N, BGB-A317, hu317-1/IgG4mt2, Sintilimab, Tyvyt, IBI-308, Toripalimab, TeRuiPuLi, Terepril, Tuoyi, JS-001, TAB-001, Camrelizumab, HR-301210, INCSHR-01210, SHR-1210, Cemiplimab, Cemiplimab-rwlc, LIBTAYOR, 6QVL057INT, H4H7798N, REGN-2810, SAR-439684, Avelumab, BAVENCIO®, 451238, KXG2PJ551I, MSB-0010682, MSB-0010718C, PF-06834635, Durvalumab, IMFINZI®, 28X28X9OKV, MEDI-4736, Lambrolizumab, Pembrolizumab, KEYTRUDA®, MK-3475, SCH-900475, h409A11, Nivolumab, Nivolumab BMS, OPDIVO®, BMS-936558, MDX-1106, ONO-4538, Prolgolimab, Forteca, BCD-100, Penpulimab, AK-105, Zimberclimab, AB-122, GLS-010, WBP-3055, Balstilimab, 1Q2QT5M7EO, AGEN-2034, AGEN-2034w, Genolimzumab, Geptanolimab, APL-501, CBT-501, GB-226, Dostarlimab, ANB-011, GSK-4057190A, POGVQ9A4S5, TSR-042, WBP-285, Serplulimab, HLX-10, CS-1003, Retifanlimab, 2Y3T5IF01Z, INCMGA-00012, INCMGA-0012, MGA-012, Sasanlimab, LZZ0IC2EWP, PF-06801591, RN-888, Spartalizumab, NVP-LZV-184, PDR-001, QOG25L6Z8Z, Relatlimab/nivolumab, BMS-986213, Cetrelimab, JNJ-3283, JNJ-63723283, LYK98WP91F, Tebotclimab, MGD-013, BCD-217, BAT-1306, HX-008, MEDI-5752, JTX-4014, Cadonilimab, AK-104, BI-754091, Pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, MAX-10181, AMG-404, IBI-318, MGD-019, INCB-086550, ONCR-177, LY-3462817, RG-7769, RO-7121661, F-520, XmAb-23104, Pd-1-pik, SG-001, S-95016, Sym-021, LZM-009, Budigalimab, 6VDO4TY300, ABBV-181, PR-1648817, CC-90006, XmAb-20717, 2661380, AMP-224, B7-DCIg, EMB-02, ANB-030, PRS-332, [89Zr]Deferoxamide-Pembrolizumab, 89Zr-Df-Pembrolizumab, [89Zr]Df-Pembrolizumab, STI-1110, STI-A1110, CX-188, mPD-1 Pb-Tx, MCLA-134, 244C8, ENUM 224C8, ENUM C8, 388D4, ENUM 388D4, ENUM D4, MEDI0680, or AMP-514, or a modified version of any one of these. In some embodiments, the anti-PD-1 polypeptide is Pembrolizumab, or a modified Pembrolizumab.

In some embodiments, an anti-PD-1 antibody or an anti-PD-1 antigen binding fragment of the disclosure comprises Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, Pidilizumab, LZM-009, or Budigalimab.

In some embodiments, the anti-PD-1 antibody is Nivolumab, Pembrolizumab, LZM-009, Dostarlimab, Sintilimab, Spartalizumab, Tislelizumab, or Cemiplimab. In some embodiment, the anti-PD-1 antibody is Dostarlimab, Sintilimab, Spartalizumab, or Tislelizumab. In some embodiments, the anti-PD-1 antibody is Nivolumab, Pembrolizumab, LZM-009, or Cemiplimab. In some embodiments, the anti-PD-1 antibody is Pembrolizumab. In some embodiments, the anti-PD-1 antibody is LZM-009.

It is contemplated that generic or biosimilar versions of the named antibodies herein which share the same amino acid sequence as the indicated antibodies are also encompassed when the name of the antibody is used. In some embodiments, the anti-PD-1 antibody is a biosimilar of Tislelizumab, Sintilimab, Toripalimab, Terepril, Camrelizumab, Cemiplimab, Pembrolizumab Nivolumab, Prolgolimab, Penpulimab, Zimberclimab, Balstilimab, Genolimzumab, Geptanolimab, Dostarlimab, Serplulimab, Retifanlimab, Sasanlimab, Spartalizumab, Cetrelimab, Tebotelimab, Cadonilimab, Pidilizumab, LZM-009, or Budigalimab. In some embodiments, the anti-PD-1 antibody is a biosimilar of any one of the antibodies provided herein.

TABLE 5 provides the sequences of exemplary anti-PD-1 polypeptides and anti-PD-1 antigen binding fragments that can be modified to prepare anti-PD-1 immunoconjugates. TABLE 5 also shows provides combinations of CDRs that can be utilized in a modified anti-PD-1 immunoconjugate. Reference to an anti-PD-1 polypeptide herein may alternatively refer to an anti-PD-1 antigen binding fragment.

TABLE 5

| Antibody or | | |
|---|---|---|
| Ag-binding | | SEQ ID |
| fragment | Sequence | NO |
| Tislelizumab, | QVQLQESGPGLVKPSETLSLTCTVSGFSLTSYGVHWIRQPPG | 125 |
| Baizean, | KGLEWIGVIYADGSTNYNPSLKSRVTISKDTSKNQVSLKLSSV | |
| OKVO411B3N, | TAADTAVYYCARAYGNYWYIDVWGQGTTVTVSSASTKGPSV | |
| BGB-A317, | FPLAPCSRSTSESTAALGCLVKDYFPEPVTVSWNSGALTSGVHTF | |
| hu317- | PAVLQSSGLYSLSSVVTVPSSSLGTKTYTCNVDHKPSNTKVDKR | |
| 1/IgG4mt2 | VESKYGPPCPPCPAPPVAGGPSVFLFPPKPKDTLMISRTPEVTCVV | |
| Heavy Chain | VAVSQEDPEVQFNWYVDGVEVHNAKTKPREEQFNSTYRVVSVL | |
| (VH in Bold) | TVVHQDWLNGKEYKCKVSNKGLPSSIEKTISKAKGQPREPQVYT | |
| | LPPSQEEMTKNQVSLTCLVKGFYPSDIAVEWESNGQPENNYKTT | |
| | PPVLDSDGSFFLYSKLTVDKSRWQEGNVFSCSVMHEALHNHYT | |
| | QKSLSLSLGK | |
| Tislelizumab, | DIVMTQSPDSLAVSLGERATINCKSSESVSNDVAWYQQKPGQ | 126 |
| Baizean, | PPKLLINYAFHRFTGVPDRFSGSGYGTDFTLTISSLQAEDVAV | |
| OKVO411B3N, | YYCHQAYSSPYTFGQGTKLEIKRTVAAPSVFIFPPSDEQLKSGT | |
| BGB-A317, | ASVVCLLNNFYPREAKVQWKVDNALQSGNSQESVTEQDSKDST | |
| hu317- | YSLSSTLTLSKADYEKHKVYACEVTHQGLSSPVTKSFNRGEC | |
| 1/IgG4mt2 | | |
| Light Chain | | |
| (VL in Bold) | | |
| Sintilimab, | QVQLVQSGAEVKKPGSSVKVSCKASGGTFSSYAISWVRQAPG | 127 |
| Tyvyt, IBI-308 | QGLEWMGLIIPMFDTAGYAQKFQGRVAITVDESTSTAYMEL | |
| Heavy Chain | SSLRSEDTAVYYCARAEHSSTGTFDYWGQGTLVTVSSASTKG | |
| (VH in Bold) | PSVFPLAPCSRSTSESTAALGCLVKDYFPEPVTVSWNSGALTSGV | |
| | HTFPAVLQSSGLYSLSSVVTVPSSSLGTKTYTCNVDHKPSNTKVD | |
| | KRVESKYGPPCPPCPAPEFLGGPSVFLFPPKPKDTLMISRTPEVTC | |
| | VVVDVSQEDPEVQFNWYVDGVEVHNAKTKPREEQFNSTYRVVS | |
| | VLTVLHQDWLNGKEYKCKVSNKGLPSSIEKTISKAKGQPREPQV | |
| | YTLPPSQEEMTKNQVSLTCLVKGFYPSDIAVEWESNGQPENNYK | |
| | TTPPVLDSDGSFFLYSRLTVDKSRWQEGNVFSCSVMHEALHNHY | |
| | TQKSLSLSLGK | |
| Sintilimab, | DIQMTQSPSSVSASVGDRVTITCRASQGISSWLAWYQQKPGK | 128 |
| Tyvyt, IBI-308 | APKLLISAASSLQSGVPSRFSGSGSGTDFTLTISSLQPEDFATY | |
| Light Chain | YCQQANHLPFTFGGGTKVEIKRTVAAPSVFIFPPSDEQLKSGTA | |
| (VL in Bold) | SVVCLLNNFYPREAKVQWKVDNALQSGNSQESVTEQDSKDSTY | |
| | SLSSTLTLSKADYEKHKVYACEVTHQGLSSPVTKSFNRGEC | |
| Toripalimab, | QGQLVQSGAEVKKPGASVKVSCKASGYTFTDYEMHWVRQA | 129 |
| TeRuiPuLi, | PIHGLEWIGVIESETGGTAYNQKFKGRVTITADKSTSTAYME | |
| Terepril, Tuoyi, | LSSLRSEDTAVYYCAREGITTVATTYYWYFDVWGQGTTVTV | |
| JS-001, TAB- | SSASTKGPSVFPLAPCSRSTSESTAALGCLVKDYFPEPVTVSWNS | |
| 001 | GALTSGVHTFPAVLQSSGLYSLSSVVTVPSSSLGTKTYTCNVDHK | |
| Heavy Chain | PSNTKVDKRVESKYGPPCPPCPAPEFLGGPSVFLFPPKPKDTLMIS | |
| (VH in Bold) | RTPEVTCVVVDVSQEDPEVQFNWYVDGVEVHNAKTKPREEQFN | |
| | STYRVVSVLTVLHQDWLNGKEYKCKVSNKGLPSSIEKTISKAKG | |
| | QPREPQVYTLPPSQEEMTKNQVSLTCLVKGFYPSDIAVEWESNG | |
| | QPENNYKTTPPVLDSDGSFFLYSRLTVDKSRWQEGNVFSCSVMH | |
| | EALHNHYTQKSLSLSLGK | |
| Toripalimab, | DVVMTQSPLSLPVTLGQPASISCRSSQSIVHSNGNTYLEWYLQ | 130 |
| TeRuiPuLi, | KPGQSPQLLIYKVSNRFSGVPDRFSGSGSGTDFTLKISRVEAE | |
| Terepril, Tuoyi, | DVGVYYCFQGSHVPLTFGQGTKLEIKRTVAAPSVFIFPPSDEQL | |
| JS-001, TAB- | KSGTASVVCLLNNFYPREAKVQWKVDNALQSGNSQESVTEQDS | |
| 001 | KDSTYSLSSTLTLSKADYEKHKVYACEVTHQGLSSPVTKSFNRG | |
| Light Chain | EC | |
| (VL in Bold) | | |
| Camrelizumab, | EVQLVESGGGLVQPGGSLRLSCAASGFTFSSYMMSWVRQAP | 131 |
| HR-301210, | GKGLEWVATISGGGANTYYPDSVKGRFTISRDNAKNSLYLQ | |
| INCSHR-01210, | MNSLRAEDTAVYYCARQLYYFDYWGQGTTVTVSSASTKGPS | |
| SHR-1210 | VFPLAPCSRSTSESTAALGCLVKDYFPEPVTVSWNSGALTSGVHT | |
| Heavy Chain | FPAVLQSSGLYSLSSVVTVPSSSLGTKTYTCNVDHKPSNTKVDKR | |
| (VH in Bold) | VESKYGPPCPPCPAPEFLGGPSVFLFPPKPKDTLMISRTPEVTCVV | |
| | VDVSQEDPEVQFNWYVDGVEVHNAKTKPREEQFNSTYRVVSVL | |
| | TVLHQDWLNGKEYKCKVSNKGLPSSIEKTISKAKGQPREPQVYT | |
| | LPPSQEEMTKNQVSLTCLVKGFYPSDIAVEWESNGQPENNYKTT | |
| | PPVLDSDGSFFLYSRLTVDKSRWQEGNVFSCSVMHEALHNHYTQ | |
| | KSLSLSLGK | |
| Camrelizumab, | DIQMTQSPSSLSASVGDRVTITCLASQTIGTWLTWYQQKPGK | 132 |
| HR-301210, | APKLLIYTATSLADGVPSRFSGSGSGTDFTLTISSLQPEDFATY | |
| INCSHR-01210, | YCQQVYSIPWTFGGGTKVEIKRTVAAPSVFIFPPSDEQLKSGTA | |
| SHR-1210 | SVVCLLNNFYPREAKVQWKVDNALQSGNSQESVTEQDSKDSTY | |
| Light Chain | SLSSTLTLSKADYEKHKVYACEVTHQGLSSPVTKSFNRGEC | |
| (VL in Bold) | | |
| Cemiplimab, | EVQLLESGGVLVQPGGSLRLSCAASGFTFSNFGMTWVRQAP | 133 |
| Cemiplimab- | GKGLEWVSGISGGGRDTYFADSVKGRFTISRDNSKNTLYLQ | |
| rwlc, | MNSLKGEDTAVYYCVKWGNIYFDYWGQGTLVTVSSASTKGP | |
| LIBTAYO ®, | SVFPLAPCSRSTSESTAALGCLVKDYFPEPVTVSWNSGALTSGVH | |
| 6QVL057INT, | TFPAVLQSSGLYSLSSVVTVPSSSLGTKTYTCNVDHKPSNTKVDK | |
| H4H7798N, | RVESKYGPPCPPCPAPEFLGGPSVFLFPPKPKDTLMISRTPEVTCV | |
| REGN-2810, | VVDVSQEDPEVQFNWYVDGVEVHNAKTKPREEQFNSTYRVVSV | |
| SAR-439684 | LTVLHQDWLNGKEYKCKVSNKGLPSSIEKTISKAKGQPREPQVY | |
| Heavy Chain | TLPPSQEEMTKNQVSLTCLVKGFYPSDIAVEWESNGQPENNYKT | |
| (VH in Bold) | TPPVLDSDGSFFLYSRLTVDKSRWQEGNVFSCSVMHEALHNHYT | |
| | QKSLSLSLGK | |
| Cemiplimab, | DIQMTQSPSSLSASVGDSITITCRASLSINTFLNWYQQKPGKAP | 134 |
| Cemiplimab- | NLLIYAASSLHGGVPSRFSGSGSGTDFTLTIRTLQPEDFATYY | |
| rwlc, | CQQSSNTPFTFGPGTVVDFRRTVAAPSVFIFPPSDEQLKSGTASV | |
| LIBTAYO ®, | VCLLNNFYPREAKVQWKVDNALQSGNSQESVTEQDSKDSTYSL | |
| 6QVL057INT, | SSTLTLSKADYEKHKVYACEVTHQGLSSPVTKSFNRGEC | |
| H4H7798N, | | |
| REGN-2810, | | |
| SAR-439684 | | |
| Light Chain | | |
| (VL in Bold) | | |
| Lambrolizumab, | QVQLVQSGVEVKKPGASVKVSCKASGYTFTNYYMYWVRQA | 135 |
| Pembrolizumab, | PGQGLEWMGGINPSNGGTNFNEKFKNRVTLTTDSSTTTAYM | |
| KEYTRUDA ®, | ELKSLQFDDTAVYYCARRDYRFDMGFDYWGQGTTVTVSSAS | |
| MK-3475, SCH- | TKGPSVFPLAPCSRSTSESTAALGCLVKDYFPEPVTVSWNSGALT | |
| 900475, | SGVHTFPAVLQSSGLYSLSSVVTVPSSSLGTKTYTCNVDHKPSNT | |
| h409A11 | KVDKRVESKYGPPCPPCPAPEFLGGPSVFLFPPKPKDTLMISRTPE | |
| Heavy Chain | VTCVVVDVSQEDPEVQFNWYVDGVEVHNAKTKPREEQFNSTYR | |
| (VH in Bold) | VVSVLTVLHQDWLNGKEYKCKVSNKGLPSSIEKTISKAKGQPRE | |
| | PQVYTLPPSQEEMTKNQVSLTCLVKGFYPSDIAVEWESNGQPEN | |
| | NYKTTPPVLDSDGSFFLYSRLTVDKSRWQEGNVFSCSVMHEALH | |
| | NHYTQKSLSLSLGK | |
| Lambrolizumab, | EIVLTQSPATLSLSPGERATLSCRASKGVSTSGYSYLHWYQQ | 136 |
| Pembrolizumab, | KPGQAPRLLIYLASYLESGVPARFSGSGSGTDFTLTISSLEPED | |
| KEYTRUDA ®, | FAVYYCQHSRDLPLTFGGGTKVEIKRTVAAPSVFIFPPSDEQLK | |
| MK-3475, SCH- | SGTASVVCLLNNFYPREAKVQWKVDNALQSGNSQESVTEQDSK | |
| 900475, | DSTYSLSSTLTLSKADYEKHKVYACEVTHQGLSSPVTKSFNRGE | |
| h409A11 | C | |
| Light Chain | | |
| (VL in Bold) | | |
| Lambrolizumab, | QVQLVQSGVEVKKPGASVKVSCKASGYTFTNYYMYWVRQAPG | 137 |
| Pembrolizumab, | QGLEWMGGFPSNGGTNFNEKFKNRVTLTTDSSTTTAYMELKSL | |
| KEYTRUDA ®, | QFDDTAVYYCARRDYRFDMGFDYWGQGTTVTVSS | |
| MK-3475, SCH- | | |
| 900475, | | |
| h409A11 | | |
| VH | | |
| Lambrolizumab, | EIVLTQSPATLSLSPGERATLSCRASKGVSTSGYSYLHWYQQKPG | 138 |
| Pembrolizumab, | QAPRLLIYLASYLESGVPARFSGSGSGTDFTLTISSLEPEDFAVYY | |
| KEYTRUDA ®, | CQHSRDLPLTFGGGTKVEIK | |
| MK-3475, SCH- | | |
| 900475, | | |
| h409A11 | | |
| VL | | |
| Nivolumab, | QVQLVESGGGVVQPGRSLRLDCKASGITFSNSGMHWVRQAPGK | 139 |
| Nivolumab | GLEWVAVIWYDGSKRYYADSVKGRFTISRDNSKNTLFLQMNSL | |
| BMS, | RAEDTAVYYCATNDDYWGQGTLVTVSS | |
| OPDIVO ®, | | |
| BMS-936558, | | |
| MDX-1106, | | |
| ONO-4538 | | |
| VH | | |
| Nivolumab, | EIVLTQSPATLSLSPGERATLSCRASQSVSSYLAWYQQKPGQAPR | 140 |
| Nivolumab | LLIYDASNRATGIPARFSGSGSGTDFTLTISSLEPEDFAVYYCQQS | |
| BMS, | SNWPRTFGQGTKVEIK | |
| OPDIVO ®, | | |
| BMS-936558, | | |
| MDX-1106, | | |
| ONO-4538 | | |
| VL | | |
| Prolgolimab, | QVQLVQSGGGLVQPGGSLRLSCAASGFTFSSYWMYWVRQVP | 141 |
| Forteca, BCD- | GKGLEWVSAIDTGGGRTYYADSVKGRFAISRVNAKNTMYLQ | |
| 100 | MNSLRAEDTAVYYCARDEGGGTGWGVLKDWPYGLDAWGQ | |
| Heavy Chain | GTLVTVSSASTKGPSVFPLAPSSKSTSGGTAALGCLVKDYFPEPV | |
| (VH in Bold) | TVSWNSGALTSGVHTFPAVLQSSGLYSLSSVVTVPSSSLGTQTYI | |
| | CNVNHKPSNTKVDKRVEPKSCDKTHTCPPCPAPEAAGGPSVFLF | |
| | PPKPKDTLMISRTPEVTCVVVDVSHEDPEVKFNWYVDGVEVHN | |
| | AKTKPREEQYNSTYRVVSVLTVLHQDWLNGKEYKCKVSNKALP | |
| | APIEKTISKAKGQPREPQVYTLPPSREEMTKNQVSLTCLVKGFYP | |
| | SDIAVEWESNGQPENNYKTTPPVLDSDGSFFLYSKLTVDKSRWQ | |
| | QGNVFSCSVMHEALHNHYTQKSLSLSPGK | |
| Prolgolimab, | QPVLTQPLSVSVALGQTARITCGGNNIGSKNVHWYQQKPGQ | 142 |
| Forteca, BCD- | APVLVIYRDSNRPSGIPERFSGSNSGNTATLTISRAQAGDEADY | |
| 100 | YCQVWDSSTAVFGTGTKLTVLQRTVAAPSVFIFPPSDEQLKSGT | |
| Light Chain | ASVVCLLNNFYPREAKVQWKVDNALQSGNSQESVTEQDSKDST | |
| (VL in Bold) | YSLSSTLTLSKADYEKHKVYACEVTHQGLSSPVTKSFNRGEC | |
| Balstilimab, | QVQLVESGGGVVQPGRSLRLSCAASGFTFSSYGMHWVRQAP | 143 |
| 1Q2QT5M7EO, | GKGLEWVAVIWYDGSNKYYADSVKGRFTISRDNSKNTLYLQ | |
| AGEN-2034, | MNSLRAEDTAVYYCASNGDHWGQGTLVTVSSASTKGPSVFPL | |
| AGEN-2034w | APCSRSTSESTAALGCLVKDYFPEPVTVSWNSGALTSGVHTFPAV | |
| Heavy Chain | LQSSGLYSLSSVVTVPSSSLGTKTYTCNVDHKPSNTKVDKRVESK | |
| (VH in Bold) | YGPPCPPCPAPEFLGGPSVFLFPPKPKDTLMISRTPEVTCVVVDVS | |
| | QEDPEVQFNWYVDGVEVHNAKTKPREEQFNSTYRVVSVLTVLH | |
| | QDWLNGKEYKCKVSNKGLPSSIEKTISKAKGQPREPQVYTLPPSQ | |
| | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLG | |
| Balstilimab, | EIVMTQSPATLSVSPGERATLSCRASQSVSSNLAWYQQKPGQ | 144 |
| 1Q2QT5M7EO, | APRLLIYGASTRATGIPARFSGSGSGTEFTLTISSLQSEDFAVY | |
| AGEN-2034, | YCQQYNNWPRTFGQGTKVEIKRTVAAPSVFIFPPSDEQLKSGT | |
| AGEN-2034w | ASVVCLLNNFYPREAKVQWKVDNALQSGNSQESVTEQDSKDST | |
| Light Chain | YSLSSTLTLSKADYEKHKVYACEVTHQGLSSPVTKSFNRGEC | |
| (VL in Bold) | | |
| Dostarlimab, | EVQLLESGGGLVQPGGSLRLSCAASGFTFSSYDMSWVRQAP | 145 |
| ANB-011, | GKGLEWVSTISGGGSYTYYQDSVKGRFTISRDNSKNTLYLQM | |
| GSK- | NSLRAEDTAVYYCASPYYAMDYWGQGTTVTVSSASTKGPSVF | |
| 4057190A, | PLAPCSRSTSESTAALGCLVKDYFPEPVTVSWNSGALTSGVHTFP | |
| P0GVQ9A4S5, | AVLQSSGLYSLSSVVTVPSSSLGTKTYTCNVDHKPSNTKVDKRV | |
| TSR-042, WBP- | ESKYGPPCPPCPAPEFLGGPSVFLFPPKPKDTLMISRTPEVTCVVV | |
| 285 | DVSQEDPEVQFNWYVDGVEVHNAKTKPREEQFNSTYRVVSVLT | |
| Heavy Chain | VLHQDWLNGKEYKCKVSNKGLPSSIEKTISKAKGQPREPQVYTL | |
| (VH in Bold) | PPSQEEMTKNQVSLTCLVKGFYPSDIAVEWESNGQPENNYKTTP | |
| | PVLDSDGSFFLYSRLTVDKSRWQEGNVFSCSVMHEALHNHYTQ | |
| | KSLSLSLGK | |
| Dostarlimab, | DIQLTQSPSFLSAYVGDRVTITCKASQDVGTAVAWYQQKPGK | 146 |
| ANB-011, | APKLLIYWASTLHTGVPSRFSGSGSGTEFTLTISSLQPEDFATY | |
| GSK- | YCQHYSSYPWTFGQGTKLEIKRTVAAPSVFIFPPSDEQLKSGTA | |
| 4057190A, | SVVCLLNNFYPREAKVQWKVDNALQSGNSQESVTEQDSKDSTY | |
| P0GVQ9A4S5, | SLSSTLTLSKADYEKHKVYACEVTHQGLSSPVTKSFNRGEC | |
| TSR-042, WBP- | | |
| 285 | | |
| Light Chain | | |
| (VL in Bold) | | |
| Serplulimab, | QVQLVESGGGLVKPGGSLRLSCAASGFTFSNYGMSWIRQAP | 147 |
| HLX-10 | GKGLEWSTISGGGSNIYYADSVKGRFTISRDNAKNSLYLQMN | |
| Heavy Chain | SLRAEDTAVYYCVSYYYGIDFWGQGTSVTVSSASKYGPSVFPLA | |
| (VH in Bold) | PCSRSTSESTAALGCLVKDYFPEPVTVSWNSGALTSGVHTFPAVL | |
| | QSSGLYSLSSVVTVPSSSLGTKTYTCNVDHKPSNTKVDKRVESK | |
| | YGPPCPPCPAPEFLGGPSVFLFPPKPKDTLMISRTPEVTCVVVDVS | |
| | QEDPEVQFNWYVDGVEVHNAKTKPREEQFNSTYRVVSVVLTVL | |
| | HQDWLNGKEYKCKVSNKGLPSSIEKTISKAKGQPREPQVYTLPPS | |
| | QEEMTKNQVSLTCLVKGFYPSDIAVEWESNGQPENNYKTTPPVL | |
| | DSDGSFFLYSRLTVDKSRWQEGNVFSCSVMHEALHNHYTQKSLS | |
| | LSLGK | |
| Serplulimab, | DIQMTQSPSSLSASVGDRVTITCKASQDVTTAVAWYQQKPGK | 148 |
| HLX-10 | APKLLIYWASTRHTGVPSRFSGSGSGTDFTLTISSLQPEDFAT | |
| Light Chain | YYCQQHYTIPWTFGGGTKLEIKRTVAAPSVFIFPPSDEQLKSGT | |
| (VL in Bold) | ASVVCLLNNFYPREAKVQWKVDNALQSGNSQESVTEQDSKDST | |
| | YSLSSTLTLSKADYEKHKVYACEVTHQGLSSPVTKSFNRGEC | |
| Retifanlimab, | QVQLVQSGAEVKKPGASVKVSCKASGYSFTSYWMNWVRQA | 149 |
| 2Y3T5IF01Z, | PGQGLEWIGVIHPSDSETWLDQKFKDRVTITVDKSTSTAYME | |
| INCMGA- | LSSLRSEDTAVYYCAREHYGTSPFAYWGQGTLVTVSSASTKG | |
| 00012, | PSVFPLAPCSRSTSESTAALGCLVKDYFPEPVTVSWNSGALTSGV | |
| INCMGA-0012, | HTFPAVLQSSGLYSLSSVVTVPSSSLGTKTYTCNVDHKPSNTKVD | |
| MGA-012 | KRVESKYGPPCPPCPAPEFLGGPSVFLFPPKPKDTLMISRTPEVTC | |
| Heavy Chain | VVVDVSQEDPEVQFNWYVDGVEVHNAKTKPREEQFNSTYRVVS | |
| (VH in Bold) | VLTVLHQDWLNGKEYKCKVSNKGLPSSIEKTISKAKGQPREPQV | |
| | YTLPPSQEEMTKNQVSLTCLVKGFYPSDIAVEWESNGQPENNYK | |
| | TTPPVLDSDGSFFLYSRLTVDKSRWQEGNVFSCSVMHEALHNHY | |
| | TQKSLSLSLG | |
| Retifanlimab, | EIVLTQSPATLSLSPGERATLSCRASESVDNYGMSFMNWFQQ | 150 |
| 2Y3T5IF01Z, | KPGQPPKLLIHAASNQGSGVPSRFSGSGSGTDFTLTISSLEPED | |
| INCMGA- | FAVYFCQQSKEVPYTFGGGTKVEIKRTVAAPSVFIFPPSDEQLK | |
| 00012, | SGTASVVCLLNNFYPREAKVQWKVDNALQSGNSQESVTEQDSK | |
| INCMGA-0012, | DSTYSLSSTLTLSKADYEKHKVYACEVTHQGLSSPVTKSFNRGE | |
| MGA-012 | C | |
| Light Chain | | |
| (VL in Bold) | | |
| Sasanlimab, | QVQLVQSGAEVKKPGASVKVSCKASGYTFTSYWINWVRQAP | 151 |
| LZZ0IC2EWP, | GQGLEWMGNIYPGSSLTNYNEKFKNRVTMTRDTSTSTVYME | |
| PF-06801591, | LSSLRSEDTAVYYCARLSTGTFAYWGQGTLVTVSSASTKGPSV | |
| RN-888 | FPLAPCSRSTSESTAALGCLVKDYFPEPVTVSWNSGALTSGVHTF | |
| Heavy Chain | PAVLQSSGLYSLSSVVTVPSSSLGTKTYTCNVDHKPSNTKVDKR | |
| (VH in Bold) | VESKYGPPCPPCPAPEFLGGPSVFLFPPKPKDTLMISRTPEVTCVV | |
| | VDVSQEDPEVQFNWYVDGVEVHNAKTKPREEQFNSTYRVVSVL | |
| | TVLHQDWLNGKEYKCKVSNKGLPSSIEKTISKAKGQPREPQVYT | |
| | LPPSQEEMTKNQVSLTCLVKGFYPSDIAVEWESNGQPENNYKTT | |
| | PPVLDSDGSFFLYSRLTVDKSRWQEGNVFSCSVMHEALHNHYTQ | |
| | KSLSLSLGK | |
| Sasanlimab, | DIVMTQSPDSLAVSLGERATINCKSSQSLWDSGNQKNFLTWY | 152 |
| LZZ0IC2EWP, | QQKPGQPPKLLIYWTSYRESGVPDRFSGSGSGTDFTLTISSLQ | |
| PF-06801591, | AEDVAVYYCQNDYFYPHTFGGGTKVEIKRTVAAPSVFIFPPSD | |
| RN-888 | EQLKSGTASVVCLLNNFYPREAKVQWKVDNALQSGNSQESVTE | |
| Light Chain | QDSKDSTYSLSSTLTLSKADYEKHKVYACEVTHQGLSSPVTKSF | |
| (VL in Bold) | NRGEC | |
| Spartalizumab, | EVQLVQSGAEVKKPGESLRISCKGSGYTFTTYWMHWVRQAT | 153 |
| NVP-LZV-184, | GQGLEWMGNIYPGTGGSNFDEKFKNRVTITADKSTSTAYME | |
| PDR-001, | LSSLRSEDTAVYYCTRWTTGTGAYWGQGTTVTVSSASTKGPS | |
| QOG25L6Z8Z | VFPLAPCSRSTSESTAALGCLVKDYFPEPVTVSWNSGALTSGVHT | |
| Heavy Chain | FPAVLQSSGLYSLSSVVTVPSSSLGTKTYTCNVDHKPSNTKVDKR | |
| (VH in Bold) | VESKYGPPCPPCPAPEFLGGPSVFLFPPKPKDTLMISRTPEVTCVV | |
| | VDVSQEDPEVQFNWYVDGVEVHNAKTKPREEQFNSTYRVVSVL | |
| | TVLHQDWLNGKEYKCKVSNKGLPSSIEKTISKAKGQPREPQVYT | |
| | LPPSQEEMTKNQVSLTCLVKGFYPSDIAVEWESNGQPENNYKTT | |
| | PPVLDSDGSFFLYSRLTVDKSRWQEGNVFSCSVMHEALHNHYTQ | |
| | KSLSLSLG | |
| Spartalizumab, | EIVLTQSPATLSLSPGERATLSCKSSQSLLDSGNQKNFLTWYQ | 154 |
| NVP-LZV-184, | QKPGQAPRLLIYWASTRESGVPSRFSGSGSGTDFTFTISSLEAE | |
| PDR-001, | DAATYYCQNDYSYPYTFGQGTKVEIKRTVAAPSVFIFPPSDEQL | |
| QOG25L6Z8Z | KSGTASVVCLLNNFYPREAKVQWKVDNALQSGNSQESVTEQDS | |
| Light Chain | KDSTYSLSSTLTLSKADYEKHKVYACEVTHQGLSSPVTKSFNRG | |
| (VL in Bold) | EC | |
| Cetrelimab, | QVQLVQSGAEVKKPGSSVKVSCKASGGTFSSYAISWVRQAPG | 155 |
| JNJ-3283, JNJ- | QGLEWMGGIIPIFDTANYAQKFQGRVTITADESTSTAYMELS | |
| 63723283, | SLRSEDTAVYYCARPGLAAAYDTGSLDYWGQGTLVTVSSAST | |
| LYK98WP91F | KGPSVFPLAPCSRSTSESTAALGCLVKDYFPEPVTVSWNSGALTS | |
| Heavy Chain | GVHTFPAVLQSSGLYSLSSVVTVPSSSLGTKTYTCNVDHKPSNTK | |
| (VH in Bold) | VDKRVESKYGPPCPPCPAPEFLGGPSVFLFPPKPKDTLMISRTPEV | |
| | TCVVVDVSQEDPEVQFNWYVDGVEVHNAKTKPREEQFNSTYRV | |
| | VSVLTVLHQDWLNGKEYKCKVSNKGLPSSIEKTISKAKGQPREP | |
| | QVYTLPPSQEEMTKNQVSLTCLVKGFYPSDIAVEWESNGQPENN | |
| | YKTTPPVLDSDGSFFLYSRLTVDKSRWQEGNVFSCSVMHEALHN | |
| | HYTQKSLSLSLGK | |
| Cetrelimab, | EIVLTQSPATLSLSPGERATLSCRASQSVRSYLAWYQQKPGQ | 156 |
| JNJ-3283, JNJ- | APRLLIYDASNRATGIPARFSGSGSGTDFTLTISSLEPEDFAVY | |
| 63723283, | YCQQRNYWPLTFGQGTKVEIKRTVAAPSVFIFPPSDEQLKSGT | |
| LYK98WP91F | ASVVCLLNNFYPREAKVQWKVDNALQSGNSQESVTEQDSKDST | |
| Light Chain | YSLSSTLTLSKADYEKHKVYACEVTHQGLSSPVTKSFNRGEC | |
| (VL in Bold) | | |
| Tebotelimab, | DIQMTQSPSSLSASVGDRVTITCRASQDVSSVVAWYQQKPGK | 157 |
| MGD-013 | APKLLIYSASYRYTGVPSRFSGSGSGTDFTLTISSLQPEDFATY | |
| Heavy Chain | YCQQHYSTPWTFGGGTKLEIKGGGSGGGGQVQLVQSGAEVK | |
| (VH in Bold) | KPGASVKVSCKASGYSFTSYWMNWVRQAPGQGLEWIGVIHPSD | |
| | SETWLDQKFKDRVTITVDKSTSTAYMELSSLRSEDTAVYYCARE | |
| | HYGTSPFAYWGQGTLVTVSSGGCGGGEVAACEKEVAALEKEVA | |
| | ALEKEVAALEKESKYGPPCPPCPAPEFLGGPSVFLFPPKPKDTLYI | |
| | TREPEVTCVVVDVSQEDPEVQFNWYVDGVEVHNAKTKPREEQF | |
| | NSTYRVVSVLTVLHQDWLNGKEYKCKVSNKGLPSSIEKTISKAK | |
| | GQPREPQVYTLPPSQEEMTKNQVSLTCLVKGFYPSDIAVEWESN | |
| | GQPENNYKTTPPVLDSDGSFFLYSRLTVDKSRWQEGNVFSCSVM | |
| | HEALHNHYTQKSLSLSLG | |
| Tebotelimab, | EIVLTQSPATLSLSPGERATLSCRASESVDNYGMSFMNWFQQ | 158 |
| MGD-013 | KPGQPPKLLIHAASNQGSGVPSRFSGSGSGTDFTLTISSLEPED | |
| Light Chain | FAVYFCQQSKEVPYTFGGGTKVEIKGGGSGGGGQVQLVQSGA | |
| (VL in Bold) | EVKKPGASVKVSCKASGYTFTDYNMDWVRQAPGQGLEWMGDI | |
| | NPDNGVTIYNQKFEGRVTMTTDTSTSTAYMELRSLRSDDTAVYY | |
| | CAREADYFYFDYWGQGTTLTVSSGGCGGGKVAACKEKVAALK | |
| | EKVAALKEKVAALKE | |
| Pidilizumab, | QVQLVQSGSELKKPGASVKISCKASGYTFTNYGMNWVRQAP | 159 |
| CT-011, MDV- | GQGLQWMGWINTDSGESTYAEEFKGRFVFSLDTSVNTAYLQ | |
| 9300 | ITSLTAEDTGMYFCVRVGYDALDYWGQGTLVTVSSASTKGPS | |
| Heavy Chain | VFPLAPSSKSTSGGTAALGCLVKDYFPEPVTVSWNSGALTSGVH | |
| (VH in Bold) | TFPAVLQSSGLYSLSSVVTVPSSSLGTQTYICNVNHKPSNTKVDK | |
| | RVEPKSCDKTHTCPPCPAPELLGGPSVFLFPPKPKDTLMISRTPEV | |
| | TCVVVDVSHEDPEVKFNWYVDGVEVHNAKTKPREEQYNSTYR | |
| | VVSVLTVLHQDWLNGKEYKCKVSNKALPAPIEKTISKAKGQPRE | |
| | PQVYTLPPSREEMTKNQVSLTCLVKGFYPSDIAVEWESNGQPEN | |
| | NYKTTPPVLDSDGSFFLYSKLTVDKSRWQQGNVFSCSVMHEAL | |
| | HNHYTQKSLSLSPGK | |
| Pidilizumab, | EIVLTQSPSSLSASVGDRVTITCSARSSVSYMHWFQQKPGKAP | 160 |
| CT-011, MDV- | KLWIYRTSNLASGVPSRFSGSGSGTSYCLTINSLQPEDFATYY | |
| 9300 | CQQRSSFPLTFGGGTKLEIKRTVAAPSVFIFPPSDEQLKSGTASV | |
| Light Chain | VCLLNNFYPREAKVQWKVDNALQSGNSQESVTEQDSKDSTYSL | |
| (VL in Bold) | SSTLTLSKADYEKHKVYACEVTHQGLSSPVTKSFNRGEC | |
| SG-001 VH | QVQLVESGGGVVQPGRSLRLTCKASGLTFSSSGMHWVRQAPGK | 161 |
| | GLEWVAVIWYDGSKRYYADSVKGRFTISRDNSKNTLFLQMNSL | |
| | RAEDTAVYYCATNNDYWGQGTLVTVSS | |
| SG-001 VL | EIVLTQSPATLSLSPGERATLSCRASQSVSSYLAWYQQKPGQAPR | 162 |
| | LLIYTASNRATGIPARFSGSGSGTDFTLTISSLEPEDFAVYYCQQY | |
| | SNWPRTFGQGTKVEIK | |
| mpLZM-009 | EVQLQQSGPVLVKPGASVKMSCKASGYTFTSYYMYWVKQSHG | 163 |
| VH | KSLEWIGGVNPSNGGTNFNEKFKSKATLTVDKSSSTAYMELNSL | |
| (Murine | TSEDSAVYYCARRDYRYDMGFDYWGQGTTLTVSS | |
| Precursor of | | |
| LZM-009) | | |
| mpLZM-009 | QIVLTQSPAIMSASPGEKVTMTCRASKGVSTSGYSYLHWYQQKP | 164 |
| VL | GSSPRLLIYLASYLESGVPVRFSGSGSGTSYSLTISRMEAEDAATY | |
| (Murine | YCQHSRELPLTFGTGTRLEIK | |
| Precursor of | | |
| LZM-009) | | |
| LZM-009 VH | QVQLVQSGAEVKKPGASVKVSCKASGYTFTSYYMYWVRQAPG | 165 |
| | QGLEWMGGVNPSNGGTNFNEKFKSRVTITADKSTSTAYMELSSL | |
| | RSEDTAVYYCARRDYRYDMGFDYWGQGTTVTVSS | |
| LZM-009 VL | EIVLTQSPATLSLSPGERATISCRASKGVSTSGYSYLHWYQQKPG | 166 |
| | QAPRLLIYLASYLESGVPARFSGSGSGTDFTLTISSLEPEDFATYY | |
| | CQHSRELPLTFGTGTKVEIK | |
| Budigalimab, | EIQLVQSGAEVKKPGSSVKVSCKASGYTFTHYGMNWVRQAP | 167 |
| 6VDO4TY300, | GQGLEWVGWVNTYTGEPTYADDFKGRLTFTLDTSTSTAYM | |
| ABBV-181, PR- | ELSSLRSEDTAVYYCTREGEGLGFGDWGQGTTVTVSSASTKG | |
| 1648817 | PSVFPLAPSSKSTSGGTAALGCLVKDYFPEPVTVSWNSGALTSGV | |
| Heavy Chain | HTFPAVLQSSGLYSLSSVVTVPSSSLGTQTYICNVNHKPSNTKVD | |
| (VH in Bold) | KKVEPKSCDKTHTCPPCPAPEAAGGPSVFLFPPKPKDTLMISRTPE | |
| | VTCVVVDVSHEDPEVKFNWYVDGVEVHNAKTKPREEQYNSTY | |
| | RVVSVLTVLHQDWLNGKEYKCKVSNKALPAPIEKTISKAKGQPR | |
| | EPQVYTLPPSREEMTKNQVSLTCLVKGFYPSDIAVEWESNGQPE | |
| | NNYKTTPPVLDSDGSFFLYSKLTVDKSRWQQGNVFSCSVMHEA | |
| | LHNHYTQKSLSLSPGK | |
| Budigalimab, | DVVMTQSPLSLPVTPGEPASISCRSSQSIVHSHGDTYLEWYLQ | 168 |
| 6VDO4TY3OO, | KPGQSPQLLIYKVSNRFSGVPDRFSGSGSGTDFTLKISRVEAE | |
| ABBV-181, PR- | DVGVYYCFQGSHIPVTFGQGTKLEIKRTVAAPSVFIFPPSDEQL | |
| 1648817 Light | KSGTASVVCLLNNFYPREAKVQWKVDNALQSGNSQESVTEQDS | |
| Chain | KDSTYSLSSTLTLSKADYEKHKVYACEVTHQGLSSPVTKSFNRG | |
| (VL in Bold) | EC | |
| Lambrolizumab, | NYYMY | 169 |
| Pembrolizumab, | | |
| KEYTRUDA ®, | | |
| MK-3475, SCH- | | |
| 900475, | | |
| h409A11 | | |
| VH CDR1 | | |
| Lambrolizumab, | GINPSNGGTNFNEKFKN | 170 |
| Pembrolizumab, | | |
| KEYTRUDA ®, | | |
| MK-3475, SCH- | | |
| 900475, | | |
| h409A11 | | |
| VH CDR2 | | |
| Lambrolizumab, | RDYRFDMGFDY | 171 |
| Pembrolizumab, | | |
| KEYTRUDA ®, | | |
| MK-3475, SCH- | | |
| 900475, | | |
| h409A11 | | |
| VH CDR3 | | |
| Lambrolizumab, | RASKGVSTSGYSYLH | 172 |
| Pembrolizumab, | | |
| KEYTRUDA ®, | | |
| MK-3475, SCH- | | |
| 900475, | | |
| h409A11 | | |
| VL CDR1 | | |
| Lambrolizumab, | LASYLES | 173 |
| Pembrolizumab, | | |
| KEYTRUDA ®, | | |
| MK-3475, SCH- | | |
| 900475, | | |
| h409A11 | | |
| VL CDR2 | | |
| Lambrolizumab, | QHSRDLPLT | 174 |
| Pembrolizumab, | | |
| KEYTRUDA ®, | | |
| MK-3475, SCH- | | |
| 900475, | | |
| h409A11 | | |
| VL CDR3 | | |
| Nivolumab, | NSGMH | 175 |
| Nivolumab | | |
| BMS, | | |
| OPDIVO ®, | | |
| BMS-936558, | | |
| MDX-1106, | | |
| ONO-4538 | | |
| VH CDR1 | | |
| Nivolumab, | VIWYDGSKRYYADSVKG | 176 |
| Nivolumab | | |
| BMS, | | |
| OPDIVO ®, | | |
| BMS-936558, | | |
| MDX-1106, | | |
| ONO-4538 | | |
| VH CDR2 | | |
| Nivolumab, | NDDY | 177 |
| Nivolumab | | |
| BMS, | | |
| OPDIVO ®, | | |
| BMS-936558, | | |
| MDX-1106, | | |
| ONO-4538 | | |
| VH CDR3 | | |
| Nivolumab, | RASQSVSSYLA | 178 |
| Nivolumab | | |
| BMS, | | |
| OPDIVO ®, | | |
| BMS-936558, | | |
| MDX-1106, | | |
| ONO-4538 | | |
| VL CDR1 | | |
| Nivolumab, | DASNRAT | 179 |
| Nivolumab | | |
| BMS, | | |
| OPDIVO ®, | | |
| BMS-936558, | | |
| MDX-1106, | | |
| ONO-4538 | | |
| VL CDR2 | | |
| Nivolumab, | QQSSNWPRT | 180 |
| Nivolumab | | |
| BMS, | | |
| OPDIVO ®, | | |
| BMS-936558, | | |
| MDX-1106, | | |
| ONO-4538 | | |
| VL CDR3 | | |
| Serplulimab, | FTFSNYGMS | 181 |
| HLX-10 | | |
| VH CDR1 | | |
| Serplulimab, | TISGGGSNIY | 182 |
| HLX-10 | | |
| VH CDR2 | | |
| Serplulimab, | VSYYYGIDF | 183 |
| HLX-10 | | |
| VH CDR3 | | |
| Serplulimab, | KASQDVTTAVA | 184 |
| HLX-10 | | |
| VL CDR1 | | |
| Serplulimab, | WASTRHT | 185 |
| HLX-10 | | |
| VL CDR2 | | |
| Serplulimab, | QQHYTIPWT | 186 |
| HLX-10 | | |
| VL CDR3 | | |
| SG-001 | GLTFSSSG | 187 |
| VH CDR1 | | |
| SG-001 | IWYDGSKR | 188 |
| VH CDR2 | | |
| SG-001 | ATNNDY | 189 |
| VH CDR3 | | |
| SG-001 | RASQSVSSYLA | 190 |
| VL CDR1 | | |
| SG-001 | TASNRAT | 191 |
| VL CDR2 | | |
| SG-001 | QQYSNWPRT | 192 |
| VL CDR3 | | |
| PD-1-Fc- | MQIPQAPWPWWAVLQLGWRPGWFLDSPDRPWNPPTFSPALLVV | 193 |
| OX40L (Code), | TEGDNATFTCSFSNTSESFVLNWYRMSPSNQTDKLAAFPEDRSQP | |
| SL-279252 | GQDCRFRVTQLPNGRDFHMSVVRARRNDSGTYLCGAISLAPKA | |
| (Code), TAK- | QIKESLRAELRVTERRAEVPTAHPSPSPRPAGQFQSKYGPPCPSCP | |
| 252 (Code) | APEFLGGPSVFLFPPKPKDTLMISRTPEVTCWVDVSQEDPEVQFN | |
| | WYVDGVEVHNAKTKPREEQFNSTYRVVSVLTVLHQDWLSGKE | |
| | YKCKVSSKGLPSSIEKTISNATGQPREPQVYTLPPSQEEMTKNQV | |
| | SLTCLVKGFYPSDIAVEWESNGQPENNYKTTPPVLDSDGSFFLYS | |
| | RLTVDKSSWQEGNVFSCSVMHEALHNHYTQKSLSLSLGKIEGR | |
| | MDQVSHRYPRIQSIKVQFTEYKKEKGFILTSQKEDEIMKVQNNSV | |
| | IINCDGFYLISLKGYFSQEVNISLHYQKDEEPLFQLKKVRSVNSLM | |
| | VASLTYKDKVYLNVTTDNTSLDDFHVNGGELILIHQNPGEFCVL | |
| | MQIPQAPWPWWAVLQLGWRPGWFLDSPDRPWNPPTFSPALLVV | |
| | TEGDNATFTCSFSNTSESFVLNWYRMSPSNQTDKLAAFPEDRSQP | |
| | GQDCRFRVTQLPNGRDFHMSVVRARRNDSGTYLCGAISLAPKA | |
| | QIKESLRAELRVTERRAEVPTAHPSPSPRPAGQFQQVSHRYPRIQS | |
| | IKVQFTEYKKEKGFILTSQKEDEIMKVQNNSVIINCDGFYLISLKG | |
| | YFSQEVNISLHYQKDEEPLFQLKKVRSVNSLMVASLTYKDKVYL | |
| | NVTTDNTSLDDFHVNGGELILIHQNPGEFCVL | |
| PD1-0103-0314, | EVQLLESGGGLVQPGGSLRLSCAASGFSFSSYTMSWVRQAPGKG | 194 |
| PD1-0103-0313, | LEWVATISGGGRDIYYPDSVKGRFTISRDNSKNTLYLQMNSLRA | |
| and PD1-0103- | EDTAVYYCVLLTGRVYFALDSWGQGTLVTVSS | |
| 0312 VH | | |
| PD1-0103-0312 | DIVMTQSPDSLAVSLGERATINCKASESVDTSDNSFIHWYQQKPG | 195 |
| VL | QSPKLLIYRSSTLESGVPDRFSGSGSGTDFTLTISSLQAEDVAVYY | |
| | CQQNYDVPWTFGQGTKVEIK | |
| PD1-0103-0313 | DVVMTQSPLSLPVTLGQPASISCRASESVDTSDNSFIHWYQQRPG | 196 |
| VL | QSPRLLIYRSSTLESGVPDRFSGSGSGTDFTLKISRVEAEDVGVYY | |
| | CQQNYDVPWTFGQGTKVEIK | |
| PD1-0103-0314 | EIVLTQSPATLSLSPGERATLSCRASESVDTSDNSFIHWYQQKPGQ | 197 |
| VL | SPRLLIYRSSTLESGIPARFSGSGSGTDFTLTISSLEPEDFAVYYCQ | |
| | QNYDVPWTFGQGTKVEIK | |
| PD1-0050 | DVQLQESGPGLVKPSQSLSLTCTVTGYSITSDYAWNWIRQFPGN | 198 |
| VH | KLEWMGYITYTGRTSYNPSLKSRISITRDTSKNQFFLQLNSVTTE | |
| | DTATYYCAREMDYYGSTLDYWGQGTTLTVSS | |
| PD1-0050 | KIVLTQSPASLAVSLRQRATISCRASESVDRYGNSFIHWYQQKPG | 199 |
| VL | QPPKVLIYRASNLESGFPARFSGSGSRTDFTLTIDPVEADDAATYY | |
| | CQQNNEDPYTFGSGTKLEIK | |
| PD1-0069 | QVQLQQSGPELVRPGVSVKISCKGSGYTFTDYAMHWVKQSHAR | 200 |
| VH | TLEWIGVISTYSGDTNYNQKFKDKATMTVDKSSSTAYLELARMT | |
| | SEDSAIYYCARLGITTGFAYWGQGTLVTVSA | |
| PD1-0069 | DIVLTQSPASLAVSLGQRATISCRASKGVSTSSYSFMHWYQQKPR | 201 |
| VL | QPPKLLIKYASYLESGVPARFSGSGSGTDFTLNIHPVEEEDAATY | |
| | YCHHSREFPWTFGGGTKLEIK | |
| PD1-0073 | EVKLVESGGGLVKPGGSLKLSCAASGFTFSNYGMSWIRQTPEKG | 202 |
| VH | LEWVATISGGGRDTYYPDSVKGRFTISRDNVKNNLYLQMSSLRS | |
| | EDTAFYYCASYYYGIDYWGQGTSVTVSS | |
| PD1-0073 | DIVMTQPHKEMSTSVGDRVRITCKASQDVTTAVAWYQQKPGQS | 203 |
| VL | PKLLIYWASTRHTGVPDRFTGSGSGTEFTLTISSVQAEDLALYYC | |
| | QQHYSIPWTFGGGTKLEIK | |
| PD1-0078 | QVQLQQPGAELVKPGASVKMSCKASGYTFTSTWMHWVKQRPG | 204 |
| VH | QGLEWIGAIDPSDSYTTYNQKFKGKATLTVDTSSTTAYMQLSSL | |
| | TSEDSAVYYCTRSPFDYWGQGTTLTVSS | |
| PD1-0078 | DIVMTQSHKEMSTSVGDRVSITCKASQDVSTAVAWYQQKPGQS | 205 |
| VL | PKLLIYSASYRYTGVPDRFTGSGSGTDFTFAISSVQAEDLAVYYC | |
| | QQHYSHPFTFGSGTKLEIK | |
| PD1-0098 | DVQLQESGPGLVKPSQSLSLTCTVTGYSITSDYAWNWIRQFPGD | 206 |
| VH | KLEWLGYITYSGFTNYNPSLKSRISISRDTSKNQFFLQLNSVATED | |
| | TATYYCARWHGSAPWYFDYWGRGTTLTVSS | |
| PD1-0098 | DVLMTQTPLSLPVSLGDQASISCRSSQNIVHSDGNTYLEWYLQKP | 207 |
| VL | GQSPNLLIYKVSRRFSGVPDRFSGSGSGTDFTLKISRVEAEDLGV | |
| | YYCFQGSHFPLTFGAGTKLELK | |
| PD1-0102 | DVQLQESGPDLVKPSQSLSLTCTVTGYSITSGYSWHWIRQFPGNK | 208 |
| VH | LEWMGFIHSSGDTNYNPSLKSRISFTRDTSKNQFFLQLSSLTDEDT | |
| | ATYYCATYRNWYFDVWGAGTTVTVSS | |
| PD1-0102 | DIVMTQSPSSLTVTAGEKVTMRCKSSQSLLNSGTQKNYLTWYQQ | 209 |
| VL | KPGQPPKLLIYWASTRESGVPNRFTGSGSGTDFTLTISSVQAEDLS | |
| | VYYCQSDYTFPLTFGGGTKLELK | |
| PD1-112 | QVQLVQSGVEVKKPGASVKVSCKASGYTFTNYYMYWVRQAPG | 210 |
| VH | QGLEWMGGINPSNGGTNFNEKFKNRVTLTTDSSTTTAYMELKSL | |
| | QFDDTAVYYCARRDYRFDMGFDYWGQGTTVTVSS | |
| PD1-112 | EIVLTQSPATLSLSPGERATLSCRASKGVSTSGYSYLHWYQQKPG | 211 |
| VL | QAPRLLIYLASYLESGVPARFSGSGSGTDFTLTISSLEPEDFAVYY | |
| | CQHSRDLPLTFGGGTKVEIK | |
| PD1-114 | QVQLVESGGGVVQPGRSLRLDCKASGITFSNSGMHWVRQAPGK | 212 |
| VH | GLEWVAVIWYDGSKRYYADSVKGRFTISRDNSKNTLFLQMNSL | |
| | RAEDTAVYYCATNDDYWGQGTLVTVSS | |
| PD1-114 | EIVLTQSPATLSLSPGERATLSCRASQSVSSYLAWYQQKPGQAPR | 213 |
| VL | LLIYDASNRATGIPARFSGSGSGTDFTLTISSLEPEDFAVYYCQQS | |
| | SNWPRTFGQGTKVEIK | |
| PD1-0103 | EVILVESGGGLVKPGGSLKLSCAASGFSFSSYTMSWVRQTPEKRL | 214 |
| (Murine) | DWVATISGGGRDIYYPDSVKGRFTISRDNAKNTLYLEMSSLMSE | |
| VH | DTALYYCVLLTGRVYFALDSWGQGTSVTVSS | |
| PD1-0103 | KIVLTQSPASLPVSLGQRATISCRASESVDTSDNSFIHWYQQRPGQ | 215 |
| (Murine) | SPKLLIYRSSTLESGVPARFSGSGSRTDFTLTIDPVEADDVATYYC | |
| VL | QQNYDVPWTFGGGTKLEIK | |
| PD1-0103-0314, | GFSFSSYT | 216 |
| PD1-0103-0313, | | |
| PD1-0103-0312, | | |
| and PD1-0103 | | |
| (Murine) | | |
| VH CDR1 | | |
| PD1-0103-0314, | ISGGGRDI | 217 |
| PD1-0103-0313, | | |
| PD1-0103-0312, | | |
| and PD1-0103 | | |
| (Murine) | | |
| VH CDR2 | | |
| PD1-0103-0314, | VLLTGRVYFALDS | 218 |
| PD1-0103-0313, | | |
| PD1-0103-0312, | | |
| and PD1-0103 | | |
| (Murine) | | |
| VH CDR3 | | |
| PD1-0103-0314, | ESVDTSDNSF | 219 |
| PD1-0103-0313, | | |
| PD1-0103-0312, | | |
| and PD1-0103 | | |
| (Murine) | | |
| VL CDR1 | | |
| PD1-0103-0314, | RSS | |
| PD1-0103-0313, | | |
| PD1-0103-0312, | | |
| and PD1-0103 | | |
| (Murine) | | |
| VL CDR2 | | |
| PD1-0103-0314, | NYDVPW | 221 |
| PD1-0103-0313, | | |
| PD1-0103-0312, | | |
| and PD1-0103 | | |
| (Murine) | | |
| VL CDR3 | | |
| PD1-0050 VH | GYSITSDY | 222 |
| CDR1 | | |
| PD1-0050 VH | YTG | |
| CDR2 | | |
| PD1-0050 VH | MDYYGSTLD | 224 |
| CDR3 | | |
| PD1-0050 VL | SESVDRYGNSF | 225 |
| CDR1 | | |
| PD1-0050 VL | RAN | |
| CDR2 | | |
| PD1-0050 VL | NNEDPY | 227 |
| CDR3 | | |
| PD1-0069 VH | GYTFTDY | 228 |
| CDR1 | | |
| PD1-0069 VH | YSG | |
| CDR2 | | |
| PD1-0069 VH | GITTGFA | 230 |
| CDR3 | | |
| PD1-0069 VL | SKGVSTSSYSF | 231 |
| CDR1 | | |
| PD1-0069 VL | YAS | |
| CDR2 | | |
| PD1-0069 VL | SREFPW | 233 |
| CDR3 | | |
| PD1-0073 VH | GFTFSNY | 234 |
| CDR1 | | |
| PD1-0073 VH | GGR | |
| CDR2 | | |
| PD1-0073 VH | YYGID | 236 |
| CDR3 | | |
| PD1-0073 VL | SQDVTTA | 237 |
| CDR1 | | |
| PD1-0073 VL | WAS | |
| CDR2 | | |
| PD1-0073 VL | HYSIPW | 239 |
| CDR3 | | |
| PD1-0078 VH | GYTFTST | 240 |
| CDR1 | | |
| PD1-0078 VH | SDS | |
| CDR2 | | |
| PD1-0078 VH | PFD | |
| CDR3 | | |
| PD1-0078 VL | SQDVSTA | 243 |
| CDR1 | | |
| PD1-0078 VL | SAS | |
| CDR2 | | |
| PD1-0078 VL | HYSHPF | 245 |
| CDR3 | | |
| PD1-0098 VH | GYSITSDY | 246 |
| CDR1 | | |
| PD1-0098 VH | YSG | |
| CDR2 | | |
| PD1-0098 VH | GSAPWYFD | 248 |
| CDR3 | | |
| PD1-0098 VL | SQNIVHSDGNTY | 249 |
| CDR1 | | |
| PD1-0098 VL | KVS | |
| CDR2 | | |
| PD1-0098 VL | SHFPL | 251 |
| CDR3 | | |
| PD1-0102 VH | GYSITSGY | 252 |
| CDR1 | | |
| PD1-0102 VH | SSG | |
| CDR2 | | |
| PD1-0102 VH | RNWYFD | 254 |
| CDR3 | | |
| PD1-0102 VL | SQSLLNSGTQKNY | 255 |
| CDR1 | | |
| PD1-0102 VL | WAS | |
| CDR2 | | |
| PD1-0102 VL | DYTFPL | 257 |
| CDR3 | | |
| PD1-112VH | GYFTNYY | 258 |
| CDR1 | | |
| PD1-112VH | INPSNGGT | 259 |
| CDR2 | | |
| PD1-112VH | ARRDYRFDMGFDY | 260 |
| CDR3 | | |
| PD1-113VL | KGVSTSGYSY | 261 |
| CDR1 | | |
| PD1-113VL | LAS | |
| CDR2 | | |
| PD1-113VL | QHSRDLPLT | 263 |
| CDR3 | | |
| PD1-114VH | GITFSNSG | 264 |
| CDR1 | | |
| PD1-114VH | IWYDGSKR | 265 |
| CDR2 | | |
| PD1-114VH | ATNDDY | 266 |
| CDR3 | | |
| PD1-115VL | QSVSSY | 267 |
| CDR1 | | |
| PD1-115VL | DAS | |
| CDR2 | | |
| PD1-115VL | QQSSNWRRT | 269 |
| CDR3 | | |
| VH_17D8 | QVQLVESGGDVVQPGGSLRLSCAASGVAFSNYGMHWVRQ | 270 |
| | APGKGLEWVAVIWYDGSNKYYADSVKGRFTISRDNSKNM | |
| | LYLQMNSLRAEDTAMYYCARNDDYWGQGTLVTVSS | |
| VL_17D8 | EIVLTQSPATLSLSPGERATLSCRASQSVSSYLAWYQQKPG | 271 |
| | QAPRLIIYDASNRATGIPARFSGSGSGTDFTLTISSLEPEDFA | |
| | VYYCQQRSNWPLTFGGGTKVEIK | |
| VH_2D3 | QVQLVESGGDVVQPGRSLRLSCAASGLTFTNYGFHWVRQ | 272 |
| | APGKGLEWVAVIWYDGSKKYYADSVKGRFTISRDNSKNT | |
| | LYLQMNNLRAEDTAVYYCATGDDYWGQGTLVTVSS | |
| VL_2D3 | EIVLTQSPATLSLSPGERATLSCRASQSVSSYLAWYQQKPG | 273 |
| | QAPRLLIYDTSNRATGIPARFSGSGSGTDFTLTISSLEPEDFA | |
| | VYYCQQRSNWPLTFGGGTKVEIK | |
| VH_4H1 | QVYLVESGGGVVQPGRSLRLSCAASGFTFSNYGMHWVRQ | 274 |
| | APGKGLEWVALIWYDGSNKYYADSVKGRFTISRDNSKNTL | |
| | YLQMTSLRVEDTAVYYCASNVDHWGQGTLVTVSS | |
| VH_4A11 | QLQLQESGPGLVKPSETLSLTCTVSGGSLSRSSFFWGWIRQP | 275 |
| | PGKGLEWIGSIYYSGSTYYNPSLKSRVTISVDTSKNQFSLKL | |
| | SSVTAADTAVYYCVRDYDILTGDEDYWGQGTLVTVSS | |
| VH_7D3 | QVQLVESGGGVVQPGRSLRLSCTTSGITFSSYGFHWVRQAP | 276 |
| | GKGLEWVAVIWYDGSKKYYADSVKGRFTLSRDDSKNTLY | |
| | LQMNSLRAEDTAVYYCVTGDDYWGQGTLVTVSS | |
| VL_7D3 | EIVLTQSPATLSLSPGERATLSCRASQSVSSYLAWYQQKPG | 277 |
| | QAPRLLIYDASNRATGIPARFSGSGSGTDFTLTISSLEPEDFA | |
| | VYYCQQRSNWPLTFGGGTKVEIK | |
| VH_5F4 | QLQLQESGPGLVKPSETLSLTCSVSGGSLSRSSYFWGWIRQ | 278 |
| | PPGKGLEWIASIFYSGETYFNPSLKSRVTISVDTSRNQFSLKL | |
| | SSVTAADTAVYYCARDYDILTGDEDYWGQGTLVTVSS | |
| VL_5F4 | DIQMTQSPSSLSASVGDRVTITCRASQGISSWLAWYQQKPE | 279 |
| | KAPKSLIYAASSLQSGVPSRFSGSGSGTDFTLTISSLQPEDFA | |
| | TYYCQQYYSYPRTFGQGTKVEIK | |
| 17D8_VH_CDR1 | NYGMH | 280 |
| 17D8_VH_CDR2 | VIWYDGSNKYYADSVKG | 281 |
| 17D8_VH_CDR3 | NDDY | 282 |
| 17D8_VL_CDR1 | RASQSVSSYLA | 283 |
| 17D8_VL_CDR2 | DASNRAT | 284 |
| 17D8_VL_CDR3 | QQRSNWPLT | 285 |
| 2D3_VH_CDR1 | NYGFH | 286 |
| 2D3_VH_CDR2 | VIWYDGSKKYYADSVKG | 287 |
| 2D3_VH_CDR3 | GDDY | 288 |
| 2D3_VL_CDR1 | RASQSVSSYLA | 289 |
| 2D3_VL_CDR2 | DTSNRAT | 290 |
| 2D3_VL_CDR3 | QQRSNWPLT | 291 |
| 4H1_VH_CDR1 | NYGMH | 292 |
| 4H1_VH_CDR2 | LIWYDGSNKYYADSVKG | 293 |
| 4H1_VH_CDR3 | NVDH | 294 |
| 4A11_VH_CDR1 | RSSFFWG | 295 |
| 4A11_VH_CDR2 | SIYYSGSTYYNPSLKS | 296 |
| 4A11_VH_CDR3 | DYDILTGDEDY | 297 |
| 7D3_VH_CDR1 | SYGFH | 298 |
| 7D3_VH_CDR2 | VIWYDGSKKYYADSVKG | 299 |
| 7D3_VH_CDR3 | GDDY | 300 |
| 7D3_VL_CDR1 | RASQSVSSYLA | 301 |
| 7D3_VL_CDR2 | DASNRAT | 302 |
| 7D3_VL_CDR3 | QQRSNWPLT | 303 |
| 5F4_VH_CDR1 | RSSYFWG | 304 |
| 5F4_VH_CDR2 | SIFYSGETYFNPSLKS | 305 |
| 5F4_VH_CDR3 | DYDILTGDEDY | 306 |
| 5F4_VL_CDR1 | RASQGISSWLA | 307 |
| 5F4_VL_CDR2 | AASSLQS | 308 |
| 5F4_VL_CDR3 | QQYYSYPRT | 309 |

In some instances, the SEQ ID NOs listed in Table 5 contain full-length heavy or light chains of the indicated antibodies with the VH or VL respectively indicated in bold. Where there is a reference herein to a VH or VL of a SEQ ID NO in Table 5 which contains a full-length heavy or light chain, it is intended to reference the bolded portion of the sequence. For example, reference to “a VH having an amino acid sequence shown in SEQ ID NO: 125” refers to the bolded portion of SEQ ID NO: 125 in Table 5.

An anti-PD-1 polypeptide or an anti-PD-1 antigen binding fragment can comprise a heavy chain or a VH having an amino acid sequence of any one of SEQ ID NOS: 125, 127, 129, 131, 133, 135, 137, 139, 141, 143, 145, 147, 149, 151, 153, 155, 157, 159, 161, 163, 165, 167, 194, 198, 200, 202, 204, 206, 208, 210, 212, 214, 270, 272, 274, 275, 276, and 278, or a portion corresponding to a VH thereof. An anti-PD-1 polypeptide or an anti-PD-1 antigen binding fragment can comprise a light chain or a VL having an amino acid sequence of any one of SEQ ID NOS: 126, 128, 130, 132, 134, 136, 138, 140, 142, 144, 146, 148, 150, 152, 154, 156, 158, 160, 162, 164, 166, 168, 195, 196, 197, 199, 201, 203, 205, 207, 209, 211, 213, 215, 271, 273, 277, and 279, or a portion corresponding to a VL thereof.

In one instance, an anti-PD-1 polypeptide or an anti-PD-1 antigen binding fragment comprises a VH having an amino acid sequence shown in SEQ ID NO: 125, and a VL having an amino acid sequence shown in SEQ ID NO: 126. In another instance, an anti-PD-1 polypeptide or an anti-PD-1 antigen binding fragment comprises a VH having an amino acid sequence shown in SEQ ID NO: 127, and a VL having an amino acid sequence shown in SEQ ID NO: 128. In another instance, an anti-PD-1 polypeptide or an anti-PD-1 antigen binding fragment comprises a VH having an amino acid sequence shown in SEQ ID NO: 129, and a VL having an amino acid sequence shown in SEQ ID NO: 130. In another instance, an anti-PD-1 polypeptide or an anti-PD-1 antigen binding fragment comprises a VH having an amino acid sequence shown in SEQ ID NO: 131, and a VL having an amino acid sequence shown in SEQ ID NO: 132. In another instance, an anti-PD-1 polypeptide or an anti-PD-1 antigen binding fragment comprises a VH having an amino acid sequence shown in SEQ ID NO: 133, and a VL having an amino acid sequence shown in SEQ ID NO: 134. In another instance, an anti-PD-1 polypeptide or an anti-PD-1 antigen binding fragment comprises a VH having an amino acid sequence shown in SEQ ID NO: 135, and a VL having an amino acid sequence shown in SEQ ID NO: 136. In another instance, an anti-PD-1 polypeptide or an anti-PD-1 antigen binding fragment comprises a VH having an amino acid sequence shown in SEQ ID NO: 137, and a VL having an amino acid sequence shown in SEQ ID NO: 138. In another instance, an anti-PD-1 polypeptide or an anti-PD-1 antigen binding fragment comprises a VH having an amino acid sequence shown in SEQ ID NO: 139, and a VL having an amino acid sequence shown in SEQ ID NO: 140. In another instance, an anti-PD-1 polypeptide or an anti-PD-1 antigen binding fragment comprises a VH having an amino acid sequence shown in SEQ ID NO: 141, and a VL having an amino acid sequence shown in SEQ ID NO: 142. In another instance, an anti-PD-1 polypeptide or an anti-PD-1 antigen binding fragment comprises a VH having an amino acid sequence shown in SEQ ID NO: 143, and a VL having an amino acid sequence shown in SEQ ID NO: 144. In another instance, an anti-PD-1 polypeptide or an anti-PD-1 antigen binding fragment comprises a VH having an amino acid sequence shown in SEQ ID NO: 145, and a VL having an amino acid sequence shown in SEQ ID NO: 146. In another instance, an anti-PD-1 polypeptide or an anti-PD-1 antigen binding fragment comprises a VH having an amino acid sequence shown in SEQ ID NO: 147, and a VL having an amino acid sequence shown in SEQ ID NO: 148. In another instance, an anti-PD-1 polypeptide or an anti-PD-1 antigen binding fragment comprises a VH having an amino acid sequence shown in SEQ ID NO: 149, and a VL having an amino acid sequence shown in SEQ ID NO: 150. In another instance, an anti-PD-1 polypeptide or an anti-PD-1 antigen binding fragment comprises a VH having an amino acid sequence shown in SEQ ID NO: 151, and a VL having an amino acid sequence shown in SEQ ID NO: 152. In another instance, an anti-PD-1 polypeptide or an anti-PD-1 antigen binding fragment comprises a VH having an amino acid sequence shown in SEQ ID NO: 153, and a VL having an amino acid sequence shown in SEQ ID NO: 154. In another instance, an anti-PD-1 polypeptide or an anti-PD-1 antigen binding fragment comprises a VH having an amino acid sequence shown in SEQ ID NO: 155, and a VL having an amino acid sequence shown in SEQ ID NO: 156. In another instance, an anti-PD-1 polypeptide or an anti-PD-1 antigen binding fragment comprises a VH having an amino acid sequence shown in SEQ ID NO: 157, and a VL having an amino acid sequence shown in SEQ ID NO: 158. In another instance, an anti-PD-1 polypeptide or an anti-PD-1 antigen binding fragment comprises a VH having an amino acid sequence shown in SEQ ID NO: 159, and a VL having an amino acid sequence shown in SEQ ID NO: 160. In another instance, an anti-PD-1 polypeptide or an anti-PD-1 antigen binding fragment comprises a VH having an amino acid sequence shown in SEQ ID NO: 161, and a VL having an amino acid sequence shown in SEQ ID NO: 162. In another instance, an anti-PD-1 polypeptide or an anti-PD-1 antigen binding fragment comprises a VH having an amino acid sequence shown in SEQ ID NO: 163, and a VL having an amino acid sequence shown in SEQ ID NO: 164. In another instance, an anti-PD-1 polypeptide or an anti-PD-1 antigen binding fragment comprises a VH having an amino acid sequence shown in SEQ ID NO: 165, and a VL having an amino acid sequence shown in SEQ ID NO: 166. In another instance, an anti-PD-1 polypeptide or an anti-PD-1 antigen binding fragment comprises a VH having an amino acid sequence shown in SEQ ID NO: 167, and a VL having an amino acid sequence shown in SEQ ID NO: 168. In another instance, an anti-PD-1 polypeptide or an anti-PD-1 antigen binding fragment comprises a VH having an amino acid sequence shown in SEQ ID NO: 194, and a VL having an amino acid sequence shown in SEQ ID NO: 195. In another instance, an anti-PD-1 polypeptide or an anti-PD-1 antigen binding fragment comprises a VH having an amino acid sequence shown in SEQ ID NO: 194, and a VL having an amino acid sequence shown in SEQ ID NO: 196. In another instance, an anti-PD-1 polypeptide or an anti-PD-1 antigen binding fragment comprises a VH having an amino acid sequence shown in SEQ ID NO: 194, and a VL having an amino acid sequence shown in SEQ ID NO: 197. In another instance, an anti-PD-1 polypeptide or an anti-PD-1 antigen binding fragment comprises a VH having an amino acid sequence shown in SEQ ID NO: 198, and a VL having an amino acid sequence shown in SEQ ID NO: 199. In another instance, an anti-PD-1 polypeptide or an anti-PD-1 antigen binding fragment comprises a VH having an amino acid sequence shown in SEQ ID NO: 200, and a VL having an amino acid sequence shown in SEQ ID NO: 201. In another instance, an anti-PD-1 polypeptide or an anti-PD-1 antigen binding fragment comprises a VH having an amino acid sequence shown in SEQ ID NO: 202, and a VL having an amino acid sequence shown in SEQ ID NO: 203. In another instance, an anti-PD-1 polypeptide or an anti-PD-1 antigen binding fragment comprises a VH having an amino acid sequence shown in SEQ ID NO: 204, and a VL having an amino acid sequence shown in SEQ ID NO: 205. In another instance, an anti-PD-1 polypeptide or an anti-PD-1 antigen binding fragment comprises a VH having an amino acid sequence shown in SEQ ID NO: 206, and a VL having an amino acid sequence shown in SEQ ID NO: 207. In another instance, an anti-PD-1 polypeptide or an anti-PD-1 antigen binding fragment comprises a VH having an amino acid sequence shown in SEQ ID NO: 208, and a VL having an amino acid sequence shown in SEQ ID NO: 209. In another instance, an anti-PD-1 polypeptide or an anti-PD-1 antigen binding fragment comprises a VH having an amino acid sequence shown in SEQ ID NO: 210, and a VL having an amino acid sequence shown in SEQ ID NO: 211. In another instance, an anti-PD-1 polypeptide or an anti-PD-1 antigen binding fragment comprises a VH having an amino acid sequence shown in SEQ ID NO: 212, and a VL having an amino acid sequence shown in SEQ ID NO: 213. In another instance, an anti-PD-1 polypeptide or an anti-PD-1 antigen binding fragment comprises a VH having an amino acid sequence shown in SEQ ID NO: 214, and a VL having an amino acid sequence shown in SEQ ID NO: 215. In another instance, an anti-PD-1 polypeptide or an anti-PD-1 antigen binding fragment comprises a VH having an amino acid sequence shown in SEQ ID NO: 270, and a VL having an amino acid sequence shown in SEQ ID NO: 271. In another instance, an anti-PD-1 polypeptide or an anti-PD-1 antigen binding fragment comprises a VH having an amino acid sequence shown in SEQ ID NO: 272, and a VL having an amino acid sequence shown in SEQ ID NO: 273. In another instance, an anti-PD-1 polypeptide or an anti-PD-1 antigen binding fragment comprises a VH having an amino acid sequence shown in SEQ ID NO: 274, and a VL having an amino acid sequence shown in SEQ ID NO: 140. In another instance, an anti-PD-1 polypeptide or an anti-PD-1 antigen binding fragment comprises a VH having an amino acid sequence shown in SEQ ID NO: 275, and a VL having an amino acid sequence shown in SEQ ID NO: 140. In another instance, an anti-PD-1 polypeptide or an anti-PD-1 antigen binding fragment comprises a VH having an amino acid sequence shown in SEQ ID NO: 276, and a VL having an amino acid sequence shown in SEQ ID NO: 277. In another instance, an anti-PD-1 polypeptide or an anti-PD-1 antigen binding fragment comprises a VH having an amino acid sequence shown in SEQ ID NO: 278, and a VL having an amino acid sequence shown in SEQ ID NO: 279.

In one instance, an anti-PD-1 polypeptide or an anti-PD-1 antigen binding fragment comprises a VH CDR I having an amino acid sequence of SEQ ID NO: 169, a VH CDR2 having an amino acid sequence of SEQ ID NO: 170, a VH CDR3 having an amino acid sequence of SEQ ID NO: 171, VL CDR1 having an amino acid sequence of SEQ ID NO: 172, a VL CDR2 having an amino acid sequence of SEQ ID NO: 173, and a VL CDR3 having an amino acid sequence of SEQ ID NO: 174. In one instance, an anti-PD-1 polypeptide or an anti-PD-1 antigen binding fragment comprises a VH CDR1 having an amino acid sequence of SEQ ID NO: 175, a VH CDR2 having an amino acid sequence of SEQ ID NO: 176, a VH CDR3 having an amino acid sequence of SEQ ID NO: 177, VL CDR1 having an amino acid sequence of SEQ ID NO: 178, a VL CDR2 having an amino acid sequence of SEQ ID NO: 179, and a VL CDR3 having an amino acid sequence of SEQ ID NO: 180. In one instance, an anti-PD-1 polypeptide or an anti-PD-1 antigen binding fragment comprises a VH CDR1 having an amino acid sequence of SEQ ID NO: 181, a VH CDR2 having an amino acid sequence of SEQ ID NO: 182, a VH CDR3 having an amino acid sequence of SEQ ID NO: 183, VL CDR1 having an amino acid sequence of SEQ ID NO: 184, a VL CDR2 having an amino acid sequence of SEQ ID NO: 185, and a VL CDR3 having an amino acid sequence of SEQ ID NO: 186. In one instance, an anti-PD-1 polypeptide or an anti-PD-1 antigen binding fragment comprises a VH CDR1 having an amino acid sequence of SEQ ID NO: 187, a VH CDR2 having an amino acid sequence of SEQ ID NO: 188, a VH CDR3 having an amino acid sequence of SEQ ID NO: 189, VL CDR1 having an amino acid sequence of SEQ ID NO: 190, a VL CDR2 having an amino acid sequence of SEQ ID NO: 191, and a VL CDR3 having an amino acid sequence of SEQ ID NO: 192. In one instance, an anti-PD-1 polypeptide or an anti-PD-1 antigen binding fragment comprises a VH CDR I having an amino acid sequence of SEQ ID NO: 216, a VH CDR2 having an amino acid sequence of SEQ ID NO: 217, a VH CDR3 having an amino acid sequence of SEQ ID NO: 218, VL CDR1 having an amino acid sequence of SEQ ID NO: 219, a VL CDR2 having an amino acid sequence of SEQ ID NO: 220, and a VL CDR3 having an amino acid sequence of SEQ ID NO: 221. In one instance, an anti-PD-1 polypeptide or an anti-PD-1 antigen binding fragment comprises a VH CDR1 having an amino acid sequence of SEQ ID NO: 222, a VH CDR2 having an amino acid sequence of SEQ ID NO: 223, a VH CDR3 having an amino acid sequence of SEQ ID NO: 224, VL CDR1 having an amino acid sequence of SEQ ID NO: 225, a VL CDR2 having an amino acid sequence of SEQ ID NO: 226, and a VL CDR3 having an amino acid sequence of SEQ ID NO: 227. In one instance, an anti-PD-1 polypeptide or an anti-PD-1 antigen binding fragment comprises a VH CDR1 having an amino acid sequence of SEQ ID NO: 228, a VH CDR2 having an amino acid sequence of SEQ ID NO: 229, a VH CDR3 having an amino acid sequence of SEQ ID NO: 230, VL CDR1 having an amino acid sequence of SEQ ID NO: 231, a VL CDR2 having an amino acid sequence of SEQ ID NO: 232, and a VL CDR3 having an amino acid sequence of SEQ ID NO: 233. In one instance, an anti-PD-1 polypeptide or an anti-PD-1 antigen binding fragment comprises a VH CDR1 having an amino acid sequence of SEQ ID NO: 234, a VH CDR2 having an amino acid sequence of SEQ ID NO: 235, a VH CDR3 having an amino acid sequence of SEQ ID NO: 236, VL CDR1 having an amino acid sequence of SEQ ID NO: 237, a VL CDR2 having an amino acid sequence of SEQ ID NO: 238, and a VL CDR3 having an amino acid sequence of SEQ ID NO: 239. In one instance, an anti-PD-1 polypeptide or an anti-PD-1 antigen binding fragment comprises a VH CDR1 having an amino acid sequence of SEQ ID NO: 240, a VH CDR2 having an amino acid sequence of SEQ ID NO: 241, a VH CDR3 having an amino acid sequence of SEQ ID NO: 242, VL CDR1 having an amino acid sequence of SEQ ID NO: 243, a VL CDR2 having an amino acid sequence of SEQ ID NO: 244, and a VL CDR3 having an amino acid sequence of SEQ ID NO: 245. In one instance, an anti-PD-1 polypeptide or an anti-PD-1 antigen binding fragment comprises a VH CDR1 having an amino acid sequence of SEQ ID NO: 246, a VH CDR2 having an amino acid sequence of SEQ ID NO: 247, a VH CDR3 having an amino acid sequence of SEQ ID NO: 248, VL CDR1 having an amino acid sequence of SEQ ID NO: 249, a VL CDR2 having an amino acid sequence of SEQ ID NO: 250, and a VL CDR3 having an amino acid sequence of SEQ ID NO: 251. In one instance, an anti-PD-1 polypeptide or an anti-PD-1 antigen binding fragment comprises a VH CDR1 having an amino acid sequence of SEQ ID NO: 252, a VH CDR2 having an amino acid sequence of SEQ ID NO: 253, a VH CDR3 having an amino acid sequence of SEQ ID NO: 254, VL CDR1 having an amino acid sequence of SEQ ID NO: 255, a VL CDR2 having an amino acid sequence of SEQ ID NO: 256, and a VL CDR3 having an amino acid sequence of SEQ ID NO: 257. In one instance, an anti-PD-1 polypeptide or an anti-PD-1 antigen binding fragment comprises a VH CDR1 having an amino acid sequence of SEQ ID NO: 258, a VH CDR2 having an amino acid sequence of SEQ ID NO: 259, a VH CDR3 having an amino acid sequence of SEQ ID NO: 260, VL CDR1 having an amino acid sequence of SEQ ID NO: 261, a VL CDR2 having an amino acid sequence of SEQ ID NO: 262, and a VL CDR3 having an amino acid sequence of SEQ ID NO: 263. In one instance, an anti-PD-1 polypeptide or an anti-PD-1 antigen binding fragment comprises a VH CDR1 having an amino acid sequence of SEQ ID NO: 264, a VH CDR2 having an amino acid sequence of SEQ ID NO: 265, a VH CDR3 having an amino acid sequence of SEQ ID NO: 266, VL CDR1 having an amino acid sequence of SEQ ID NO: 267, a VL CDR2 having an amino acid sequence of SEQ ID NO: 268, and a VL CDR3 having an amino acid sequence of SEQ ID NO: 269. In one instance, an anti-PD-1 polypeptide or an anti-PD-1 antigen binding fragment comprises a VH CDR1 having an amino acid sequence of SEQ ID NO: 280, a VH CDR2 having an amino acid sequence of SEQ ID NO: 281, a VH CDR3 having an amino acid sequence of SEQ ID NO: 282, VL CDR1 having an amino acid sequence of SEQ ID NO: 283, a VL CDR2 having an amino acid sequence of SEQ ID NO: 284, and a VL CDR3 having an amino acid sequence of SEQ ID NO: 285. In one instance, an anti-PD-1 polypeptide or an anti-PD-1 antigen binding fragment comprises a VH CDR1 having an amino acid sequence of SEQ ID NO: 286, a VH CDR2 having an amino acid sequence of SEQ ID NO: 287, a VH CDR3 having an amino acid sequence of SEQ ID NO: 288, VL CDR1 having an amino acid sequence of SEQ ID NO: 289, a VL CDR2 having an amino acid sequence of SEQ ID NO: 290, and a VL CDR3 having an amino acid sequence of SEQ ID NO: 291. In one instance, an anti-PD-1 polypeptide or an anti-PD-1 antigen binding fragment comprises a VH CDR1 having an amino acid sequence of SEQ ID NO: 292, a VH CDR2 having an amino acid sequence of SEQ ID NO: 293, a VH CDR3 having an amino acid sequence of SEQ ID NO: 294, VL CDR1 having an amino acid sequence of SEQ ID NO: 178, a VL CDR2 having an amino acid sequence of SEQ ID NO: 179, and a VL CDR3 having an amino acid sequence of SEQ ID NO: 180. In one instance, an anti-PD-1 polypeptide or an anti-PD-1 antigen binding fragment comprises a VH CDR1 having an amino acid sequence of SEQ ID NO: 295, a VH CDR2 having an amino acid sequence of SEQ ID NO: 296, a VH CDR3 having an amino acid sequence of SEQ ID NO: 297, VL CDR1 having an amino acid sequence of SEQ ID NO: 178, a VL CDR2 having an amino acid sequence of SEQ ID NO: 179, and a VL CDR3 having an amino acid sequence of SEQ ID NO: 180. In one instance, an anti-PD-1 polypeptide or an anti-PD-1 antigen binding fragment comprises a VH CDR1 having an amino acid sequence of SEQ ID NO: 298, a VH CDR2 having an amino acid sequence of SEQ ID NO: 299, a VH CDR3 having an amino acid sequence of SEQ ID NO: 300, VL CDR1 having an amino acid sequence of SEQ ID NO: 301, a VL CDR2 having an amino acid sequence of SEQ ID NO: 302, and a VL CDR3 having an amino acid sequence of SEQ ID NO: 303. In one instance, an anti-PD-1 polypeptide or an anti-PD-1 antigen binding fragment comprises a VH CDR1 having an amino acid sequence of SEQ ID NO: 304, a VH CDR2 having an amino acid sequence of SEQ ID NO: 305, a VH CDR3 having an amino acid sequence of SEQ ID NO: 306, VL CDR1 having an amino acid sequence of SEQ ID NO: 307, a VL CDR2 having an amino acid sequence of SEQ ID NO: 308, and a VL CDR3 having an amino acid sequence of SEQ ID NO: 309.

In one instance, an anti-PD-1 polypeptide comprises a fusion protein. Such fusion protein can be, for example, a two-sided Fc fusion protein comprising the extracellular domain (ECD) of programmed cell death 1 (PD-1) and the ECD of tumor necrosis factor (ligand) superfamily member 4 (TNFSF4 or OX40L) fused via hinge-CH2-CH3 Fc domain of human IgG4, expressed in CHO-KI cells, where the fusion protein has an exemplary amino acid sequence of SEQ ID NO: 193.

### Modification to Fc Region of an Immune Checkpoint Inhibitor Molecule (e.g., Anti-PD-1 Antibody) in Immunocytokines

Disclosed herein are immune checkpoint inhibitor molecules (e.g., anti-PD-1 antibodies or antigen binding fragments thereof), wherein the immune checkpoint inhibitor molecules comprise an Fc region, and the Fc region comprises at least one covalently attached linker. The linker can be covalently attached to a tyrosine, aspartic acid, glutamic acid, lysine, serine, threonine, cysteine, asparagine, or glutamine residue of the Fc region. In some embodiments, the linker is covalently attached to an aspartate, asparagine, glutamate, glutamine, cysteine, or lysine residue. In some embodiments, the linker is covalently attached to a lysine or cysteine residue. In some embodiments, the linker is covalently attached to a lysine residue.

In some embodiments, the immune checkpoint inhibitor molecule (e.g., an antibody or antigen binding fragment, such as an anti-PD-1 antibody or antigen binding fragment thereof) comprises an Fc region. In some embodiments, the Fc region is an IgG Fc region, an IgA Fc region, an IgD Fc region, an IgM Fc region, or an IgE Fc region. In some embodiments, the Fc region is an IgG Fc region, an IgA Fc region, or an IgD Fc region. In some embodiments, the Fc region is a human Fc region. In some embodiments, the Fc region is a humanized. Fc region. In some embodiments, the Fc region is an IgG Fc region. In some instances, an IgG Fc region is an IgG1 Fc region, an IgG2a Fc region, or an IgG4 Fc region. In some instances, an IgG Fc region is an IgG1 Fc region, an IgG2a Fc region, or an IgG4 Fc region.

One or more mutations may be introduced in an Fc region to reduce Fc-mediated effector functions of an antibody or antigen-binding fragment such as, for example, antibody-dependent cellular cytotoxicity (ADCC) and/or complement function. In some instances, a modified Fc comprises a humanized IgG4 kappa isotype that contains a S229P Fc mutation. In some instances, a modified Fc comprises a human IgG1 kappa where the heavy chain CH2 domain is engineered with a triple mutation such as, for example: (a) L238P, L239E, and P335S; or (2) K248; K288; and K317.

In some embodiments, the Fc region comprises an amino acid sequence at least 80%, at least 85%, at least 90%, at least 91%, at least 92%, at least 93%, at least 94%, at least 95%, at least 96%, at least 97%, at least 98%, or at least 99% identical to a sequence as set forth in SEQ ID NO: 360 (Pro Lys Ser Cys Asp Lys Thr His Thr Cys Pro Pro Cys Pro Ala Pro Glu Xaa Xaa Gly Xaa Pro Ser Val Phe Leu Phe Pro Pro Lys Pro Lys Asp Thr Leu Met Ile Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asp Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Xaa Glu Xaa Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Xaa Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly, where Xaa can be any naturally occurring amino acid).

In some embodiments, the Fc region comprises one or more mutations which make the Fc region susceptible to modification or conjugation at a particular residue, such as by incorporation of a cysteine residue at a position which does not contain a cysteine in SEQ ID NO: 360. Alternatively, the Fc region could be modified to incorporate a modified natural amino acid or an unnatural amino acid which comprises a conjugation handle, such as one connected to the modified natural amino acid or unnatural amino acid through a linker. In some embodiments, the Fc region does not comprise any mutations which facilitate the attachment of a linker to an IL-7 polypeptide. In some embodiments, the linker is attached to a native residue as set forth in SEQ ID NO: 360. In some embodiments, the chemical linker is attached to a native lysine residue of SEQ ID NO: 360.

In some embodiments, the linker can be covalently attached to one amino acid residue of an Fc region of the immune checkpoint inhibitor molecule (e.g., an antibody or antigen binding fragment, such as an anti-PD-1 antibody or antigen binding fragment thereof). In some embodiments, the chemical linker is covalently attached to a non-terminal residue of the Fc region. In some embodiments, the non-terminal residue is in the CH1, CH2, or CH3 region of the immune checkpoint inhibitor molecule. In some embodiments, the non-terminal residue is in the CH2 region of the immune checkpoint inhibitor molecule.

In some embodiments, the linker is attached to the Fc region at an amino acid residue at any one of positions 10-200 of SEQ ID NO: 360. In some embodiments, the linker is attached to the Fc region at an amino acid residue at any one of positions 10-20, 10-30, 10-40, 10-50, 10-60, 10-70, 10-80, 10-90, 10-100, 10-110, 10-120, 10-130, 10-140, 10-150, 10-160, 10-170, 10-180, 10-190, or 10-200 of SEQ ID NO: 360. In some embodiments, the linker is attached to the Fc region at an amino acid residue at any one of positions 20-40, 65-85, or 90-110 of SEQ ID NO: 360. In some embodiments, the linker is attached to the Fc region at an amino acid residue at one of positions 10-30, 50-70, or 80-100 of SEQ ID NO: 360. In some embodiments, the linker is attached to the Fc region at an amino acid residue at one of positions 15-26, 55-65, or 85-90 of SEQ ID NO: 360. In some embodiments, the linker is attached to the Fc region at an amino acid residue at any one of positions 25-35, 70-80, or 95-105 of SEQ ID NO: 360. In some embodiments, the linker is attached to the Fc region at an amino acid residue at any one of positions 30, 32, 72, 74, 79 or 101 of SEQ ID NO: 360. In some embodiments, the linker is attached to the Fc region at an amino acid residue at any one of positions K30, K32, K72, K74, Q79, or K101 of SEQ ID NO: 360. In some embodiments, the linker is attached to the Fc region at amino acid residue 30 of SEQ ID NO: 360. In some embodiments, the linker is attached to the Fc region at amino acid residue 32 of SEQ ID NO: 360. In some embodiments, the linker is attached to the Fc region at amino acid residue 72 of SEQ ID NO: 360. In some embodiments, the linker is attached to the Fc region at amino acid residue 74 of SEQ ID NO: 360. In some embodiments, the linker is attached to the Fc region at amino acid residue 79 of SEQ ID NO: 360. In some embodiments, the linker is attached to the Fc region at amino acid residue 101 of SEQ ID NO: 360.

In some embodiments, the linker is covalently attached at an amino acid residue of the immune checkpoint inhibitor molecule (e.g., an antibody or antigen binding fragment, such as an anti-PD-1 antibody or antigen binding fragment thereof) such that the function of the immune checkpoint inhibitor molecule is maintained (e.g., without denaturing the polypeptide). For example, when the immune checkpoint inhibitor molecule is an antibody such as a human IgG (e.g., human IgG1), exposed lysine residues exposed glutamine residues and exposed tyrosine residues are present at the following positions (refer to web site imgt.org/IMGTScientificChart/Numbering/Hu_IGHGnber.html by EU numbering). Exemplary exposed Lysine Residues: CH2 domain (position 246, position 248, position 274, position 288, position 290, position 317, position 320, position 322, and position 338) CH3 domain (position 360, position 414, and position 439). Exemplary exposed Glutamine Residues: CH2 domain (position 295). Exemplary exposed Tyrosine Residues: CH2 domain (position 278, position 296, and position 300) CH3 domain (position 436).

The human IgG, such as human IgG1, may also be modified with a lysine, glutamine, or tyrosine residue at any one of the positions listed above in order provide a residue which is ideally surface exposed for subsequent modification.

In some embodiments, the linker is covalently attached at an amino acid residue in the constant region of the immune checkpoint inhibitor molecule, wherein the immune checkpoint inhibitor molecule is an antibody (e.g., an anti-PD-1 antibody). In some embodiments, the linker is covalently attached at an amino acid residue in the CH1, CH2, or CH3 region. In some embodiments, the linker is covalently attached at an amino acid residue in the CH2 region. In some embodiments, the linker may be covalently attached to one residue selected from the following groups of residues following EU numbering in human IgG Fc: amino acid residues 1-478, amino acid residues 2-478, amino acid residues 1-477, amino acid residues 2-477, amino acid residues 10-467, amino acid residues 30-447, amino acid residues 50-427, amino acid residues 100-377, amino acid residues 150-327, amino acid residues 200-327, amino acid residues 240-327, and amino acid residues 240-320.

In some embodiments, the linker is covalently attached to one lysine or glutamine residue of a human IgG Fc region (e.g., a human IgG Fc region of an anti-PD-1 antibody). In some embodiments, the linker is covalently attached at Lys 246 of an Fc region of the immune checkpoint inhibitor molecule (e.g., the anti-PD-1 antibody), wherein amino acid residue position number is based on Eu numbering. In some embodiments, the linker is covalently attached at Lys 248 of an Fc region of the immune checkpoint inhibitor molecule (e.g., the anti-PD-1 antibody), wherein amino acid residue position number is based on Eu numbering. In some embodiments, the linker is covalently attached at Lys 288 of an Fc region of the immune checkpoint inhibitor molecule (e.g., the anti-PD-1 antibody), wherein amino acid residue position number is based on Eu numbering. In some embodiments, the linker is covalently attached at Lys 290 of an Fc region of the immune checkpoint inhibitor molecule (e.g., the anti-PD-1 antibody), wherein amino acid residue position number is based on Eu numbering. In some embodiments, the linker is covalently attached at Gln 295 of an Fc region of the immune checkpoint inhibitor molecule (e.g., the anti-PD-1 antibody), wherein amino acid residue position number is based on Eu numbering. In some embodiments, the linker is covalently attached at Lys 317 of the immune checkpoint inhibitor molecule (e.g., the anti-PD-1 antibody), wherein amino acid residue position number is based on Eu numbering.

In some embodiments, the linker can be covalently attached to an amino acid residue selected from a subset of amino acid residues. In some embodiments, the subset comprises two three, four, five, six, seven, eight, nine, or ten amino acid residues of an Fc region of the antibody (e.g., the anti-PD-1 antibody). In some embodiments, the chemical linker can be covalently attached to one of two lysine residues of an Fc region of the antibody (e.g., the anti-PD-1 antibody).

In some embodiments, the immune checkpoint inhibitor molecule (e.g., the anti-PD-1 antibody) will comprise two linkers covalently attached to the Fc region of the molecule. In some embodiments, each of the two linkers will be covalently attached to a different heavy chain of the immune checkpoint inhibitor molecule (e.g., the anti-PD-1 antibody). In some embodiments, each of the two linkers will be covalently attached to a different heavy chain of t the immune checkpoint inhibitor molecule (e.g., the anti-PD-1 antibody) at a residue position which is the same. In some embodiments, each of the two linkers will be covalently attached to a different heavy chain of the immune checkpoint inhibitor molecule (e.g., the anti-PD-1 antibody) at a residue position which is different. When the two linkers are covalently attached to residue positions which differ, any combination of the residue positions provided herein may be used in combination.

In some embodiments, a first linker is covalently attached at Lys 248 of a first Fc region of the immune checkpoint inhibitor molecule (e.g., the anti-PD-1 antibody), and a second linker is covalently attached at Lys 288 of a second Fc region of the immune checkpoint inhibitor molecule (e.g., the anti-PD-1 antibody), wherein residue position number is based on Eu numbering. In some embodiments, a first linker is covalently attached at Lys 246 of a first Fc region of the immune checkpoint inhibitor molecule (e.g., the anti-PD-1 antibody), and a second linker is covalently attached at Lys 288 of a second Fc region of the immune checkpoint inhibitor molecule (e.g., the anti-PD-1 antibody), wherein residue position number is based on Eu numbering. In some embodiments, a first linker is covalently attached at Lys 248 of a first Fc region of the immune checkpoint inhibitor molecule (e.g., the anti-PD-1 antibody), and a second linker is covalently attached at Lys 317 of a second Fc region of the immune checkpoint inhibitor molecule (e.g., the anti-PD-1 antibody), wherein residue position number is based on Eu numbering. In some embodiments, a first linker is covalently attached at Lys 246 of a first Fc region of the immune checkpoint inhibitor molecule (e.g., the anti-PD-1 antibody), and a second linker is covalently attached at Lys 317 of a second Fc region of the immune checkpoint inhibitor molecule (e.g., the anti-PD-1 antibody), wherein residue position number is based on Eu numbering. In some embodiments, a first linker is covalently attached at Lys 288 of a first Fc region of the immune checkpoint inhibitor molecule (e.g., the anti-PD-1 antibody), and a second linker is covalently attached at Lys 317 of a second Fc region of the immune checkpoint inhibitor molecule (e.g., the anti-PD-1 antibody), wherein residue position number is based on Eu numbering.

### Method of Modifying an Fc Region

Also provided herein are methods of preparing a modified Fc region of an immune checkpoint inhibitor molecule (e.g., an anti-PD-1 antibody), such as for the attachment of a linker, a conjugation handle, or the IL-7 polypeptide of the immunocytokine. A variety of methods for site-specific modification of Fc regions of antibodies or other polypeptides which bind to PD-1 are known in the art.

Modification with an Affinity Peptide Configured to Site-Specifically Attach Linker to an Fc Region (e.g., of an Antibody Such as an Anti-PD-1 Antibody)

In some embodiments, an Fc region of an immune checkpoint inhibitor molecule (e.g., an antibody or antigen binding fragment such as an anti-PD-1 antibody or antigen binding fragment thereof) is modified to incorporate a linker, a conjugation handle, or a combination thereof, which forms or is used to form the immunocytokine. In some embodiments, the modification is performed by contacting the Fc region with an affinity peptide bearing a payload configured to attach a linker or other group to the Fc region, such as at a specific residue of the Fc region. In some embodiments, the linker is attached using a reactive group (e.g., a N-hydroxysuccinimide ester) which forms a bond with a residue of the Fc region. In some embodiments, the affinity peptide comprises a cleavable linkage. The cleavable linkage is configured on the affinity peptide such that after the linker or portion thereof or other group is attached to the Fc region, the affinity peptide can be removed, leaving behind only the desired linker or portion thereof or other group attached to the Fc region. The linker or portion thereof or other group can then be used further to add attach the IL-7 polypeptide to the Fc region.

Non-limiting examples of such affinity peptides can be found at least in PCT Publication No. WO2018199337A1, PCT Publication No. WO2019240288A1, PCT Publication No. WO2019240287A1, and PCT Publication No. WO2020090979A1, each of which is incorporated by reference as if set forth herein in its entirety. In some embodiments, the affinity peptide is a peptide which has been modified to deliver the linker/conjugation handle payload one or more specific residues of the Fc region of the antibody. In some embodiments, the affinity peptide has at least 80%, 85%, 90%, 91%, 92%, 93%, 94%, 95%, 96%, 97%, 98%, 99%, or 100% sequence identify to a peptide selected from among (1) QETNPTENLYFQQKNMQCQRRFYEALHDPNLNEEQRNARIRSIRDDDC (SEQ ID NO: 361); (2) QTADNQKNMQCQRRFYEALHDPNLNEEQRNARIRSIRDDCSQSANLLAEAQQLNDAQA PQA (SEQ ID NO: 362); (3) QETKNMQCQRRFYEALHDPNLNEEQRNARIRSIRDDDC (SEQ ID NO: 363); (4) QETFNKQCQRRFYEALHDPNLNEEQRNARIRSIRDDDC (SEQ ID NO: 364); (5) QETFNMQCQRRFYEALHDPNLNKEQRNARIRSIRDDDC (SEQ ID NO: 365); (6) QETFNMQCQRRFYEALHDPNLNEEQRNARIRSIKDDC (SEQ ID NO: 366); (7) QETMQCQRRFYEALHDPNLNEEQRNARIRSIKDDC (SEQ ID NO: 367); (8) QETQCQRRFYEALHDPNLNEEQRNARIRSIKDDC (SEQ ID NO: 368); (9) QETCQRRFYEALHDPNLNEEQRNARIRSIKDDC (SEQ ID NO: 369); (10) QETRGNCAYHKGQLVWCTYH (SEQ ID NO: 370); and (11) QETRGNCAYHKGQIIWCTYH (SEQ ID NO: 371), or a corresponding peptide which has been truncated at the N-terminus by one, two, three, four, or five residues. An exemplary affinity peptide with cleavable linker and conjugation handle payload capable of attaching the payload to residue K248 of an antibody as provided herein is shown below (SEQ ID NO: 375) (as reported in Matsuda et al., “Chemical Site-Specific Conjugation Platform to Improve the Pharmacokinetics and Therapeutic Index of Antibody-Drug Conjugates,” Mol. Pharmaceutics 2021, 18, 11, 4058-4066).

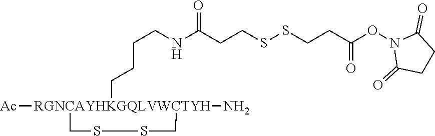

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide a sulfhydryl protecting group as a cleavable portion of the affinity peptide (e.g., the relevant portion of the affinity peptide would have a structure of

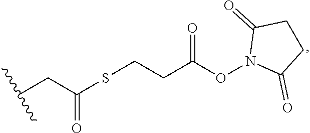 [SMILES: *CC(=O)SCCC(=O)ON1C(=O)CCC1=O]

or another of the cleavable linkers discussed below). Such alternative affinity peptides include those described in, for example “AJICAP Second Generation: Improved Chemical Site-Specific Conjugation Technology for Antibody-Drug Conjugation Technology for Antibody-Drug Conjugate Production” (Bioconjugate Chem. 2023, 34, 4, 728-738). Exemplary affinity peptides provided therein include those shown below, wherein the left structure (SEQ ID NO: 376) targets K248 of the Fc region and the right structure (SEQ ID NO: 377) targets K288 of the Fc region (EU numbering).

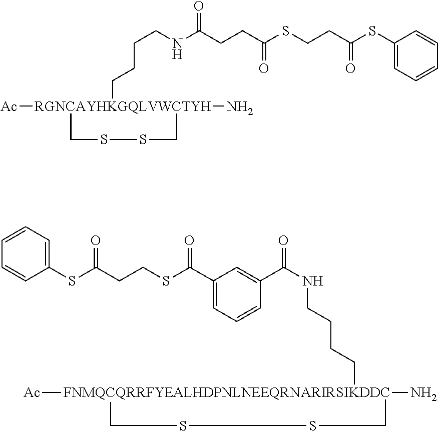

The affinity peptide of the disclosure can comprise a cleavable linkage. In some embodiments, the cleavable linkage of the affinity peptide connects the affinity peptide to the group which is to be attached to the Fc region and is configured such that the peptide can be cleaved after the group comprising the linker (or portion thereof) or conjugation handle has been attached. In some embodiments, the cleavable linkage is a divalent group. In some embodiments, the cleavable linkage can comprise a thioester group, an ester group, a sulfane group; a methanimine group; an oxyvinyl group; a thiopropanoate group; an ethane-1,2-diol group; an (imidazole-1-yl) methan-1-one group; a seleno ether group; a silylether group; a di-oxysilane group; an ether group; a di-oxymethane group; a tetraoxospiro[5.5]undecane group; an acetamidoethyl phosphoramidite group; a bis(methylthio)-pyrazolopyrazole-dione group; a 2-oxo-2-phenylethyl formate group; a 4-oxybenzylcarbamate group; a 2-(4-hydroxy-oxyphenyl)diazinyl)benzoic acid group; a 4-amino-2-(2-amino-2-oxoethyl)-4-oxobut-2-enoic acid group; a 2-(2-methylenehydrazineyl)pyridine group; an N′-methyleneformohydrazide group; or an isopropylcarbamate group, any of which is unsubstituted or substituted. Composition and points of attachment of the cleavable linkage to the affinity peptide, as well as related methods of use, are described in, at least, PCT Publication No. WO2018199337A1, PCT Publication No. WO2019240288A1, PCT Publication No. WO2019240287A1, and PCT Publication No. WO2020090979A1.

In some embodiments, the cleavable linkage is:

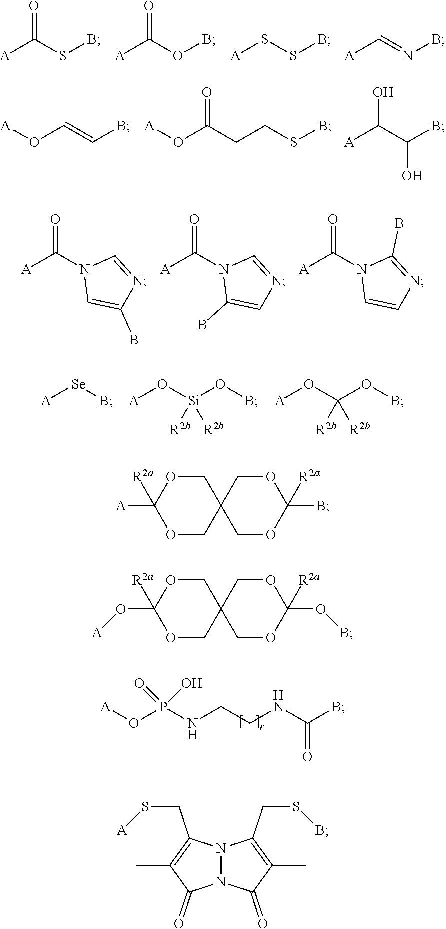

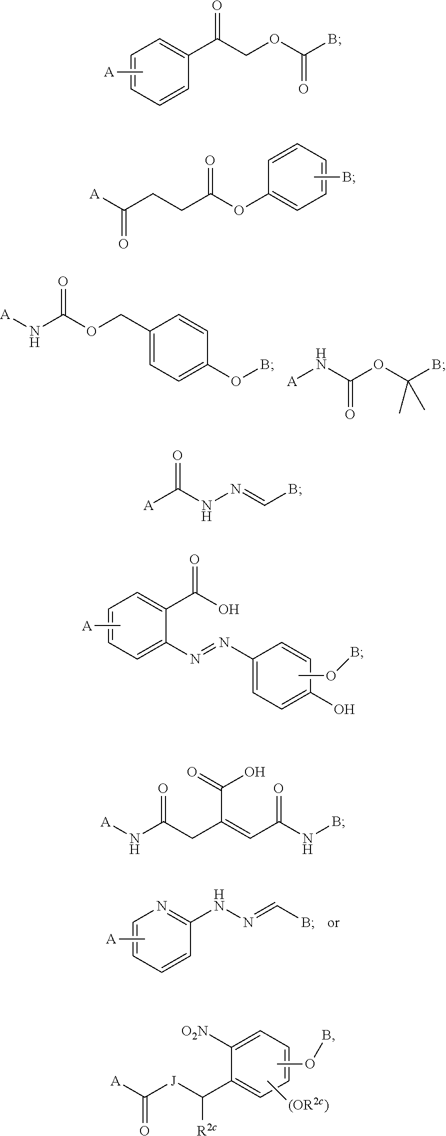

wherein:
one of A or B is a point of attachment the linker and the other of A or B is a point of attachment to the affinity peptide;
each R^{2a} is independently H or optionally substituted alkyl;
each R^{2b} is independently H or optionally substituted alkyl;
R^{2c} is a H or optionally substituted alkyl;
J is a methylene, a N, a S, a Si, or an O atom; and
r is 1, 2, 3, 4, 5, 6, 7, 8, 9, or 10.

The affinity peptide comprises a reactive group which is configured to enable the covalent attachment of the linker (or portion thereof)/conjugation handle to the Fc region. In some embodiments, the reactive group is selective for a functional group of a specific amino acid residue, such as a lysine residue, tyrosine residue, serine residue, cysteine residue, or an unnatural amino acid residue of the Fc region incorporated to facilitate the attachment of the linker. The reactive group may be any suitable functional group, such as an activated ester for reaction with a lysine (e.g., N-hydroxysuccinimide ester or a derivate thereof, a pentafluorophenyl ester, etc.) or a sulfhydryl reactive group for reaction with a cysteine (e.g., a Michael acceptor, such as an alpha-beta unsaturated carbonyl or a maleimide, an alpha-halo carbonyl, etc.). In some embodiments, the reactive group is:

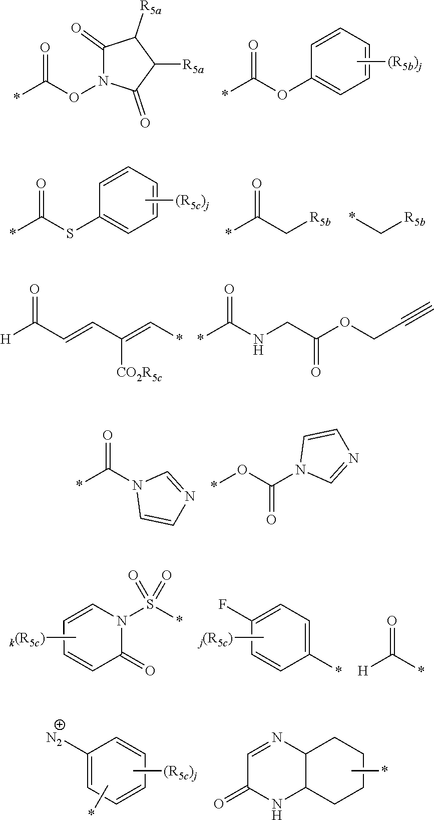

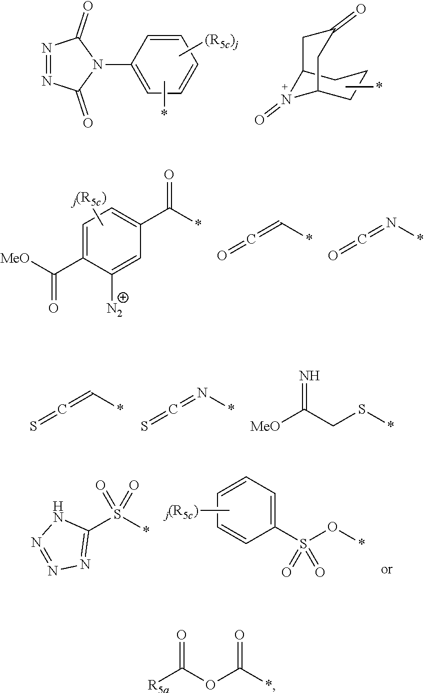

wherein:
each R₅ₐ, R_{5b}, and R_{5c} is independently H, halogen, or optionally substituted alkyl;
wherein each j is 1, 2, 3, 4, or 5; and
each k is 1, 2, 3, 4, or 5.

In some embodiments, the affinity peptide is used to deliver a reactive moiety to the desired amino acid residue such that the reactive moiety is exposed upon cleavage of the cleavable linkage. By way of non-limiting example, the reactive group forms a covalent bond with a desired residue of the Fc region of the immune checkpoint inhibitor molecule (e.g., anti-PD-1 antibody or antigen binding fragment thereof) due to an interaction between the affinity peptide and the Fc region. Following this covalent bond formation, the cleavable linkage is cleaved under appropriate conditions to reveal a reactive moiety (e.g., if the cleavable linkage comprises a thioester, a free sulfhydryl group is attached to the Fc region following cleavage of the cleavable linkage). This new reactive moiety can then be used to subsequently add an additional moiety, such as a conjugation handle, by way of reagent comprising the conjugation handle tethered to a sulfhydryl reactive group (e.g., alpha-halogenated carbonyl group, alpha-beta unsaturated carbonyl group, maleimide group, etc.).

In some embodiments, an affinity peptide is used to deliver a free sulfhydryl group to a lysine of the Fc region. In some embodiments, the free sulfhydryl group is then reacted with a bifunctional linking reagent to attach a new conjugation handle to the Fc region. In some embodiments, the new conjugation handle is then used to form the linker to the attached cytokine. In some embodiments, the new conjugation handle is an alkyne functional group. In some embodiments, the new conjugation handle is a DBCO functional group.

Exemplary bifunctional linking reagents useful for this purpose are of a formula A-B-C, wherein A is the sulfhydryl reactive conjugation handle (e.g., maleimide, α,β-unsaturated carbonyl, α-halogenated carbonyl), B is a linkage group, and C is the new conjugation handle (e.g., an alkyne such as DBCO). Specific non-limiting examples of bifunctional linking reagents include

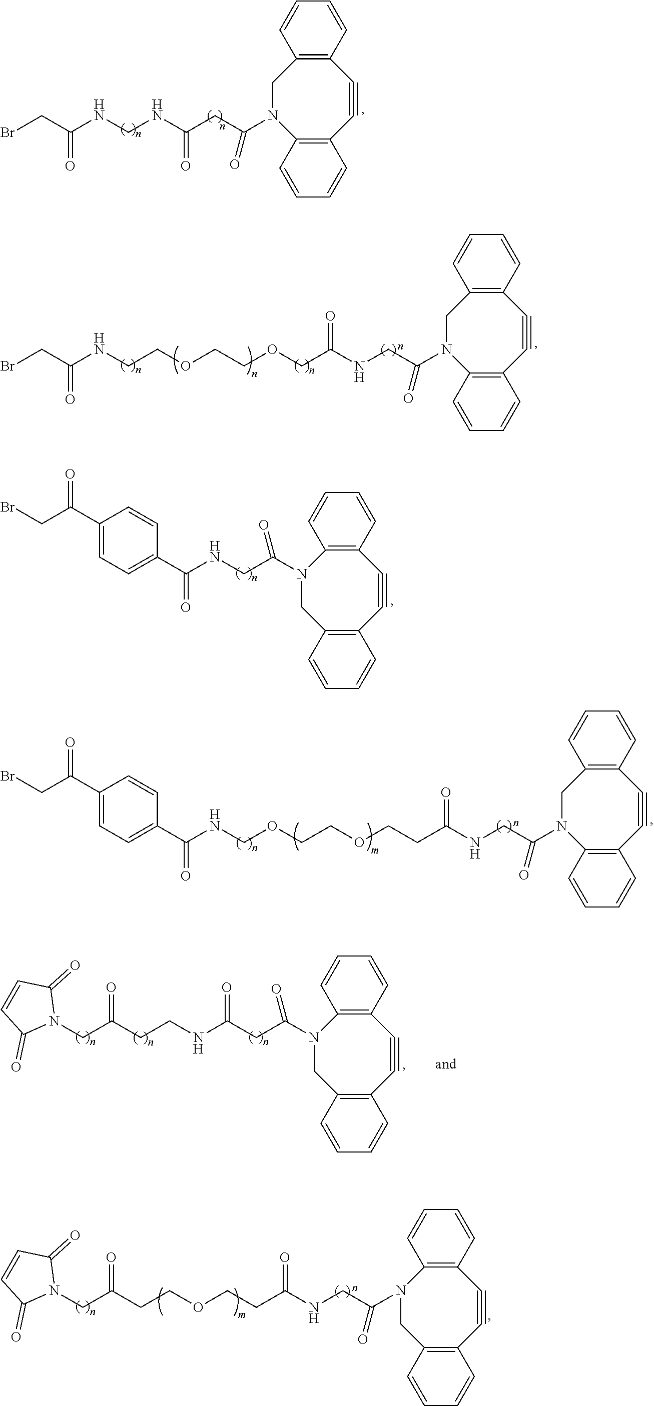

wherein each n is independently an integer from 1-6 and each m is independently an integer from 1-30, and related molecules (e.g., isomers).

Alternatively, the affinity peptide can be configured such that a conjugation handle is added to the Fc region (such as by a linker group) immediately after covalent bond formation between the reactive group and a residue of the Fc region. In such cases, the affinity peptide is cleaved and the conjugation handle is immediately ready for subsequent conjugation to the IL-7 polypeptide.

### Alternative Methods of Attachment of Linker (or Portion Thereof) to Immune Checkpoint Inhibitor Molecules-Enzyme Mediated

While the affinity peptide mediated modification of an immune checkpoint inhibitor molecule (e.g., an Fc region of an antibody such as an anti-PD-1 antibody) provided supra possesses many advantages over other methods which can be used to site-specifically modify the immune checkpoint inhibitor molecule (e.g., ease of use, ability to rapidly generate many different antibody conjugates, ability to use many “off-the-shelf” commercial antibodies without the need to do time consuming protein engineering, etc.), other methods of performing the modification are also contemplated as being within the scope of the present disclosure.

In some embodiments, the present disclosure relates generally to transglutaminase-mediated site-specific antibody-drug conjugates (ADCs) comprising: 1) glutamine-containing tags, endogenous glutamines (e.g., native glutamines without engineering, such as glutamines in variable domains, CDRs, etc.), and/or endogenous glutamines made reactive by antibody engineering or an engineered transglutaminase; and 2)amine donor agents comprising amine donor units, linkers, and agent moieties. Non-limiting examples of such transglutaminase mediated site-specific modifications can be found at least in publications WO2020188061, US2022133904, US2019194641, US2021128743, U.S. Pat. Nos. 9,764,038, 10,675,359, 9,717,803, 10,434,180, 9,427,478, which are incorporated by reference as if set forth herein in their entirety.

In another aspect, the disclosure provides an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or different amino acid), and wherein the engineered Fc-containing polypeptide conjugate comprises an amino acid substitution from glutamine to asparagine at position 295 (Q295N; EU numbering scheme).

In some embodiments, the acyl donor glutamine-containing tag is not spatially adjacent to a reactive Lys (e.g., the ability to form a covalent bond as an amine donor in the presence of an acyl donor and a transglutaminase) in the polypeptide or the Fc-containing polypeptide. In some embodiments, the polypeptide or the Fc-containing polypeptide comprises an amino acid modification at the last amino acid position in the carboxyl terminus relative to a wild-type polypeptide at the same position. The amino acid modification can be an amino acid deletion, insertion, substitution, mutation, or any combination thereof.

In some embodiments, the polypeptide conjugate comprises a full length antibody heavy chain and an antibody light chain, wherein the acyl donor glutamine-containing tag is located at the carboxyl terminus of a heavy chain, a light chain, or both the heavy chain and the light chain.

In some embodiments, the polypeptide conjugate comprises an antibody, wherein the antibody is a monoclonal antibody, a polyclonal antibody, a human antibody, a humanized antibody, a chimeric antibody, a bispecific antibody, a minibody, a diabody, or an antibody fragment. In some embodiments, the antibody is an IgG.

In another aspect, described herein is a method for preparing an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or a different amino acid), and wherein the engineered Fc-containing polypeptide conjugate comprises an amino acid substitution from glutamine to asparagine at position 295 (Q295N; EU numbering scheme), comprising the steps of: a) providing an engineered (Fc-containing polypeptide)-T molecule comprising the Fc-containing polypeptide and the acyl donor glutamine-containing tag; b) contacting the amine donor agent with the engineered (Fc-containing polypeptide)-T molecule in the presence of a transglutaminase; and c) allowing the engineered (Fc-containing polypeptide)-T to covalently link to the amine donor agent to form the engineered Fc-containing polypeptide conjugate.

In another aspect, described herein is a method for preparing an engineered polypeptide conjugate comprising the formula: polypeptide-T-A, wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the polypeptide, and wherein the acyl donor glutamine-containing tag comprises an amino acid sequence LLQGPX (SEQ ID NO: 372), wherein X is A or P, or GGLLQGPP (SEQ ID NO: 373), comprising the steps of: a) providing an engineered polypeptide-T molecule comprising the polypeptide and the acyl donor glutamine-containing tag; b) contacting the amine donor agent with the engineered polypeptide-T molecule in the presence of a transglutaminase; and c) allowing the engineered polypeptide-T to covalently link to the amine donor agent to form the engineered Fc-containing polypeptide conjugate.

In some embodiments, the engineered polypeptide conjugate (e.g., the engineered Fc-containing polypeptide conjugate, the engineered Fab-containing polypeptide conjugate, or the engineered antibody conjugate) as described herein has conjugation efficiency of at least about 51%. In another aspect, the invention provides a pharmaceutical composition comprising the engineered polypeptide conjugate as described herein (e.g., the engineered Fc-containing polypeptide conjugate, the engineered Fab-containing polypeptide conjugate, or the engineered antibody conjugate) and a pharmaceutically acceptable excipient.

In some embodiments, described herein is a method for conjugating a linker or portion thereof to an immune checkpoint inhibitor molecule to form an immunocytokine, comprising the steps of: (a) providing an antibody (e.g., an anti-PD-1 antibody) having (e.g., within the primary sequence of a constant region) at least one acceptor amino acid residue (e.g., a naturally occurring amino acid) that is reactive with a linking reagent (linker) in the presence of a coupling enzyme, e.g., a transamidase; and (b) reacting said antibody with a linking reagent (e.g., a linker comprising a primary amine) comprising a reactive group (R), optionally a protected reactive group or optionally an unprotected reactive group, in the presence of an enzyme capable of causing the formation of a covalent bond between the acceptor amino acid residue and the linking reagent (other than at the R moiety), under conditions sufficient to obtain an antibody comprising an acceptor amino acid residue linked (covalently) to a reactive group (R) via the linking reagent. Optionally, said acceptor residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH₂ domain. Optionally, the antibody is free of heavy chain N297-linked glycosylation. Optionally, the acceptor glutamine is at position 295 and the residue at the +2 position is the residue at position 297 (EU index numbering) of an antibody heavy chain.

In one aspect, described herein is a method for conjugating a moiety of interest (Z) to immune checkpoint inhibitor molecule to form an immunocytokine, comprising the steps of: (a) providing an antibody (e.g., an anti-PD-1 antibody) having at least one acceptor glutamine residue; and (b) reacting said antibody with a linker comprising a primary amine (a lysine-based linker) comprising a reactive group (R), preferably a protected reactive group, in the presence of a transglutaminase (TGase), under conditions sufficient to obtain an antibody comprising an acceptor glutamine linked (covalently) to a reactive group (R) via said linker. Optionally, said acceptor glutamine residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain. Optionally, the antibody is free of heavy chain N297-linked glycosylation. Optionally, the acceptor glutamine is at position 295 and the residue at the +2 position is the residue at position 297 (EU index numbering) of an antibody heavy chain.

The antibody (e.g., the anti-PD-1 antibody) comprising an acceptor residue or acceptor glutamine residue linked to a reactive group (R) via a linker comprising a primary amine (a lysine-based linker) can thereafter be reacted with a reaction partner comprising a moiety of interest (Z) to generate an antibody comprising an acceptor residue or acceptor glutamine residue linked to a moiety of interest (Z) via the linker. Thus, in one embodiment, the method further comprises a step (c): reacting (i) an antibody of step b) comprising an acceptor glutamine linked to a reactive group (R) via a linker comprising a primary amine (a lysine-based linker), optionally immobilized on a solid support, with (ii) a compound comprising a moiety of interest (Z) and a reactive group (R′) capable of reacting with reactive group R, under conditions sufficient to obtain an antibody comprising an acceptor glutamine linked to a moiety of interest (Z) via a linker comprising a primary amine (a lysine-based linker). Preferably, said compound comprising a moiety of interest (Z) and a reactive group (R′) capable of reacting with reactive group R is provided at a less than 80 times, 40 times, 20 times, 10 times, 5 times or 4 molar equivalents to the antibody. In one embodiment, the antibody comprises two acceptor glutamines and the compound comprising a moiety of interest (Z) and a reactive group (R′) is provided at 10 or less molar equivalents to the antibody. In one embodiment, the antibody comprises two acceptor glutamines and the compound comprising a moiety of interest (Z) and a reactive group (R′) is provided at 5 or less molar equivalents to the antibody. In one embodiment, the antibody comprises four acceptor glutamines and the compound comprising a moiety of interest (Z) and a reactive group (R′) is provided at 20 or less molar equivalents to the antibody. In one embodiment, the antibody comprises four acceptor glutamines and the compound comprising a moiety of interest (Z) and a reactive group (R′) is provided at 10 or less molar equivalents to the antibody. In one embodiment, steps (b) and/or (c) are carried out in aqueous conditions. Optionally, step (c) comprises: immobilizing a sample of an antibody comprising a functionalized acceptor glutamine residue on a solid support to provide a sample comprising immobilized antibodies, reacting the sample comprising immobilized antibodies with a compound, optionally recovering any unreacted compound and re-introducing such recovered compound to the solid support for reaction with immobilized antibodies, and eluting the antibody conjugates to provide a composition comprising a Z moiety.

### Linker Structure

In some embodiments, the linker used to attach the immune checkpoint inhibitor molecule (e.g., the anti-PD-1 antibody or antigen binding fragment thereof) and the IL-7 polypeptide comprises points of attachment at both groups. The points of attachment can be any of the residues for facilitating the attachment as provided herein. The linker structure can be any suitable structure for creating the spatial attachment between the two moieties. In some embodiments, the linker provides covalent attachment of both moieties (e.g., the IL-7 polypeptide and the immune checkpoint inhibitor molecule, such as the anti-PD-1 antibody or antigen binding fragment thereof). In some embodiments, the linker is a chemical linker (e.g., not an expressed polypeptide as in a fusion protein).

In some embodiments, the linker is a chemical linker group. In some embodiments, the linker comprises at least one portion which is not comprised of amino acid residues. In some embodiments, the linker comprises a polymer. In some embodiments, the linker comprises a non-polymer. In some embodiments, the linker comprises a polymer and a non-polymer (e.g., a polymeric portion and a non-polymeric portion).

In some embodiments, the linker comprises a polymer. In some embodiments, the linker comprises a water soluble polymer. In some embodiments, the linker comprises poly(alkylene oxide), polysaccharide, poly(vinyl pyrrolidone), poly(vinyl alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol.

In some embodiments, the linker is a bifunctional linker. In some embodiments, the bifunctional linker comprises an amide group, an ester group, an ether group, a thioether group, or a carbonyl group. In some embodiments, the linker comprises a non-polymer linker. In some embodiments, the linker comprises a non-polymer, bifunctional linker. In some embodiments, the non-polymer, bifunctional linker comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminocthyl)-4-{2-[4-(3-azidopropoxy)phenyl]diazenyl}benzamide hydrochloride.

The linker can be branched or linear. In some embodiments, the linker is linear. In some embodiments, the linker is branched. In some embodiments, the linker comprises a linear portion (e.g., between the first point of attachment and the second point of attachment) of a chain of at least 10, 20, 50, 100, 500, 1000, 2000, 3000, or 5000 atoms. In some embodiments, the linker comprises a linear portion of a chain of at least 10, 20, 30, 40, or 50 atoms. In some embodiments, the linker comprises a linear portion of at least 10 atoms. In some embodiments, the linker is branched and comprises a linear portion of a chain of at least 10, 20, 50, 100, 500, 1000, 2000, 3000, or 5000 atoms. In some embodiments, the linker comprises a linear chain of at most 5000, 3000, 2000, 1000, 500, 400, 300, 200, or 100 atoms.

In some embodiments, the linker has a molecular weight of about 100 Daltons to about 2000 Daltons. In some embodiments, the linker has a molecular weight of about 100 Daltons to about 5000 Daltons. In some embodiments, the linker has a molecular weight of 100 Daltons to 100,000 Daltons. In a preferred embodiments, the linker has a molecular weight of less than 5000 Daltons, less than 4000 Daltons, less than 3000 Daltons, or less than 2000 Daltons, and the linker is monodisperse (e.g., for a population of conjugate compositions herein, there is a high degree of uniformity of the linker between the immune checkpoint inhibitor molecule (e.g., the anti-PD-1 antibody or antigen binding fragment thereof) and the IL-7 polypeptide.

In some embodiments, the linker comprises a reaction product one or more pairs of conjugation handles and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole. The reaction product can be separated from the first point of attachment and the second point of attachment by any portion of the linker. In some embodiments, the reaction product is substantially in the center of the linker. In some embodiments, the reaction product is substantially closer to one point of attachment than the other.

In some embodiments, the linker comprises a structure of Formula (X)

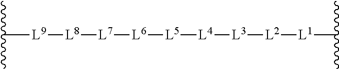

wherein each of L¹, L², L³, L⁴, L⁵, L⁶, L⁷, L⁸, and L⁹ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —C(═O)(C₁-C₆ alkylene)S—, —S(C₁-C₆ alkylene)C(═O)— —S(═O)—, —S(═O)₂—, —C(═O)—, —(C₁-C₆ alkylene)C(═O)—, —C(═O)(C₁-C₆ alkylene)-, —C(═O)(C₁-C₆ alkylene)C(═O)—, —C(═O)O—, —OC(═O)—, —OC(═O)O—, —C(═O)NR^{L}—, —C(═O)NR(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}—N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}, (C₁-C₆ alkyl)(CH₂—CH₂—O)_{qa}—, —(C₁-C₆ alkyl)(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene)
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

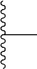

is a point of attachment to the immune checkpoint inhibitor molecule (e.g., the anti-PD-1 antibody or antigen binding fragment thereof) or the IL-7 polypeptide.

In some embodiments, the linker comprises a structure of Formula (X′)

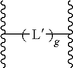

wherein each L′ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene) C(═O)—, —C(═O)(C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}, —NR^{L}C(═S)NR^{L}—, —CR^{L}═N—, —N═CR^{L}, —NR^{L}S(—O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl;
each of qa, qb, qc and qd is independently an integer from 1-100, and,
g is an integer from 1-100,
wherein each

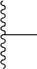

is a point of attachment to the IL-7 polypeptide or immune checkpoint inhibitor molecule (e.g., the anti-PD-1 antibody or antigen binding fragment thereof).

In some embodiments, the linker of Formula (X) or of Formula (X′) comprises the structure:

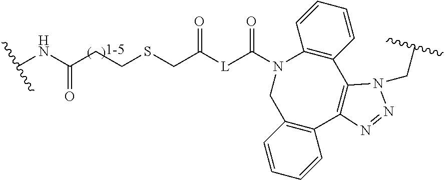

wherein

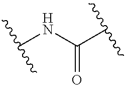 [SMILES: *NC(*)=O]

is the point of attachment to a lysine residue of the immune checkpoint inhibitor molecule (e.g., the anti-PD-1 antibody or antigen binding fragment thereof);
L is a tether group; and

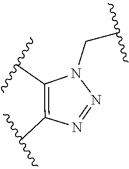 [SMILES: *Cn1nnc(*)c1*]

is a point of attachment to a tether group which connects to the IL-7 polypeptide, or a regioisomer thereof.

In some embodiments, L has a structure

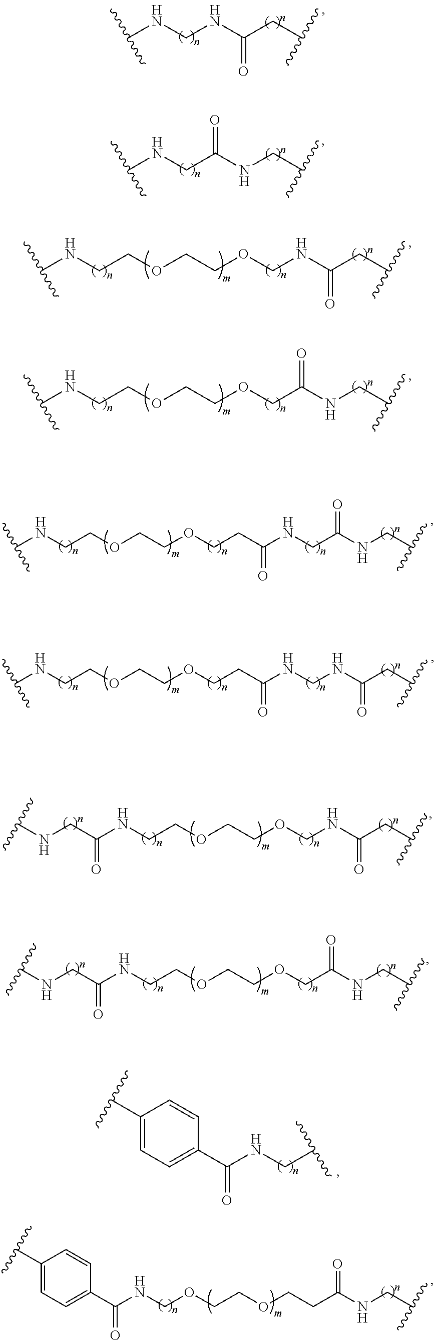

wherein each n is independently an integer from 1-6 and each m is an integer from 1-30. In some embodiments, each m is independently 2 or 3. In some embodiments, each n is an integer from 1-24, from 1-18, from 1-12, or from 1-6.

In some embodiments, the linker of Formula (X) or of Formula (X′) comprises the structure:

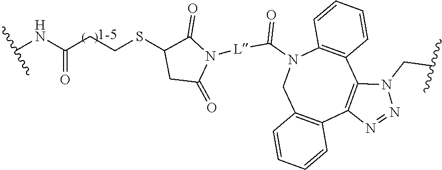

wherein

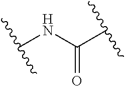 [SMILES: *NC(*)=O]

is the first point of attachment to a lysine residue of the immune checkpoint inhibitor molecule (e.g., the anti-PD-1 antibody or antigen binding fragment thereof);
L″ is a tether group; and

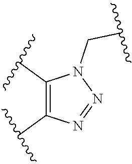 [SMILES: *Cn1nnc(*)c1*]

is a point of attachment to a tether group which connects to the IL-7 polypeptide, or a regioisomer thereof. In the structure above, in some embodiments, the succinimide is hydrolyzed at one of the N—C(═O) bonds.

In some embodiments, L″ has a structure

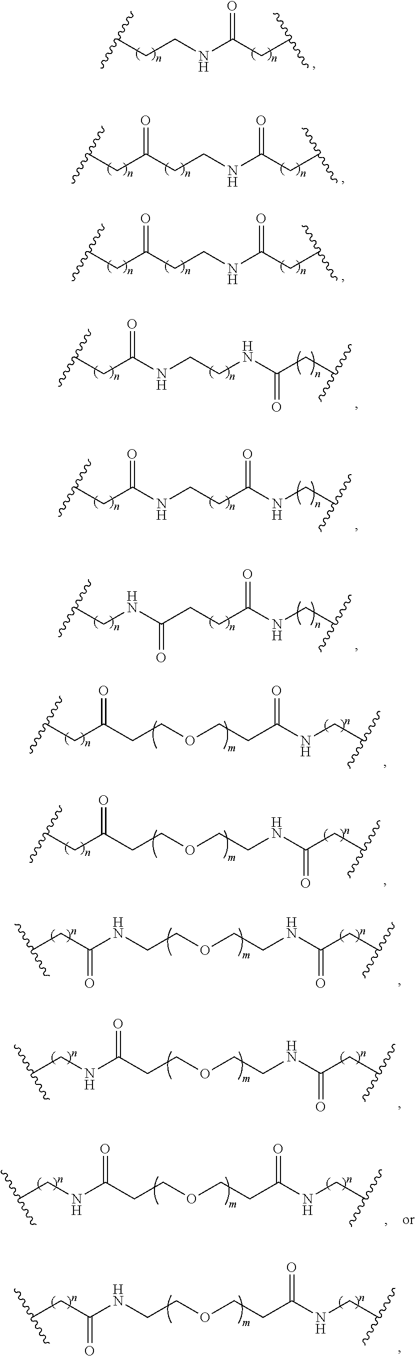

wherein each n is independently an integer from 1-6 and each m is independently an integer from 1-30. In some embodiments, each m is independently 2 or 3. In some embodiments, each m is an integer from 1-24, from 1-18, from 1-12, or from 1-6.

In some embodiments, L or L″ comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more subunits each independently selected from

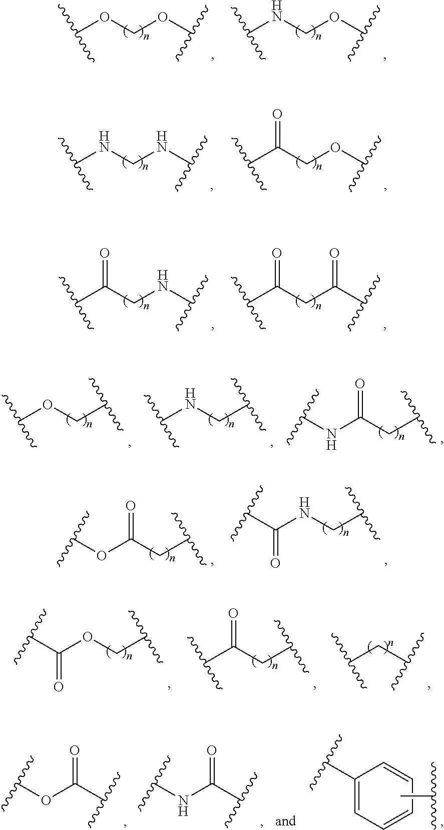

wherein each n is independently an integer from 1-30. In some embodiments, each n is independently an integer from 1-6. In some embodiments, L or L″ comprise 1, 2, 3, 4, 5, 6, 7, 8, 9, or 10 of the subunits.

In some embodiments, L or L″ is a structure of Formula (X″)

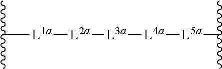

wherein each of L^{1a}, L^{2a}, L^{3a}, L^{4a}, L^{5a}, is independently —O—, —NR^{La}—, —(C₁-C₆ alkylene)NR^{La}—, —NR^{La}(C₁-C₆ alkylene)-, —N(R^{La})₂⁺—, —(C₁-C₆ alkylene)-, —N(R^{L})₂⁺(C₁-C₆ alkylene)-, —N(R^{L})₂⁺—, —OP(═O)(OR^{La})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O)(C₁-C₆ alkylene)-, —C(═O)O—, —OC(═O)—, —OC(═O)O—, —C(═O)NR^{La}—, —C(═O) NR^{La}(C₁-C₆ alkylene)-, —(C₁-C₆ alkylene)C(═O)NR^{La}—, —NR^{La}C(═O)—, —(C₁-C₆ alkylene)NR^{La}C(═O)—, —NR^{La}C(═O)(C₁-C₆ alkylene)-, —OC(═O)NR^{La}—, —NR^{La}C(═O)O—, —NR^{La}C(═O)NR^{La}—, —NR^{La}C(═S)NR^{La}—, —CR^{La}═N—, —N═CR^{La}, —NR^{La}S(═O)₂—, —S(═O)₂NR^{La}—, —C(═O)NR^{La}S(═O)₂—, —S(═O)₂NR^{La}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qe}—, —(O—CH₂—CH₂)_{qf}—, —(CH₂—CH(CH₃)—O)_{qg}—, —(O—CH(CH₃)—CH₂)_{qh}—, a reaction product of a conjugation handle and a complementary conjugation handle, or absent; each R^{La} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and each of qe, qf, qg and qh is independently an integer from 1-100.

In some embodiments, L or L″ comprises a linear chain of 2 to 10, 2 to 15, 2 to 20, 2 to 25, or 2 to 30 atoms. In some embodiments, the linear chain comprises one or more alkyl groups (e.g., lower alkyl(C₁-C₄)), one or more aromatic groups (e.g., phenyl), one or more amide groups, one or more ether groups, one or more ester groups, or any combination thereof.

In some embodiments, the tether group which connects to the first point of attachment (e.g., the point of attachment to the IL-7 polypeptide) comprises poly(ethylene glycol). In some embodiments, the tether group comprises about 2 to about 30 poly(ethylene glycol) units. In some embodiments, the tether group which connects to the first point of attachment (e.g., the point of attachment to the IL-7 polypeptide) is a functionality attached to an IL-7 polypeptide provided herein which comprises an azide (e.g., the triazole is the reaction product of the azide).

In some embodiments, each reaction product of a conjugation handle and a complementary conjugation handle independently comprises a triazole, a hydrazone, pyridazine, a sulfide, a disulfide, an amide, an ester, an ether, an oxime, or an alkene. In some embodiments, each reaction product of a conjugation handle and a complementary conjugation handle comprises a triazole. In some embodiments, each reaction product of a conjugation handle and a complementary conjugation handle independently comprises a structure of

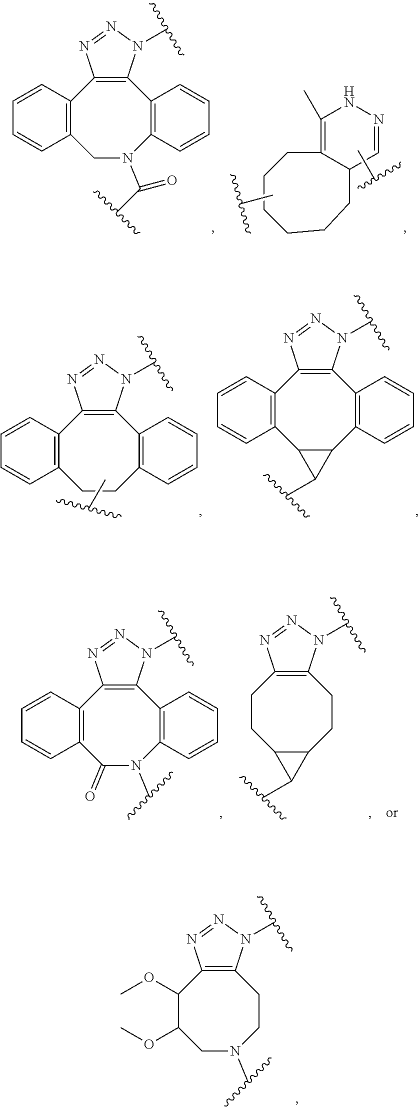

or a regioisomer or derivative thereof.

In some embodiments, the linker is a cleavable linker. In some embodiments, the cleavable linker is cleaved at, near, or in a tumor microenvironment. In some embodiments, the tumor is mechanically or physically cleaved at, near, or in the tumor microenvironment. In some embodiments, the tumor is chemically cleaved at, near, or in a tumor microenvironment. In some embodiments, the cleavable linker is a reduction sensitive linker. In some embodiments, the cleavable linker is an oxidation sensitive linker. In some embodiments, the cleavable linker is cleaved as a result of pH at, near, or in the tumor microenvironment. In some embodiments, the linker by a tumor metabolite at, near, or in the tumor microenvironment. In some embodiments, the cleavable linker is cleaved by a protease at, near, or in the tumor microenvironment.

### Stoichiometry of Immunocytokines

In some embodiments, an immunocytokine as provided herein can have a specified ratio of the number of immune checkpoint inhibitor molecules (e.g., anti-PD-1 antibodies or antigen binding fragments thereof) and IL-7 polypeptides. In some embodiments, the ratio of immune checkpoint inhibitor molecules to IL-7 polypeptides is 1 to 1 or 1 to 2. In some embodiments, a population of immunocytokines provided herein has a ratio of immune checkpoint inhibitor molecules to IL-7 polypeptides of from about 1 to 1 to about 1 to 2 (e.g., can include individual immunocytokines which comprise 1 IL-7 polypeptide and individual immunocytokines which comprises 2 IL-7 polypeptides).

In some embodiments, wherein the immune checkpoint inhibitor molecule is an antibody or an antigen binding fragment thereof (e.g., an anti-PD-1 antibody or antigen binding fragment thereof), the ratio of immune checkpoint inhibitor molecule to IL-7 polypeptide in the immunocytokine can be referred as a drug-antibody ration (DAR). In some embodiments, an immunocytokine has a DAR of 1 or 2. In some embodiments, a population of immunocytokines has a DAR of from 1 to 2.

### Points of Attachment of Linkers to IL-7 Polypeptides

Immunocytokines provided herein comprise linkers which are attached to the IL-7 polypeptide. As discussed supra, the linker can be attached to immune checkpoint inhibitor molecule (e.g., the anti-PD-1 antibody or antigen binding fragment thereof) at any of the positions provide herein. The second point of attachment of the linker is attached to an IL-7 polypeptide (including a synthetic IL-7) as provided herein.

In some embodiments, the linker is attached to the IL-7 polypeptide at an amino acid residue. In some embodiments, the linker is attached at an amino acid residue corresponding to any one of amino acid residues 1-152 of SEQ ID NO: 1 (e.g., any one of amino acid residues 1-152 of SEQ ID NO: 1).

In some embodiments, the linker is attached to a terminal amino acid residue of the IL-7 polypeptide. In some embodiments, the linker is attached to the N-terminal residue or the C-terminal residue of the IL-7 polypeptide. In some embodiments, the linker is attached to the N-terminal amino group of the IL-7 polypeptide or the C-terminal carboxyl group of the IL-7 polypeptide. In some embodiments, the N-terminal residue is a residue corresponding to position 1 of SEQ ID NO: 1. In some embodiments, the IL-7 polypeptide comprises a truncation of one or more amino acid residues from the N-terminus of SEQ ID NO: 1 (e.g., a deletion of 1, 2, 3, 4, 5, 6, 7, 8, 9, 10, or more amino acid residues) and the linker is attached to the residue which now comprises the N-terminus (e.g., for a truncation of one amino acid, the linker is attached to a residue at a position corresponding to residue 2 of SEQ ID NO: 1). In some embodiments, the IL-7 polypeptide comprises an extension of one or more amino acid residues from the N-terminus of SEQ ID NO: 1 (e.g., an extension of 1, 2, 3, 4, 5, 6, 7, 8, 9, 10, or more amino acid residues) and the linker is attached to the residue which now comprises the N-terminus. In some embodiments, the IL-7 polypeptide comprises a truncation of one or more amino acid residues from the C-terminus of SEQ ID NO: 1 (e.g., a deletion of 1, 2, 3, 4, 5, 6, 7, 8, 9, 10, or more amino acid residues) and the linker is attached to the residue which now comprises the C-terminus (e.g., for a truncation of one amino acid, the linker is attached to a residue at a position corresponding to residue 151 of SEQ ID NO: 1). In some embodiments, the IL-7 polypeptide comprises an extension of one or more amino acid residues from the C-terminus of SEQ ID NO: 1 (e.g., an extension of 1, 2, 3, 4, 5, 6, 7, 8, 9, 10, or more amino acid residues) and the linker is attached to the residue which now comprises the C-terminus.

In some embodiments, the linker is attached to the N-terminal amino acid residue of the IL-7 polypeptide. In some embodiments, the linker is attached to the N-terminal amino group of the IL-7 polypeptide. In some embodiments, the linker is attached to the N-terminal amino group of the IL-7 polypeptide through by a reaction with an adduct attached to the N-terminal amino group having a structure

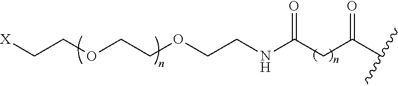

wherein each n is independently an integer from 1-30 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30), and wherein X is a conjugation handle (e.g., an azide or other conjugation handle provided herein, such as a DBCO group). In some embodiments, the adduct has the structure

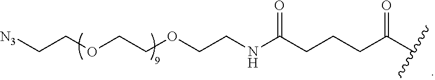

In some embodiments, the IL-7 polypeptide comprises a conjugation handle attached to one or more residues to facilitate attachment of the linker to the polypeptide which selectively binds to PD-1. The conjugation handle may be any such conjugation handle provided herein and may be attached at any residue to which the linker may be attached. In some embodiments, the conjugation handle is attached to the N-terminal residue of the polypeptide. In some embodiments, the conjugation handle comprises an azide or an alkyne.

### Activity of Immunocytokines

In some embodiments, the IL-7 polypeptide conjugated to the immune checkpoint inhibitor molecule (e.g., the anti-PD-1 antibody or antigen binding fragment) exhibits a half maximal effective concentration (EC50) for inducing STAT5 phosphorylation in at least one T-cell subtype which is comparable to wild type IL-7 when the IL-7 polypeptide is attached to the immune checkpoint inhibitor molecule. In some embodiments, the EC50 of the IL-7 in the immunocytokine for inducing STAT5 phosphorylation in the at least one T-cell subtype is no more than 2-fold greater than, 3-fold greater than, 4-fold greater than, 5-fold greater than, 6-fold greater than, 7-fold greater than, 8-fold greater than, 9-fold greater than, 10-fold greater than, 20-fold greater than, 50-fold greater than, or 100-fold greater than that of wild type IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

In some embodiments, the IL-7 polypeptide conjugated to the immune checkpoint inhibitor molecule (e.g., the anti-PD-1 antibody or antigen binding fragment) exhibits a half maximal effective concentration (EC50) for inducing STAT5 phosphorylation in at least one T-cell subtype which is lower compared to wild type IL-7 when the IL-7 polypeptide is attached to the immune checkpoint inhibitor molecule (e.g., is more potent than wild type IL-7). In some embodiments, the EC50 of the IL-7 in the immunocytokine for inducing STAT5 phosphorylation in the at least one T-cell subtype is at least 2-fold lower than, 3-fold lower than, 4-fold lower than, 5-fold lower than, 6-fold lower than, 7-fold lower than, 8-fold lower than, 9-fold lower than, 10-fold lower than, 20-fold lower than, 50-fold lower than, or 100-fold lower than that of wild type IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

In some embodiments, the IL-7 polypeptide conjugated to the immune checkpoint inhibitor molecule (e.g., the anti-PD-1 antibody or antigen binding fragment) exhibits a half maximal effective concentration (EC50) for inducing STAT5 phosphorylation in at least one T-cell subtype which is comparable to the unconjugated IL-7 polypeptide (e.g., attaching the IL-7 polypeptide to the polypeptide which binds specifically to PD-1 does not substantially diminish the activity of the IL-7 polypeptide). In some embodiments, the EC50 of the IL-7 polypeptide in the immunocytokine for inducing STAT5 phosphorylation in the at least one T-cell subtype is no more than 2-fold greater than, 3-fold greater than, 4-fold greater than, 5-fold greater than, 6-fold greater than, 7-fold greater than, 8-fold greater than, 9-fold greater than, 10-fold greater, 20-fold greater than, 50-fold greater than, or 100-fold greater than that for the unconjugated IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

In some embodiments, the IL-7 polypeptide conjugated to the immune checkpoint inhibitor molecule (e.g., the anti-PD-1 antibody or antigen binding fragment) exhibits a half maximal effective concentration (EC50) for inducing STAT5 phosphorylation in at least one T-cell subtype which is lower compared to the unconjugated IL-7 polypeptide (e.g., attaching the IL-7 polypeptide to the polypeptide which binds specifically to PD-1 enhances the activity of the IL-7 polypeptide). In some embodiments, the EC50 of the IL-7 polypeptide in the immunocytokine for inducing STAT5 phosphorylation in the at least one T-cell subtype is at least 2-fold lower than, 3-fold lower than, 4-fold lower than, 5-fold lower than, 6-fold lower than, 7-fold lower than, 8-fold lower than, 9-fold lower than, 10-fold lower, 20-fold lower than, 50-fold lower than, or 100-fold lower than that for the unconjugated IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

In some embodiments, an immunocytokine provided herein (e.g., a polypeptide which binds to PD-1 (e.g., an anti-PD-1 antibody) attached to an IL-7 polypeptide through a linker as provided herein) maintains binding affinity associated with at least one of the components after formation of the linkage between the two groups. For example, in an immunocytokine comprising an anti-PD-1 antibody or antigen binding fragment linked to an IL-7 polypeptide, in some embodiments the anti-PD-1 antibody or antigen binding fragment thereof retains binding to one or more Fc receptors. In some embodiments, the immunocytokine displays binding to one or more Fc receptors which is reduced by no more than about 5-fold, no more than about 10-fold, no more than about 15-fold, or no more than about 20-fold compared to the unconjugated antibody. In some embodiments, the one or more Fc receptors is the FcRn receptor, CD16a, the FcgRI receptor (CD64), the FcgRIIa receptor (CD32a), the FcgRIIb receptor (CD32b), or any combination thereof. In some embodiments, binding of the immunocytokine to each of the FcRn receptor, CD16a, the FcgRI receptor (CD64), the FcgRIIa receptor (CD32a), and the FcgRIIb receptor (CD32b) is reduced by no more than about 10-fold compared to the unconjugated antibody.

In some embodiments, binding of the immune checkpoint inhibitor molecule (e.g., the anti-PD-1 antibody or antigen binding fragment) is substantially unaffected by the conjugation with the IL-7 polypeptide. In some embodiments, the binding of the immune checkpoint inhibitor molecule to its ligand (e.g., PD-1) is reduced by no more than about 5% compared to the unconjugated antibody. In some embodiments, the binding of the immune checkpoint inhibitor molecule (e.g., the anti-PD-1 antibody or antigen binding fragment) to its ligand (e.g., PD-1) is reduced by no more than about 2-fold, 3-fold, 4-fold, 5-fold, 6-fold, 7-fold, 8-fold, 9-fold, 10-fold, 20-fold, 50-fold, or 100-fold compared to the unconjugated immune checkpoint inhibitor molecule.

### III. Pharmaceutical Compositions

In one aspect, described herein is a pharmaceutical composition comprising: an IL-7 polypeptide described herein or an immunocytokine described herein; and a pharmaceutically acceptable carrier or excipient. In some embodiments, the pharmaceutical composition further comprises one or more excipients, wherein the one or more excipients include, but are not limited to, a carbohydrate, an inorganic salt, an antioxidant, a surfactant, a buffer, or any combination thereof. In some embodiments the pharmaceutical composition further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical composition further comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical composition comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical composition further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical composition further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical composition is formulated for parenteral or enteral administration. In some embodiments, the pharmaceutical composition is formulated for intravenous (IV) or subcutaneous (SQ) administration. In some embodiments, the pharmaceutical composition is in a lyophilized form.

In one aspect, described herein is a liquid or lyophilized composition that comprises a described an IL-7 polypeptide described herein or an immunocytokine described herein. In some embodiments, the IL-7 polypeptide described herein or the immunocytokine described herein is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising about 10 mM Na₂HPO₄ buffer, about 0.022% SDS, and about 50 mg/mL mannitol, and having a pH of about 7.5.

### Dosage Forms

The IL-7 polypeptide described herein or the immunocytokine described herein can be in a variety of dosage forms. In some embodiments, the IL-7 polypeptide described herein or the immunocytokine described herein is dosed as rehydrated from a lyophilized powder. In some embodiments, the IL-7 polypeptide described herein or the immunocytokine described herein is dosed as a suspension. In some embodiments, the IL-7 polypeptide described herein or the immunocytokine described herein is dosed as a solution. In some embodiments, the IL-7 polypeptide described herein or the immunocytokine described herein is dosed as an injectable solution. In some embodiments, the IL-7 polypeptide described herein or the immunocytokine described herein is dosed as an IV solution.

### IV. Methods of Treatment

In one aspect, described herein, is a method of treating cancer in a subject in need thereof, comprising: administering to the subject an effective amount of an IL-7 polypeptide described herein or an immunocytokine described herein. In some embodiments, the cancer is a solid cancer. A cancer or tumor can be, for example, a primary cancer or tumor or a metastatic cancer or tumor. Cancers and tumors to be treated include, but are not limited to, a melanoma, a lung cancer (e.g., a non-small cell lung cancer (NSCLC), a small cell lung cancer (SCLC), etc.), a carcinoma (e.g., a cutaneous squamous cell carcinoma (CSCC), a urothelial carcinoma (UC), a renal cell carcinoma (RCC), a hepatocellular carcinoma (HCC), a head and neck squamous cell carcinoma (HNSCC), an esophageal squamous cell carcinoma (ESCC), a gastroesophageal junction (GEJ) carcinoma, an endometrial carcinoma (EC), a Merkel cell carcinoma (MCC), etc.), a bladder cancer (BC), a microsatellite instability high (MSI-H)/mismatch repair-deficient (dMMR) solid tumor (e.g., a colorectal cancer (CRC)), a tumor mutation burden high (TMB-H) solid tumor, a triple-negative breast cancer (TNBC), a gastric cancer (GC), a cervical cancer (CC), a pleural mesothelioma (PM), classical Hodgkin's lymphoma (cHL), or a primary mediastinal large B cell lymphoma (PMBCL).

Combination therapies with one or more additional active agents are contemplated herein. In some embodiments, the second therapeutic agent is selected based on tumor type, tumor tissue of origin, tumor stage, or mutations in genes expressed by the tumor. For example, an anti-PD-1 antibody can be administered in combination with one or more of the following: a chemotherapeutic agent, a checkpoint inhibitor, a biologic cancer agent, a cancer-specific agent, a cytokine therapy, an anti-angiogenic drug, a drug that targets cancer metabolism, an antibody that marks a cancer cell surface for destruction, an antibody-drug conjugate, a cell therapy, a commonly used anti-neoplastic agent, a CAR-T therapy, an oncolytic virus, a non-drug therapy, a neurotransmission blocker, or a neuronal growth factor blocker.

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms or cancer burden remain static and do not worsen. Further indications of treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the IL-7 polypeptide described herein or the immunocytokine described herein is administered in a single dose of the effective amount of the IL-7 polypeptide or the immunocytokine, including further embodiments in which (i) the IL-7 polypeptide described herein or the immunocytokine described herein is administered once a day; or (ii) the IL-7 polypeptide described herein or the immunocytokine described herein is administered to the subject multiple times over the span of one day. In some embodiments, the IL-7 polypeptide described herein or the immunocytokine described herein is administered daily, every other day, 3 times a week, once a week, every 2 weeks, every 3 weeks, every 4 weeks, every 5 weeks, every 3 days, every 4 days, every 5 days, every 6 days, bi-weekly, 3 times a week, 4 times a week, 5 times a week, 6 times a week, once a month, twice a month, 3 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.).

### V. Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive usc.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used herein, an “alpha-keto amino acid” or the phrase “alpha-keto” before the name of an amino acid refers to an amino acid or amino acid derivative having a ketone functional group positioned between the carbon bearing the amino group and the carboxylic acid of an amino acid. Alpha-keto amino acids of the instant disclosure have a structure as set forth in the following formula:

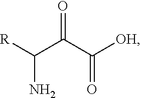

wherein R is the side chain of any natural or unnatural amino acid. The R functionality can be in either the L or D orientation in accordance with standard amino acid nomenclature. In preferred embodiments, alpha-keto amino acids are in the L orientation. When the phrase “alpha-keto” is used before the name of a traditional natural amino acid (e.g., alpha-keto leucine, alpha-keto phenylalanine, etc.) or a common unnatural amino acid (e.g., alpha-keto norleucine, alpha-keto O-methyl-homoserine, etc.), it is intended that the alpha-keto amino acid referred to matches the above formula with the side chain of the referred to amino acid. When an alpha-keto amino acid residue is set forth in a peptide or polypeptide sequence herein, it is intended that a protected version of the relevant alpha-keto amino acid is also encompassed (e.g., for a sequence terminating in a C-terminal alpha-keto amino acid, the terminal carboxylic acid group may be appropriately capped with a protecting group such as a tert-butyl group, or the ketone group with an acetal protecting group). Other protecting groups encompassed are well known in the art.

Binding affinity refers to the strength of a binding interaction between a single molecule and its ligand/binding partner. A higher binding affinity refers to a higher strength bond than a lower binding affinity. In some instances, binding affinity is measured by the dissociation constant (KD) between the two relevant molecules. When comparing KD values, a binding interaction with a lower value will have a higher binding affinity than a binding interaction with a higher value. For a protein-ligand interaction, KD is calculated according to the following formula:

$${K}_{D} = \frac{[ L ] [ P ]}{[ LP ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension: 1, and Compositional Adjustments Conditional Compositional Score Matrix Adjustment. This alignment algorithm is also used to assess if a residue is a “corresponding” position through an analysis of the alignment of the two sequences being compared.

Unless otherwise specified, is contemplated that “protected” versions of amino acids (e.g., those containing a chemical protecting group affixed to a functionality of the amino acid, particularly a side chain of the amino acid but also at another point of the amino acid) qualify as the same amino acid as the “unprotected” version for sequence identity purposes, particularly for chemically synthesized polypeptides. It is also contemplated that such protected versions are also encompassed by the SEQ ID NOs provided herein. Non-limiting examples of protecting groups which may be encompassed include fluorenylmethyloxycarbonyl (Fmoc), triphenylmethyl (trityl or trt), tert-Butyloxycarbonyl (Boc), 2,2,4,6,7-pentamethyldihydrobenzofuran-5-sulfonyl (Pbf), acetamidomethyl (Acm), tert-butyl (tBu or OtBu), 2,2-dimethyl-1-(4-methoxyphenyl) propane-1,3-diol ketal or acetal, and 2,2-dimethyl-1-(2-nitrophenyl) propane-1,3-diol ketal or acetal. Other protecting groups well known in the art are also encompassed. Similarly, modified versions of natural amino acids are also intended to qualify as natural version of the amino acid for sequence identity purposes. For example, an amino acid comprising a side chain heteroatom which can be covalently modified (e.g., to add a conjugation handle, optionally through a linker), such as a lysine, glutamine, glutamic acid, asparagine, aspartic acid, cysteine, or tyrosine, which has been covalently modified would be counted as the base amino acid (see, e.g., Structure 2 below, which would be counted as a lysine for sequence identity and SEQ ID purposes). Similarly, an amino acid comprising another group added to the C or N-terminus would be counted as the base amino acid.

In some instances, peptides provided herein may be depsipeptides. For example, a depsipeptide linkage result from certain ligation reactions described herein (e.g., KAHA ligations) during the synthesis of synthetic IL-7s and relevant precursor peptides. In particular, hydroxyl containing amino acids (e.g., serine, threonine, and homoserine) form depsipeptide linkages with the adjacent amino acid on the N-terminal side. Thus, when a sequence ID lists an amino acid sequence, it is also contemplated that a depsipeptide version of the sequence is also encompassed, particularly at homoserine residues.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0, 2, 3, 4, or 4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, the term “number average molecular weight” (Mn) means the statistical average molecular weight of all the individual units in a sample, and is defined by Formula (1):

$$Mn = \frac{Σ {N}_{i} {M}_{i}}{Σ {N}_{i}} \qquad Formula ( 1 )$$

where Mi is the molecular weight of a unit and Ni is the number of units of that molecular weight.

As used herein, the term “weight average molecular weight” (Mw) means the number defined by Formula (2):

$$Mw = \frac{Σ {N}_{i} {M}_{i}^{2}}{Σ {N}_{i} {M}_{i}} \qquad Formula ( 2 )$$

where Mi is the molecular weight of a unit and Ni is the number of units of that molecular weight.

As used herein, “peak molecular weight” (Mp) means the molecular weight of the highest peak in a given analytical method (e.g. mass spectrometry, size exclusion chromatography, dynamic light scattering, analytical centrifugation, etc.).

“Unnatural” amino acids can refer to amino acid residues in D- or L-form that are not among the 20 canonical amino acids generally incorporated into naturally occurring proteins. Exemplary unnatural amino acids also include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl) alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, p-amino-L-phenylalanine, isopropyl-L-phenylalanine, an analogue of a tyrosine amino acid; an analogue of a glutamine amino acid; an analogue of a phenylalanine amino acid; an analogue of a serine amino acid; an analogue of a threonine amino acid; an analogue of a lysine amino acid, an alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, a β-amino acid; a cyclic amino acid other than proline or histidine; an aromatic amino acid other than phenylalanine, tyrosine or tryptophan; or a combination thereof. In some embodiments, the unnatural amino acids are selected from β-amino acids, homoamino acids, cyclic amino acids and amino acids with derivatized side chains. In some embodiments, the unnatural amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, isodesmosine, allo-isoleucine, ω-methylarginine, N^{α}-methylglycine, N^{α}-methylisoleucine, N^{α}-methylvaline, γ-carboxyglutamate, O-phosphoserine, N^{α}-acetylserine, N^{α}-formylmethionine, 3-methylhistidine, and/or other similar amino acids.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

TABLE of
Conjugation Handles

| | | Reaction |
|---|---|---|
| Conjugation Handle | Complementary Conjugation Handle | Product |
| Sulfhydryl | alpha-halo-carbonyl (e.g., bromoacetamide), alpha- | thioether |
| | beta unsaturated carbonyl (e.g., maleimide, | |
| | acrylamide) | |
| Azide | alkyne (e.g., terminal alkyne, substituted | triazole |
| | cyclooctyne (e.g., dibenzocyclooctyne (DBCO), | |
| | difluorocyclooctynes, bicyclo[6.1.0]nonyne, etc.) ) | |
| Phosphine | Azide/ester pair | amide |
| Tetrazine | trans-cyclooctene | dihydropyrida |
| | | zine |
| Amine | Activated ester (e.g., N-hydroxysuccinimide ester, | amide |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., O- | amide |
| trifluoroborate | carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, -propyl, 1-methyl ethyl, -butyl, -pentyl, 1,1-dimethyl ethyl, 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkynyl group has the formula —C≡C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH, and —CH₂C≡CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopipcrazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 0 atoms, 0-1 P atoms, and 0-1 S atoms in the ring. In some embodiments, a heteroaryl contains 1-4 N atoms, 0-1 0 atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH2, —NH (alkyl), —N(alkyl)₂, —OH, —CO₂H, —CO₂alkyl, —C(═O)NH₂, —C(═O)NH(alkyl), —C(═O)N (alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

As used herein, “AJICAP™ technology,” “AJICAP™ methods,” and similar terms refer to systems and methods (currently produced by Ajinomoto Bio-Pharma Services (“Ajinomoto”)) for the site specific functionalization of antibodies and related molecules using affinity peptides to deliver the desired functionalization to the desired site. General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066; Yamada et al., AJICAP: Affinity Peptide Mediated Regiodivergent Functionalization of Native Antibodies. Angew. Chem., Int. Ed. 2019, 58, 5592-5597, and Fujii et al., Bioconjugate Chem. 2023, 34, 4, 728-738. In some embodiments, such methodologies site specifically incorporate the desired functionalization at lysine residues at a position selected from position 246, position 248, position 288, position 290, and position 317 of an antibody Fc region (e.g., an IgG1 Fc region) (EU numbering). In some embodiments, the desired functionalization is incorporated at residue position 248 of an antibody Fc region (EU numbering). In some embodiments, position 248 corresponds to the 18th residue in a human IgG CH2 region (EU numbering).

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1: General Methods of IL-7 Synthesis

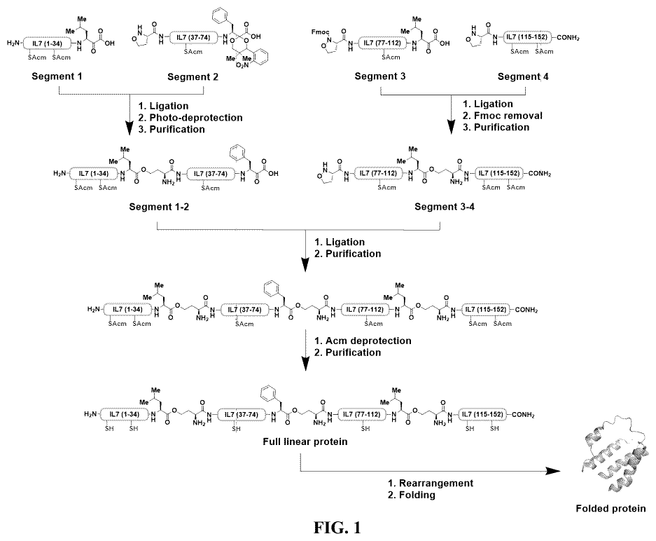

General strategy: Synthetic IL-7 polypeptides are synthesized by ligating individual peptide segments prepared by solid phase peptide synthesis (SPPS). FIG. 1 shows the general synthesis scheme used to produce IL-7 linear proteins. Briefly, peptide segments (Seg1, Seg2, Seg3 and Seg4) were prepared using SPPS, and any desired modification to the amino acid sequence of wild-type IL-7 (SEQ ID NO:1) was incorporated during the synthesis. After purification of the individual fragments, IL-7-Seg1 and IL-7-Seg2 were ligated together, as well as IL-7-Seg3 and IL-7-Seg4. The resulting IL-7-Seg12 and IL-7-Seg34 were purified and ligated together to afford IL-7-Seg1234 with cysteines protected with Acm groups (IL-7-Seg1234-Acm). The Acm groups of IL-7-Seg1234-Acm were then universally deprotected and purified to afford synthetic IL-7 linear protein. The resulting synthetic IL-7 linear proteins were then rearranged and folded. Individual peptides are synthesized on an automated peptide synthesizer using the methods described below.

Materials and solvents: Fmoc-amino acids with suitable side chain protecting groups for Fmoc-SPPS, resins polyethylene glycol derivatives used for peptide functionalization and reagents were commercially available and were used without further purification. The following Fmoc-amino acids with side-chain protecting groups were used: Fmoc-Ala-OH, Fmoc-Arg (Pbf)-OH, Fmoc-Asn (Trt)-OH, Fmoc-Asp (OrBu)-OH, Fmoc-Cys (Acm)-OH, Fmoc-Gln (Trt)-OH, Fmoc-Glu (OrBu)-OH, Fmoc-Gly-OH, Fmoc-His (Trt)-OH, Fmoc-Ile-OH, Fmoc-Leu-OH, Fmoc-Lys (Boc)-OH, Fmoc-Nle-OH, Fmoc-Phe-OH, Fmoc-Pro-OH, Fmoc-Ser (tBu)-OH, Fmoc-Thr (?Bu)-OH, Fmoc-Tyr ((Bu), Fmoc-Trp (Boc)-OH, Fmoc-Val-OH and Fmoc or Boc-Opr-OH (Opr=5-(S)-oxaproline). Fmoc-pseudoproline dipeptides were incorporated in the synthesis if necessary. HPLC grade CH₃CN was used for analytical and preparative RP-HPLC purification. HPLC grade CH₃CN from was used for analytical and preparative RP-HPLC purification.

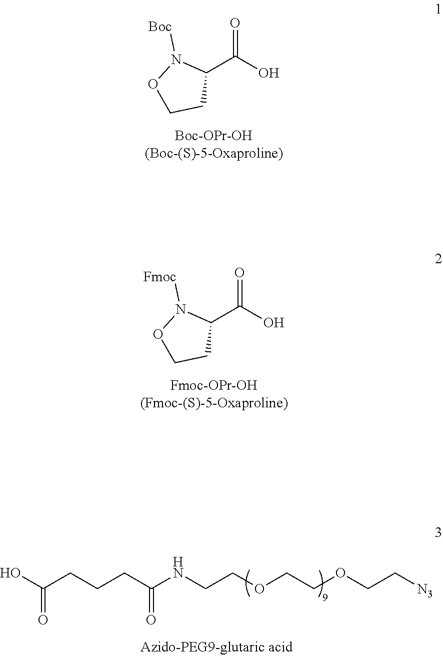

### Special Building Blocks Structures

Protocol 1: Loading of protected ketoacid derivatives (segment 1-3) on amine-based resin: 5 g of Rink-amide MBHA or ChemMatrix resin (1.8 mmol scale) was swollen in DMF for 30 min. Fmoc-deprotection was performed by treating the resin twice with 20% piperidine in DMF (v/v) at r.t. for 10 min. followed by several washes with DMF. Fmoc-AA-protected-α-ketoacid (1.8 mmol, 1.00 equiv.) was dissolved in 20 mL DMF and pre-activated with HATU (650 mg, 1.71 mmol, 0.95 equiv.) and DIPEA (396 μL, 3.6 mmol, 2.00 equiv.). The reaction mixture was added to the swollen resin. It was let to react for 6 h at r.t. under gentle agitation. The resin was rinsed thoroughly with DMF. Capping of unreacted amines on the resin was performed by addition of a solution of acetic anhydride and DIPEA in DMF (20 mL). In an experiment 1.17 ml acetic anhydride and 2.34 ml of DIPEA was used. In another experiment, 1.06 mL, 6 equiv., acetic anhydride and 1.88 mL, 6 equiv., of DIPEA was used. It was let to react at r.t. for 15 min under gentle agitation. The resin was rinsed thoroughly with DCM followed by diethyl ether and dried. The loading of the resin was measured (0.25 mmol/g) following the method described in M. Gude, et al. (2003) Lett. Pept. Sci., 9, 203. In an experiment, the loading of the resin was determined by UV quantification of dibenzofulvene to be 0.25 mmol/g.

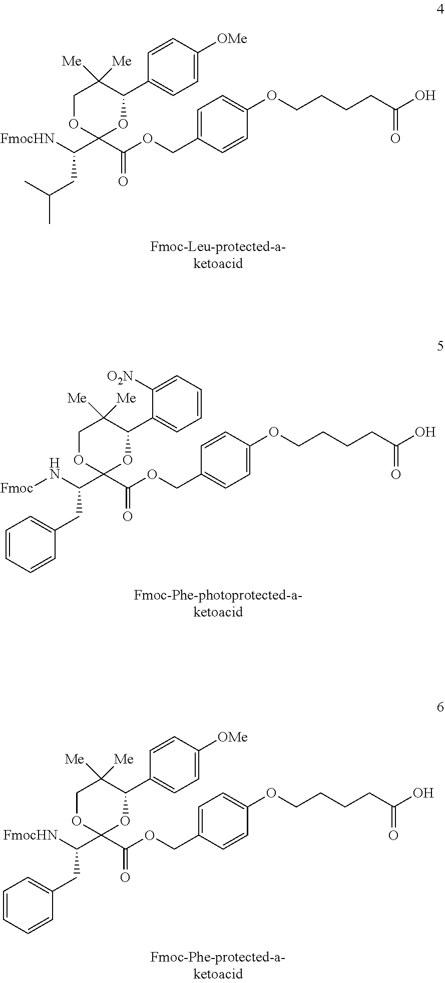

### Protected Ketoacid Used

Protocol 2: Solid-phase peptide synthesis (SPPS): The peptide segments were synthesized on an automated peptide synthesizer using Fmoc-SPPS chemistry. The following Fmoc-amino acids with side-chain protecting groups were used: Fmoc-Ala-OH, Fmoc-Arg (Pbf)-OH, Fmoc-Asn (Trt)-OH, Fmoc-Asp (OrBu)-OH, Fmoc-Cys (Acm)-OH, Fmoc-Gln (Trt)-OH, Fmoc-Glu (OtBu)-OH, Fmoc-Gly-OH, Fmoc-His (Trt)-OH, Fmoc-Ile-OH, Fmoc-Leu-OH, Fmoc-Lys (Boc)-OH, Fmoc-Nle-OH, Fmoc-Phe-OH, Fmoc-Pro-OH, Fmoc-Ser (tBu)-OH, Fmoc-Thr (tBu)-OH, Fmoc-Tyr (tBu), Fmoc-Trp (Boc)-OH, Fmoc-Val-OH and Fmoc or Boc-Opr-OH (Opr=5-(S)-oxaproline). Fmoc-pseudoproline dipeptides were incorporated in the synthesis if necessary. Fmoc deprotection reactions were performed with 20% piperidine in DMF or NMP containing 0.1 M Cl-HOBt (2×2 min). Coupling reactions were performed with Fmoc-amino acid (3.0-8.0 equiv to resin substitution), HCTU or HATU (2.9-8 equiv) as coupling reagents and DIPEA or NMM (6-16 equiv) in DMF or NMP at room temperature. The solution containing the reagents was added to the resin and allowed to react for 15 min, 30 min, or 2 h depending on the amino acid. Double coupling reactions were performed as needed. Unreacted free amines were capped using 20% acetic anhydride in DMF or NMP and 0.8 M NMM in DMF or NMP for 6 min.

Protocol 3: Alloc deprotection on resin: DMBA (20 equiv.) dissolved in DMSO (25 mL/mmol) was added to the resin preswollen in DCM followed by Pd[PPh₃]₄ (0.2-0.5 equiv.) dissolved in 8-10 mL of dry DCM. The resin was shaken for 15-30 min and then washed twice with DCM (50 mL/mmol resin substitution), twice with DMF (50 mL/mmol resin substitution), twice with DCM (50 mL/mmol resin substitution), twice with IPA (50 mL/mmol resin substitution), and twice with Et₂O (50 mL/mmol resin substitution). The reaction and the wash were repeated once.

Protocol 4: Glutaric acid incorporation: glutaric anhydride (5 equiv.) was dissolved in DMF (37.5 mL/mmol resin substitution) and DIPEA (7 equiv.) was added. The mixture was transferred to the resin and let it shake for 2 h at rt. The resin was washed twice with DMF (10V), twice with DCM (10V) and twice with IPA (10V).

Protocol 5: Resin cleavage and side chain deprotection. Once the peptide synthesis was completed, the peptides were cleaved from the resin using a cleavage cocktail at room temperature for 2 h. The resin was filtered off, and the filtrate was concentrated and treated with cold diethyl ether, triturated and centrifuged. The ether layer was carefully decanted, the residue was suspended again in diethyl ether, triturated and centrifuged. Ether washings were repeated twice. The resulting crude peptide was dried under vacuum and stored at −20° C. An aliquot of the solid obtained was solubilized in 1:1 CH₃CN/H₂O with 0.1% TFA (v/v) and analyzed by analytical RP-HPLC using C18 column (3.6 μm, 150×4.6 mm) at 50° C.-60° C. The molecular weight of the product was identified using MALDI-TOF or LC-MS (ESI).

| Segment | Cleavage cocktail |
|---|---|
| Segment 1 | TFA:DTT:H₂O:TIS (87.5:5:5:2.5) (v/v/v/v) |
| Segment 2 | TFA:DODT:H₂O (95:2.5:2.5) (v/v/v) |
| Segment 3 | TFA:DODT:H₂O (95:2.5:2.5) (v/v/v) |
| Segment 4 | TFA:DTT:H₂O:TIS (87.5:5:5:2.5) (v/v/v/v) |

Protocol 6: Ligation of IL-2 segments 1 and 2 and photodeprotection: IL-7 Seg1 (1.2 equiv.) and IL-7 Seg2 (1 equiv.) were dissolved in DMSO: H₂O (9.5:0.5, v/v) containing 0.1 M oxalic acid (15 mM peptide concentration) and allowed to react at 60° C. for 22 h. The ligation vial was protected from light by wrapping it in aluminum foil. The progress of the KAHA ligation was analyzed by HPLC using a C18 column (4.6×150 mm) at 60° C. or 50° C. with CH₃CN/H₂O containing 0.1% TFA as mobile phase. After completion of the ligation, the mixture was diluted with CH₃CN:H₂O (1:1) containing 0.1% TFA, or DMSO, and irradiated at a wavelength of 365 nm for 1.5 h. The completion of photolysis reaction was confirmed by analytical RP-HPLC. The sample was then purified by preparative HPLC.

Protocol 7: Ligation of IL-7 segments 3 and 4 and Fmoc deprotection: IL7-Seg3 (1.2 equiv.) and IL7-Seg4 (1 equiv.) were dissolved in DMSO/H₂O (9:1) (v/v) containing 0.1 M oxalic acid (20 mM) and allowed to react for 20 h at 60° C. The progress of the KAHA ligation was monitored by HPLC using a C18 column (4.6×150 mm) at 50° C. using CH₃CN/H₂O containing 0.1% TFA as mobile phase. After completion of ligation, the reaction mixture was diluted with DMSO (6 mL) and 5% of diethylamine (300 mL) or 5% DBU were added. The reaction mixture was shaken for 15-30 min at room temperature. To prepare the sample for purification, it was diluted with DMSO containing TFA. The sample was purified by preparative HPLC.

Protocol 8: Final ligation: IL7-Seg12 (1.2 equiv.) and IL7-Seg34 (1 equiv.) were dissolved in DMSO/H₂O (9.5:0.5) (v/v) containing 0.1 M oxalic acid (15 mM peptide concentration) and the ligation was allowed to proceed for 24 h at 60° C. The progress of the KAHA ligation was monitored by analytical HPLC using a C18 column (4.6×250 mm) at 50° C. and CH₃CN/H₂O containing 0.1% TFA as mobile phase. After completion of ligation, the reaction mixture was diluted with DMSO followed by further dilution with a mixture of (1:1) CH₃CN:H₂O containing 0.1% TFA. The sample was purified by preparative HPLC.

Protocol 9: Acm deprotection: IL7 linear protein with Acm was dissolved in AcOH/H₂O (1:1) (0.25 mM protein concentration) and AgOAc (1% m/v) was added to the solution. The mixture was shaken for 2.5 h at 50° C. protected from light. After completion of reaction as ascertained by analytical HPLC, the sample was diluted with CH₃CN:H₂O (1:9) containing 0.1% TFA (v/v), and purified by preparative HPLC.

### Protocol 10: Rearrangement and Folding:

Folding Step 1: The linear protein was dissolved in 50 mM Tris buffer, containing 6 M GnHCl, 50 mM NaCl, 1 mM EDTA and 2-10 mM CysHCl (15-40 μM protein concentration), which was adjusted to pH 8.0 by adding a solution of 6 M aqueous HCl. The mixture was gently shaken for 3 h at r.t. The rearrangement was monitored by analytical reverse phase HPLC at 25° C., with a gradient of 30 to 85% acetonitrile with 0.1% TFA in 12 min.

Folding Step 2: The solution with the rearranged protein was cooled to 4° C. and diluted (×3-8) with 50 mM Tris buffer containing 50 mM NaCl, 0.11 M Arg, 1 mM EDTA and 0-0.142 mM cystine, which was adjusted to pH 8.0 by adding a solution of 6 M aqueous HCl. The folding was performed for 20 h at 4° C. and monitored according to the rearrangement monitoring conditions. After completion of the folding reaction as ascertained by HPLC, the sample was acidified with TFA to pH=3, and purified by preparative HPLC using C4 column (20×250 mm) at a flow rate of 10 mL/min at r.t. using CH₃CN/H₂O with 0.1% TFA (v/v) as mobile phase, with a gradient of 30 to 85% CH₃CN with 0.1% TFA (v/v) in 50 min. The fractions containing the desired product were pooled and lyophilized with 5% sucrose (w/v) to obtain the folded IL-7 protein. The lyophilized protein was reconstituted in PBS buffer pH 7.4 containing 5% sucrose (m/v). The purity and identity of the pure folded protein was further confirmed by analytical HPLC and LC/ESI/MS/MS or HRMS.

Protocol 11: PEGylation and purification of folded IL-7 proteins: the corresponding folded IL-7 protein was dissolved in 20 mM His buffer pH 5.2 containing 5% sucrose (50 μM protein concentration) and 30 kDa DBCO-PEG (structure 6, 3 equiv.) was added to the previous solution. The resulting mixture was stirred overnight at room temperature. After completion of reaction as ascertained by HPLC, the sample was purified by preparative HPLC using a C4 column (20×250 mm) at a flow rate of 10 mL/min at r.t. using CH₃CN/H₂O with 0.1% TFA (v/v) as mobile phase, with a gradient of 10 to 75% CH₃CN with 0.1% TFA (v/v) in 30 min. The fractions containing the purified product were pooled and lyophilized. The resulting protein was reconstituted in 20 mM sodium acetate buffer pH 5.5 and purified by cation exchange chromatography using a HiTrap Capto-S Impact 5 mL column with 20 mM sodium acetate buffer pH 5.6 containing 1 M NaCl as running buffer. The fraction containing the desired product were pooled and concentrated. The purity and identity of the PEGylated protein was further confirmed by analytical HPLC and MALDI-TOF.

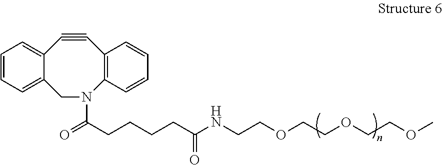

Protocol 12: Purification of the peptides and proteins: Peptide segments, ligated peptides and linear proteins were purified by RP-HPLC. Different gradients were applied for the different peptides. The mobile phase was MilliQ-H₂O with 0.1% TFA (v/v) (Buffer A) and HPLC grade CH₃CN with 0.1% TFA (v/v) (Buffer B). Preparative HPLC was performed on a C4 (50×250 mm) or on a C18 column (50×250 mm) at a flow rate of 40 or 55 mL/min at 40° C. or 60° C.

Purification: The peptide fragments purification was performed on standard preparative HPLC instruments. Preparative HPLC was performed on C18 column (5 μm, 110 Å, 50×250 mm) at a flow rate of 40 mL/min on C18 column (5 μm, 110 Å, 20×250 mm) or C4 column (5 μm, 300 Å, 20.0×250 mm) at a flow rate of 10 mL/min. For both columns, room temperature, 40° C., or 60° C. were used during the purification. The mobile phase was MilliQ-H₂O with 0.1% TFA (v/v) (Buffer A) and HPLC grade CH₃CN with 0.1% TFA (v/v) (Buffer B).

A representative gradient used for the purification is given below:
Buffers: A: H₂O 0.1% TFA (v/v), B: ACN 0.1% TFA (v/v)
Column: C18 5 μm; 50×250 mm
Temperature: 50° C.

| Time (min) | A % | B % | Flow (ml/min) |
|---|---|---|---|
| 0 | 95 | 5 | 55 |
| 2 | 90 | 10 | 55 |
| 10 | 90 | 10 | 55 |
| 15.1 | 68 | 32 | 55 |
| 51 | 58 | 42 | 55 |
| 52 | 5 | 95 | 55 |
| 60 | 5 | 95 | 55 |
| 61 | 95 | 5 | 55 |
| 65 | 95 | 5 | 55 |

Characterization of the peptides: Peptides, proteins, peptide segments, ligated peptides, linear proteins and folded proteins were characterized by high resolution Fourier-transform mass spectrometry (FTMS) using a SolariX (9.4T magnet) spectrometer (Bruker, Billerica, USA) equipped with a dual ESI/MALDI-FTICR source, using 4-hydroxy-α-cyanocinnamic acid (HCCA) as matrix. Peptides segments, ligated peptides and linear proteins were analyzed by RP-HPLC using analytical HPLC instruments. In certain methods, HPLC was performed using standard C4 column (3.6 μm, 150×4.6 mm) at room temperature or standard C18 column (3.6 μm, 150×4.6 mm) with a flow rate of 1 mL/min at 50° C. The peptide fragments were analyzed using a gradient of 20% B to 95% B in 12 min (Method A), 10% B to 85% B in 12 min (Method B) or 10% B to 95% B in 12 min (Method C).

### HPLC Methods

Buffers used: A=H₂O with 0.1% TFA (v/v), B=ACN with 0.1% TFA (v/v)

### Method 1:

Column: Phenomenex Aeris 3.6 μm um Widepore XB-C18; 4.6×150 mm
Temperature: 50° C.
Gradient:

| Time | Flow | | |
|---|---|---|---|
| (min) | (mL/min) | % A | % B |
| 1.40 | 1 | 80 | 20 |
| 1.50 | 1 | 80 | 20 |
| 7.50 | 1 | 5 | 95 |
| 10 | 1 | 5 | 95 |
| 10.50 | 1 | 80 | 20 |
| 12 | 1 | 80 | 20 |

### Method 2:

Column: Phenomenex Acris 3.6 μm um Widepore XB-C18; 4.6×150 mm
Temperature: 50° C.

### Gradient:

| Time | Flow | | |
|---|---|---|---|
| (min) | (mL/min) | % A | % B |
| 1.90 | 1 | 90 | 10 |
| 2.00 | 1 | 90 | 10 |
| 7.00 | 1 | 15 | 85 |
| 7.20 | 1 | 5 | 95 |
| 10 | 1 | 5 | 95 |
| 10.20 | 1 | 90 | 10 |
| 11.80 | 1 | 90 | 10 |

### Method 3:

Column: Phenomenex Acris 3.6 μm um Widepore XB-C18; 4.6×150 mm
Temperature: 50° C.
Gradient:

| Time | Flow | | |
|---|---|---|---|
| (min) | (mL/min) | % A | % B |
| 1.40 | 1 | 70 | 30 |
| 1.50 | 1 | 70 | 30 |
| 7.50 | 1 | 5 | 95 |
| 10 | 1 | 5 | 95 |
| 10.50 | 1 | 95 | 5 |
| 12 | 1 | 95 | 5 |

### Method 4:

Column: Biozen 3.6 μm intact C4, 4.6×150 mm
Temperature: 25° C.
Gradient:

| Time | Flow | | |
|---|---|---|---|
| (min) | (mL/min) | % A | % B |
| 0.05 | 1 | 95 | 5 |
| 2.50 | 1 | 95 | 5 |
| 3.50 | 1 | 70 | 30 |
| 13.00 | 1 | 5 | 95 |
| 15.00 | 1 | 5 | 95 |
| 16.00 | 1 | 95 | 5 |
| 18.00 | 1 | 95 | 5 |

### Method 5:

Column: Biozen 3.6 μm intact C4, 4.6×150 mm
Temperature: 25° C.
Gradient:

| Time | Flow | | |
|---|---|---|---|
| (min) | (mL/min) | % A | % B |
| 0.05 | 1 | 70 | 30 |
| 2.50 | 1 | 70 | 30 |
| 12.00 | 1 | 30 | 70 |
| 12.50 | 1 | 5 | 95 |
| 16.00 | 1 | 5 | 95 |
| 17.90 | 1 | 70 | 30 |
| 18.00 | 1 | 70 | 30 |

### Method 6:

Column: Biozen 3.6 μm intact C4, 4.6×150 mm
Temperature: 25° C.
Gradient:

| Time | Flow | | |
|---|---|---|---|
| (min) | (mL/min) | % A | % B |
| 0.05 | 1 | 95 | 5 |
| 2.50 | 1 | 95 | 5 |
| 12.00 | 1 | 15 | 85 |
| 12.50 | 1 | 5 | 95 |
| 16.00 | 1 | 5 | 95 |
| 17.90 | 1 | 95 | 5 |
| 18.00 | 1 | 95 | 5 |

### Method 7:

Column: Biozen 3.6 μm intact C4. 4.6×150 mm
Temperature: 25° C.
Gradient:

| Time | Flow | | |
|---|---|---|---|
| (min) | (mL/min) | % A | % B |
| 0.10 | 1 | 95 | 5 |
| 2.00 | 1 | 95 | 5 |
| 5.00 | 1 | 70 | 30 |
| 12.80 | 1 | 25 | 75 |
| 12.90 | 1 | 5 | 95 |
| 15.00 | 1 | 5 | 95 |
| 16.00 | 1 | 95 | 5 |
| 20.00 | 1 | 95 | 5 |

### Method 8:

Column: Phenomenex Acris 3.6 μm um Widepore XB-C18: 4.6×150 mm
Temperature: 50° C.
Gradient:

| Time | Flow | | |
|---|---|---|---|
| (min) | (mL/min) | % A | % B |
| 1.40 | 1 | 70 | 20 |
| 1.50 | 1 | 70 | 20 |
| 7.50 | 1 | 30 | 70 |
| 7.60 | 1 | 5 | 95 |
| 10 | 1 | 5 | 95 |
| 10.50 | 1 | 95 | 5 |
| 11.80 | 1 | 95 | 5 |

### Method 9:

Column: Phenomenex Acris 3.6 μm um Widepore XB-C18; 4.6×150 mm
Temperature: 50° C.
Gradient:

| Time | Flow | | |
|---|---|---|---|
| (min) | (mL/min) | % A | % B |
| 1.40 | 1 | 70 | 30 |
| 1.50 | 1 | 70 | 30 |
| 7.50 | 1 | 30 | 70 |
| 7.60 | 1 | 5 | 95 |
| 10 | 1 | 5 | 95 |
| 10.50 | 1 | 95 | 5 |
| 11.80 | 1 | 95 | 5 |

### Method 10:

Column: Phenomenex Acris 3.6 μm um Widepore XB-C18; 4.6×150 mm
Temperature: 50° C.
Gradient:

| Time | Flow | | |
|---|---|---|---|
| (min) | (mL/min) | % A | % B |
| 1.40 | 1 | 60 | 40 |
| 1.50 | 1 | 60 | 40 |
| 7.50 | 1 | 10 | 90 |
| 7.60 | 1 | 5 | 95 |
| 10 | 1 | 5 | 95 |
| 10.50 | 1 | 95 | 5 |
| 11.80 | 1 | 95 | 5 |

### Method 11:

Column: Waters Xbridge Protein BEH; C4 300 Å; 2.5 μm: 3×150 mm
Temperature: room temperature
Gradient:

| Time | Flow | | |
|---|---|---|---|
| (min) | (mL/min) | % A | % B |
| 0 | 0.8 | 90 | 10 |
| 1.9 | 0.8 | 90 | 10 |
| 2 | 0.8 | 80 | 20 |
| 17 | 0.8 | 30 | 70 |
| 17.1 | 0.8 | 5 | 95 |
| 19 | 0.8 | 5 | 95 |
| 19.1 | 0.8 | 90 | 10 |
| 21 | 0.8 | 90 | 10 |

### Method 12:

Column: Waters Xbridge Protein BEH; C4 300 Å; 2.5 μm: 3×150 mm
Temperature: room temperature
Gradient:

| Time | Flow | | |
|---|---|---|---|
| (min) | (mL/min) | % A | % B |
| 0 | 1 | 90 | 10 |
| 1.0 | 1 | 90 | 10 |
| 9.9 | 1 | 40 | 60 |
| 10.0 | 1 | 5 | 95 |
| 11.4 | 1 | 5 | 95 |
| 11.5 | 1 | 90 | 10 |
| 13.9 | 1 | 90 | 10 |

### Method 13:

Column: Phenomenex Acris 3.6 μm um Widepore XB-C18; 4.6×150 mm
Temperature: 50° C.
Gradient:

| Time | Flow | | |
|---|---|---|---|
| (min) | (mL/min) | % A | % B |
| 1.40 | 1 | 70 | 30 |
| 1.50 | 1 | 70 | 30 |
| 7.50 | 1 | 15 | 85 |
| 7.60 | 1 | 5 | 95 |
| 10 | 1 | 5 | 95 |
| 10.50 | 1 | 70 | 30 |
| 11.80 | 1 | 70 | 30 |

### Method 14:

Column: Phenomenex Acris 3.6 μm um Widepore XB-C18; 4.6×150 mm
Temperature: 50° C.
Gradient:

| Time | Flow | | |
|---|---|---|---|
| (min) | (mL/min) | % A | % B |
| 1.40 | 1 | 80 | 20 |
| 1.50 | 1 | 80 | 20 |
| 7.50 | 1 | 40 | 60 |
| 7.60 | 1 | 5 | 95 |
| 10 | 1 | 5 | 95 |
| 10.50 | 1 | 80 | 20 |
| 11.80 | 1 | 80 | 20 |

### Method 15:

Column: Phenomenex Acris 3.6 μm Widepore C4 200 Å: 4.6×150 mm
Temperature: 50° C.
Gradient:

| Time | Flow | | |
|---|---|---|---|
| (min) | (mL/min) | % A | % B |
| 0 | 2 | 80 | 20 |
| 1.5 | 2 | 80 | 20 |
| 5.4 | 2 | 30 | 70 |
| 5.5 | 2 | 5 | 95 |
| 6.9 | 2 | 5 | 95 |
| 7 | 2 | 80 | 20 |
| 9 | 2 | 80 | 20 |
| 9.1 | 2 | 80 | 20 |

### Method 16:

Column: Waters XBridge C18 3.5 μm; 3×150 mm
Temperature: 50° C.
Gradient:

| Time | Flow | | |
|---|---|---|---|
| (min) | (mL/min) | % A | % B |
| 0 | 0.8 | 90 | 10 |
| 2 | 0.8 | 90 | 10 |
| 17 | 0.8 | 45 | 55 |
| 17.1 | 0.8 | 5 | 95 |
| 19 | 0.8 | 5 | 95 |
| 19.1 | 0.8 | 90 | 10 |
| 21 | 0.8 | 90 | 10 |

### Example 2A: Preparation of a Synthetic IL-7 Polypeptide of SEQ ID NO: 3

### Synthesis Method 1

Segment 1: IL-7 (1-34)-Leu-α-ketoacid (SEQ ID NO: 101)

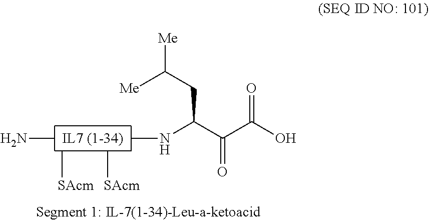

SEQ ID NO: 101 was synthesized on a 0.2 mmol scale on Rink Amide MBHA resin pre-loaded with Fmoc-Leu-protected-α-ketoacid (description in the general methods) (0.8 g) with a substitution capacity of ˜0.25 mmol/g.

Automated Fmoc-SPPS of SEQ ID NO: 101: The peptide elongation cycles including amino acid coupling, capping and Fmoc deprotection were performed as described in the general methods.

After the peptide elongation, the resin was washed with DCM and dried under vacuum. The mass of the dried peptidyl resin was 1.6 g. The peptide was cleaved from the resin using a mixture of 95:2.5:2.5 TFA:DODT:H₂O (10 mL/g resin) at room temperature for 2.0 h. The compound was precipitated as described in the general methods. 702 mg of crude peptide were obtained.

Purification of crude SEQ ID NO: 101 was performed by preparative HPLC using a C18 column (5 μm, 110 Å, 250×50 mm) at a flow rate of 40 mL/min at 60° C. with a gradient of 30 to 80% B in 25 min. The fractions containing the purified product were pooled and lyophilized to obtain SEQ ID NO: 101 as a white solid in 94% purity. The isolated yield based on the resin loading was 17% (135 mg). MS (ESI): C₁₇₁H₂₈₁N₄₃O₆₃S₂; Average isotope calculated 1337.9940 Da [M+H]³⁺; found: 1337.9933 Da [M+H]³⁺. Retention time (analytical method A): 5.66 min.

### Segment 2: Opr-IL-7 (37-74)-Phe-Photoprotected-α-Ketoacid (SEQ ID NO: 106)

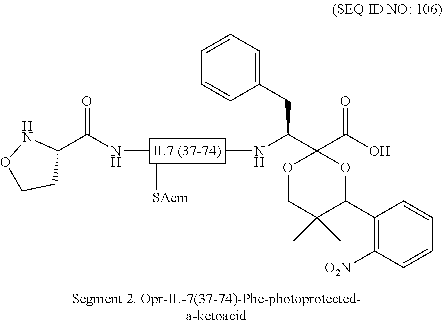

SEQ ID NO: 104 was synthesized on a 0.2 mmol scale on Rink Amide MBHA resin pre-loaded with Fmoc-Phe-photoprotected-α-ketoacid (description in the general methods) with a substitution capacity of 0.25 mmol/g.

The peptide elongation cycles including amino acid coupling, capping and Fmoc deprotection were performed as described in the general methods.

After the peptide elongation, the resin was washed with DCM and diethyl ether and dried under vacuum. The mass of the dried peptidyl resin was 2.2 g. The peptide was cleaved from the resin using a mixture of 95:2.5:2.5 TFA/DODT/H₂O (15 mL/g resin) at room temperature for 2.0 h. The compound was precipitated as described in the general methods. 1.2 g of crude peptide were obtained.

Purification of crude SEQ ID NO: 106 was performed by preparative HPLC using a C18 column (5 μm, 110 Å, 250×50 mm) at a flow rate of 40 mL/min at 40° C. using CH₃CN/H₂O with a gradient of 10 to 60% B in 30 min. The fractions containing the purified product were pooled and lyophilized to obtain SEQ ID NO: 106 as a white solid in 97% purity. The isolated yield based on the resin loading was 20% (203 mg). RMS (ESI): C₂₃₄H₃₅₂N₆₅O₆₂S; m/z calculated: 5098.6114 Da [M+H]⁺; found: 5098.6026 Da [M+H]⁺. retention time (analytical method A): 6.30 min.

### Segment 3: Fmoc-Opr-IL-7 (77-112)-Leu-α-Ketoacid (SEQ ID NO: 109)

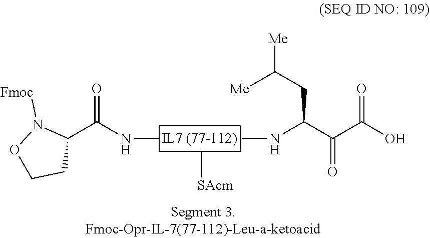

SEQ ID NO: 106 was synthesized on a 0.1 mmol scale on Rink Amide resin pre-loaded with Fmoc-Leu-protected-α-ketoacid (description in the general methods) with a substitution capacity of ˜0.29 mmol/g. 345 mg of resin was swollen in DMF for 15 min.

Automated Fmoc-SPPS of SEQ ID NO: 109: The peptide elongation cycles including amino acid coupling, capping and Fmoc deprotection were performed as described in the general methods.

After the peptide elongation, the resin was washed with DCM and dried under vacuum. The mass of the dried peptidyl resin was 0.94 g. The peptide was cleaved from the resin using a mixture of 95:2.5:2.5 TFA:DODT:H₂O (10 mL/g resin) at room temperature for 2.0 h. The compound was precipitated as described in the general methods. 473 mg of crude peptide were obtained.

Purification of crude SEQ ID NO: 109 was performed by preparative HPLC using a C18 column (5 μm, 110 Å, 50×250 mm) at a flow rate of 40 mL/min at 40° C. with a gradient of 10 to 50% B in 40 min. The fractions containing the purified product were pooled and lyophilized to obtain SEQ ID NO: 106 as a white solid in 98% purity. The isolated yield based on the resin loading was 25% (107 mg). HRMS (ESI): C₁₉₃H₃₁₅N₅₁O₅₇S; Average isotope calculated 4294.0030 Da [M+H]; found: 4293.2962 Da. Retention time (analytical method B): 6.29 min.

### Segment 4: Opr-IL-7 (115-152) (SEQ ID NO: 116)

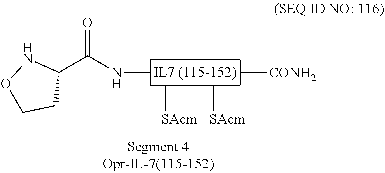

SEQ ID NO: 116 was synthesized on a 0.1 mmol scale on Rink Amide MBHA resin with a substitution capacity of ˜0.34 mmol/g. 294 mg of resin was swollen in DMF for 15 min.

Automated Fmoc-SPPS of SEQ ID NO: 116: The peptide elongation cycles including amino acid coupling, capping and Fmoc deprotection were performed as described in the general methods.

The resin was washed with DCM and dried under vacuum. The mass of the dried peptidyl resin was 725 mg. The peptide was cleaved from the resin using a mixture of 92.5:2.5:2.5:2.5 TFA:TIPS:DODT:H₂O (10 mL/g resin) at room temperature for 2.0 h. The compound was precipitated as described in the general methods. 145 mg of crude peptide were obtained.

Purification of crude SEQ ID NO: 116 was performed by preparative HPLC using a Gemini NX-C18 110 Å column (5 μm, 50×250 mm) at a flow rate of 40 mL/min at 40° C. with a gradient of 10 to 50% B in 40 min. The fractions containing the purified product were pooled and lyophilized to obtain SEQ ID NO: 110 as a white solid in 98% purity. The isolated yield based on the resin loading was 8% (40 mg). HRMS (ESI): C₂₁₅H₃₆₁N₆₁O₆₀S₂; Average isotope calculated 4823.6568 Da [M+H]; found: 4823.6542 Da. Retention time (analytical method B): 6.15 min.

### Segment 12: IL-7-Seg12 Preparation (Residues 1-75 of SEQ ID NO: 3)

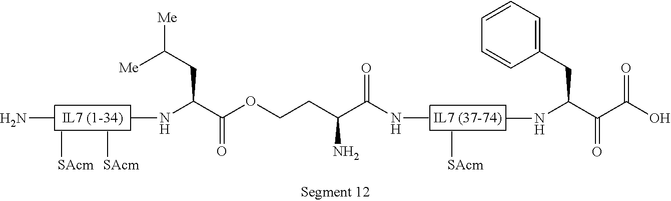

SEQ ID NO: 101 (17.5 mg; 4.36 μmol; 1.1 equiv) ketoacid and SEQ ID NO: 106 (20 mg; 3.92 μmol; 1.0 equiv) were dissolved in 15 mM DMSO:H₂O (9.5:0.5) containing 0.1 M oxalic acid (241 μL). A very homogeneous liquid solution was obtained. The ligation vial was protected from light and the mixture was heated overnight at 60° C. After completion of the ligation, the mixture was diluted with 1:1 CH₃CN:H₂O with 0.1% TFA (v/v) (4 mL), and the mixture was irradiated at a wavelength of 365 nm for 1.5 h to allow photodeprotection of the C-terminal ketoacid. The reaction mixture was further diluted with 1:1 CH₃CN/H₂O (q.s. 10 mL) with TFA (0.1%, v/v).

The diluted mixture was filtered and injected into preparative HPLC. Crude ligated peptide was purified by preparative HPLC using aC18 column (5 μm, 50×250 mm) at a flow rate of 40 mL/min at 60° C., with a 2-step gradient: 10 to 40% B in 5 min, then 40 to 70% Bin 30 min. The fractions containing the purified product were pooled and lyophilized to obtain Segment 12 as a white solid in 98% purity. The isolated yield was 38% (13.1 mg). MS (ESI): C₃₉₃H₆₁₉N₁₀₇O₁₂₀S₃; m/z calculated: 8858.4917 Da [M+H]⁺; found: 8858.4928 Da [M+H]⁺. Retention time (analytical method B): 5.41 min.

### Segment 34: IL-7-Seg34 Preparation (Residues 76-152 of SEQ ID NO: 3)

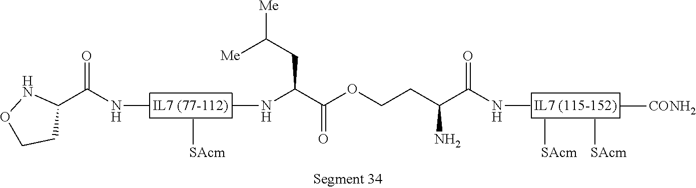

Peptide ketoacid SEQ ID NO: 109 (55.0 mg; 12.8 μmol; 1.2 equiv) and hydroxylamine peptide SEQ ID NO: 116 (51.5 mg; 10.6 μmol; 1.0 equiv) were dissolved in 9:1 DMSO/H₂O containing 0.1 M oxalic acid (530 μL). A very homogeneous liquid solution was obtained. It was let to react. The reaction was heated overnight at 60° C. Upon completion of the ligation reaction, the mixture was diluted with DMSO (1060 μL). Fmoc deprotection was performed initiated by adding diethylamine (80 μL, 5%, v/v) at room temperature for 15 min. A second portion of diethylamine (80 μL) in DMSO (1590 μL) was added to the reaction mixture, and the resulting mixture was reacted that was stirred at room temperature for another 15 min.

Trifluoroacetic acid (160 μL) was added in order to neutralize the reaction mixture. A very homogeneous and colorless liquid solution was obtained. The resulting mixture was further diluted with 1:1 CH₃CN/H₂O (q.s. 15 mL) with TFA (0.1%, v/v). The diluted mixture was filtered and purified by preparative HPLC using a C18 column (5 μm, 250×50 mm) at a flow rate of 40 mL/min at 40° C. with a gradient of 10% to 50% B in 40 min. The fractions containing the purified product were pooled and lyophilized to obtain Segment 34 as a white solid in 92% purity. The isolated yield was 51.5 mg (55%). HRMS (ESI): C₃₉₂H₆₆₆N₁₁₂O₁₁₃S₃; Average isotope calculated 8851.9104 Da [M+H]; found: 8851.8897 Da. Retention time (analytical method B): 6.08 min.

Preparation of SEQ ID NO: 3-Seg1234 with Acm (SEQ ID NO: 3)

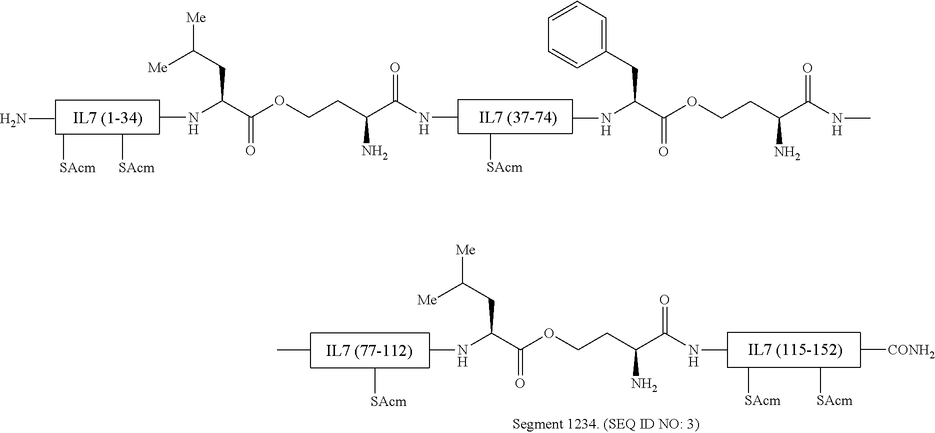

Peptide ketoacid Segment 12 (17.4 mg; 1.96 μmol; 1.2 equiv) and hydroxylamine peptide Segment 34 (14.5 mg; 1.64 μmol; 1.0 equiv) were dissolved in DMSO:H₂O (9.5:0.5) containing 0.1 M oxalic acid (110 μL, 15 mM peptide concentration). A homogeneous liquid solution was obtained, and the solution was heated overnight at 60° C.

After completion of the ligation the mixture was diluted with 1:1 H₂O/CH₃CN (q.s. 8 mL) containing TFA (0.1%, v/v). The diluted mixture was filtered and injected into preparative HPLC. Crude ligated peptide was purified by preparative HPLC using a C18 column (5 μm, 250×50 mm) at a flow rate of 40 mL/min at 60° C. using with a gradient of 30 to 80% B in 30 min. The fractions containing the purified product were pooled and lyophilized to obtain SEQ ID NO: 3 (tri-depsipeptide) with Acm as a white solid in 99% purity. The isolated yield was 28% (8 mg). HRMS (ESI): C₇₈₄H₁₂₈₅N₂₁₉O₂₃₁S₆; Average isotope calculated 17666.4121 Da [M]; found: 17666.4233 Da. Retention time (analytical method C): 5.33 min.

### Acm Deprotection for the Preparation of SEQ ID NO: 3-Linear Protein: IL-7-Linear Protein (SEQ ID NO: 3)

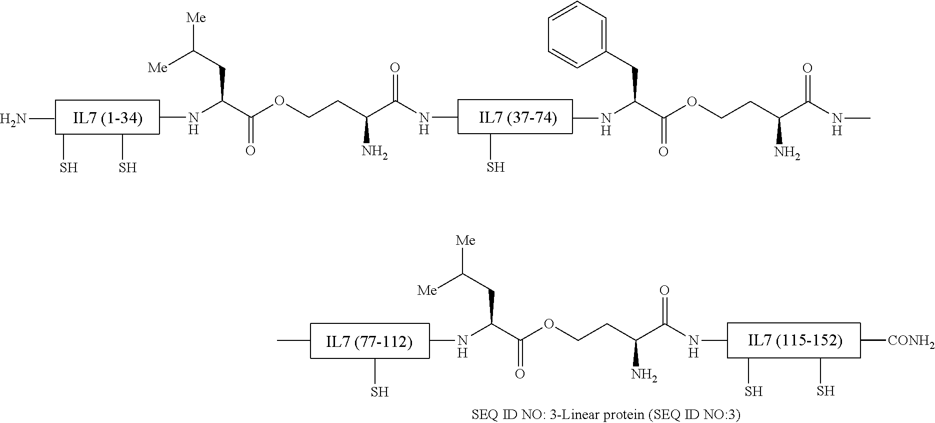

SEQ ID NO: 3 (5.8 mg; 0.33 μmol) was dissolved in AcOH:H₂O (1:1) (1.3 mL, 0.25 mM protein concentration) and silver acetate (13 mg, 1%, m/v) was added to the solution. The mixture was shaken for 2.5 h at 50° C. protected from light.

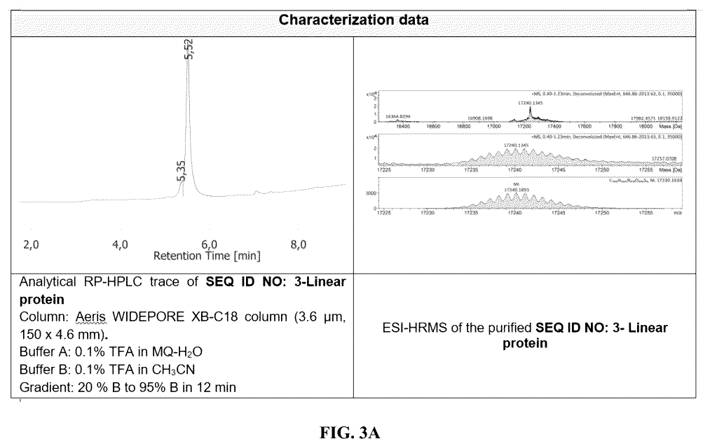

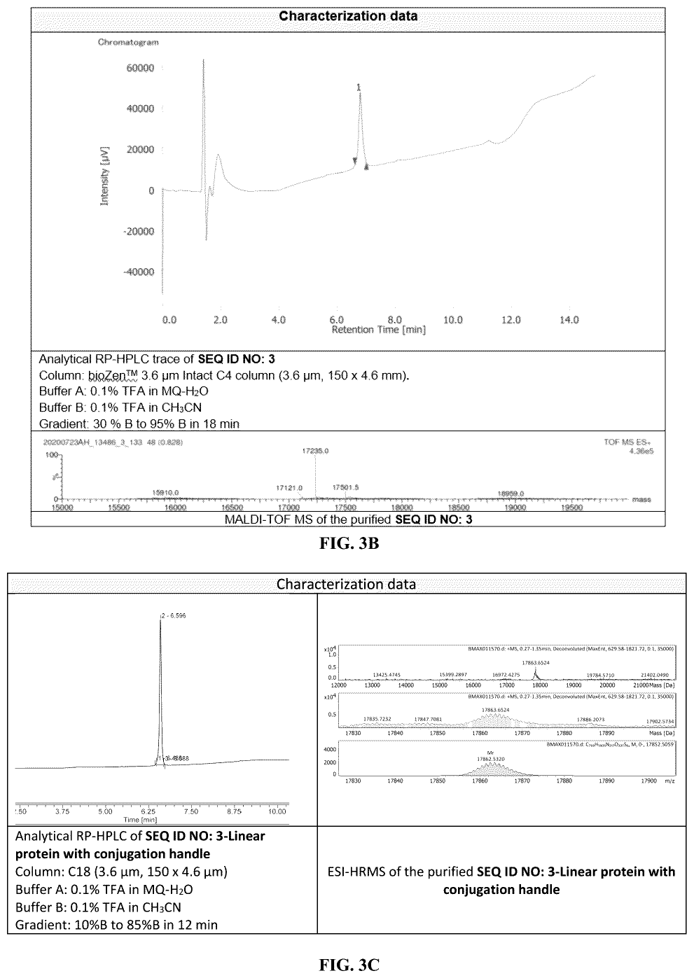

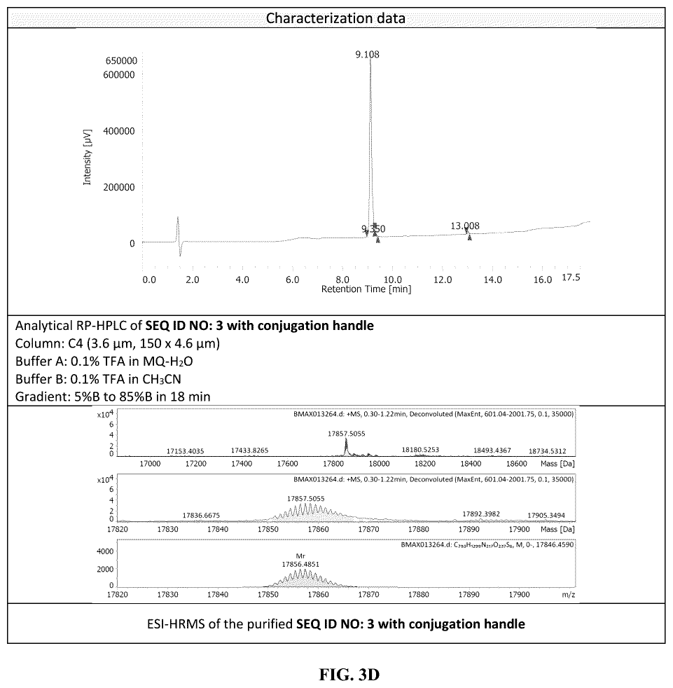

After completion of reaction, the sample was diluted with 1:1 CH₃CN:H₂O with 0.1% TFA (v/v). The sample was purified by preparative HPLC on a C18 column (5 μm, 110 Å, 250×20 mm) at a flow rate of 10 mL/min at room temperature using CH₃CN/H₂O with 0.1% TFA (v/v) as mobile phase, with a two-step gradient: 10 to 30% CH3CN in 5 min and 30 to 95% CH3CN in 20 min. The fractions containing the purified product were pooled and lyophilized to obtain 2.8 mg SEQ ID NO: 3-Linear protein as a white powder in 98% purity. (49% yield for Acm deprotection and purification steps). MS (ESI): C₇₆₆H₁₂₅₅N₂₁₃O₂₂₅S₆; m/z calculated: 17240.1893 Da [M+H]; found: 17240.1636 Da [M+H]. FIG. 3A shows characterization data (HPLC, ESI-HRMS) for SEQ ID NO: 3-Linear protein. Retention time (analytical method A): 5.52 min.

### SEQ ID NO: 3-Folded Protein: Rearrangement and Folding of IL-7 Linear Protein.

2.3 mg (0.133 μmol) of the linear IL-7 protein were dissolved in 7.5 mL of 50 mM Tris buffer containing 6 M GnHCl, 50 mM NaCl, 1 mM EDTA and 2 mM CysHCl (18 μM protein concentration), which was adjusted to pH 8.0 by adding a solution of 6 M aqueous HCl. The mixture was gently shaken at rt for 2 h. The rearrangement was monitored by analytical reverse phase HPLC.

The solution with the rearranged protein was cooled to 4° C. and diluted (×3) with 15 mL of 50 mM Tris buffer containing 50 mM NaCl and 0.1 M Arg, which was adjusted to pH 8.0 by adding a solution of 6 M aqueous HCl. The folding was allowed to proceed for 48 h at 4° C. The folding was monitored according to the rearrangement monitoring conditions.

After completion of folding reaction as ascertained by HPLC, the sample was acidified with TFA to pH=3, and purified by preparative HPLC using a C4 column (5 μm, 20×250 mm) at a flow rate of 10 mL/min at room temperature with a gradient of 30 to 85% B in 50 min. The fractions containing the purified product were pooled and lyophilized to obtain 0.8 mg of the folded IL-7 polypeptide (35% yield) as a white powder. The purity and identity of the pure folded protein was further confirmed by analytical HPLC and ESI/MS. MS (ESI): C₇₆₆H₁₂₄₉N₂₁₃O₂₂₅S₆; m/z calculated: 17235.0 Da [M+H]⁺; found: 17235.0 Da [M+H]⁺. FIG. 3B shows characterization data of folded SEQ ID NO: 3 IL-7 protein.

Other synthetic IL-7 polypeptides as provided herein are synthesized according methods analogous with those described above.

### Synthesis Method 2

CMP-036 provided below is the synthesis of an IL-7 polypeptide of SEQ ID NO: 3. The method provided below can be adapted to produce any desired variant of IL-7, including the IL-7 variants provided herein.

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 (aspartic acid). After the peptide elongation, the resin was washed with DCM and dried under vacuum.

Segment 1 was then released from the resin and the side chain deprotected following protocol 5. Purification of crude peptide was performed by preparative HPLC (protocol 12). The fractions containing the purified product were pooled and lyophilized to obtain CMP-036-Seg1 as a white solid in 94% purity. The isolated yield based on the resin loading was 17%. HRMS (ESI): calculated for C₁₇₁H₂₈₄N₄₃O₆₃S₂: 1337.9940 Da [M+3]³⁺; found: 1337.9933 Da. Retention time (analytical method 1): 5.66 min.
Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).
After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-036-Seg2 as a white solid in 98% purity. The isolated yield based on the resin loading was 20%. HRMS (ESI): calculated for C₂₃₄H₃₅₁N₆₅O₆₂S: 5098.6114 Da; found: 5098.6026 Da. Retention time (analytical method 1): 5.24 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-036-Seg3 as a white solid in 98% purity. The isolated yield based on the resin loading was 25%. HRMS (ESI): calculated for C₁₉₃H₃₁₅N₅₁O₅₇S: 4293.3094 Da; found: 4293.3564 Da. Retention time (analytical method L): 10.458 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-036-Seg4 as a white solid in 98% purity. The isolated yield based on the resin loading was 8%. HRMS (ESI): calculated for C₂₁₅H₃₆₆N₆₁O₆₀S₂: 965.7386 Da [M+5]⁺⁵; found: 965.7380 Da. Retention time (analytical method 2): 6.154 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-036-Seg12 as a white solid in 98% purity. The isolated yield was 38%. HRMS (ESI): calculated for C₃₉₃H₆₁₉N₁₀₇O₁₂₀S₃: 8858.4917 Da; found: 8858.4928 Da. Retention time (analytical method A): 5.41 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-036-Seg34 as a white solid in 96% purity. The isolated yield was 55%. HRMS (ESI): calculated for C₃₉₂H₆₆₆N₁₁₂O₁₁₃S₃: 8851.9104 Da; found: 8851.8975 Da. Retention time (analytical method 2): 6.210 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-036-Seg1234-Acm as a white solid in 98% purity. The isolated yield was 28%. HRMS (ESI): calculated for C₇₈₄H₁₂₈₅N₂₁₉O₂₃₁S₆: 17666.4121 Da; found: 17666.4233 Da. Retention time (analytical method 1): 5.330 min.

Linear protein: Acm deprotection: Acm protection on cysteine residues were removed using protocol 9. The ligation sample was purified by preparative HPLC (protocol 12). The fractions containing the purified product were pooled and lyophilized to obtain CMP-036-Linear protein as a white powder in 92% purity. (49% yield for Acm deprotection and purification steps). HRMS (ESI): calculated for C₇₆₆H₁₂₅₅N₂₁₃O₂₂₅S⁶: 17240.1893 Da; found: 17240.1636 Da. FIG. 3A shows characterization data (HPLC, ESI-HRMS) for CMP-036-Linear protein. Retention time (analytical method 1): 5.520 min.

Folding: Rearrangement and folding CMP-036 linear protein. The linear protein was rearranged, folded and purified following protocol 10. The fractions containing the purified product were pooled and lyophilized to obtain CMP-036 as a white powder in 93.8% purity.

The purity and identity of the pure folded protein was further confirmed by analytical HPLC and ESI/MS. MS (ESI): calculated for C₇₆₆H₁₂₄₉N₂₁₃O₂₂₅S₆: 17235.0 Da; found: 17235.0 Da. FIG. 3B shows characterization data of folded SEQ ID NO: 3 IL-7 protein (CMP-036). Retention time (analytical method 4): 6.767 min.

### Example 2B: Synthesis of CMP-107

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-107-Seg1 as a white solid with 94% purity. The isolated yield based on the resin loading was 17%. HRMS (ESI): calculated for C₂₀₄H₃₃₂N₄₈O₇₅S₂: 4720.3137 Da; found: 4720.3264 Da. Retention time (analytical method 3): 5.270 min.

Segment 2, 3, 4 and ligated segment 34 were synthesized as described in Example 2A, synthesis method 2.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-107-Seg12 as a white solid in 98% purity. The isolated yield was 22%. HRMS (ESI): calculated for C₄₂₆H₆₇₀N₁₁₂O₁₃₂S₃: 9567.8454 Da; found: 9567.8876 Da. Retention time (analytical method 2): 6.604 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-107-Seg1234-Acm as a white solid in 99% purity. The isolated yield was 48%. HRMS (ESI): calculated for C₈₁₇H₁₃₃₆N₂₂₄O₂₄₃S₆: 18376.7681 Da; found: 18376.8490 Da. Retention time (analytical method 2): 6.471 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-107-Linear protein as a white powder with 97% purity (67% yield for Acm deprotection and purification steps). HRMS (ESI): calculated for C₇₉₉H₁₃₀₆N₂₁₈O₂₃₇S₆: 17950.5453 Da; found: 17950.6047 Da. FIG. 3A shows characterization data (HPLC, ESI-HRMS) for CMP-107-Linear protein. Retention time (analytical method 2): 6.596 min.

Rearrangement and folding. The linear protein was rearranged, folded and purified following protocol 10. The fractions containing the folded protein were pooled and lyophilized to obtain CMP-107 as a white solid in 99% purity. The purity and identity of the pure folded protein was further confirmed by analytical HPLC and HRMS (ESI). The isolated yield was 13%. HRMS (ESI): calculated for C₇₉₉H₁₃₀₀N₂₁₈O₂₃₇S₆: 17944.4984 Da; found: 17944.5621 Da. Retention time (analytical method 4): 11.125 min FIG. 3B shows characterization data of folded SEQ ID NO: x IL-7 protein.

### Example 2C: Synthesis of CMP-203

Pegylation of Example 2B: Example 2B folded protein was conjugated with polydisperse 30 kDa DBCO-PEG (structure 6) following protocol 11. The fractions containing the pegylated protein were pooled and lyophilized to obtain CMP-203 as a white solid in 99% purity. The purity and identity of CMP-203 was further confirmed by analytical HPLC and MALDI-TOF. The isolated yield was 42%. Retention time (analytical method 5): 10.708 min. m/z calculated: ˜ 47945 Da, found: 49410.520 Da.

### Example 2D: Synthesis of CMP-108

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-108-Seg1 as a white solid in 97% purity. The isolated yield based on the resin loading was 4%. HRMS (ESI): calculated for C₁₉₈H₃₃₄N₄₇O₇₅S₂: 1545.4415 [M+3]³⁺ Da; found: 1545.4396 Da. Retention time (analytical method 2): 6.771 min.

Segment 2, 3, 4 and Ligated Segment 34 were Synthesized as Described in Example 2A, Synthesis Method 2.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-108-Seg12 as a white solid in 99% purity. The isolated yield was 28%. HRMS (ESI): calculated for C₄₂₀H₆₆₉N₁₁₁O₁₃₂S₃: 9480.8344 Da; found: 9480.8358 Da. Retention time (analytical method 2): 6.563 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-108-Seg1234-Acm as a white solid in 99% purity. The isolated yield was 39%. HRMS (ESI): calculated for C₈₁₁H₁₃₃₅N₂₂₃O₂₄₃S₆: 18289.7572 Da; found: 18289.8336 Da. Retention time (analytical method 2): 6.446 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-108-Linear protein as a white powder with 99% purity (57% yield for Acm deprotection and purification steps). HRMS (ESI): calculated for C₇₉₃H₁₃₀₅N₂₁₇O₂₃₇S₆: 17862.5320 Da; found: 17862.6030 Da. FIG. 3A shows characterization data (HPLC, ESI-HRMS) for CMP-108-Linear protein. Retention time (analytical method 2): 6.596 min.

Rearrangement and folding. The linear protein was rearranged, folded and purified following protocol 10. The fractions containing the folded protein were pooled and lyophilized to obtain CMP-108 as a white solid in 97.4% purity. The purity and identity of the pure folded protein was further confirmed by analytical HPLC and HRMS (ESI). The isolated yield was 15%. HRMS (ESI): calculated for C₇₉₃H₁₂₉₉N₂₁₇O₂₃₇S₆: 17856.4851 Da; found: 17856.4995 Da. Retention time (analytical method 6): 14.525 min. FIG. 3B shows characterization data of folded SEQ ID NO: x IL-7 protein.

### Example 2E: Synthesis of CMP-114

Pegylation of Example 2D: The example 2D folded protein was conjugated with polydisperse 30 kDa DBCO-PEG (structure 6) following protocol 11. The fractions containing the pegylated protein were pooled and lyophilized to obtain CMP-114 as a white solid in 97% purity. The purity and identity of CMP-114 was further confirmed by analytical HPLC and MALDI. The isolated yield was 24%. Retention time (analytical method 3): 6.863 min. m/z calculated: ˜47945, found: 48405.834.

### Example 2F: Synthesis of CMP-204

Segment 1 and segment 12 were synthesized as described in Example 2D.

Segments 2 and 4 were synthesized as described in Example 2A, synthesis method 2.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-204-Seg3 as a white solid in 98% purity. The isolated yield based on the resin loading was 13%. HRMS (ESI): calculated for C₁₉₈H₃₁₈N₅₁O₅₇S: 871.2660 Da [M+5]⁵⁺; found: 871.2656 Da. Retention time (analytical method 2): 6.571 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-204-Seg34 as a white solid in 98% purity. The isolated yield was 39%. HRMS (ESI): calculated for C₃₉₇H₆₆₄N₁₁₂O₁₁₃S₃: 8909.8948 Da; found: 8909.8898 Da. Retention time (analytical method 8): 6.383 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-204-Seg1234-Acm as a white solid in 98% purity. The isolated yield was 34%. HRMS (ESI): C₈₁₆H₁₃₃₃N₂₂₃O₂₄₃S₆; Average isotope calculated 18348.32 Da [M]; found: Not found. Retention time (analytical method 2): 6.463 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-204-Linear protein as a white powder in 94% purity (57% yield for Acm deprotection and purification steps). HRMS (ESI): calculated for C₇₉₈H₁₃₀₃N₂₁₇O₂₃₇S₆: 17920.5164 Da; found: 17920.5449 Da. Retention time (analytical method 2): 6.629 min.

Rearrangement and folding. The linear protein was rearranged, folded and purified following protocol 10. The fractions containing the folded protein were pooled and lyophilized to obtain CMP-204 as a white solid in 96.7% purity. The purity and identity of the pure folded protein was further confirmed by analytical HPLC and HRMS (ESI). The isolated yield was 3%. HRMS (ESI): calculated for C₇₉₈H₁₂₉₇N₂₁₇O₂₃₇S₆: 17914.4695 Da; found: Not found. Retention time (analytical method 6): 9.175 min.

### Example 2G: Synthesis of CMP-115

Segment 1 and segment 12 were synthesized as described in Example 2D.

Segments 2 and 4 were synthesized as described in Example 2A, synthesis method 2.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). Side chain Alloc deprotection of Lys (Lys81) was performed following protocol 3 and elongation with 2-(2-(2-methoxyethoxy) ethoxy) acetic acid (structure 7) was pursued on the free amine side chain following protocol 2. After the peptide elongation, the resin was washed with DCM and diethyl ether and dried under vacuum. The peptide was cleaved from the resin following protocol 5.

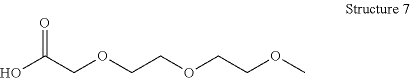

Purification of crude peptide was performed by preparative HPLC (protocol 12). The fractions containing the purified product were pooled and lyophilized to obtain CMP-115-Seg3 as a white solid in 96% purity. The isolated yield based on the resin loading was 10%. HRMS (ESI): calculated for C₂₀₀H₃₂₇N₅₁O₆₁S: 4453.3830 Da; found: 4453.4077 Da. Retention time (analytical method 2): 6.471 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-115-Seg34 as a white solid in 95% purity. The isolated yield was 34%. HRMS (ESI): calculated for C₃₉₉H₆₇₈N₁₁₂O₁₁₇S₃: 9011.9840 Da; found: 9012.0160 Da. Retention time (analytical method 8): 6.293 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-115-Seg1234-Acm as a white solid in 99% purity. The isolated yield was 32%. HRMS (ESI): calculated for C₈₁₈H₁₃₄₇N₂₂₃O₂₄₇S₆: 18449.8308 Da; found: 18449.9455 Da. Retention time (analytical method 2): 6.404 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-115-Linear protein as a white powder in 99% purity (61% yield for Acm deprotection and purification steps). HRMS (ESI): calculated for C₈₀₀H₁₃₁₇N₂₁₇O₂₄₁S₆: 18023.6080 Da; found: 18023.6465 Da. Retention time (analytical method 2): 6.596 min.

Rearrangement and folding. The linear protein was rearranged, folded and purified following protocol 10. The fractions containing the folded protein were pooled and lyophilized to obtain CMP-115 as a white solid in 79.2% purity. The purity and identity of the pure folded protein was further confirmed by analytical HPLC and HRMS (ESI). The isolated yield was 19%. HRMS (ESI): calculated for C₈₀₀H₁₃₁₁N₂₁₇O₂₄₁S₆: 18017.5611 Da; found: 18017.6301. Retention time (analytical method 6): 9.283 min.

### Example 2H: Synthesis of CMP-109

Segment 1 and segment 12 were synthesized as described in Example 2D.

Segments 2 and 4 were synthesized as described in Example 2A, synthesis method 2.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). Side chain Alloc deprotection of Lys (Lys81) was performed following protocol 3 and elongation with O—(N-Boc-2-aminoethyl)-O′—(N-diglycolyl-2-aminoethyl)-hexaethyleneglycol (structure 8) was pursued on the free amine side chain following protocol 2. After the peptide elongation, the resin was washed with DCM and diethyl ether and dried under vacuum. The peptide was cleaved from the resin following protocol 5.

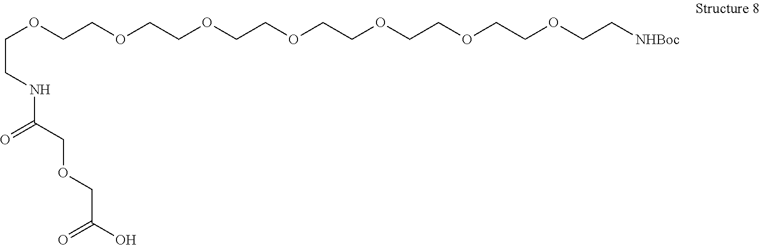 [SMILES: CC(C)(C)OC(=O)NCCOCCOCCOCCOCCOCCOCCOCCNC(=O)COCC(=O)O]

Purification of crude peptide was performed by preparative HPLC (protocol 12). The fractions containing the purified product were pooled and lyophilized to obtain CMP-109-Seg3 as a white solid in 88% purity. The isolated yield based on the resin loading was 16%. HRMS (ESI): calculated for C₂₁₃H₃₅₃N₅₃O₆₇S: 4759.5622 Da; found: 4759.5996 Da. Retention time (analytical method 13): 5.129 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-109-Seg34 as a white solid in 85% purity. The isolated yield was 17%. HRMS (ESI): calculated for C₄₁₂H₇₀₄N₁₁₄O₁₂₃S₃: 9318.1632 Da; found: 9318.1966 Da. Retention time (analytical method 8): 6.223 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-109-Seg1234-Acm as a white solid in 97% purity. The isolated yield was 24%. HRMS (ESI): calculated for C₈₃₁H₁₃₇₃N₂₂₅O₂₅₃S₆: 18756.0100 Da; found: 18756.0532 Da. Retention time (analytical method 2): 6.629 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-109-Linear protein as a white powder in 96% purity (54% yield for Acm deprotection and purification steps). HRMS (ESI): calculated for C₈₁₃H₁₃₄₃N₂₁₉O₂₄₇S₆: 18329.7872 Da; found: 18328.8394 Da. Retention time (analytical method 2): 6.504 min.

Rearrangement and folding. The linear protein was rearranged, folded and purified following protocol 10. The fractions containing the folded protein were pooled and lyophilized to obtain CMP-109 as a white solid in 96.6% purity. The purity and identity of the pure folded protein was further confirmed by analytical HPLC and HRMS (ESI). The isolated yield was 16%. HRMS (ESI): calculated for C₈₁₃H₁₃₃₇N₂₁₉O₂₄₇S₆: 18323.7403 Da; found: 18323.7814 Da. Retention time (analytical method 7): 9.275 min.

### Example 21: Synthesis of CMP-110

Segment 1 and segment 12 were synthesized as described in Example 2D.

Segments 2 and 4 were synthesized as described in Example 2A, synthesis method 2.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-110-Seg3 as a white solid in 96% purity. The isolated yield based on the resin loading was 34%. HRMS (ESI): calculated for C₁₉₅H₃₁₈N₅₂O₅₈S: 4350.3309 Da; found: 4350.3487 Da. Retention time (analytical method 2): 6.179 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-110-Seg34 as a white solid in 94% purity. The isolated yield was 30%. HRMS (ESI): calculated for C₃₉₄H₆₆₉N₁₁₃O₁₁₄S₃: 8908.9318 Da; found: 8908.9568 Da. Retention time (analytical method 8): 6.137 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-110-Seg1234-Acm as a white solid in 96.5% purity. The isolated yield was 28%. HRMS (ESI): calculated for C₈₁₃H₁₃₃₈N₂₂₄O₂₄₄S₆: 18346.7786 Da; found: 18346.8115 Da. Retention time (analytical method 2): 6.371 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-110-Linear protein as a white powder in 97% purity (52% yield for Acm deprotection and purification steps). HRMS (ESI): calculated for C₇₉₅H₁₃₀₈N₂₁₈O₂₃₈S₆: 17919.5535 Da; found: 17919.5920 Da. Retention time (analytical method 2): 6.504 min.

Rearrangement and folding. The linear protein was rearranged, folded and purified following protocol 10. The fractions containing the folded protein were pooled and lyophilized to obtain CMP-110 as a white solid in 98% purity. The purity and identity of the pure folded protein was further confirmed by analytical HPLC and HRMS (ESI). The isolated yield was 11%. HRMS (ESI): calculated for C₇₉₅H₁₃₀₂N₂₁₈O₂₃₈S₆: 17913.5065 Da; found: 17913.5233. Retention time (analytical method 7): 10.417 min.

### Example 2J: Synthesis of CMP-112

Segment 1 and segment 12 were synthesized as described in Example 2D.

Segments 2 and 4 were synthesized as described in Example 2A, synthesis method 2.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). Side chain Alloc deprotection of Lys (Lys81) was performed following protocol 3 and elongation with O-(2-Carboxyethyl)-O′-methyl-undecacthylene glycol (structure 9) was pursued on the free amine side chain following protocol 2. After the peptide elongation, the resin was washed with DCM and diethyl ether and dried under vacuum. The peptide was cleaved from the resin following protocol 5.

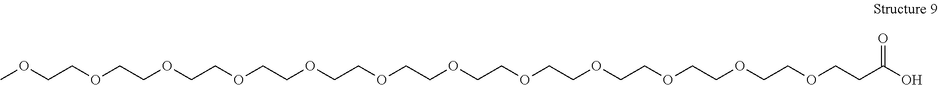 [SMILES: COCCOCCOCCOCCOCCOCCOCCOCCOCCOCCOCCOCCC(=O)O]

Purification of crude peptide was performed by preparative HPLC (protocol 12). The fractions containing the purified product were pooled and lyophilized to obtain CMP-112-Seg3 as a white solid in 91% purity. The isolated yield based on the resin loading was 3.7%. HRMS (ESI): calculated for C₂₁₉H₃₆₅N₅₁O₇₀S: 4863.6347 Da; found: 4863.6380 Da. Retention time (analytical method 10): 4.521 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-112-Seg34 as a white solid in 92% purity. The isolated yield was 14%. HRMS (ESI): calculated for C₃₉₄H₆₆₉N₁₁₃O₁₁₄S₃: 9422.2358 Da; found: 9422.2441 Da. Retention time (analytical method 9): 5.490 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-112-Seg1234-Acm as a white solid in 94.6% purity. The isolated yield was 30%. HRMS (ESI): calculated for C₈₃₇H₁₃₈₅N₂₂₃O₂₅₆S₆: 18860.0826 Da; found: 18860.0852 Da. Retention time (analytical method 9): 4.696 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-112-Linear protein as a white powder in 93% purity (90% yield for Acm deprotection and purification steps). HRMS (ESI): calculated for C₈₁₉H₁₃₅₅N₂₁₇O₂₅₀S₆: 18433.8598 Da; found: 18433.8448 Da. Retention time (analytical method 9): 4.429 min.

Rearrangement and folding. The linear protein was rearranged, folded and purified following protocol 10. The fractions containing the folded protein were pooled and lyophilized to obtain CMP-112 as a white solid in 96% purity. The purity and identity of the pure folded protein was further confirmed by analytical HPLC and HRMS (ESI). The isolated yield was 28%. HRMS (ESI): calculated for C₈₁₉H₁₃₄₉N₂₁₇O₂₅₀S₆: 18427.8129 Da; found: 18427.7907 Da. Retention time (analytical method 7): 10.475 min.

### Example 2K: Synthesis CMP-205

Segment 1 and segment 12 were synthesized as described in Example 2D.

Segments 2 and 4 were synthesized as described in Example 2A, synthesis method 2.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). Side chain Alloc deprotection of Lys (Lys81) was performed following protocol 3 and elongation with 2-((3r,5r,7r)-adamantan-1-yl) acetic acid (structure 10) was pursued on the free amine side chain following protocol 2. After the peptide elongation, the resin was washed with DCM and diethyl ether and dried under vacuum. The peptide was cleaved from the resin following protocol 5.

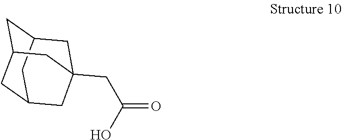 [SMILES: O=C(O)CC12CC3CC(CC(C3)C1)C2]

Purification of crude peptide was performed by preparative HPLC (protocol 12). The fractions containing the purified product were pooled and lyophilized to obtain CMP-205-Seg3 as a white solid in 97% purity. The isolated yield based on the resin loading was 15%. HRMS (ESI): calculated for C₂₀₅H₃₃₁N₅₁O₅₈S: 4469.4296 Da; found: 4469.4245 Da. Retention time (analytical method 10): 5.229 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-205-Seg34 as a white solid in 91% purity. The isolated yield was 22%. HRMS (ESI): calculated for C₄₀₄H₆₈₂N₁₁₂O₁₁₄S₃: 9028.0306 Da; found: 9028.0462 Da. Retention time (analytical method 8): 6.680 min. Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-205-Seg1234-Acm as a white solid in 99% purity. The isolated yield was 33%. HRMS (ESI): calculated for C₈₂₃H₁₃₅₁N₂₂₃O₂₄₄S₆: 18465.8774 Da; found: 18465.8915. Retention time (analytical method 2): 6.521 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP ROS Linear protein as a white powder in 93% purity (44% yield for Acm deprotection and purification steps). HRMS (ESI): calculated for C₈₀₅H₁₃₂₁N₂₁₇O₂₃₈S₆: 18039.6546 Da; found: 18039.6541 Da. Retention time (analytical method 2): 6.729 min.

Rearrangement and folding. The linear protein was rearranged, folded and purified following protocol 10. The fractions containing the folded protein were pooled and lyophilized to obtain CMP-205 as a white solid in 96% purity. The purity and identity of the pure folded protein was further confirmed by analytical HPLC and HRMS (ESI). The isolated yield was 6%. HRMS (ESI): calculated for C₈₀₅H₁₃₁₅N₂₁₇O₂₃₈S₆: 18033.6077 Da; found: 18033.6366 Da. Retention time (analytical method 7): 10.583.

### Example 2L: Synthesis of CMP-111

Segment 1, 2, 4 and ligated segment 12 were synthesized as described in Example 2D.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). Side chain Alloc deprotection of Lys (Lys81) was performed following protocol 3 and elongation with O—(N-Boc-2-aminoethyl)-O′—(N-diglycolyl-2-aminoethyl)-hexaethyleneglycol (structure 8) was pursued on the free amine side chain following protocol 2. After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-111-Seg3 as a white solid in 95% purity. The isolated yield based on the resin loading was 7%. HRMS (ESI): calculated for C₂₁₅H₃₅₆N₅₄O₆₈S: 4816.5836 Da; found: 4816.6293 Da. Retention time (analytical method 2): 6.313 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-111-Seg34 as a white solid in 96% purity. The isolated yield was 25%. HRMS (ESI): calculated for C₄₁₄H₇₀₇N₁₁₅O₁₂₄S₃: 9375.1846 Da; found: 9375.2124 Da. Retention time (analytical method 12): 8.327 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-111-Seg1234-Acm as a white solid in 98% purity. The isolated yield was 19%. HRMS (ESI): calculated for C₈₃₃H₁₃₇₆N₂₂₆O₂₅₄S₆: 18813.0314 Da; found: 18813.0165 Da. Retention time (analytical method 12): 9.199 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-111-Linear protein as a white powder in 90% purity (23% yield for Acm deprotection and purification steps). HRMS (ESI): calculated for C₈₁₅H₁₃₄₆N₂₂₀O₂₄₈S₆: 18386.8087 Da; found: 18386.8223 Da. Retention time (analytical method 12): 9.583 min.

Folding: Rearrangement and folding CMP-111 linear protein. The linear protein was rearranged, folded and purified following protocol 10. The fractions containing the purified product were pooled and lyophilized to obtain CMP-111 as a white powder in 93.8% purity. The isolated yield was 34%. The purity and identity of the pure folded protein was further confirmed by analytical HPLC and HRMS. HRMS (ESI): calculated for C₈₁₅H₁₃₄₀N₂₂₀O₂₄₈S₆: 18380.7617 Da; found: 18381.7426 Da. Retention time (analytical method 7): 10.492 min.

### Example 2M: Synthesis of CMP-113

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-113-Seg1 as a white solid in 97% purity. The isolated yield based on the resin loading was 9%. HRMS (ESI): calculated for C₁₉₅H₃₂₆N₄₆O₇₄S: 4530.2938 Da; found: 4530.3001 Da. Retention time (analytical method 2): 6.796 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).

After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-113-Seg2 as a white solid in 98% purity. The isolated yield based on the resin loading was 20%. HRMS (ESI): calculated for C₂₃₄H₃₅₁N₆₅O₆₂S: 5098.6113 Da; found: 5098.6386 Da. Retention time (analytical method 13): 5.271 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-113-Seg3 as a white solid in 98% purity. The isolated yield based on the resin loading was 33%. HRMS (ESI): calculated for C₁₉₀H₃₁₀N₅₀O₅₆: 4190.3004 Da; found: 4190.3064 Da. Retention time (analytical method 2): 6.179 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-113-Seg4 as a white solid in 98% purity. The isolated yield based on the resin loading was 8%. HRMS (ESI): calculated for C₂₁₅H₃₆₆N₆₁O₆₀S₂: 965.7386 Da [M+5]⁺⁵; found: 965.7380 Da. Retention time (analytical method 2): 6.154 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-113-Seg12 as a white solid in 96% purity. The isolated yield was 18%. HRMS (ESI): calculated for C₄₁₇H₆₆₄N₁₁₀O₁₃₁S₂: 9377.8255 Da; found: 9377.8554 Da. Retention time (analytical method 8): 7.22 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-113-Seg34 as a white solid in 88% purity. The isolated yield was 22%. HRMS (ESI): calculated for C₃₈₉H₆₆₁N₁₁₁O₁₁₂S₂: 8748.9015 Da; found: 8748.9141 Da. Retention time (analytical method 14): 6.873 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-113-Seg1234-Acm as a white solid in 96% purity. The isolated yield was 16%. HRMS (ESI): calculated for C₈₀₅H₁₃₂₅N₂₂₁O₂₄₁S₄: 18082.737 Da; found: 18083.7344 Da. Retention time (analytical method 8): 7.023 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-113-Linear protein as a white powder in 93% purity (81% yield for Acm deprotection and purification steps). HRMS (ESI): calculated for C₇₉₃H₁₃₀₅N₂₁₇O₂₃₇S₄: 17798.5886 Da; found: 17799.5709 Da. Retention time (analytical method 8): 7.327 min.

Folding: Rearrangement and folding CMP-113 linear protein. The linear protein was rearranged, folded and purified following protocol 10. The fractions containing the purified product were pooled and lyophilized to obtain CMP-113 as a white powder in 93% purity. The isolated yield was 23%. The purity and identity of the pure folded protein was further confirmed by analytical HPLC and HRMS. HRMS (ESI): calculated for C₇₉₃H₁₃₀₁N₂₁₇O₂₃₇S₄: 17794.5572 Da; found: 17795.5663 Da. Retention time (analytical method 7): 10.583 min.

### Example 2N: Synthesis of CMP-206

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-206-Seg1 as a white solid in 89% purity. The isolated yield based on the resin loading was 9%. HRMS (ESI): calculated for C₁₉₅H₃₂₆N₄₆O₇₄S: 4530.2938 Da; found: 4530.3246 Da. Retention time (analytical method 11): 11.858 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-206-Seg2 as a white solid in 94% purity. The isolated yield based on the resin loading was 40%. HRMS (ESI): calculated for C₂₃₄H₃₅₁N₆₅O₆₂S: 5098.6113 Da; found: 5098.6386 Da. Retention time (analytical method 13): 5.271 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-206-Seg3 as a white solid in 98% purity. The isolated yield based on the resin loading was 25%. HRMS (ESI): calculated for C₁₉₃H₃₁₅N₅₁O₅₇S: 4293.3094 Da; found: 4293.2962 Da. Retention time (analytical method 11): 8.181 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-206-Seg4 as a white solid in 97% purity. The isolated yield based on the resin loading was 19%. HRMS (ESI): calculated for C₂₁₂H₃₅₆N₆₀O₅₉S: 4720.6477 Da; found: 4720.6947 Da. Retention time (analytical method 12): 8.838 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-206-Seg12 as a white solid in 92% purity. The isolated yield was 22%. HRMS (ESI): calculated for C₄₁₇H₆₆₄N₁₁₀O₁₃₁S: 9377.8255 Da; found: 9377.8336 Da. Retention time (analytical method 11): 12.442 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-206-Seg34 as a white solid in 96% purity. The isolated yield was 29%. HRMS (ESI): calculated for C₃₈₉H₆₆₁N₁₁₁O₁₁₂S₂: 8748.9015 Da; found: 8747.9056 Da. Retention time (analytical method 11): 9.625 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-206-Seg1234-Acm as a white solid in 87% purity. The isolated yield was 25%. HRMS (ESI): calculated for C₈₀₅H₁₃₂₅N₂₂₁O₂₄₁S₄: 18082.737 Da; found: 18083.6724 Da. Retention time (analytical method 11): 11.925 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-206-Linear protein as a white powder in 92% purity (30% yield for Acm deprotection and purification steps). HRMS (ESI): calculated for C₇₉₃H₁₃₀₅N₂₁₇O₂₃₇S₄: 17798.5886 Da; found: 17798.6542 Da. Retention time (analytical method 11): 12.58 min.

Folding: Rearrangement and folding CMP-206 linear protein. The linear protein was rearranged, folded and purified following protocol 10. The fractions containing the purified product were pooled and lyophilized to obtain CMP-206 as a white powder in 97% purity. The isolated yield was 7%. The purity and identity of the pure folded protein was further confirmed by analytical HPLC and HRMS. HRMS (ESI): calculated for C₇₉₃H₁₃₀₁N₂₁₇O₂₃₇S₄: 17794.5572 Da; found: 17797.5925 Da. Retention time (analytical method 16): 15.742 min.

### Example 20: Synthesis of CMP-207

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-207-Seg1 as a white solid in 97% purity. The isolated yield based on the resin loading was 18%. HRMS (ESI): calculated for C₁₉₈H₃₃₁N₄₇O₇₅S₂: 4633.3028 Da; found: 4633.3183 Da. Retention time (analytical method 2): 6.821 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).

After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-207-Seg2 as a white solid in 90% purity. The isolated yield based on the resin loading was 26%. HRMS (ESI): calculated for C₂₃₁H₃₄₆N₆₄O₆₁: 4994.5997 Da; found: 4994.6430 Da. Retention time (analytical method 12): 8.841 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-207-Seg3 as a white solid in 98% purity. The isolated yield based on the resin loading was 25%. HRMS (ESI): calculated for C₁₉₃H₃₁₅N₅₁O₅₇S: 4293.3094 Da; found: 4293.2962 Da. Retention time (analytical method 12): 8.181 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-207-Seg4 as a white solid in 97% purity. The isolated yield based on the resin loading was 17%. HRMS (ESI): calculated for C₂₁₂H₃₅₆N₆₀O₅₉S: 4720.6477 Da; found: 4720.6917 Da. Retention time (analytical method 12): 8.845 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-207-Seg12 as a white solid in 89% purity. The isolated yield was 17%. HRMS (ESI): calculated for C₄₁₇H₆₆₄N₁₁₀O₁₃₁S₂: 9377.8255 Da; found: 9377.9318 Da. Retention time (analytical method 11): 12.258 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-207-Seg34 as a white solid in 89% purity. The isolated yield was 24%. HRMS (ESI): calculated for C₃₈₉H₆₆₁N₁₁₁O₁₁₂S₂: 8748.9015 Da; found: 8747.9082 Da. Retention time (analytical method 11): 10.558 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-207-Seg1234-Acm as a white solid in 90% purity. The isolated yield was 20%. HRMS (ESI): calculated for C₈₀₅H₁₃₂₅N₂₂₁O₂₄₁S₄: 18082.7370 Da; found: 18082.6525 Da. Retention time (analytical method 11): 11.908 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-207-Linear protein as a white powder in 88% purity (60% yield for Acm deprotection and purification steps). HRMS (ESI): calculated for C₇₉₃H₁₃₀₅N₂₁₇O₂₃₇S₄: 17798.5886 Da; found: 17798.6706 Da. Retention time (analytical method 11): 12.192 min.

Folding: Rearrangement and folding CMP-207 linear protein. The linear protein was rearranged, folded and purified following protocol 10. The fractions containing the purified product were pooled and lyophilized to obtain CMP-207 as a white powder in 94% purity. The isolated yield was 8%. The purity and identity of the pure folded protein was further confirmed by analytical HPLC and HRMS. HRMS (ESI): calculated for C₇₉₃H₁₃₀₁N₂₁₇O₂₃₇S₄: 17794.5572 Da; found: 17795.6533 Da. Retention time (analytical method 16): 15.742 min.

### Example 2P: Synthesis of CMP-200

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-200-Seg1 as a white solid in 93% purity. The isolated yield based on the resin loading was 9.4%. HRMS (ESI): calculated for C₁₉₅H₃₂₆N₄₆O₇₅S: 4546.2887 Da; found: 4546.319 Da. Retention time (analytical method 11): 11.625 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-200-Seg2 as a white solid in 94% purity. The isolated yield based on the resin loading was 40%. HRMS (ESI): calculated for C₂₃₄H₃₅₁N₆₅O₆₂S: 5098.6113 Da; found: 5098.6386 Da. Retention time (analytical method 13): 5.271 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-200-Seg3 as a white solid in 93% purity. The isolated yield based on the resin loading was 15%. HRMS (ESI): calculated for C₁₉₀H₃₁₀N₅₀O₅₇: 4206.2953 Da; found: 4206.3445 Da. Retention time (analytical method 12): 8.153 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-200-Seg4 as a white solid in 96% purity. The isolated yield based on the resin loading was 5%. HRMS (ESI): calculated for C₂₁₅H₃₆₁N₆₁O₆₀S₂: 4823.6568 Da; found: 4823.6937 Da. Retention time (analytical method 9): 5.50 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-200-Seg12 as a white solid in 90% purity. The isolated yield was 23%. HRMS (ESI): calculated for C₄₁₇H₆₆₄N₁₁₀O₁₃₂S₂: 9393.8204 Da; found: 9393.8222 Da. Retention time (analytical method 11): 12.208 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-200-Seg34 as a white solid in 89% purity. The isolated yield was 19%. HRMS (ESI): calculated for C₃₈₉H₆₆₁N₁₁₁O₁₁₃S₂: 8764.8964 Da; found: 8764.9052 Da. Retention time (analytical method 11): 10.025 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-200-Seg1234-Acm as a white solid in 91% purity. The isolated yield was 27%. HRMS (ESI): calculated for C₈₀₅H₁₃₂₅N₂₂₁O₂₄₃S₄: 18114.7269 Da; found: 18114.798 Da. Retention time (analytical method 11): 11.692 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-200-Linear protein as a white powder in 93% purity (42% yield for Acm deprotection and purification steps). HRMS (ESI): calculated for C₇₉₃H₁₃₀₅N₂₁₇O₂₃₉S₄: 17830.5784 Da; found: 17831.6123 Da. Retention time (analytical method 11): 12.175 min.

Folding: Rearrangement and folding CMP-200 linear protein. The linear protein was rearranged, folded and purified following protocol 10. The fractions containing the purified product were pooled and lyophilized to obtain CMP-200 as a white powder in 98.7% purity. The isolated yield was 24%. The purity and identity of the pure folded protein was further confirmed by analytical HPLC and HRMS. HRMS (ESI): calculated for C₇₉₃H₁₃₀₁N₂₁₇O₂₃₉S₄: 17826.5471 Da; found: 17826.6017 Da. Retention time (analytical method 16): 14.775 min.

### Example 2Q: Synthesis of CMP-208

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-208-Seg1 as a white solid in 93% purity. The isolated yield based on the resin loading was 7%. HRMS (ESI): calculated for C₁₉₅H₃₂₆N₄₆O₇₅S: 4546.2887 Da; found: 4546.3171 Da. Retention time (analytical method 11): 11.592 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).

After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-208-Seg2 as a white solid in 94% purity. The isolated yield based on the resin loading was 40%. HRMS (ESI): calculated for C₂₃₄H₃₅₁N₆₅O₆₂S: 5098.6113 Da; found: 5098.6386 Da. Retention time (analytical method 13): 5.271 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-208-Seg3 as a white solid in 98% purity. The isolated yield based on the resin loading was 25%. HRMS (ESI): calculated for C₁₉₃H₃₁₅N₅₁O₅₇S: 4293.3094 Da; found: 4293.2962 Da. Retention time (analytical method 12): 8.181 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-208-Seg4 as a white solid in 97% purity. The isolated yield based on the resin loading was 14%. HRMS (ESI): calculated for C₂₁₂H₃₅₆N₆₀O₆₀S: 4736.6426 Da; found: 4736.6852 Da. Retention time (analytical method 12): 8.701 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-208-Seg12 as a white solid in 94% purity. The isolated yield was 24%. HRMS (ESI): calculated for C₄₁₇H₆₆₄N₁₁₀O₁₃₂S₂: 9393.8204 Da; found: 9393.8101 Da. Retention time (analytical method 11): 12.158 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-208-Seg34 as a white solid in 91% purity. The isolated yield was 26%. HRMS (ESI): calculated for C₃₈₉H₆₆₁N₁₁₁O₁₁₃S₂: 8764.8964 Da; found: 8764.9084 Da. Retention time (analytical method 11): 10.225 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-208-Seg1234-Acm as a white solid in 96% purity. The isolated yield was 25%. HRMS (ESI): calculated for C₈₀₅H₁₃₂₅N₂₂₁O₂₄₃S₄: 18114.7269 Da; found: 18114.7464 Da. Retention time (analytical method 11): 11.558 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-208-Linear protein as a white powder in 92% purity (54% yield for Acm deprotection and purification steps). HRMS (ESI): calculated for C₇₉₃H₁₃₀₅N₂₁₇O₂₃₉S₄: 17830.5784 Da; found: 17830.6472 Da. Retention time (analytical method 11): 11.875 min.

Folding: Rearrangement and folding CMP-208 linear protein. The linear protein was rearranged, folded and purified following protocol 10. The fractions containing the purified product were pooled and lyophilized to obtain CMP-208 as a white powder in 93.8% purity. The isolated yield was 24%. The purity and identity of the pure folded protein was further confirmed by analytical HPLC and HRMS. HRMS (ESI): calculated for C₇₉₃H₁₃₀₁N₂₁₇O₂₃₉S₄: 17826.5471 Da; found: 17826.5915 Da. Retention time (analytical method 16): 15.442 min.

### Example 2R: Synthesis of CMP-209

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2.

After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-209-Seg1 as a white solid in 93% purity. The isolated yield based on the resin loading was 9%. HRMS (ESI): calculated for C₁₉₅H₃₂₆N₄₆O₇₅S: 4546.2887 Da; found: 4546.319 Da. Retention time (analytical method 11): 11.625 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).

After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-209-Seg2 as a white solid in 84% purity. The isolated yield based on the resin loading was 22%. HRMS (ESI): calculated for C₂₃₁H₃₄₆N₆₄O₆₂: 5010.5946 Da; found: 5010.6347 Da. Retention time (analytical method 12): 8.749 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-209-Seg3 as a white solid in 98% purity. The isolated yield based on the resin loading was 25%. HRMS (ESI): calculated for C₁₉₃H₃₁₅N₅₁O₅₇S: 4293.3094 Da; found: 4293.2962 Da. Retention time (analytical method 12): 8.181 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-209-Seg4 as a white solid in 96% purity. The isolated yield based on the resin loading was 18%. HRMS (ESI): calculated for C₂₁₂H₃₅₆N₆₀O₆₀S: 4736.6426 Da; found: 4736.6833 Da. Retention time (analytical method 12): 8.531 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-209-Seg12 as a white solid in 90% purity. The isolated yield was 22%. HRMS (ESI): calculated for C₄₁₇H₆₆₄N₁₁₀O₁₃₂S₂: 9393.8204 Da; found: 9394.8482 Da. Retention time (analytical method 11): 12.108 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-209-Seg34 as a white solid in 89% purity. The isolated yield was 19%. HRMS (ESI): calculated for C₃₈₉H₆₆₁N₁₁₁O₁₁₃S₂: 8764.8964 Da; found: 8764.9026 Da. Retention time (analytical method 11): 9.992 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-209-Seg1234-Acm as a white solid in 87% purity. The isolated yield was 24%. HRMS (ESI): calculated for C₈₀₅H₁₃₂₅N₂₂₁O₂₄₃S₄: 18114.7269 Da; found: 18114.642 Da. Retention time (analytical method 11): 11.558 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-209-Linear protein as a white powder in 91% purity (45% yield for Acm deprotection and purification steps). HRMS (ESI): calculated for C₇₉₃H₁₃₀₅N₂₁₇O₂₃₉S₄: 17830.5784 Da; found: 17830.6474 Da. Retention time (analytical method 11): 12.058 min.

Folding: Rearrangement and folding CMP-209 linear protein. The linear protein was rearranged, folded and purified following protocol 10. The fractions containing the purified product were pooled and lyophilized to obtain CMP-209 as a white powder in 94% purity. The isolated yield was 29%. The purity and identity of the pure folded protein was further confirmed by analytical HPLC and HRMS. HRMS (ESI): calculated for C₇₉₃H₁₃₀₁N₂₁₇O₂₃₉S₄: 17826.5471 Da; found: 17827.6697 Da. Retention time (analytical method 16): 15.392 min.

### Example 2S: Synthesis of CMP-210

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2.

After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-210-Seg1 as a white solid in 93% purity. The isolated yield based on the resin loading was 7%. HRMS (ESI): calculated for C₁₉₅H₃₂₆N₄₆O₇₅S: 4546.2887 Da; found: 4546.3171 Da. Retention time (analytical method 11): 11.592 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).

After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-210-Seg2 as a white solid in 84% purity. The isolated yield based on the resin loading was 22%. HRMS (ESI): calculated for C₂₃₁H₃₄₆N₆₄O₆₂: 5010.5946 Da; found: 5010.6347 Da. Retention time (analytical method 12): 8.749 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-210-Seg3 as a white solid in 98% purity. The isolated yield based on the resin loading was 25%. HRMS (ESI): calculated for C₁₉₃H₃₁₅N₅₁O₅₇S: 4293.3094 Da; found: 4293.2962 Da. Retention time (analytical method 12): 8.181 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-210-Seg4 as a white solid in 95% purity. The isolated yield based on the resin loading was 13%. HRMS (ESI): calculated for C₂₀₉H₃₅₁N₅₉O₆₀: 4649.6285 Da; found: 4649.6457 Da. Retention time (analytical method 2): 6.21 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-210-Seg12 as a white solid in 90% purity. The isolated yield was 24%. HRMS (ESI): calculated for C₄₁₄H₆₅₉N₁₀₉O₁₃₂S: 9306.8065 Da; found: 9306.8343 Da. Retention time (analytical method 2): 6.57 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-210-Seg34 as a white solid in 90% purity. The isolated yield was 18%. HRMS (ESI): calculated for C₃₈₆H₆₅₆N₁₁₀O₁₃S: 8677.8825 Da; found: 8677.9092 Da. Retention time (analytical method 2): 6.15 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-210-Seg1234 with Acm as a white solid in 90% purity. The isolated yield was 33%. HRMS (ESI): calculated for C₇₉₉H₁₃₁₅N₂₁₉O₂₄₃S₂: 17940.6990 Da; found: 17940.7380 Da. Retention time (analytical method 2): 6.46 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-210-Linear protein as a white powder in 92% purity (57% yield for Acm deprotection and purification steps). HRMS (ESI): calculated for C₇₉₃H₁₃₀₅N₂₁₇O₂₄₁S₂: 17798.6248 Da; found: 17798.6076 Da. Retention time (analytical method 2): 6.520 min.

Folding: Rearrangement and folding CMP-210 linear protein. The linear protein was rearranged, folded and purified following protocol 10. The folded protein was not obtained.

### Example 2T: Synthesis of CMP-211

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-211-Seg1 as a white solid in 93% purity. The isolated yield based on the resin loading was 9%. HRMS (ESI): calculated for C₁₉₅H₃₂₆N₄₆O₇₅S: 4546.2887 Da; found: 4546.319 Da. Retention time (analytical method 11): 11.625 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).

After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-211-Seg2 as a white solid in 84% purity. The isolated yield based on the resin loading was 22%. HRMS (ESI): calculated for C₂₃₁H₃₄₆N₆₄O₆₂: 5010.5946 Da; found: 5010.6347 Da. Retention time (analytical method 12): 8.749 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-211-Seg3 as a white solid in 93% purity. The isolated yield based on the resin loading was 15%. HRMS (ESI): calculated for C₁₉₀H₃₁₀N₅₀O₅₇: 4206.2953 Da; found: 4206.3445 Da. Retention time (analytical method 12): 8.153 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-211-Seg4 as a white solid in 96% purity. The isolated yield based on the resin loading was 18%. HRMS (ESI): calculated for C₂₁₂H₃₅₆N₆₀O₆₀S: 4736.6426 Da; found: 4736.6833 Da. Retention time (analytical method 12): 8.531 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-211-Seg12 as a white solid in 88% purity. The isolated yield was 21%. HRMS (ESI): calculated for C₄₁₄H₆₅₉N₁₀₉O₁₃₂S: 9306.8065 Da; found: 9306.8205 Da. Retention time (analytical method 11): 12.142 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-211-Seg34 as a white solid in 93% purity. The isolated yield was 14%. HRMS (ESI): calculated for C₃₈₆H₆₅₆N₁₁₀O₁₁₃S: 8677.8825 Da; found: 8676.9099 Da. Retention time (analytical method 11): 9.892 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-211-Seg1234-Acm as a white solid in 90% purity. The isolated yield was 28%. HRMS (ESI): calculated for C₇₉₉H₁₃₁₅N₂₁₉O₂₄₃S₂: 17940.699 Da; found: 17940.6991 Da. Retention time (analytical method 11): 11.575 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-211-Linear protein as a white powder in 92% purity (50% yield for Acm deprotection and purification steps). HRMS (ESI): calculated for C₇₉₃H₁₃₀₅N₂₁₇O₂₄₁S₂: 17798.6248 Da; found: 17799.6425 Da. Retention time (analytical method 11): 12.008 min.

Folding: Rearrangement and folding CMP-211 linear protein. The linear protein was rearranged, folded and purified following protocol 10. The fractions containing the purified product were pooled and lyophilized to obtain CMP-211 as a white powder in 94% purity. The isolated yield was 37%. The purity and identity of the pure folded protein was further confirmed by analytical HPLC and HRMS. HRMS (ESI): calculated for C₇₉₃H₁₃₀₃N₂₁₇O₂₄₁S₂: 17796.6091 Da; found: 17796.6433 Da. Retention time (analytical method 16): 15.092 min.

### Example 2U: Synthesis of CMP-212

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-212-Seg1 as a white solid in 90% purity. The isolated yield based on the resin loading was 11%. HRMS (ESI): calculated for C₁₉₂H₃₂₁N₄₅O₇₅: 4459.2746 Da; found: 4459.2882 Da. Retention time (analytical method 2): 6.82 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-212-Seg2 as a white solid in 94% purity. The isolated yield based on the resin loading was 40%. HRMS (ESI): calculated for C₂₃₄H₃₅₁N₆₅O₆₂S: 5098.6113 Da; found: 5098.6386 Da. Retention time (analytical method 13): 5.271 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-212-Seg3 as a white solid in 98% purity. The isolated yield based on the resin loading was 15%. HRMS (ESI): calculated for C₁₉₀H₃₁₀N₅₀O₅₇: 4206.2953 Da; found: 4206.3445 Da. Retention time (analytical method 12): 8.153 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-212-Seg4 as a white solid in 97% purity. The isolated yield based on the resin loading was 14%. HRMS (ESI): calculated for C₂₁₂H₃₅₆N₆₀O₆₀S: 4736.6426 Da; found: 4736.6852 Da. Retention time (analytical method 12): 8.701 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-212-Seg12 as a white solid in 87% purity. The isolated yield was 29%. HRMS (ESI): calculated for C₄₁₄H₆₅₉N₁₀₉O₁₃₂S: 9306.8065 Da; found: 9306.8347 Da. Retention time (analytical method 11): 12.092 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-212-Seg34 as a white solid in 90% purity. The isolated yield was 20%. HRMS (ESI): calculated for C₃₈₆H₆₅₆N₁₁₀O₁₃S: 8677.8825 Da; found: 8676.9054 Da. Retention time (analytical method 2): 6.13 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-212-Seg1234-Acm as a white solid in 90% purity. The isolated yield was 37%. HRMS (ESI): calculated for C₇₉₉H₁₃₁₅N₂₁₉O₂₄₃S₂: 17940.699 Da; found: 17940.6807 Da. Retention time (analytical method 2): 6.47 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-212-Linear protein as a white powder in 90% purity (65% yield for Acm deprotection and purification steps). HRMS (ESI): calculated for C₇₉₃H₁₃₀₅N₂₁₇O₂₄₁S₂: 17798.6248 Da; found: 17798.5879 Da. Retention time (analytical method 2): 6.46 min.

Folding: Rearrangement and folding CMP-212 linear protein. The linear protein was rearranged, folded and purified following protocol 10. The folded protein was not obtained.

### Example 2V: Synthesis of CMP-201

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-201-Seg1 as a white solid in 93% purity. The isolated yield based on the resin loading was 9%. HRMS (ESI): calculated for C₁₉₅H₃₂₆N₄₆O₇₅S: 4546.2887 Da; found: 4546.319 Da. Retention time (analytical method 11): 11.625 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).

After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-201-Seg2 as a white solid in 94% purity. The isolated yield based on the resin loading was 40%. HRMS (ESI): calculated for C₂₃₄H₃₅₁N₆₅O₆₂S: 5098.6113 Da; found: 5098.6386 Da. Retention time (analytical method 13): 5.271 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-201-Seg3 as a white solid in 95% purity. The isolated yield based on the resin loading was 28%. HRMS (ESI): calculated for C₁₉₂H₃₁₃N₅₁O₅₈: 4263.3168 Da; found: 4263.3684 Da. Retention time (analytical method 15): 4.172 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-201-Seg4 as a white solid in 95% purity. The isolated yield based on the resin loading was 5%. HRMS (ESI): calculated for C₂₁₅H₃₆₁N₆₁O₆₀S₂: 4823.6568 Da; found: 4823.6937 Da. Retention time (analytical method 9): 5.50 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-201-Seg12 as a white solid in 90% purity. The isolated yield was 35%. HRMS (ESI): calculated for C₄₁₇H₆₆₄N₁₁₀O₁₃₂S₂: 9393.8204 Da; found: 9393.8572 Da. Retention time (analytical method 2): 6.56 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-201-Seg34 as a white solid in 92% purity. The isolated yield was 30%. HRMS (ESI): calculated for C₃₉₁H₆₆₄N₁₁₂O₁₁₄S₂: 8821.9179 Da; found: 8820.9532 Da. Retention time (analytical method 2): 6.07 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-201-Seg1234-Acm as a white solid in 96% purity. The isolated yield was 22%. HRMS (ESI): calculated for C₈₀₇H₁₃₂₈N₂₂₂O₂₄₄S₄: 18171.7483 Da; found: 18172.7694 Da. Retention time (analytical method 2): 6.49 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-201-Linear protein as a white powder in 94% purity (65% yield for Acm deprotection and purification steps). HRMS (ESI): calculated for C₇₉₅H₁₃₀₈N₂₁₈O₂₄₀S₄: 17887.5998 Da; found: 17887.5588 Da. Retention time (analytical method 2): 6.58 min.

Folding: Rearrangement and folding CMP-201 linear protein. The linear protein was rearranged, folded and purified following protocol 10. The fractions containing the purified product were pooled and lyophilized to obtain CMP-201 as a white powder in 99% purity. The isolated yield was 32%. The purity and identity of the pure folded protein was further confirmed by analytical HPLC and HRMS (ESI). HRMS (ESI): calculated for C₇₉₅H₁₃₀₄N₂₁₈O₂₄₀S₄: 17883.5685 Da; found: 17883.6144 Da. Retention time (analytical method 16): 14.508 min.

### Example 2W: Synthesis of CMP-202

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-202-Seg1 as a white solid in 94% purity. The isolated yield based on the resin loading was 7%. HRMS (ESI): calculated for C₁₉₅H₃₂₆N₄₆O₇₅S: 4546.2887 Da; found: 4546.3258 Da. Retention time (analytical method 15): 4.785 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-202-Seg2 as a white solid in 98% purity. The isolated yield based on the resin loading was 20%. HRMS (ESI): calculated for C₂₃₄H₃₅₁N₆₅O₆₂S: 5098.6113 Da; found: 5098.6386 Da. Retention time (analytical method 13): 5.271 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). Side chain Alloc deprotection of Lys (Lys81) was performed following protocol 3 and elongation with O—(N-Boc-2-aminoethyl)-O′—(N-diglycolyl-2-aminoethyl)-hexaethyleneglycol (structure 8) was pursued on the free amine side chain following protocol 2. After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-202-Seg3 as a white solid in 92% purity. The isolated yield based on the resin loading was 9%. HRMS (ESI): calculated for C₂₁₂H₃₅₁N₅₃O₆₈: 4729.5695 Da; found: 4729.6243 Da. Retention time (analytical method 15): 4.234 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-202-Seg4 as a white solid in 98% purity. The isolated yield based on the resin loading was 95% purity. The isolated yield based on the resin loading was 5%. HRMS (ESI): calculated for C₂₁₅H₃₆₁N₆₁O₆₀S₂: 4823.6568 Da; found: 4823.6937 Da. Retention time (analytical method 9): 5.50 min

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-202-Seg12 as a white solid in 92% purity. The isolated yield was 30%. HRMS (ESI): calculated for C₄₁₇H₆₆₄N₁₁₀O₁₃₂S₂: 9393.8204 Da; found: 9393.8524 Da. Retention time (analytical method 2): 6.62 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-202-Seg34 as a white solid in 92% purity. The isolated yield was 19%. HRMS (ESI): calculated for C₄₁₁H₇₀₂N₁₁₄O₁₂₄S₂: 9288.1707 Da; found: 9288.2030 Da. Retention time (analytical method 2): 6.18 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-202-Seg1234-Acm as a white solid in 98% purity. The isolated yield was 28%. HRMS (ESI): calculated for C₈₂₇H₁₃₆₆N₂₂₄O₂₅₄S₄: 18639.0036 Da; found: 18639.0284 Da. Retention time (analytical method 2): 6.48 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-202-Linear protein as a white powder in 92% purity (38% yield for Acm deprotection and purification steps). HRMS (ESI): calculated for C₈₁₅H₁₃₄₆N₂₂₀O₂₅₀S₄: 18354.8551 Da; found: 18354.8532 Da. Retention time (analytical method 15): 4.655 min.

Folding: Rearrangement and folding CMP-202 linear protein. The linear protein was rearranged, folded and purified following protocol 10. The fractions containing the purified product were pooled and lyophilized to obtain CMP-202 as a white powder in 93.8% purity. The isolated yield was 42%.

The purity and identity of the pure folded protein was further confirmed by analytical HPLC and HRMS (ESI). HRMS (ESI): calculated for C₈₁₅H₁₃₄₂N₂₂₀O₂₅₀S₄: 18350.8238 Da; found: 18350.8378 Da. Retention time (analytical method 16): 14.558 min.

### Example 2X: Synthesis of CMP-213

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-213-Seg1 as a white solid in 97% purity. The isolated yield based on the resin loading was 18%. HRMS (ESI): calculated for C₁₉₈H₃₃₁N₄₇O₇₅S₂: 4633.3028 Da; found: 4633.3183 Da. Retention time (analytical method 2): 6.821 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr).

After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-213-Seg2 as a white solid in 84% purity. The isolated yield based on the resin loading was 22%. HRMS (ESI): calculated for C₂₃₁H₃₄₆N₆₄O₆₂: 5010.5946 Da; found: 5010.6347 Da. Retention time (analytical method 12): 8.749 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-213-Seg3 as a white solid in 96% purity. The isolated yield based on the resin loading was 34%. HRMS (ESI): calculated for C₁₉₅H₃₁₈N₅₂O₅₈S: 4350.3309 Da; found: 4350.3487 Da. Retention time (analytical method 2): 6.179 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-213-Seg4 as a white solid in 96% purity. The isolated yield based on the resin loading was 18%. HRMS (ESI): calculated for C₂₁₂H₃₅₆N₆₀O₆₀S: 4736.6426 Da; found: 4736.6833 Da. Retention time (analytical method 12): 8.531 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-213-Seg12 as a white solid in 86% purity. The isolated yield was 24%. HRMS (ESI): calculated for C₄₁₇H₆₆₄N₁₁₀O₁₃₂S₂: 9393.8204 Da; found: 9393.8326 Da. Retention time (analytical method 11): 12.058 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-213-Seg34 as a white solid in 96% purity. The isolated yield was 55%. HRMS (ESI): calculated for C₃₉₁H₆₆₄N₁₁₂O₁₁₄S₂; Average isotope calculated Da; found: Da. Retention time (analytical method 11): 4.139 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-213-Seg1234-Acm as a white solid in 88% purity. The isolated yield was 26%. HRMS (ESI): calculated for C₈₀₇H₁₃₂₈N₂₂₂O₂₄₄S₄: 18171.7483 Da; found: 18172.7514 Da. Retention time (analytical method 2): 6.40 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-213-Linear protein as a white powder in 93% purity (62% yield for Acm deprotection and purification steps). HRMS (ESI): calculated for C₇₉₅H₁₃₀₈N₂₁₈O₂₄₀S₄: 17887.5998 Da; found: 17888.6176 Da. Retention time (analytical method 2): 6.60 min.

Folding: Rearrangement and folding CMP-213 linear protein. The linear protein was rearranged, folded and purified following protocol 10. The fractions containing the purified product were pooled and lyophilized to obtain CMP-213 as a white powder in 94% purity. The isolated yield was 2%. The purity and identity of the pure folded protein was further confirmed by analytical HPLC and HRMS (ESI). HRMS (ESI): calculated for C₇₉₅H₁₃₀₈N₂₁₈O₂₄₀S₄: 17883.5685 Da; found: 17883.5439 Da. Retention time (analytical method 16): 15.092 min.

### Example 2Y: Synthesis of CMP-214

Segment 1: Loading of the KAHA monomer Fmoc-Leu-protected-α-ketoacid was performed following protocol 1. The peptide elongation cycles including amino acid coupling, capping and Fmoc deprotection were performed following protocol 2 until position 1 aspartic acid. The N-terminus was elongated with azido PEG9-glutaric acid (structure 3) by coupling glutaric anhydride according to protocol 4, followed by coupling of azido-PEG9 amine using protocol 2. After the peptide elongation, the resin was washed with DCM and dried under vacuum. Segment 1 was then released from the resin and the side chain deprotected following protocol 5. Purification of crude peptide was performed by preparative HPLC (protocol 12). The fractions containing the purified product were pooled and lyophilized to obtain CMP-214-Seg1 as a white solid in 97% purity. The isolated yield based on the resin loading was 18%. HRMS (ESI): calculated for C₁₉₈H₃₃₁N₄₇O₇₅S₂: 4633.3028 Da; found: 4633.3183 Da. Retention time (analytical method 2): 6.821 min.

Segment 2: The loading of Fmoc-Phe-photoprotected-α-ketoacid was performed following protocol 1. The peptide elongation cycles including amino acid coupling, capping and Fmoc deprotection were performed as described in protocol 2 until position 36 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-214-Seg2 as a white solid in 84% purity. The isolated yield based on the resin loading was 22%. HRMS (ESI): calculated for C₂₃₁H₃₄₆N₆₄O₆₂: 5010.5946 Da; found: 5010.6347 Da. Retention time (analytical method 12): 8.749 min.

Segment 3: the loading of Fmoc-Leu-protected-α-ketoacid was performed following protocol 1. The peptide elongation cycles including amino acid coupling, capping and Fmoc deprotection were performed as described in protocol 2 until position 76 (Fmoc-Opr). Side chain Alloc deprotection of Lys (Lys81) was performed following protocol 3 and elongation with O—(N-Boc-2-aminoethyl)-O′—(N-diglycolyl-2-aminoethyl)-hexaethyleneglycol (structure 8) was pursued on the free amine side chain following protocol 2. After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-214-Seg3 as a white solid in 97% purity. The isolated yield based on the resin loading was 8%. HRMS (ESI): calculated for C₂₁₅H₃₅₆N₅₄O₆₈S: 4816.5836 Da; found: 4816.6267 Da. Retention time (analytical method 11): 10.375 min.

Segment 4: the peptide was synthesized on Rink amide MBHA or Ramage amide PS resin. The peptide elongation cycles including amino acid coupling, capping and Fmoc deprotection were performed as described in protocol 2 until position 114 (Boc-Opr). After the peptide elongation, the resin was washed with DCM and diethyl ether and dried under vacuum. The peptide was cleaved from the resin following protocol 5.

Purification of crude peptide was performed by preparative HPLC (protocol 12). The fractions containing the purified product were pooled and lyophilized to obtain CMP-214-Seg4 as a white solid in 96% purity. The isolated yield based on the resin loading was 18%. HRMS (ESI): calculated for C₂₁₂H₃₅₆N₆₀O₆₀S: 4736.6426 Da; found: 4736.6833 Da. Retention time (analytical method 12): 8.531 min.

Segment 12: Ligation of segment 1 and 2 and photodeprotection were performed as described in protocol 6. The ligated/deprotected sample was purified by preparative HPLC (protocol 12). The fractions containing the purified product were pooled and lyophilized to obtain CMP-214-Seg12 as a white solid in 93% purity. The isolated yield was 27%. HRMS (ESI): calculated for C₄₁₇H₆₆₄N₁₁₀O₁₃₂S₂: 9393.8204 Da; found: 9393.8423 Da. Retention time (analytical method 2): 6.55 min.

Segment 34: Ligation of segment 3 and 4 and Fmoc-deprotection were performed as described in protocol 7. The ligation/deprotection sample was purified by preparative HPLC (protocol 12). The fractions containing the purified product were pooled and lyophilized to obtain CMP-214-Seg34 as a white solid in 95% purity. The isolated yield was 32%. HRMS (ESI): calculated for C₄₁₁H₇₀₂N₁₁₄O₁₂₄S₂: 9288.1707 Da; found: 9288.1846 Da. Retention time (analytical method 2): 6.05 min.

Final ligation: Segment 1234 (Acm protected cysteines): Ligation of segment 12 and 34 was performed as described in protocol 8. The ligation sample was purified by preparative HPLC (protocol 12). The fractions containing the purified product were pooled and lyophilized to obtain CMP-214-Seg1234-Acm as a white solid in 89% purity. The isolated yield was 24%. HRMS (ESI): calculated for C₈₂₇H₁₃₆₆N₂₂₄O₂₅₄S₄: 18639.0036 Da; found: 18639.0342 Da. Retention time (analytical method 2): 6.40 min.

Linear protein: Acm deprotection: Acm protection on cysteine residues were removed using protocol 9. The deprotected sample was purified by preparative HPLC (protocol 12). The fractions containing the purified product were pooled and lyophilized to obtain CMP-214-Linear protein as a white powder in 92% purity (60% yield for Acm deprotection and purification steps). HRMS (ESI): calculated for C₈₁₅H₁₃₄₆N₂₂₀O₂₅₀S₄: 18354.8551 Da; found: 18354.8342 Da. Retention time (analytical method 1): 6.59 min.

Folding: Rearrangement and folding CMP-214 linear protein. The linear protein was rearranged, folded and purified following protocol 10. The fractions containing the purified product were pooled and lyophilized to obtain CMP-214 as a white powder in 94% purity. The isolated yield was 6%. The purity and identity of the pure folded protein was further confirmed by analytical HPLC and HRMS. HRMS (ESI): calculated for C₈₁₅H₁₃₄₂N₂₂₀O₂₅₀S₄: 18350.8082 Da; found: 18350.8232 Da. Retention time (analytical method 4): 15.075 min.

### Example 3: N-Terminal Modification of Synthetic Substituted IL-7 with Azide Conjugation Handle at N-Terminus

The method for synthesizing IL-7 according to Example 2A is modified in order to prepare construct SEQ ID NO: 3 with azide conjugation handle in N-terminus (CMP-108) as shown in FIG. 5B. CMP-108 differs from the IL-7 polypeptide of SEQ ID NO: 3 prepared in Example 2A (i.e., SEQ ID NO: 3) in that CMP-108 contains a modified N-terminal amine having a structure

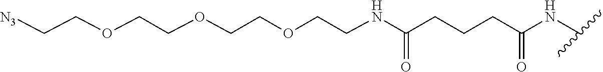 [SMILES: *NC(=O)CCCC(=O)NCCOCCOCCOCCN=[N+]=[N-]]

This version is prepared analogously to the IL-7 of SEQ ID NO: 3 in Example 2A above with the following modification performed after final Fmoc deprotection of the N-terminal residue. Manual coupling reaction is performed at r.t. for 2 h by addition of glutaric anhydride (CAS RN 108-55-4, 114.10 mg, 5 equiv.) and DIPEA (242 μL, 7 equiv.) in DMF to the resin. Secondly, coupling with commercially available O-(2-Aminoethyl)-O′-(2-azidoethyl) nonaethylene glycol (Compound 1, 421 mg, 4 equiv) in DMF is performed at r.t. for 3 hours by addition of DIPEA (276 μL, 8 equiv) and HATU (300.4 mg, 3.95 equiv) in DMF to the resin.

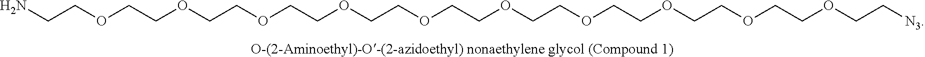 [SMILES: [N-]=[N+]=NCCOCCOCCOCCOCCOCCOCCOCCOCCOCCOCCN]

The resin is then washed and cleaved as per the normal protocol, and the modified N-terminal fragment is used in the remaining ligation steps as described in Example 2A. The linear protein was analyzed by RP-HPLC and ESI-HMRS FIG. 3C. Representative data of the folded IL-7 protein is shown in FIG. 3D.

### Example 4: Additional Protocol for Folding of IL-7

The following additional protocol can be used for folding of an IL-7 polypeptide as provided herein (e.g., a synthetic IL-7 polypeptide)

Folding Step 1: The linear protein (e.g., tri-depsipeptide version of the final sequence) is dissolved in 50 mM Tris buffer, containing 6 M GnHCl, 50 mM NaCl, 1 mM EDTA and 10 mM CysHCl (40 μM protein concentration), which is adjusted to pH 8.0 by adding a solution of 6 M aqueous HCl. The mixture is gently shaken at rt for 3 h. The rearrangement is monitored by analytical reverse phase HPLC.

Folding Step 2: The solution with the rearranged protein is cooled to 4C. and diluted (×8) with 50 mM Tris buffer containing 50 mM NaCl, 0.11 M Arg, 1 mM EDTA and 0.142 mM cystine, which is adjusted to pH 8.0 by adding a solution of 6 M aqueous HCl. The folding is performed for 20 h at 4° C. and monitored by HPLC.

Purification: After completion of the folding reaction as ascertained by HPLC, the sample is acidified with TFA to pH=3, and purified by preparative HPLC using a C4 column (20×250 mm) at a flow rate of 10 mL/min at rt using CH3CN/H₂O with 0.1% TFA (v/v) as mobile phase, with a gradient of 30 to 85% CH3CN with 0.1% TFA (v/v) in 50 min. The fractions containing the purified product are pooled and lyophilized with 5% (w/v) sucrose to obtain the folded IL-7 syntein (e.g., FIG. 5A or FIG. 5B). The purity and identity of the pure folded protein is further confirmed by analytical HPLC and LC/ESI/MS/MS.

### Example 5: Determination of IL-7-Induced pSTAT5 Phosphorylation of Synthetic IL-7

Primary pan T-cells were obtained from healthy donor buffy coats by peripheral blood mononuclear cell (PBMC) purification using Ficoll gradient centrifugation, followed by negative isolation with magnetic beads and then cryopreserved until use. Pan T-cells were thawed, allowed to recover overnight in T-cell medium (RPMI 10% FCS, 1% Glutamine, 1% NEAA, 25 μM bMcoH, 1% NaPyrovate). After two washing steps with PBS, cells were resuspended in PBS. Cells were then distributed at 200,000 cells per well and stimulated with serial dilutions of wild type or modified IL-7 polypeptides for 40 min at 37° C./5% CO₂. After incubation, cells were fixed and permeabilized using the Transcription Factor Phospho Buffer kit followed by staining of surface and intracellular staining markers (CD4, CD8, CD25, FoxP3, CD45RA, pStat5) to enable the identification of cell subsets and to measure levels of STAT5 phosphorylation. FACS measurement was accomplished either with a NovoCyte or a Quanteon Flow Cytometer from Acea Biosciences. Flow-Jo is used for all FACS analyses.

The percentage of pSTAT5-positive T-cell subsets was plotted against concentrations of either wild type or modified IL-7 polypeptides. The half maximal effective concentration (EC50) was calculated based on a variable slope, four parameter analysis. TABLE 6 shows the gating strategy for T-cell subset identification.

TABLE 6

| | |
|---|---|
| CD4 Treg | CD4+, CD45RA−, CD25^{Hi}, FoxP3+ |
| CD4 Memory | CD4+, CD45RA− |
| CD4 Naïve | CD4+, CD45RA+ |
| CD8 Memory | CD8+, CD45RA− |
| CD8 Naïve | CD8+, CD45RA+ |

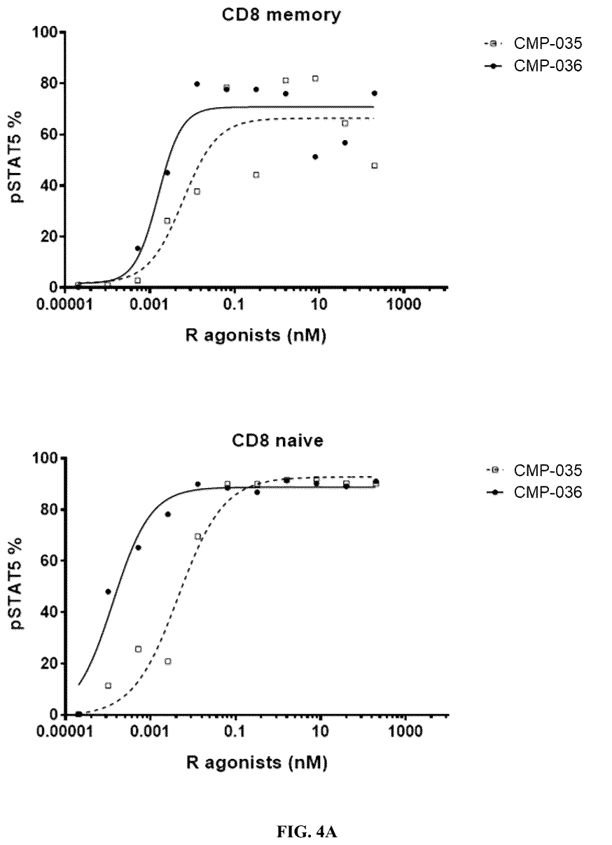

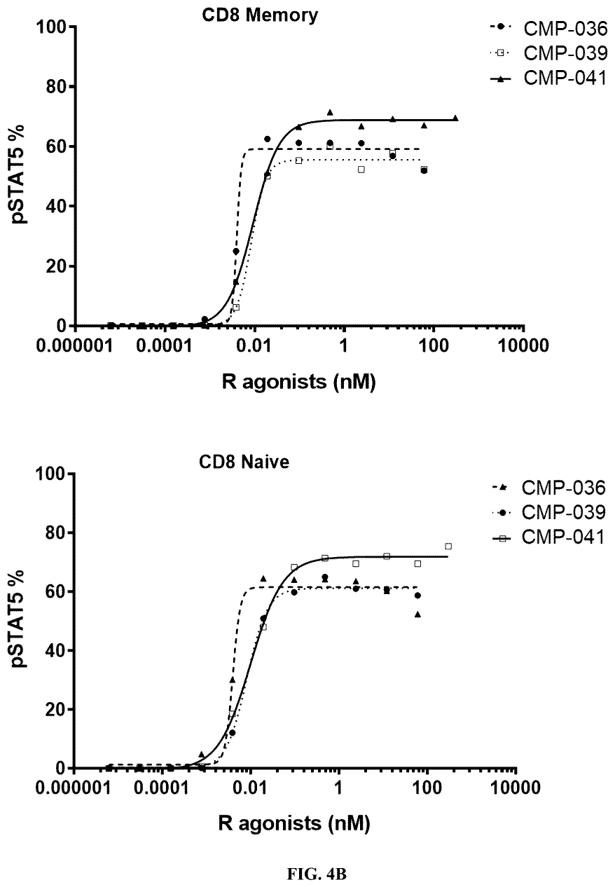

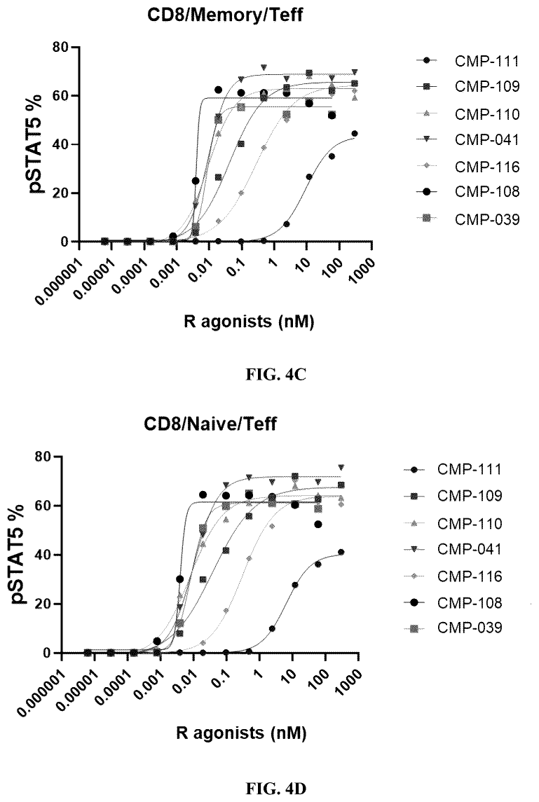

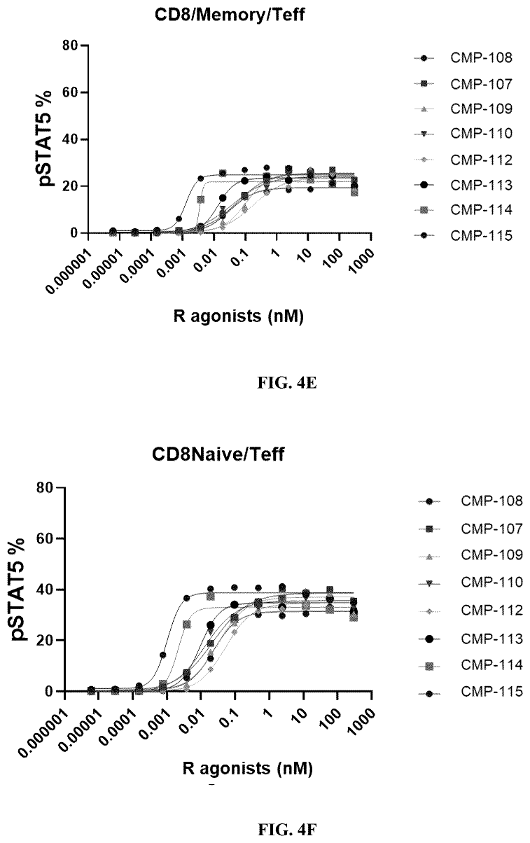

pSTAT5 induction in primary human T cells for WT IL-7 His (SEQ ID NO: 2) and synthetic IL-7 (SEQ ID NO: 3) is shown in FIG. 4A show dose dependent pSTAT5 induction in CD8 naïve, and CD8 memory T cells when treated with WT IL-7 His (SEQ ID NO: 2) or synthetic IL-7 of SEQ ID NO: 3. WT IL-7 His and synthetic IL-7 exhibit similar ability to induce pSTAT5, indicating that synthetic IL-7 behaves similar to wild type. FIG. 4B shows dose dependent pSTAT5 induction in CD8 naïve and CD8 memory T cells treated with SEQ. ID. NO 3, CMP-039 and CMP-041. Both CMP-039 and CMP-041 show similar activity to SEQ. ID. NO 3, indicating that conjugation does not impact the IL-7 functionality. Similar data is shown in FIG. 4C, FIG. 4D, FIG. 4E, and FIG. 4F for naïve and antigen experiences CD8+ cells for additional IL-7 polypeptides and immunocytokines thereof. EC50 values for various T cell subtypes are shown in Table 7 below.

TABLE 7

| | | EC₅₀ pSTAT5 (average values of all | | | | |
|---|---|---|---|---|---|---|
| | | experiments)(nM) | | | | |
| | | CD4 | CD8 | CD8 | CD4 | CD4 |
| Identifiers | Modifications | Treg | Memory | Naive | Memory | Naive |
| SEQ ID | IL-7 None (Recomb- | 0.032 | 0.006 | 0.002 | 0.001 | 0.001 |
| NO: 2, | HIS6 (“HHHHHH” | | | | | |
| CMP-035 | disclosed as SEQ ID | | | | | |
| | NO: 374)) | | | | | |
| SEQ ID | IL-7 Q11F (Recom- | 0.010 | 0.002 | 0.001 | 0.001 | 0.001 |
| NO: 56, | HIS6 (“HHHHHH” | | | | | |
| CMP-117 | disclosed as SEQ ID | | | | | |
| | NO: 374)) | | | | | |
| SEQ ID | IL-7 V15W | 27.652 | 3.22 | 1.276 | 0.930 | 0.814 |
| NO: 51, | (Recomb-HIS6 | | | | | |
| CMP-106 | (“HHHHHH” | | | | | |
| | disclosed as SEQ ID | | | | | |
| | NO: 374)) | | | | | |
| SEQ ID | IL-7 M17Nle, | 0.015 | 0.006 | 0.006 | 0.005 | 0.005 |
| NO: 3, | M27Nle, N36Hse, | | | | | |
| CMP-036 | M54Nle, M69Nle, | | | | | |
| | D76Hse, E114Hse, | | | | | |
| | M147Nle (synthetic) | | | | | |
| CMP-107 | IL-7 N-terminal | 1.204 | 0.034 | 0.006 | 0.0004 | 0.001 |
| | PEG₉-azide, V15W, | | | | | |
| | M17Nle, M27Nle, | | | | | |
| | N36Hse, M54Nle, | | | | | |
| | M69Nle, D76Hse, | | | | | |
| | E114Hse, M147Nle | | | | | |
| | (synthetic) | | | | | |
| CMP-039 | IL-7 N-terminal | 0.011 | 0.006 | 0.004 | 0.004 | 0.004 |
| | PEG₉-azide, | | | | | |
| | M17Nle, M27Nle, | | | | | |
| | N36Hse, M54Nle, | | | | | |
| | M69Nle, D76Hse, | | | | | |
| | E114Hse, M147Nle | | | | | |
| | (synthetic), | | | | | |
| | pembrolizumab | | | | | |
| | K248 DAR1 | | | | | |
| | conjugate | | | | | |
| CMP-040 | IL-7 N-terminal | 0.017 | 0.007 | 0.007 | 0.005 | 0.003 |
| | PEG₉-azide, | | | | | |
| | M17Nle, M27Nle, | | | | | |
| | N36Hse, M54Nle, | | | | | |
| | M69Nle, D76Hse, | | | | | |
| | E114Hse, M147Nle | | | | | |
| | (synthetic), | | | | | |
| | pembrolizumab | | | | | |
| | K248 DAR2 | | | | | |
| | conjugate | | | | | |
| CMP-041 | IL-7 N-terminal | 0.034 | 0.032 | 0.015 | 0.018 | 0.016 |
| | PEG₉-azide, | | | | | |
| | M17Nle, M27Nle, | | | | | |
| | N36Hse, M54Nle, | | | | | |
| | M69Nle, D76Hse, | | | | | |
| | E114Hse, M147Nle | | | | | |
| | (synthetic) | | | | | |
| | LZM-009, DAR1 | | | | | |
| CMP-108 | N-terminal PEG₉- | 0.02 | 0.008 | 0.008 | 0.006 | 0.006 |
| | azide, M17Nle, | | | | | |
| | M27Nle, N36Hse, | | | | | |
| | M54Nle, M69Nle, | | | | | |
| | D76Hse, E114Hse, | | | | | |
| | M147Nle (synthetic) | | | | | |
| CMP-109 | N-terminal PEG₉- | 3.9 | 0.15 | 0.05 | 0.09 | 0.05 |
| | azide, M17Nle, | | | | | |
| | M27Nle, N36Hse, | | | | | |
| | M54Nle, M69Nle, | | | | | |
| | D76Hse, K81(L6P), | | | | | |
| | E114Hse, M147Nle | | | | | |
| | (synthetic) | | | | | |
| CMP-110 | N-terminal PEG₉- | 67 | 0.08 | 0.05 | 0.03 | 0.03 |
| | azide, M17Nle, | | | | | |
| | M27Nle, N36Hse, | | | | | |
| | M54Nle, M69Nle, | | | | | |
| | D76Hse, G85N, | | | | | |
| | E114Hse, M147Nle | | | | | |
| | (synthetic) | | | | | |
| CMP-111 | N-terminal PEG₉- | 82.3 | 16.2 | 12.3 | 11.2 | 11.6 |
| | azide, M17Nle, | | | | | |
| | M27Nle, N36Hse, | | | | | |
| | M54Nle, M69Nle, | | | | | |
| | D76Hse, K81(L6P), | | | | | |
| | G85N, E114Hse, | | | | | |
| | M147Nle (synthetic) | | | | | |
| CMP-112 | N-terminal PEG₉- | 1.02 | 0.2 | 0.09 | 0.09 | 0.05 |
| | azide, M17Nle, | | | | | |
| | M27Nle, N36Hse, | | | | | |
| | M54Nle, M69Nle, | | | | | |
| | D76Hse, | | | | | |
| | K81(L11P), | | | | | |
| | E114Hse, M147Nle | | | | | |
| | (synthetic) | | | | | |
| CMP-113 | N-terminal PEG₉- | 0.18 | 0.06 | 0.05 | 0.04 | 0.04 |
| | azide, C2A, | | | | | |
| | M17Nle, M27Nle, | | | | | |
| | N36Hse, M54Nle, | | | | | |
| | M69Nle, D76Hse, | | | | | |
| | C92A, E114Hse, | | | | | |
| | M147Nle (synthetic) | | | | | |
| CMP-114 | M17Nle, M27Nle, | 0.01 | 0.004 | 0.005 | 0.004 | 0.002 |
| | N36Hse, M54Nle, | | | | | |
| | M69Nle, D76Hse, | | | | | |
| | E114Hse, M147Nle | | | | | |
| | (synthetic) | | | | | |
| CMP-115 | M17Nle, M27Nle, | 0.09 | 0.01 | 0.3 | 0.008 | 0.007 |
| | N36Hse, M54Nle, | | | | | |
| | M69Nle, D76Hse, | | | | | |
| | K81L2P, E114Hse, | | | | | |
| | M147Nle (synthetic) | | | | | |
| CMP-116 | N-terminal PEG₉- | 1.5 | 0.2 | 0.2 | 0.1 | 0.3 |
| | azide, V15W, | | | | | |
| | M17Nle, M27Nle, | | | | | |
| | N36Hse, M54Nle, | | | | | |
| | M69Nle, D76Hse, | | | | | |
| | E114Hse, M147Nle | | | | | |
| | (synthetic) | | | | | |
| | conjugated to LZM- | | | | | |
| | 009, DAR1 | | | | | |
| | Conjugate | | | | | |
| CMP-200 | IL-7 syntein missing | 0.14 | 0.04 | 0.03 | 0.03 | 0.02 |
| | C2-C92disulphide | | | | | |
| | bond, N-terminus | | | | | |
| | with glutaric acid | | | | | |
| | and (O- | | | | | |
| | AcetamidoPEG0.5K | | | | | |
| | azide) | | | | | |
| CMP-201 | | 4.45 | 1.7 | 1.2 | 0.7 | 0.8 |
| CMP-202 | | 32.7 | 37.4 | 20.4 | 38.1 | 18.9 |

### Example 6: Preparation of Anti-PD-1 Antibody-Conjugated IL-7 Immunocytokine

Antibodies Pembrolizumab and LZM-009 were used to prepare immunocytokines with IL-7 polypeptides CMP-108 (SEQ ID NO: 3 with N-terminal PEG9-azide) or CMP-107 (SEQ ID NO: 3 with V15W substitution and N-terminal PEG9-azide) (FIG. 5B) according to the following general methods. The general methods provided below are also used to prepare immunocytokines of antibodies Pembrolizumab and LZM-009 conjugated to other variant IL-7 polypeptides provided herein, including CMP-109, CMP-110, and CMP-111.

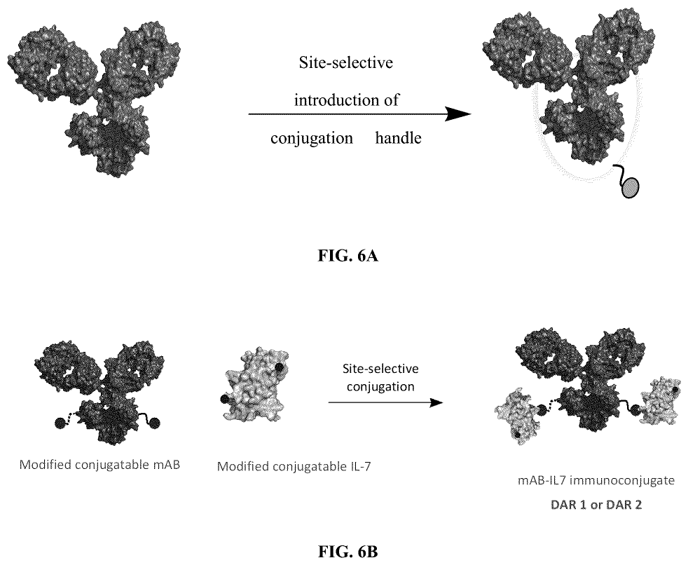

FIG. 6A illustrates site selective introduction of a conjugation handle on a Fc domain. A conjugatable variant antibody is prepared using an AJICAP™ method (Ajinomoto Bio-Pharma Services). This method allows production of >50 mg of conjugatable antibody within weeks. The conjugatable product harbors one or two chemical handles for further modifications (FIG. 6B). Alternatively, a mixture of DAR1 and DAR2 antibody immunoconjugates is generated, and the average cytokine loading is used to determine an intermediate DAR (e.g., DAR 1.1, 1.2, 1.3, 1.4, 1.5, 1.6, 1.7, 1.8, or 1.9). General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1. A general protocol for this methodology is provided below:

A modified antibody (e.g., an anti-PD-1 antibody such as Pembrolizumab or LZM-009) comprising a DBCO conjugation handle is prepared using a protocol modified from Examples 2-4 of US Patent Publication No. US20200190165A1. Briefly, the antibody with a free sulfhydryl group attached to a lysine residue side chain in the Fc region is prepared by contacting the antibody with an affinity peptide configured to deliver a protected version of the sulfhydryl group (e.g., a thioester or disulfide) to the lysine residue. An exemplary peptide capable of performing this reaction is shown below (SEQ ID NO: 375), as reported in Matsuda et al., Mol. Pharmaceutics 2021, 18, 4058-4066, which selectively attached the sulfhydryl group via the NHS ester at residue K248 of the Fc region of the antibody:

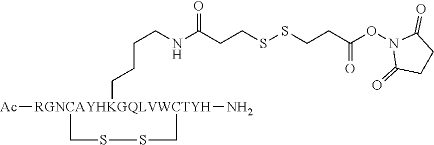

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide an sulfhydryl protecting group (e.g., the relevant portion of the affinity peptide would have a structure of

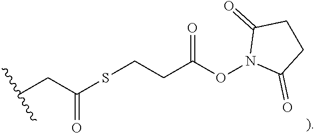 [SMILES: *CC(=O)SCCC(=O)ON1C(=O)CCC1=O]

Such alternative affinity peptides include those described in, for example “AJICAP Second Generation: Improved Chemical Site-Specific Conjugation Technology for Antibody-Drug Conjugation Technology for Antibody-Drug Conjugate Production” (Bioconjugate Chem. 2023, 34, 4, 728-738). Exemplary affinity peptides provided therein include those shown below, wherein the left structure (SEQ ID NO: 376) targets K248 of the Fc region and the right structure (SEQ ID NO: 377) targets K288 of the Fc region (EU numbering).

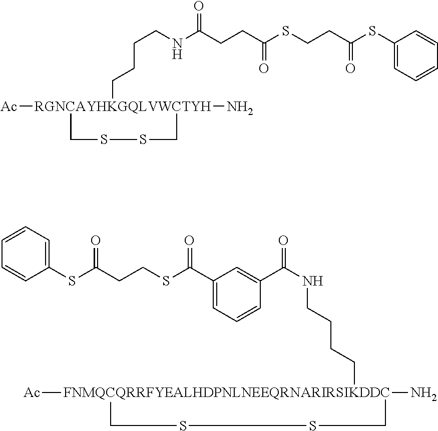

The protecting group (e.g., the disulfide or thioester) is then removed to reveal the free sulfhydryl (e.g., by reduction of a disulfide with TCEP or hydrolysis). The free sulfhydryl is then reacted with a bifunctional reagent comprising a bromoacetamide group (or other suitable group, such as a maleimide) connected to the DBCO conjugation handle through a linking group (e.g., bromoacetamido-PEGx-amido-DBCO, bromoacetamido-DBCO, maleimido-PEGx-amido-DBCO, maleimido-DBCO, p-(2-bromoacetyl)benzoyl-PEGx-amido-DBCO, p-(2-bromoacetyl)benzoyl-DBCO, etc.). In particular, the bifunctional reagent

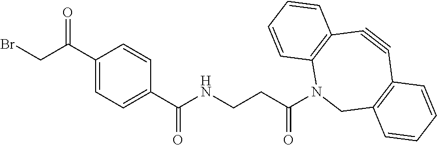 [SMILES: O=C(CBr)c1ccc(C(=O)NCCC(=O)N2Cc3ccccc3C#Cc3ccccc32)cc1]

is used to prepare the constructs provided herein. The method can be used to produce an antibody with one DBCO group present (DAR1) and/or two DBCO groups attached to the antibody (DAR2, one DBCO group linked to each Fc of the antibody). The desired azide modified IL-7 polypeptide (e.g., CMP-107 or CMP-108) is then reacted with the DBCO modified antibody to produce the immunocytokine.

In another embodiment, antibody comprising a single DBCO conjugation handle is prepared by first reacting excess antibody with appropriately loaded affinity peptide to introduce a single sulfhydryl after appropriate removal of protecting group (e.g., disulfide reduction or thioester cleavage). A bifunctional linking group with a sulfhydryl reactive conjugation handle and DBCO conjugation handle (e.g., bromoacetamido-PEGx-amido-DBCO, bromoacetamido-DBCO, maleimido-PEGx-amido-DBCO, maleimido-DBCO, p-(2-bromoacetyl)benzoyl-PEGx-amido-DBCO, p-(2-bromoacetyl)benzoyl-DBCO, etc.) is then reacted with the single sulfhydryl to produce the single DBCO containing antibody. In the exemplified constructs provided herein, the bifunctional linking reagent used is

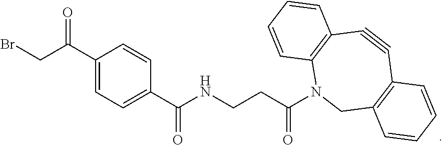 [SMILES: O=C(CBr)c1ccc(C(=O)NCCC(=O)N2Cc3ccccc3C#Cc3ccccc32)cc1]

The single DBCO containing antibody is then conjugated with a suitable azide containing IL-7 (e.g., CMP-108) to achieve an anti-PD-1-IL-7 immunoconjugate with a DAR of 1.

### Conjugation of Antibody to IL-7 Polypeptide

The DBCO modified antibody is then conjugated to an IL-7 polypeptide comprising an azide moiety at a desired point of attachment (e.g., CMP-107). DBCO modified antibody with one (DAR1) or two (DAR2) reactive handles are reacted with 2-10 equivalents of azide containing IL-7 (pH 5.2 buffer, 5% trehalose, rt, 24 h). In an alternative embodiment, antibody comprising two DBCO conjugation handles is reacted either as an excess reagent (e.g., 5-10 equivalents) with 1 equivalent of IL-7 polypeptide comprising an azide functionality (e.g., CMP-107, CMP-108, CMP-109, CMP-110, or CMP-111) to produce a DAR1 antibody or the antibody comprising two DBCO conjugation handles is reacted with 1 equivalent of antibody with excess reagent (e.g., 5-10 equivalents) of IL-7 polypeptide comprising an azide (e.g., CMP-107, CMP-108, CMP-109, CMP-110, or CMP-111) to produce a DAR2 antibody.

Conjugatable variants of anti-PD-1 antibody with one (DAR1) or two (DAR2) reactive handles are reacted with 1 equivalent, 2-10 equivalents, or 5-10 equivalents of a capped mAB (pH 5.2 buffer, 5% trehalose, rt, 24 h). The resulting conjugate is purified by cation-exchange chromatography and/or size exclusion chromatography approximately 50-60% yield.

The anti-PD-1 antibody-IL7 conjugate is purified from unreacted starting product and aggregates using a desalting column, CIEX and SEC (GE Healthcare Life Sciences AKTA pure, mobile phase: Histidine 5.2/150 mM NaCl/5% Trehalose, column: GE Healthcare Life Sciences SUPERDEX™ 200 increase 3.2/300, flow rate: 0.5 mL/min).

The purity and identity of the conjugate is confirmed by RP-HPLC (HPLC: ThermoFisher Scientific UHPLC Ultimate 3000, column: Waters BEH C-4 300A, 3.0 μm, 4.6 mm, 250 mm, mobile phase A: 0.05% TFA in Water, mobile phase B: 0.05% TFA in mixture of ACN:IPA:ETOH:H₂O (5:1.5:2:1.5), flow rate: 0.5 mL/min, injection amount: 10 μg (10 μL Injection of 1 mg/mL), gradient: 0% to 20% mobile phase B in 50 min) and SDS-PAGE.

The above described methods and variations thereof were used to prepare the following variants set forth in the Table 8 below.

TABLE 8

| | | | | Point of |
|---|---|---|---|---|
| | | | | Attachment (Fc |
| Composition | | IL-7 | | region, EU |
| Number | Antibody | Polypeptide | DAR | numbering) |
| CMP-039 | Pembrolizumab | CMP-108 | 1 | K248 |
| CMP-040 | Pembrolizumab | CMP-108 | 2 | K248 |
| CMP-116 | LZM-009 | CMP-107 | 1 | K248 |
| | | (V15W) | | |
| CMP-118 | LZM-009 | CMP-107 | 2 | K248 |
| | | (V15W) | | |
| CMP-041 | LZM-009 | CMP-108 | 1 | K248 |
| CMP-119 | LZM-009 | CMP-108 | 2 | K248 |

The above methods and variations thereof are also used to prepare the conjugates in Table 9.

TABLE 9

| | | | | Point of |
|---|---|---|---|---|
| | | | | Attachment |
| Composition | | | | (Fc region, |
| Number | Antibody | IL-7 Polypeptide | DAR | EU numbering) |
| CMP-120 | LZM-009 | CMP-109 (K81L6P) | 1 | K248 |
| CMP-121 | LZM-009 | CMP-109 (K81L6P) | 2 | K248 |
| CMP-122 | LZM-009 | CMP-110 (G85N) | 1 | K248 |
| CMP-123 | LZM-009 | CMP-110 (G85N) | 2 | K248 |
| CMP-124 | LZM-009 | CMP-111 (K81L6P | 1 | K248 |
| | | and G85N) | | |
| CMP-125 | LZM-009 | CMP-111 (K81L6P | 2 | K248 |
| | | and G85N) | | |

### Example 7: ELISA Assays with PD-1 Antibodies and Conjugates to IL-7

Synthetic IL-7 (CMP-108) conjugated to Pembrolizumab with DAR1 (CMP-039) and DAR2 (CMP-040) and LZM009 with DAR1 (CMP-041) were prepared using methods provided above. Synthetic IL-7 having the V15W substitution which reduces affinity to the IL-7 receptor (CMP-107) was also conjugated to LZM-009 to produce an immunocytokine with DAR of 1 (CMP-116). Each of these variant conjugates and unmodified antibodies are assayed by ELISA for their ability to bind to human PD-1 according to the following protocol.

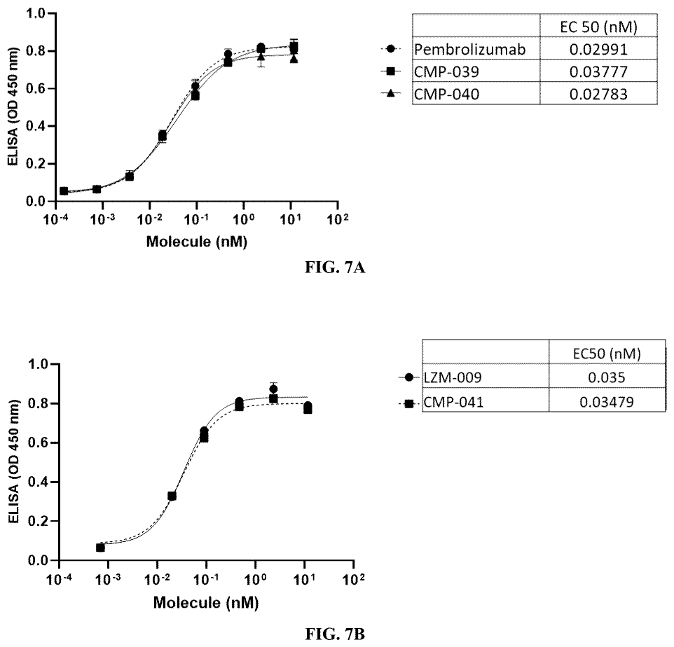

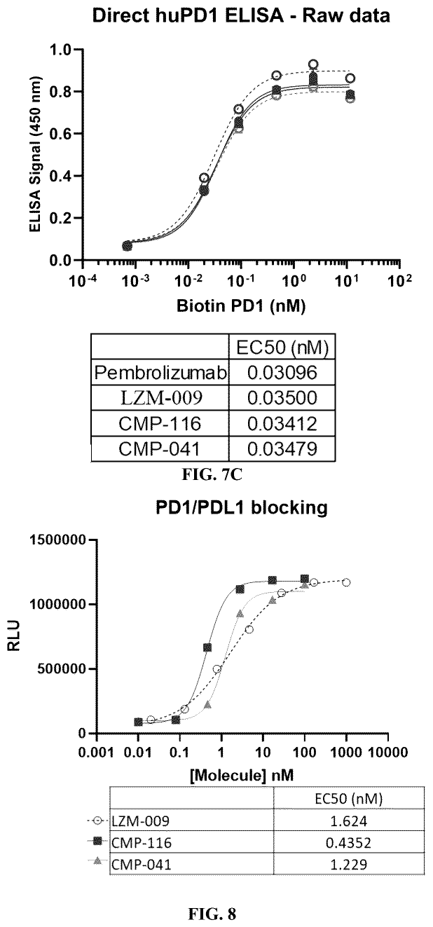

Materials: ELISA plate are Costar Assay plate 96 well clear Flat bottom Half Area High binding Polystyrene, CORNING #3690. Biotinylated Recombinant Human PD-1 is (CD279)-Fc Chimera (carrier-free), Biolegend #789406. Streptavidin-HRP is Sigma #RABHRP3. TMB solution is 3,3′,5,5′-Tetramethylbenzidine (Sigma T0440). Stop solution is Sigma #CL07STOP solution (0.5M H₂SO₄). Buffers are: Coating buffer is PBS. Wash buffer is PBS-0.02% Tween20. Blocking buffer is PBS-0.02% Tween20 1% BSA. Protein diluent is PBS-0.02% Tween20 0.1% BSA. STOP solution is 0.5M H₂SO₄. Procedure: Immunocytokines and parental antibody are coated overnight at 4° C. The ELISA plates are washed 4 times with 100 μl PBS-0.02% Tween20. and blocked with PBS-1% BSA. A serial dilution of h-PD1 Fc is prepared and plates are incubated for two hours at 37° C., with shaking (600 rpm). ELISA plates are washed and incubated with Streptavidin-HRP 30 min at RT, with shaking (600 rpm). ELISA plates are washed and incubated with ready-to-use TMB solution. Reaction is stopped and plates are read by OD450 on an Enspire plate reader. The results from this experiment are shown in FIG. 7A, which shows that ability of the pembrolizumab DAR1 (CMP-039) and DAR2 (CMP-040) immunocytokine to bind PD-1 after conjugation of IL7 is not substantially altered compared to unmodified antibody. Similarly, conjugation of IL7 to anti-PD-1 antibody LZM-009 (CMP-041) also did not substantially affect binding to PD-1 as shown in FIG. 7B and FIG. 7C. Similarly, use of an IL-7 with reduced binding affinity for the IL-7 receptor also did not impact binding of the immunocytokine to PD-1 (see CMP-116 in FIG. 7C).

### Example 8: PD-1/PD-L1 Blockade Assay

Using the PD1/PDL1 blocking assay from Invivogen (Cat No: rajkt-hpd1), the ability to still block the PD1/PDL1 interaction after conjugation of IL-7 was assessed using a PD-1/PD-L1 blockade assay. This was accomplished according to the protocol provided by Invivogen (Cat No: rajkt-hpd1) below.

Materials were Flat 96 well, Corning #3596 culture plates. Read out plates were flat white 96 well plates, ThermoScientific #136102. Luciferase substrate assay solution was QUANTI-Luc (Invivogen #rep-qlc). Test cells were Jurkat-Lucia™ TCR-hPD-1 cells, (Invivogen #rajkt-hpd1). Target cells are Raji-APC-hPD-L1 cells (Invivogen #rajkt-hpd1).

For the assay procedure, the parental antibody, cytokine conjugates of LZM-009 to IL-7 (V15W) CMP-016 or IL-7 (WT) (CMP-041) were diluted from a top concentration of 1 μM in assay medium. A total of seven dilution steps were made by diluting the next higher antibody concentration 1:6. For each well 20 μl of molecules dilution was added, according to the assay layout. As a negative control, 20 μl of assay media containing no antibody or immunoconjugate was used.

Jurkat-Lucia™ TCR-hPD-1 cells and Raji-APC-hPD-L1 cells were seeded and treated with a dose titration of the test molecules. After 24 hrs sample supernatants were analyzed for luciferase activity by adding 50 μl of QUANTI-Luc. The bioluminescence was measured immediately after the addition of the luciferase substrate using an Enspire plate reader. The results of this experiment comparing LZM-009, CMP-016 and CMP-041 are shown in FIG. 8. The two LZM-009 cytokine conjugates and LZM-009 exhibited similar ability to block the interaction of PD-1 and PD-L1.

### Example 9: FcRN Binding Assay

The ability of LZM-009 and two IL-7 polypeptide conjugates of LZM-009 to bind human and mouse FcRN was determined using an AlphaLISA assay according to the below protocol. Samples were assayed using AlphaLISA kit from PerkinElmer, cat #AL3095C. LZM-009 CMP-116 and CMP-041 were serially diluted. Human FcRn (4× concentrated) was diluted to a final concentration of 50 ng/ml in 1×MES buffer. Human IgG Conjugated Acceptor Beads (2×) and Streptavidin (SA) Donor Beads were diluted to reach final concentrations of 5 μg/ml.

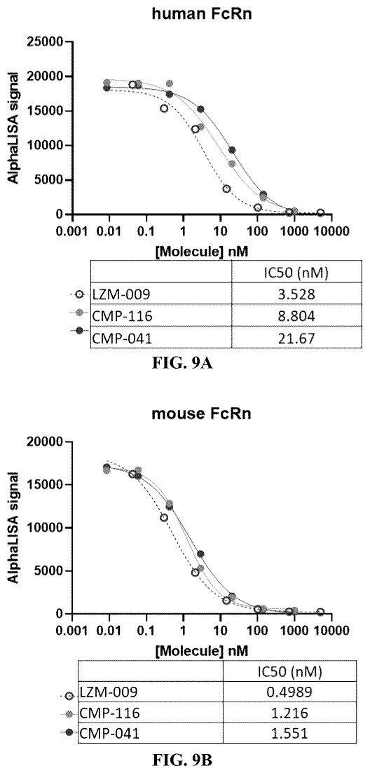

To wells in the assay plates, 10 μl of diluted antibodies, 10 μl of diluted FcRn, 20 μl of human IgG conjugated Acceptor beads, and SA-Donor beads were added. Plates were incubated for 90 minutes at room temperature then measured using 680 nm excitation and 615 nm emission on a plate reader with the appropriate capabilities. The results of this assay are shown in FIG. 9A for human and in FIG. 9B for binding to mouse FcRn. The antibody LZM-009 and cytokine conjugates to LZM-009 (CMP-116 [IL-7, (V15W)] and CMP-041 [IL-7, WT]) demonstrated similar binding affinity for human and mouse FcRn in the assay.

### Example 10: In Vivo Tumor Growth Inhibition

An experiment was performed as described below in order to assess the anti-tumor properties of LZM-009 and IL-7 conjugates to LZM-009: CMP-041 (WT) and CMP-116 (V15W), Transgenic BALBc-hPD1 mice (BALB/cJGpt-Pdcdlem1Cin (hPDCD1)/Gpt), which were genetically modified with knock-in of human PD-1 expression constructs (GemPharmatech (Cat #T002726)). Cells were implanted in mice with the syngeneic CT26 tumor model and treated with LZM-009 (10 mg/kg, once per week), CMP-041 (1,3 and 10 mg/kg, once per week) CMP-116 (3 mg/kg, once per week).

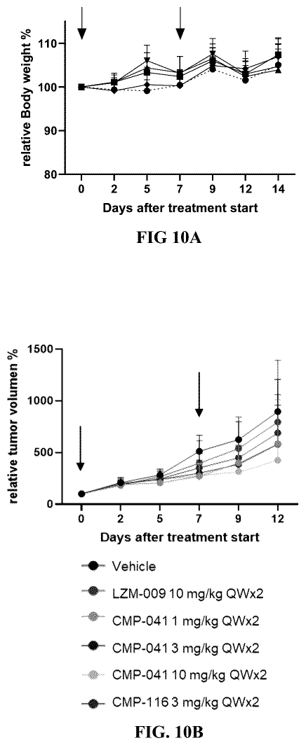

Body weight measurements and relative tumor volume for the various groups during the course of the study are shown in FIGS. 10A and 10B, respectively. As shown in FIG. 10A none of the dose levels of CMP-041 or CMP-116 induced any body weight loss indicating favorable safety profiles. As shown in FIG. 10B CMP-041 and CMP-116 induce a dose dependent tumor growth inhibition that were superior to LZM-009. In summary the in vivo data provided show that CMP-041 and CMP-116 are safe, effective, and superior to the parental antibody.

### Example 11: Recombinant IL-7 Expression and Purification

The following general protocol or variations thereof can be used to express recombinant IL-7 and recombinant IL-7 variants provided herein. E. coli strain BL21 (DE3) harboring plasmid expression constructs of an IL-7 variant with a c-terminal HIS6 tag (SEQ ID NO: 374) are inoculated into 3 L LB culture medium and induced with 0.4 mM IPTG at 30° C. for 6 h. Cells are pelleted by centrifugation. Cell lysis is accomplished by sonication in lysis buffer: PBS, pH 7.4. Soluble protein is purified by passing clarified lysates over Ni-NTA beads 6FF then wash with 10 column volumes of Phosphate Buffered Saline (PBS) containing, 20 mM imidazole, pH7.4; then wash with 10 column volumes PBS containing 50 mM Imidazole, pH7.4; then elute with PBS containing 500 mM imidazole, pH7.4. Fractions are evaluated for protein content and purity by SDS-PAGE gel analysis. High purity peak fractions are pooled and further purified on a Superdex S100 10×300 column that is equilibrated with PBS elution buffer. Fractions are evaluated for protein content and purity by SDS-PAGE gel analysis. Peak fractions containing high purity protein are pooled and concentrated. Samples are analyzed for purity by HPLC-SEC and LC-MS assays.

### Example 12: pSTAT5 Assay

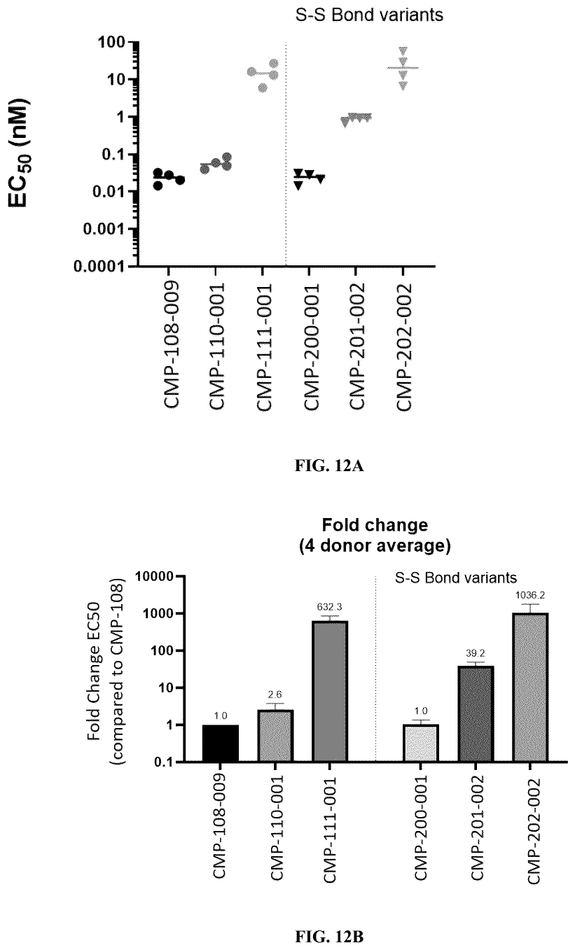

Materials and Method: Frozen pan T cells were thawed and cultured overnight in RPMI 10% FCS, 1% Glutamine, 1% NEAA, 25 μM 2BME, 1% NaPyrovate at 37° C., 5% CO₂, 95% humidity. The next day cells were washed and resuspended in PBS. Cells were stimulated with serial dilutions of 117 for 40 minutes at 37° C., 5% CO₂, 95% humidity followed by cell fixation and permeabilization with Transcription Factor Phospho Buffer Set (BD Biosciences). Then, cells were stained for 1 hour on ice with antibodies detecting human Phospho-Stat5-PE (Tyr694; 1:50, clone 47/Stat5pY694), CD25-BV421 (1:100, clone M-A251), CD45RA-BV711 (1:100, clone HI100), CD4-FITC (1:400, clone RPA-T4), CD8-APC/Cy7 (1:100, clone SK1), FOXP3-AF647 (1:50, clone 259D). Then cells were washed twice with ice cold FACS buffer. Data were acquired on a multi-color flow cytometer and data were analyzed with FlowJo software. Ec50s were determined with GraphPadPrism. FIGS. 12A-B: pSTAT5 human pan T cells S—S bond variants. A: EC50 and B: fold change EC50 compared to WT syntein, gated on CD4 memory T cells

### Example 13: CD127 Internalization on Human T Cells

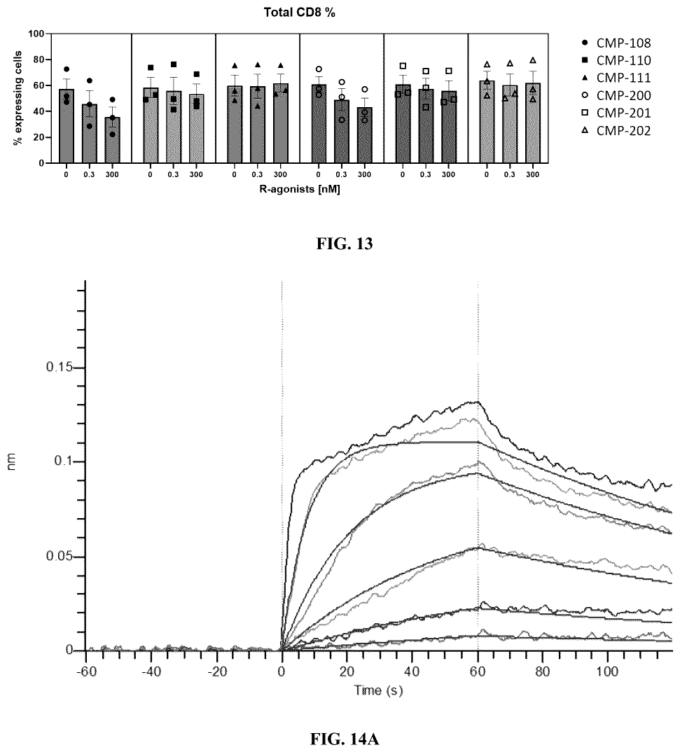

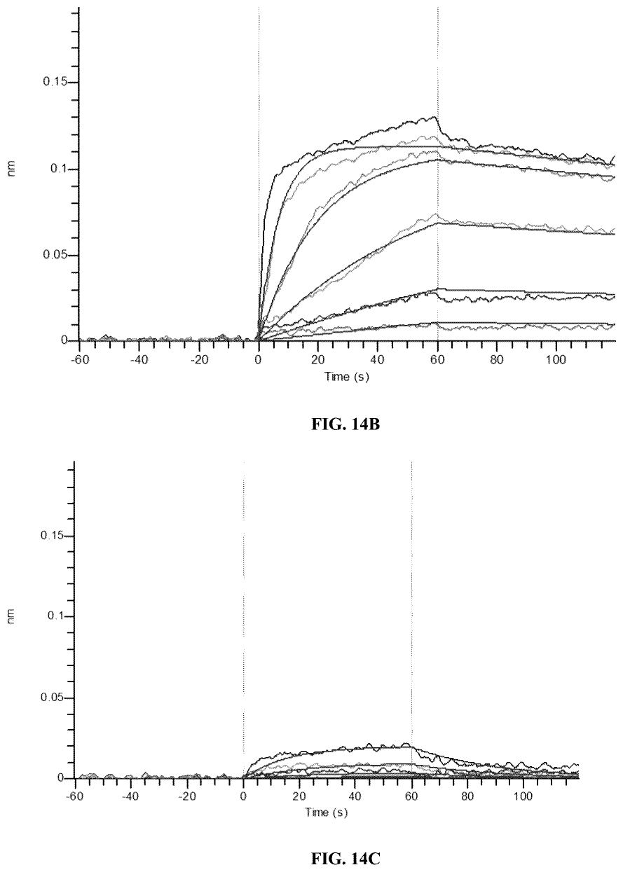

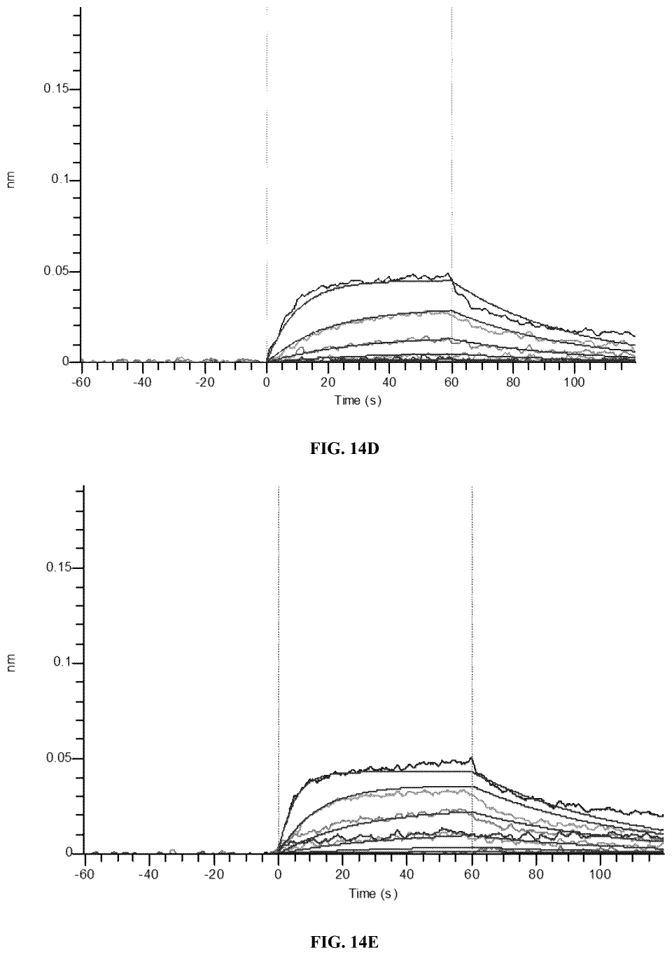

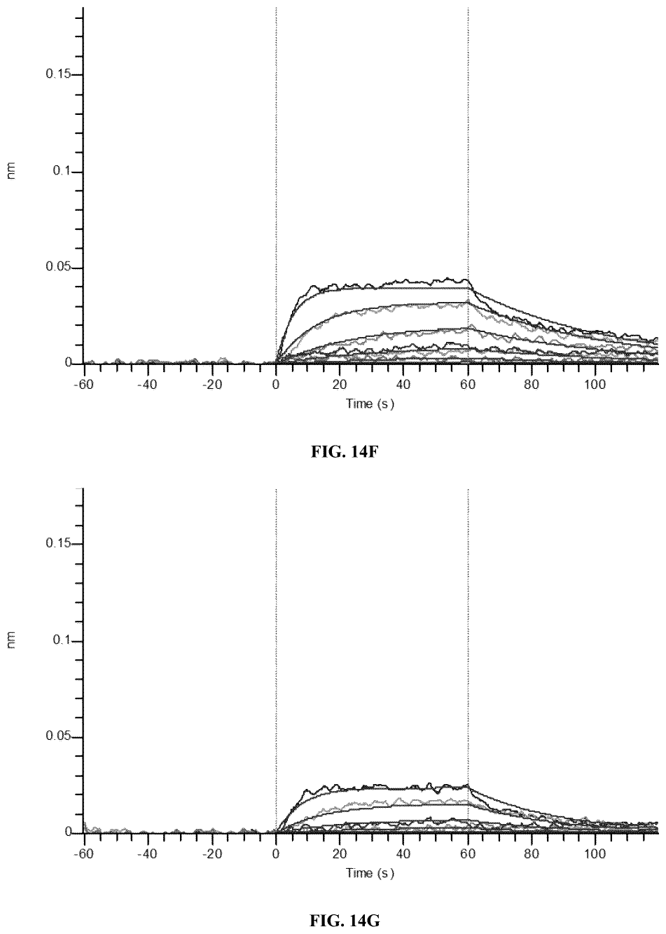

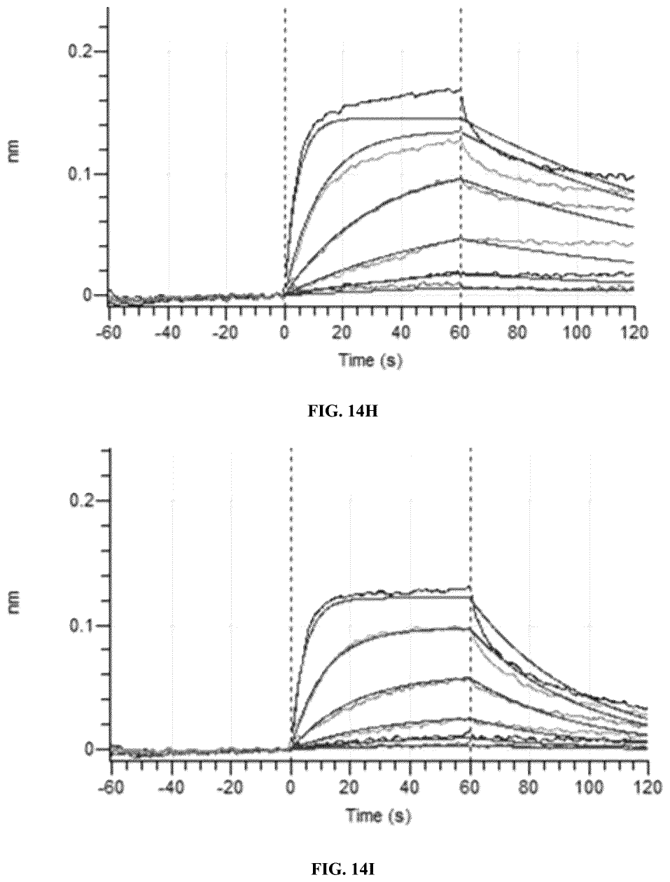

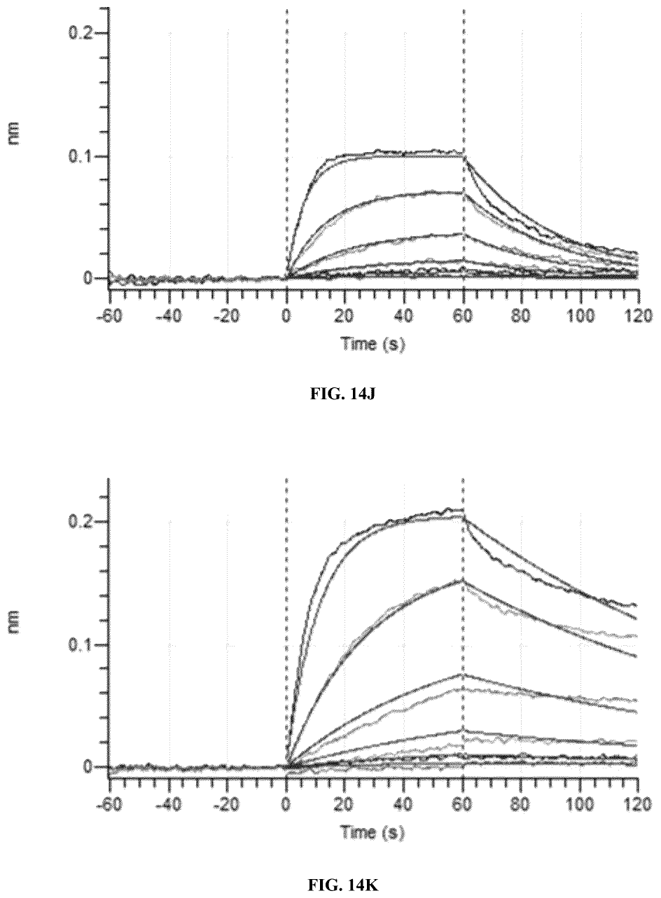

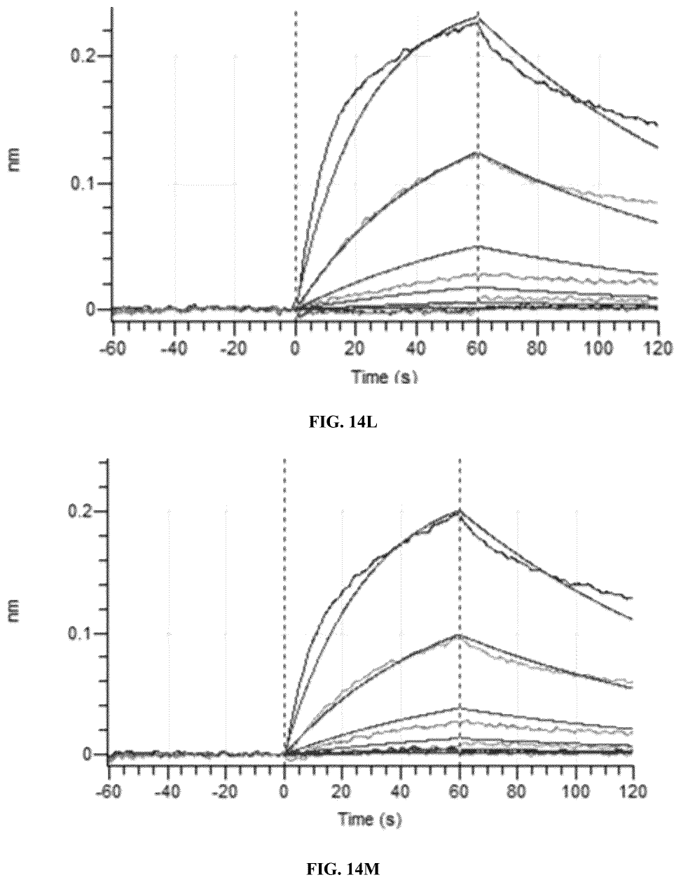

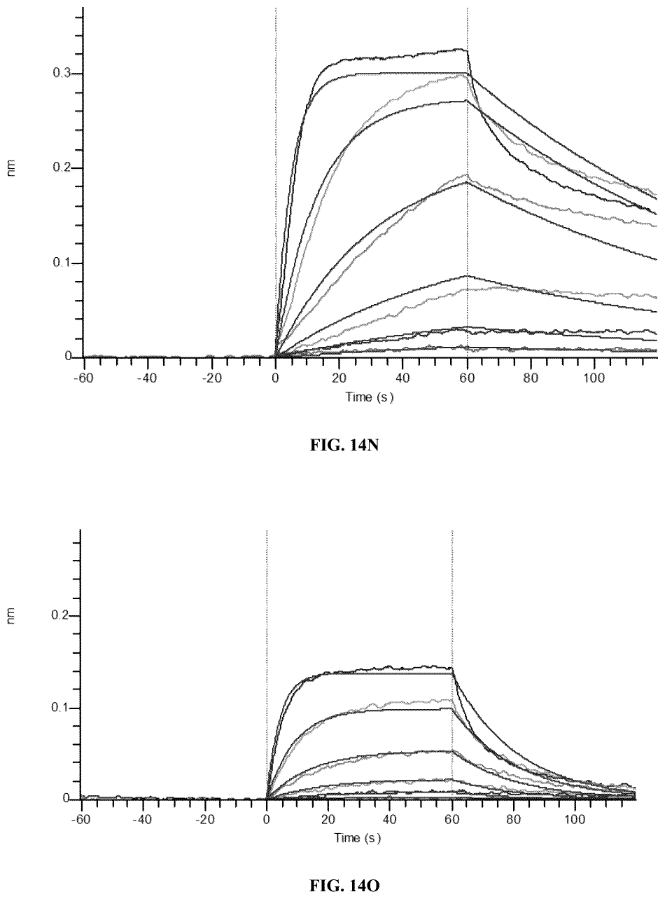

FIG. 13 shows how IL7R is rapidly internalized after its engagement with any wt IL7variant (CMP-108 and CMP-200). Positive CD8 T cells for IL7R are decreasing with increasing concentrations of wt IL7 (0.3 and 300 nM) when compared to untreated cells (0 nM). On the contrary, internalization is heavily impaired if T cells are stimulated with IL7 mutants (CMP-110 and CMP-111). The absence of S—S bonds in variants CMP-200, CMP-201 and CMP-202 does not influence the results obtained with the variants containing all the S—S bonds.

Protocol: Frozen pan T cells were thawed and cultured overnight in RPMI 10% FCS, 1% Glutamine, 1% NEAA, 25 μM 2BME, 1% NaPyrovate at 37° C., 5% CO₂, 95% humidity. The next day cells were washed and resuspended in FACS buffer. Cells were stimulated with serial dilutions of IL-7 for 40 minutes at 37° C., 5% CO₂, 95% humidity. After, cells are washed with ice cold FACS buffer and stained for 30 minutes on ice with antibodies detecting human CD4 (1:400, clone RPA-T4), CD8 (1:100, clone SK1), CD45RA (1:100, clone HI100) and CD127 (1:100, clone hIL-7R-M21, that has been reported to be non-competitive). Then cells were washed twice with ice cold FACS buffer. Data were acquired on a multi-color flow cytometer and data were analyzed with FlowJo software. Percentage of positive CD8 T cells for CD127 is shown in FIG. 13.

### Example 14: Biolayer Interferometry (BLI)-Binding to CD127

### Materials

Assay plate: Microplate, 96 well, PP, F-bottom (chimney well) Black, Greiner Bio-one #655209

Biosensors: Octet SAX2 Biosensors, Sartorius #18-5136 or Octet SAX Biosensors, Sartorius #18-5117

Assay Buffer: Kinetic buffer 10×, Sartorius #18-1105 or prepared inhouse: PBS+0.02% Tween20+0.1% BSA

Regeneration buffer: 10 mM Glycine pH 2.7

CD127 Recombinant Human IL-7R alpha/CD127 Fc Avi-tag Protein, CF, R&D Systems #AVI10317-050

Instrument: Octet R8, Sartorius

### Preparation of the Ligands:

Receptors were loaded on SAX2 sensors (CD127)

### Preparation of the Molecules (Analytes):

Dilute analytes to 200 nM (highest concentration) and make 1:3 serial dilutions down the column (7 dilution points+zero) in Kinetic buffer 1×.

### Measurement:

Follow manufacture's recommendation.

The CD127 binding parameters of various IL-7 polypeptides are shown in FIGS. 13A, and in the Tables below.

CD127 binding parameters of CMP-035

| CMP-035 | |
|---|---|
| KD (M) | 1.28E−09 |
| ka (1/Ms) | 5.47E+06 |
| kdis (1/s) | 0.00698359 |

CD127 binding parameters of CMP-108

| CMP-108 | |
|---|---|
| KD (M) | 2.54E−10 |
| ka (1/Ms) | 6.59E+06 |
| kdis (1/s) | 0.0016738 |

CD127 binding parameters of CMP-106

| CMP-106 | |
|---|---|
| KD (M) | 6.63E−08 |
| ka (1/Ms) | 4.85E+05 |
| kdis (1/s) | 0.032137993 |

CD127 binding parameters of CMP-107

| CMP-107 | |
|---|---|
| KD (M) | 2.25E−08 |
| ka (1/Ms) | 1.15E+06 |
| kdis (1/s) | 0.025935746 |

CD127 binding parameters of CMP-109

| CMP-109 | |
|---|---|
| KD (nM) | 7.45E−09 |
| ka (1/Ms) | 2.81E+06 |
| kdis (1/s) | 0.020948973 |

CD127 binding parameters of CMP-110

| CMP-110 | |
|---|---|
| KD (nM) | 8.94E−09 |
| ka (1/Ms) | 2.51E+06 |
| kdis (1/s) | 0.02248398 |

CD127 binding parameters of CMP-111

| CMP-111 | |
|---|---|
| KD (nM) | 2.51E−08 |
| ka (1/Ms) | 1.41E+06 |
| kdis (1/s) | 0.035336151 |

| CMP-108-009 | |
|---|---|
| KD (nM) | 2.75 |
| ka (1/Ms) | 3275894 |
| kdis (1/s) | 0.009007 |

CD127 binding parameters of CMP-110-001

| CMP-110-001 | |
|---|---|
| KD (nM) | 9.47 |
| ka (1/Ms) | 2838297 |
| kdis (1/s) | 0.026877 |

CD127 binding parameters of CMP-111-001

| CMP-111-001 | |
|---|---|
| KD (nM) | 16.53 |
| ka (1/Ms) | 1924914 |
| kdis (1/s) | 0.031827 |

CD127 binding parameters of CMP-200-001

| CMP-200-001 | |
|---|---|
| KD (nM) | 7.18 |
| ka (1/Ms) | 1224184 |
| kdis (1/s) | 0.00879 |

CD127 binding parameters of CMP-201-002

| CMP-201-002 | |
|---|---|
| KD (nM) | 19.21 |
| ka (1/Ms) | 518420 |
| kdis (1/s) | 0.009959 |

CD127 binding parameters of CMP-202-002

| CMP-202-002 | |
|---|---|
| KD (nM) | 25.38 |
| ka (1/Ms) | 392723.8 |
| kdis (1/s) | 0.009966 |

CD127 binding parameters of CMP-041

| CMP-041 | |
|---|---|
| KD (nM) | 3.31E−09 |
| ka (1/Ms) | 2.95E+06 |
| kdis (1/s) | 0.009787293 |

CD127 binding parameters of CMP-110

| CMP-116 | |
|---|---|
| KD (nM) | 1.63E−08 |
| ka (1/Ms) | 2.90E+06 |
| kdis (1/s) | 0.047336268 |

TABLE 10
Additional IL-7 polypeptides as provided herein are described in the table below.

| | CMP | |
|---|---|---|
| | # | |
| | (SEQ | |
| | ID | |
| Ex. | NO) | Sequence |
| 2A | 036 | |
| | (SEQ | |
| | ID | |
| | NO: | |
| | 3) | |
| 2B | 107 (SEQ ID NO: 5) | 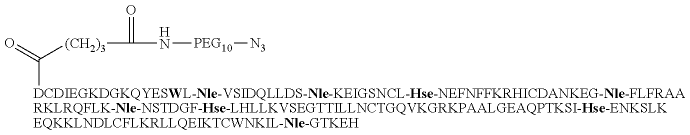 |
| 2C | 203 (SEQ ID NO: 5) | 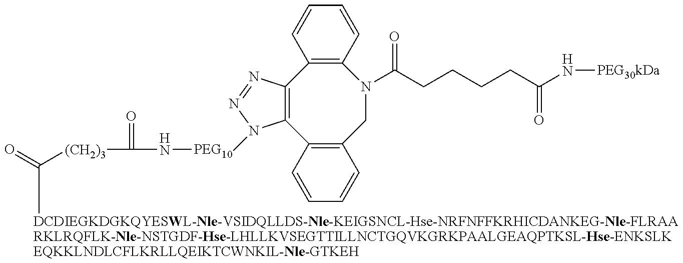 |
| 2D | 108 (SEQ ID NO: 3) | 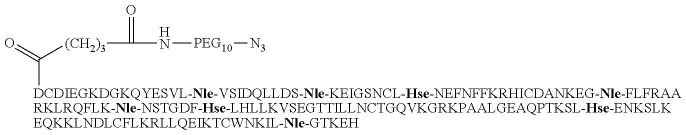 |
| 2E | 114 (SEQ ID NO: 5) | 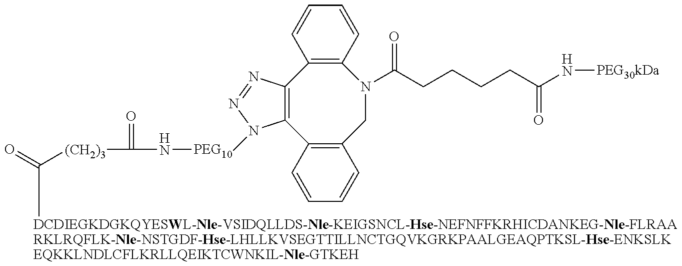 |
| 2F | 204 (SEQ ID NO: 378) | 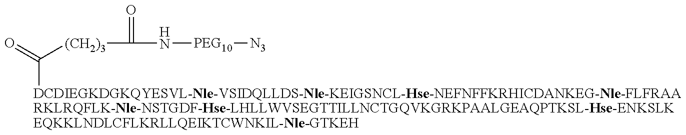 |
| 2G | 115 (SEQ ID NO: 12) | 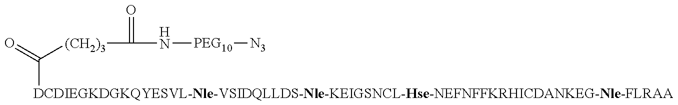 |
| | | 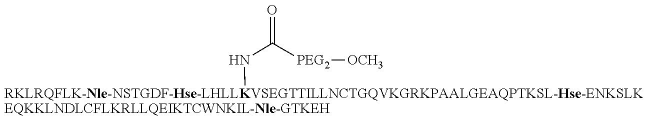 |
| 2H | 109 (SEQ ID NO: 6) | 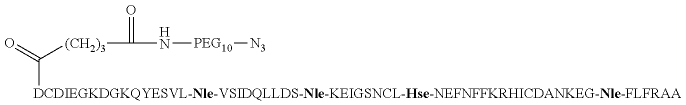 |
| | | 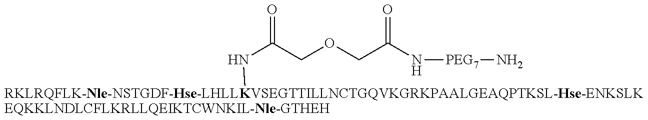 |
| 2I | 110 | |
| | (SEQ | |
| | ID | |
| | NO: | |
| | 7) | |
| 2J | 112 (SEQ ID NO: 9) | 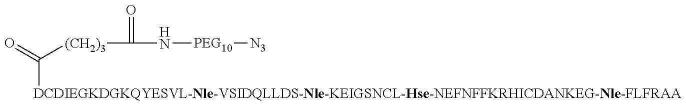 |
| | | 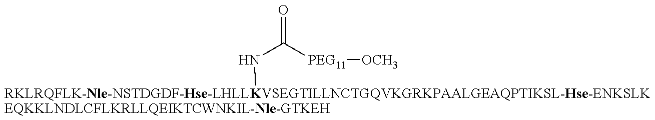 |
| 2K | 205 (SEQ ID NO: 379) | 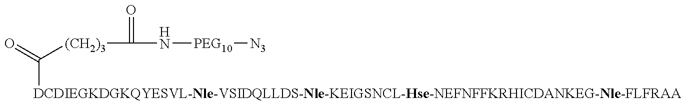 |
| | | 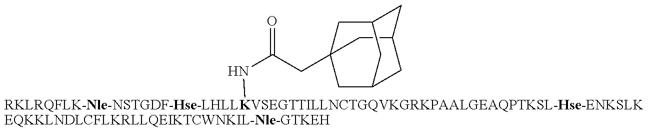 |
| 2L | 111 (SEQ ID NO: 8) | 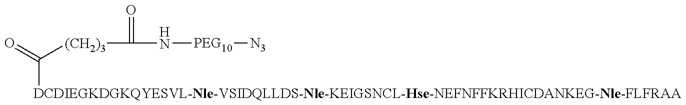 |
| | | 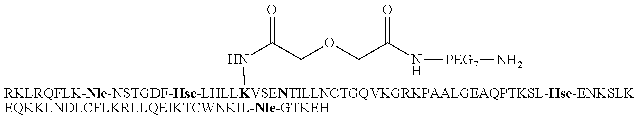 |
| 2M | 113 (SEQ ID NO: 10) | 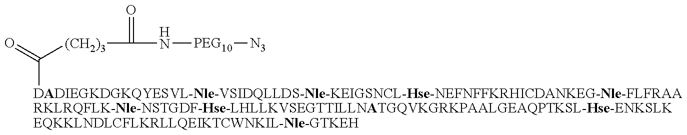 |
| 2N | 206 (SEQ ID NO: 380) | 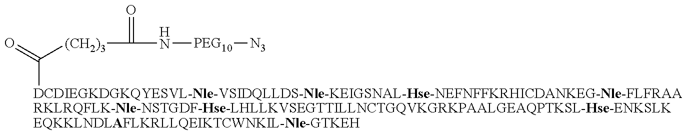 |
| 2O | 207 (SEQ ID NO: 381) | 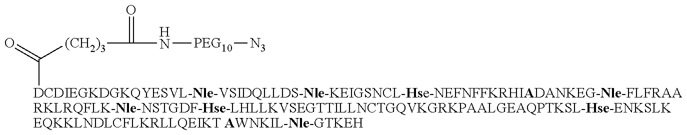 |
| 2P | 200 (SEQ ID NO: 382) | 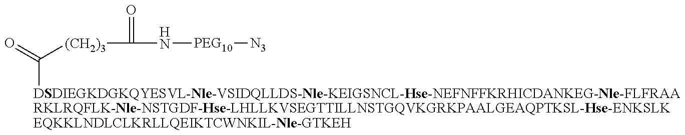 |
| 2Q | 208 (SEQ ID NO: 383) | 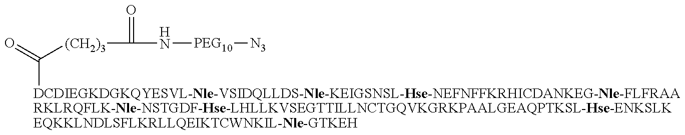 |
| 2R | 209 (SEQ ID NO: 384) | 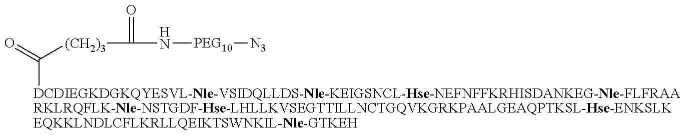 |
| 2S | 210 (SEQ ID NO: 385) | 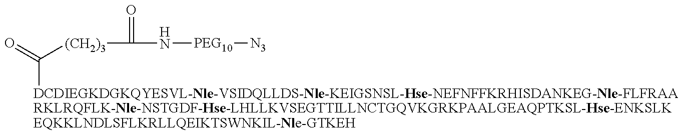 |
| 2T | 211 (SEQ ID NO: 386) | 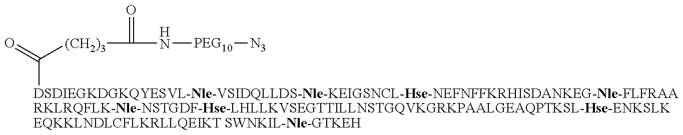 |
| 2U | 212 (SEQ ID NO: 387) | 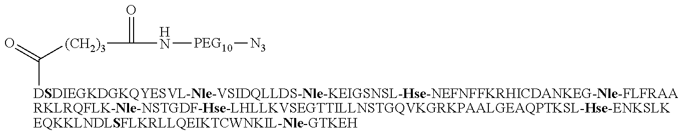 |
| 2V | 201 (SEQ ID NO: 388) | 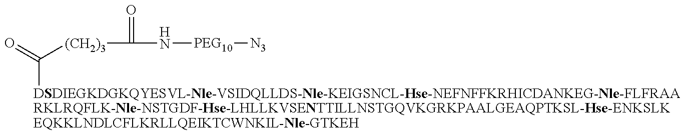 |
| 2W | 202 (SEQ ID NO: 389) | 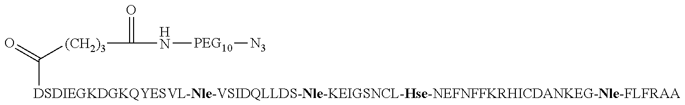 |
| | | 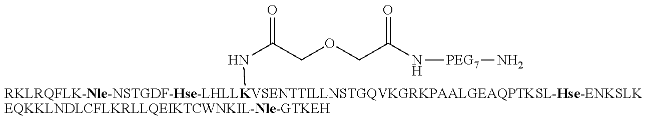 |
| 2X | 213 (SEQ ID NO: 390) | 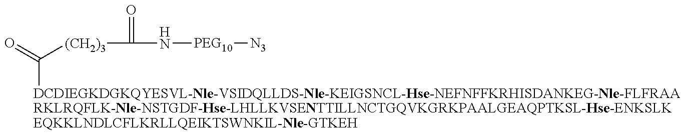 |
| 2Y | 214 (SEQ ID NO: 391) | 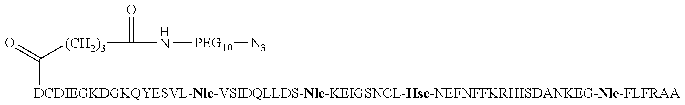 |
| | | 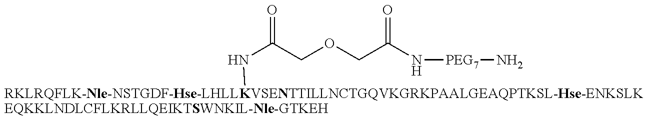 |

1. An IL-7 polypeptide comprising:
at least one modification to the amino acid sequence as set forth in SEQ ID NO: 1, wherein the at least one modification is a natural amino acid substitution or an additional group covalently attached to a side chain of an amino acid residue of the IL-7 polypeptide; and
wherein the IL-7 polypeptide is synthetic.

2. (canceled)

3. (canceled)

4. The IL-7 polypeptide claim 2, wherein the IL-7 polypeptide comprises Hse residues at each of positions 36, 76, and 114.

5. (canceled)

6. (canceled)

7. (canceled)

8. The IL-7 polypeptide of claim 1, comprising Nle substitutions at each of residues M17, M27, M54, M69, and M147.

9. (canceled)

10. The IL-7 polypeptide of claim 1, wherein the modification comprises an amino acid substitution selected from C2S, C2A, K7A, Q11F, S14H, V15A, V15W, V18A, C34S, C34A, N36S, C47S, C47A, N70K, N70Y, S71N, S71R, S71V, T72H, T72N, T72W, D74A, D74G, D74N, D74Q, D74W, D76S, L77A, L77D, L77E, L77H, L77Q, L77T, L77V, H78A, H78R, H78Y, L79A, L80K, L80Q, L80W, K81A, K81E, K81M, K81Q, K81W, E84F, E84N, E84R, E84W, E84Y, G85A, G85N, G85Q, G85W, 188A, 188D, 188E, 188F, 188R, 188T, C92S, C92A, E114S, C129S, C129A, C141S, and C141A.

11. (canceled)

12. (canceled)

13. The IL-7 polypeptide of claim 1, wherein the IL-7 polypeptide comprises a V15W amino acid substitution.

14. The IL-7 polypeptide of claim 1, wherein the IL-7 polypeptide comprises a G85N amino acid substitution.

15. The IL-7 polypeptide of claim 1, wherein the IL-7 polypeptide comprises the additional group covalently attached to the side chain of the amino acid residue of the IL-7 polypeptide.

16. The IL-7 polypeptide of claim 15, wherein the additional group is covalently attached to the amino acid residue at position 81 of the IL-7 polypeptide.

17. (canceled)

18. (canceled)

19. (canceled)

20. The IL-7 polypeptide of claim 15, wherein the additional group comprises a polymer.

21. The IL-7 polypeptide of claim 20, wherein the polymer has a molecule weight of from about 50 Da to about 2000 Da.

22. (canceled)

23. (canceled)

24. (canceled)

25. The IL-7 polypeptide of claim 20, wherein the polymer is polyethylene glycol (PEG).

26. The IL-7 polypeptide of claim 20, wherein the polymer comprises a structure of 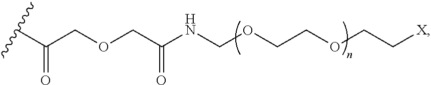
wherein n is an integer from 2-20; and
X is a capping group selected from an amino group, an amido group, an alkoxyl group, and a hydroxyl group.

27. The IL-7 polypeptide of claim 1, comprising a modification or set of modifications selected from:
a) a G85N substitution;
b) a polymer attached to an amino acid residue at position 81 of the IL-7 polypeptide;
c) a G85N substitution and a polymer attached to an amino acid residue at position 81 of the IL-7 polypeptide; and
d) a V15W substitution.

28. The IL-7 polypeptide of claim 1, wherein the modified IL-7 polypeptide has reduced binding affinity to the IL-7 receptor alpha as compared to wild-type IL-7.

29. (canceled)

30. (canceled)

31. (canceled)

32. (canceled)

33. The IL-7 polypeptide of claim 1, comprising a sequence as set forth in any one of SEQ ID NOs: 5-8.

34. (canceled)

35. (canceled)

36. (canceled)

37. (canceled)

38. (canceled)

39. An immunocytokine comprising:
an antibody or antigen binding fragment thereof specific for programmed cell death protein 1 (PD-1);
the IL-7 polypeptide of claim 1; and
a linker, wherein the linker comprises:
a first point of attachment covalently attached to the IL-7 polypeptide; and
a second point of attachment covalently attached to the antibody or antigen binding fragment thereof.

40. The immunocytokine of claim 39, wherein the first point of attachment is at an N-terminal residue of the IL-7 polypeptide.

41. (canceled)

42. The immunocytokine of claim 39, wherein the antibody or antigen binding fragment comprises an Fc region, and wherein the second point of attachment is at lysine residue in the Fc region.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. The immunocytokine of claim 39, wherein the linker comprises a structure 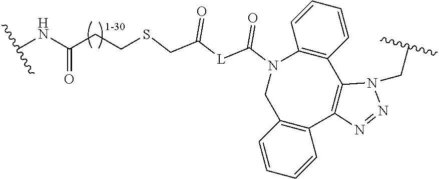
wherein 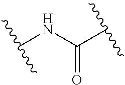 [SMILES: *NC(*)=O]
is the first point of attachment to a lysine residue of the antibody or antigen binding fragment;
L is a linking group; and 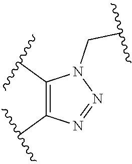 [SMILES: *Cn1nnc(*)c1*]
is a point of attachment to a linking group which connects to the first point of attachment.

59. (canceled)

60. (canceled)

61. (canceled)

62. (canceled)

63. (canceled)

64. (canceled)

65. A method of making an IL-7 polypeptide, comprising:
a) synthesizing two or more fragments of the IL-7 polypeptide;
b) ligating the fragments; and
c) folding the ligated fragments,
wherein the IL-7 polypeptide comprises at least one modification to the amino acid sequence as set forth in SEQ ID NO: 1, wherein the at least one modification is a natural amino acid substitution or an additional group covalently attached to a side chain of an amino acid residue of the IL-7 polypeptide.

66-84. (canceled)


SEQUENCE LISTING (391 sequences)

SEQ ID NO: 1 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 160 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHVDHHHHHH

SEQ ID NO: 3 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNCLXNEFNFFKRHICDANKEGXFLFRAARKLRQFLKXNSTGDFXLHLLKVSEGTTILLNCTGQVKGRKPAALGEAQPTKSLXENKSLKEQKKLNDLCFLKRLLQEIKTCWNKILXGTKEH

SEQ ID NO: 4 - protein, 152 aa, synthetic construct
  SITE 36: note = Homoserine
  SITE 76: note = Homoserine
  SITE 114: note = Homoserine
DCDIEGKDGKQYESVLMVSIDQLLDSMKEIGSNCLXNEFNFFKRHICDANKEGMFLFRAARKLRQFLKMNSTGDFXLHLLKVSEGTTILLNCTGQVKGRKPAALGEAQPTKSLXENKSLKEQKKLNDLCFLKRLLQEIKTCWNKILMGTKEH

SEQ ID NO: 5 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESWLXVSIDQLLDSXKEIGSNCLXNEFNFFKRHICDANKEGXFLFRAARKLRQFLKXNSTGDFXLHLLKVSEGTTILLNCTGQVKGRKPAALGEAQPTKSLXENKSLKEQKKLNDLCFLKRLLQEIKTCWNKILXGTKEH

SEQ ID NO: 6 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 81: note = N-(7x Polyethylene glycol-amino)-L-lysine
  SITE 114: note = Homoserine
  SITE 147: note = Norleucine
DCDIEGKDGKQYESVLXVSIDQLLDSXKEIGSNCLXNEFNFFKRHICDANKEGXFLFRAARKLRQFLKXNSTGDFXLHLLXVSEGTTILLNCTGQVKGRKPAALGEAQPTKSLXENKSLKEQKKLNDLCFLKRLLQEIKTCWNKILXGTKEH

SEQ ID NO: 7 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNCLXNEFNFFKRHICDANKEGXFLFRAARKLRQFLKXNSTGDFXLHLLKVSENTTILLNCTGQVKGRKPAALGEAQPTKSLXENKSLKEQKKLNDLCFLKRLLQEIKTCWNKILXGTKEH

SEQ ID NO: 8 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 81: note = N-(7x Polyethylene glycol-amino)-L-lysine
  SITE 114: note = Homoserine
  SITE 147: note = Norleucine
DCDIEGKDGKQYESVLXVSIDQLLDSXKEIGSNCLXNEFNFFKRHICDANKEGXFLFRAARKLRQFLKXNSTGDFXLHLLXVSENTTILLNCTGQVKGRKPAALGEAQPTKSLXENKSLKEQKKLNDLCFLKRLLQEIKTCWNKILXGTKEH

SEQ ID NO: 9 - protein, 152 aa, synthetic construct; identical to SEQ ID NO: 6
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 81: note = N-(11x Polyethylene glycol-amino-L-lysine)
  SITE 114: note = Homoserine
  SITE 147: note = Norleucine

SEQ ID NO: 10 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DADIEGKDGKQYESVLXVSIDQLLDSXKEIGSNCLXNEFNFFKRHICDANKEGXFLFRAARKLRQFLKXNSTGDFXLHLLKVSEGTTILLNATGQVKGRKPAALGEAQPTKSLXENKSLKEQKKLNDLCFLKRLLQEIKTCWNKILXGTKEH

SEQ ID NO: 11 - protein, 152 aa, synthetic construct; identical to SEQ ID NO: 3
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine

SEQ ID NO: 12 - protein, 152 aa, synthetic construct; identical to SEQ ID NO: 6
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 81: note = N-(2x Polyethylene glycol-methoxy)-L-lysine
  SITE 114: note = Homoserine
  SITE 147: note = Norleucine

SEQ ID NO: 13-49 - skipped (37 entries)

SEQ ID NO: 50 - protein, 152 aa, synthetic construct
DCDIEGKDGKQYESWLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 51 - protein, 160 aa, synthetic construct
DCDIEGKDGKQYESWLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHVDHHHHHH

SEQ ID NO: 52 - protein, 152 aa, synthetic construct
  SITE 81: note = N-(7x Polyethylene glycol-amino)-L-lysine
DCDIEGKDGKQYESVLMVSIDQLLDSMKEIGSNCLNNEFNFFKRHICDANKEGMFLFRAARKLRQFLKMNSTGDFDLHLLXVSENTTILLNCTGQVKGRKPAALGEAQPTKSLEENKSLKEQKKLNDLCFLKRLLQEIKTCWNKILMGTKEH

SEQ ID NO: 53 - protein, 152 aa, synthetic construct
DCDIEGKDGKQYESWLMVSIDQLLDSMKEIGSNCLNNEFNFFKRHICDANKEGMFLFRAARKLRQFLKMNSTGDFDLHLLKVSENTTILLNCTGQVKGRKPAALGEAQPTKSLEENKSLKEQKKLNDLCFLKRLLQEIKTCWNKILMGTKEH

SEQ ID NO: 54 - protein, 152 aa, synthetic construct
  SITE 81: note = N-(7x Polyethylene glycol-amino)-L-lysine
DCDIEGKDGKQYESWLMVSIDQLLDSMKEIGSNCLNNEFNFFKRHICDANKEGMFLFRAARKLRQFLKMNSTGDFDLHLLXVSENTTILLNCTGQVKGRKPAALGEAQPTKSLEENKSLKEQKKLNDLCFLKRLLQEIKTCWNKILMGTKEH

SEQ ID NO: 55 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSENTTILLNCTGQVKGRKPAALGEAQPTKSLEENKSLKEQKKLNDLCFLKRLLQEIKTCWNKILMGTKEH

SEQ ID NO: 56 - protein, 160 aa, synthetic construct
DCDIEGKDGKFYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHVDHHHHHH

SEQ ID NO: 57 - protein, 152 aa, synthetic construct
DCDIEGKDGKFYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 58-100 - skipped (43 entries)

SEQ ID NO: 101 - protein, 35 aa, synthetic construct
  SITE 17: note = Norleucine
  SITE 27: note = Norleucine
  SITE 35: note = Alpha-keto-leucine
DCDIEGKDGKQYESVLXVSIDQLLDSXKEIGSNCX

SEQ ID NO: 102 - protein, 35 aa, synthetic construct
  SITE 35: note = Alpha-keto-leucine
DCDIEGKDGKQYESVLMVSIDQLLDSMKEIGSNCX

SEQ ID NO: 103 - protein, 35 aa, synthetic construct
  SITE 17: note = Norleucine
  SITE 27: note = Norleucine
  SITE 35: note = Alpha-keto-leucine
DCDIEGKDGKQYESWLXVSIDQLLDSXKEIGSNCX

SEQ ID NO: 104 - protein, 35 aa, synthetic construct
  SITE 17: note = Norleucine
  SITE 27: note = Norleucine
  SITE 35: note = Alpha-keto-leucine
DSDIEGKDGKQYESVLXVSIDQLLDSXKEIGSNCX

SEQ ID NO: 105 - protein, 35 aa, synthetic construct
  SITE 17: note = Norleucine
  SITE 27: note = Norleucine
  SITE 35: note = Alpha-keto-leucine
DCDIEGKDGKQYESVLXVSIDQLLDSXKEIGSNSX

SEQ ID NO: 106 - protein, 40 aa, synthetic construct
  SITE 1: note = 5-oxaproline
  SITE 19: note = Norleucine
  SITE 34: note = Norleucine
  SITE 40: note = Alpha-keto-phenylalanine
XNEFNFFKRHICDANKEGXFLFRAARKLRQFLKXNSTGDX

SEQ ID NO: 107 - protein, 40 aa, synthetic construct
  SITE 1: note = 5-oxaproline
  SITE 40: note = Alpha-keto-phenylalanine
XNEFNFFKRHICDANKEGMFLFRAARKLRQFLKMNSTGDX

SEQ ID NO: 108 - protein, 40 aa, synthetic construct
  SITE 1: note = 5-oxaproline
  SITE 19: note = Norleucine
  SITE 34: note = Norleucine
  SITE 40: note = Alpha-keto-phenylalanine
XNEFNFFKRHISDANKEGXFLFRAARKLRQFLKXNSTGDX

SEQ ID NO: 109 - protein, 38 aa, synthetic construct
  SITE 1: note = 5-oxaproline
  SITE 38: note = Alpha-keto-leucine
XLHLLKVSEGTTILLNCTGQVKGRKPAALGEAQPTKSX

SEQ ID NO: 110 - protein, 38 aa, synthetic construct
  SITE 1: note = 5-oxaproline
  SITE 6: note = N-(7x Polyethylene glycol-amino)-L-lysine
  SITE 38: note = Alpha-keto-leucine
XLHLLXVSEGTTILLNCTGQVKGRKPAALGEAQPTKSX

SEQ ID NO: 111 - protein, 38 aa, synthetic construct
  SITE 1: note = 5-oxaproline
  SITE 6: note = N-(7x Polyethylene glycol-amino)-L-lysine
  SITE 38: note = Alpha-keto-leucine
XLHLLXVSENTTILLNCTGQVKGRKPAALGEAQPTKSX

SEQ ID NO: 112 - protein, 38 aa, synthetic construct; identical to SEQ ID NO: 110
  SITE 1: note = 5-oxaproline
  SITE 6: note = N-(2x Polyethylene glycol-methoxy)-L-lysine
  SITE 38: note = Alpha-keto-leucine

SEQ ID NO: 113 - protein, 38 aa, synthetic construct; identical to SEQ ID NO: 110
  SITE 1: note = 5-oxaproline
  SITE 6: note = N-(11x Polyethylene glycol-amino-L-lysine)
  SITE 38: note = Alpha-keto-leucine

SEQ ID NO: 114 - protein, 38 aa, synthetic construct
  SITE 1: note = 5-oxaproline
  SITE 38: note = Alpha-keto-leucine
XLHLLKVSENTTILLNCTGQVKGRKPAALGEAQPTKSX

SEQ ID NO: 115 - protein, 38 aa, synthetic construct
  SITE 1: note = 5-oxaproline
  SITE 38: note = Alpha-keto-leucine
XLHLLKVSEGTTILLNSTGQVKGRKPAALGEAQPTKSX

SEQ ID NO: 116 - protein, 39 aa, synthetic construct
  SITE 1: note = 5-oxaproline
  SITE 34: note = Norleucine
XENKSLKEQKKLNDLCFLKRLLQEIKTCWNKILXGTKEH

SEQ ID NO: 117 - protein, 39 aa, synthetic construct
  SITE 1: note = 5-oxaproline
XENKSLKEQKKLNDLCFLKRLLQEIKTCWNKILMGTKEH

SEQ ID NO: 118 - protein, 39 aa, synthetic construct
  SITE 1: note = 5-oxaproline
  SITE 34: note = Norleucine
XENKSLKEQKKLNDLSFLKRLLQEIKTCWNKILXGTKEH

SEQ ID NO: 119 - protein, 39 aa, synthetic construct
  SITE 1: note = 5-oxaproline
  SITE 34: note = Norleucine
XENKSLKEQKKLNDLCFLKRLLQEIKTSWNKILXGTKEH

SEQ ID NO: 120-124 - skipped (5 entries)

SEQ ID NO: 125 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 126 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 132 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 134 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 119 aa, synthetic construct
QVQLVQSGVEVKKPGASVKVSCKASGYTFTNYYMYWVRQAPGQGLEWMGGFPSNGGTNFNEKFKNRVTLTTDSSTTTAYMELKSLQFDDTAVYYCARRDYRFDMGFDYWGQGTTVTVSS

SEQ ID NO: 138 - protein, 111 aa, synthetic construct [sequence omitted]

SEQ ID NO: 139 - protein, 113 aa, synthetic construct [sequence omitted]

SEQ ID NO: 140 - protein, 107 aa, synthetic construct [sequence omitted]

SEQ ID NO: 141 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 144 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 443 aa, synthetic construct
QVQLVESGGGLVKPGGSLRLSCAASGFTFSNYGMSWIRQAPGKGLEWSTISGGGSNIYYADSVKGRFTISRDNAKNSLYLQMNSLRAEDTAVYYCVSYYYGIDFWGQGTSVTVSSASKYGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 150 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 152 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 154 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 156 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 158 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 159 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 113 aa, synthetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 162 - protein, 107 aa, synthetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 163 - protein, 120 aa, synthetic construct
EVQLQQSGPVLVKPGASVKMSCKASGYTFTSYYMYWVKQSHGKSLEWIGGVNPSNGGTNFNEKFKSKATLTVDKSSSTAYMELNSLTSEDSAVYYCARRDYRYDMGFDYWGQGTTLTVSS

SEQ ID NO: 164 - protein, 111 aa, synthetic construct
QIVLTQSPAIMSASPGEKVTMTCRASKGVSTSGYSYLHWYQQKPGSSPRLLIYLASYLESGVPVRFSGSGSGTSYSLTISRMEAEDAATYYCQHSRELPLTFGTGTRLEIK

SEQ ID NO: 165 - protein, 120 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 166 - protein, 111 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 167 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 5 aa, synthetic construct
NYYMY

SEQ ID NO: 170 - protein, 17 aa, synthetic construct
GINPSNGGTNFNEKFKN

SEQ ID NO: 171 - protein, 11 aa, synthetic construct
RDYRFDMGFDY

SEQ ID NO: 172 - protein, 15 aa, synthetic construct
RASKGVSTSGYSYLH

SEQ ID NO: 173 - protein, 7 aa, synthetic construct
LASYLES

SEQ ID NO: 174 - protein, 9 aa, synthetic construct
QHSRDLPLT

SEQ ID NO: 175 - protein, 5 aa, synthetic construct
NSGMH

SEQ ID NO: 176 - protein, 17 aa, synthetic construct
VIWYDGSKRYYADSVKG

SEQ ID NO: 177 - protein, 4 aa, synthetic construct
NDDY

SEQ ID NO: 178 - protein, 11 aa, synthetic construct
RASQSVSSYLA

SEQ ID NO: 179 - protein, 7 aa, synthetic construct
DASNRAT

SEQ ID NO: 180 - protein, 9 aa, synthetic construct
QQSSNWPRT

SEQ ID NO: 181 - protein, 9 aa, synthetic construct
FTFSNYGMS

SEQ ID NO: 182 - protein, 10 aa, synthetic construct
TISGGGSNIY

SEQ ID NO: 183 - protein, 9 aa, synthetic construct
VSYYYGIDF

SEQ ID NO: 184 - protein, 11 aa, synthetic construct
KASQDVTTAVA

SEQ ID NO: 185 - protein, 7 aa, synthetic construct
WASTRHT

SEQ ID NO: 186 - protein, 9 aa, synthetic construct
QQHYTIPWT

SEQ ID NO: 187 - protein, 8 aa, synthetic construct
GLTFSSSG

SEQ ID NO: 188 - protein, 8 aa, synthetic construct
IWYDGSKR

SEQ ID NO: 189 - protein, 6 aa, synthetic construct
ATNNDY

SEQ ID NO: 190 - protein, 11 aa, synthetic construct
RASQSVSSYLA

SEQ ID NO: 191 - protein, 7 aa, synthetic construct
TASNRAT

SEQ ID NO: 192 - protein, 9 aa, synthetic construct
QQYSNWPRT

SEQ ID NO: 193 - protein, 831 aa, synthetic construct
MQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPSCPAPEFLGGPSVFLFPPKPKDTLMISRTPEVTCWVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLMQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 194 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 195 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 196 - protein, 111 aa, synthetic construct
DVVMTQSPLSLPVTLGQPASISCRASESVDTSDNSFIHWYQQRPGQSPRLLIYRSSTLESGVPDRFSGSGSGTDFTLKISRVEAEDVGVYYCQQNYDVPWTFGQGTKVEIK

SEQ ID NO: 197 - protein, 111 aa, synthetic construct
EIVLTQSPATLSLSPGERATLSCRASESVDTSDNSFIHWYQQKPGQSPRLLIYRSSTLESGIPARFSGSGSGTDFTLTISSLEPEDFAVYYCQQNYDVPWTFGQGTKVEIK

SEQ ID NO: 198 - protein, 120 aa, synthetic construct
DVQLQESGPGLVKPSQSLSLTCTVTGYSITSDYAWNWIRQFPGNKLEWMGYITYTGRTSYNPSLKSRISITRDTSKNQFFLQLNSVTTEDTATYYCAREMDYYGSTLDYWGQGTTLTVSS

SEQ ID NO: 199 - protein, 111 aa, synthetic construct
KIVLTQSPASLAVSLRQRATISCRASESVDRYGNSFIHWYQQKPGQPPKVLIYRASNLESGFPARFSGSGSRTDFTLTIDPVEADDAATYYCQQNNEDPYTFGSGTKLEIK

SEQ ID NO: 200 - protein, 118 aa, synthetic construct
QVQLQQSGPELVRPGVSVKISCKGSGYTFTDYAMHWVKQSHARTLEWIGVISTYSGDTNYNQKFKDKATMTVDKSSSTAYLELARMTSEDSAIYYCARLGITTGFAYWGQGTLVTVSA

SEQ ID NO: 201 - protein, 111 aa, synthetic construct
DIVLTQSPASLAVSLGQRATISCRASKGVSTSSYSFMHWYQQKPRQPPKLLIKYASYLESGVPARFSGSGSGTDFTLNIHPVEEEDAATYYCHHSREFPWTFGGGTKLEIK

SEQ ID NO: 202 - protein, 116 aa, synthetic construct
EVKLVESGGGLVKPGGSLKLSCAASGFTFSNYGMSWIRQTPEKGLEWVATISGGGRDTYYPDSVKGRFTISRDNVKNNLYLQMSSLRSEDTAFYYCASYYYGIDYWGQGTSVTVSS

SEQ ID NO: 203 - protein, 107 aa, synthetic construct
DIVMTQPHKEMSTSVGDRVRITCKASQDVTTAVAWYQQKPGQSPKLLIYWASTRHTGVPDRFTGSGSGTEFTLTISSVQAEDLALYYCQQHYSIPWTFGGGTKLEIK

SEQ ID NO: 204 - protein, 114 aa, synthetic construct
QVQLQQPGAELVKPGASVKMSCKASGYTFTSTWMHWVKQRPGQGLEWIGAIDPSDSYTTYNQKFKGKATLTVDTSSTTAYMQLSSLTSEDSAVYYCTRSPFDYWGQGTTLTVSS

SEQ ID NO: 205 - protein, 107 aa, synthetic construct
DIVMTQSHKEMSTSVGDRVSITCKASQDVSTAVAWYQQKPGQSPKLLIYSASYRYTGVPDRFTGSGSGTDFTFAISSVQAEDLAVYYCQQHYSHPFTFGSGTKLEIK

SEQ ID NO: 206 - protein, 120 aa, synthetic construct
DVQLQESGPGLVKPSQSLSLTCTVTGYSITSDYAWNWIRQFPGDKLEWLGYITYSGFTNYNPSLKSRISISRDTSKNQFFLQLNSVATEDTATYYCARWHGSAPWYFDYWGRGTTLTVSS

SEQ ID NO: 207 - protein, 112 aa, synthetic construct
DVLMTQTPLSLPVSLGDQASISCRSSQNIVHSDGNTYLEWYLQKPGQSPNLLIYKVSRRFSGVPDRFSGSGSGTDFTLKISRVEAEDLGVYYCFQGSHFPLTFGAGTKLELK

SEQ ID NO: 208 - protein, 117 aa, synthetic construct
DVQLQESGPDLVKPSQSLSLTCTVTGYSITSGYSWHWIRQFPGNKLEWMGFIHSSGDTNYNPSLKSRISFTRDTSKNQFFLQLSSLTDEDTATYYCATYRNWYFDVWGAGTTVTVSS

SEQ ID NO: 209 - protein, 113 aa, synthetic construct
DIVMTQSPSSLTVTAGEKVTMRCKSSQSLLNSGTQKNYLTWYQQKPGQPPKLLIYWASTRESGVPNRFTGSGSGTDFTLTISSVQAEDLSVYYCQSDYTFPLTFGGGTKLELK

SEQ ID NO: 210 - protein, 120 aa, synthetic construct [sequence omitted]

SEQ ID NO: 211 - protein, 111 aa, synthetic construct [sequence omitted]

SEQ ID NO: 212 - protein, 113 aa, synthetic construct [sequence omitted]

SEQ ID NO: 213 - protein, 107 aa, synthetic construct [sequence omitted]

SEQ ID NO: 214 - protein, 120 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSS

SEQ ID NO: 215 - protein, 111 aa, synthetic construct
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIK

SEQ ID NO: 216 - protein, 8 aa, synthetic construct
GFSFSSYT

SEQ ID NO: 217 - protein, 8 aa, synthetic construct
ISGGGRDI

SEQ ID NO: 218 - protein, 13 aa, synthetic construct
VLLTGRVYFALDS

SEQ ID NO: 219 - protein, 10 aa, synthetic construct
ESVDTSDNSF

SEQ ID NO: 220 - skipped

SEQ ID NO: 221 - protein, 6 aa, synthetic construct
NYDVPW

SEQ ID NO: 222 - protein, 8 aa, synthetic construct
GYSITSDY

SEQ ID NO: 223 - skipped

SEQ ID NO: 224 - protein, 9 aa, synthetic construct
MDYYGSTLD

SEQ ID NO: 225 - protein, 11 aa, synthetic construct
SESVDRYGNSF

SEQ ID NO: 226 - skipped

SEQ ID NO: 227 - protein, 6 aa, synthetic construct
NNEDPY

SEQ ID NO: 228 - protein, 7 aa, synthetic construct
GYTFTDY

SEQ ID NO: 229 - skipped

SEQ ID NO: 230 - protein, 7 aa, synthetic construct
GITTGFA

SEQ ID NO: 231 - protein, 11 aa, synthetic construct
SKGVSTSSYSF

SEQ ID NO: 232 - skipped

SEQ ID NO: 233 - protein, 6 aa, synthetic construct
SREFPW

SEQ ID NO: 234 - protein, 7 aa, synthetic construct
GFTFSNY

SEQ ID NO: 235 - skipped

SEQ ID NO: 236 - protein, 5 aa, synthetic construct
YYGID

SEQ ID NO: 237 - protein, 7 aa, synthetic construct
SQDVTTA

SEQ ID NO: 238 - skipped

SEQ ID NO: 239 - protein, 6 aa, synthetic construct
HYSIPW

SEQ ID NO: 240 - protein, 7 aa, synthetic construct
GYTFTST

SEQ ID NO: 241-242 - skipped (2 entries)

SEQ ID NO: 243 - protein, 7 aa, synthetic construct
SQDVSTA

SEQ ID NO: 244 - skipped

SEQ ID NO: 245 - protein, 6 aa, synthetic construct
HYSHPF

SEQ ID NO: 246 - protein, 8 aa, synthetic construct
GYSITSDY

SEQ ID NO: 247 - skipped

SEQ ID NO: 248 - protein, 8 aa, synthetic construct
GSAPWYFD

SEQ ID NO: 249 - protein, 12 aa, synthetic construct
SQNIVHSDGNTY

SEQ ID NO: 250 - skipped

SEQ ID NO: 251 - protein, 5 aa, synthetic construct
SHFPL

SEQ ID NO: 252 - protein, 8 aa, synthetic construct
GYSITSGY

SEQ ID NO: 253 - skipped

SEQ ID NO: 254 - protein, 6 aa, synthetic construct
RNWYFD

SEQ ID NO: 255 - protein, 13 aa, synthetic construct
SQSLLNSGTQKNY

SEQ ID NO: 256 - skipped

SEQ ID NO: 257 - protein, 6 aa, synthetic construct
DYTFPL

SEQ ID NO: 258 - protein, 7 aa, synthetic construct
GYFTNYY

SEQ ID NO: 259 - protein, 8 aa, synthetic construct
INPSNGGT

SEQ ID NO: 260 - protein, 13 aa, synthetic construct
ARRDYRFDMGFDY

SEQ ID NO: 261 - protein, 10 aa, synthetic construct
KGVSTSGYSY

SEQ ID NO: 262 - skipped

SEQ ID NO: 263 - protein, 9 aa, synthetic construct
QHSRDLPLT

SEQ ID NO: 264 - protein, 8 aa, synthetic construct
GITFSNSG

SEQ ID NO: 265 - protein, 8 aa, synthetic construct
IWYDGSKR

SEQ ID NO: 266 - protein, 6 aa, synthetic construct
ATNDDY

SEQ ID NO: 267 - protein, 6 aa, synthetic construct
QSVSSY

SEQ ID NO: 268 - skipped

SEQ ID NO: 269 - protein, 9 aa, synthetic construct
QQSSNWRRT

SEQ ID NO: 270 - protein, 113 aa, synthetic construct
QVQLVESGGDVVQPGGSLRLSCAASGVAFSNYGMHWVRQAPGKGLEWVAVIWYDGSNKYYADSVKGRFTISRDNSKNMLYLQMNSLRAEDTAMYYCARNDDYWGQGTLVTVSS

SEQ ID NO: 271 - protein, 107 aa, synthetic construct
EIVLTQSPATLSLSPGERATLSCRASQSVSSYLAWYQQKPGQAPRLIIYDASNRATGIPARFSGSGSGTDFTLTISSLEPEDFAVYYCQQRSNWPLTFGGGTKVEIK

SEQ ID NO: 272 - protein, 113 aa, synthetic construct
QVQLVESGGDVVQPGRSLRLSCAASGLTFTNYGFHWVRQAPGKGLEWVAVIWYDGSKKYYADSVKGRFTISRDNSKNTLYLQMNNLRAEDTAVYYCATGDDYWGQGTLVTVSS

SEQ ID NO: 273 - protein, 107 aa, synthetic construct
EIVLTQSPATLSLSPGERATLSCRASQSVSSYLAWYQQKPGQAPRLLIYDTSNRATGIPARFSGSGSGTDFTLTISSLEPEDFAVYYCQQRSNWPLTFGGGTKVEIK

SEQ ID NO: 274 - protein, 113 aa, synthetic construct
QVYLVESGGGVVQPGRSLRLSCAASGFTFSNYGMHWVRQAPGKGLEWVALIWYDGSNKYYADSVKGRFTISRDNSKNTLYLQMTSLRVEDTAVYYCASNVDHWGQGTLVTVSS

SEQ ID NO: 275 - protein, 121 aa, synthetic construct
QLQLQESGPGLVKPSETLSLTCTVSGGSLSRSSFFWGWIRQPPGKGLEWIGSIYYSGSTYYNPSLKSRVTISVDTSKNQFSLKLSSVTAADTAVYYCVRDYDILTGDEDYWGQGTLVTVSS

SEQ ID NO: 276 - protein, 113 aa, synthetic construct
QVQLVESGGGVVQPGRSLRLSCTTSGITFSSYGFHWVRQAPGKGLEWVAVIWYDGSKKYYADSVKGRFTLSRDDSKNTLYLQMNSLRAEDTAVYYCVTGDDYWGQGTLVTVSS

SEQ ID NO: 277 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 278 - protein, 121 aa, synthetic construct
QLQLQESGPGLVKPSETLSLTCSVSGGSLSRSSYFWGWIRQPPGKGLEWIASIFYSGETYFNPSLKSRVTISVDTSRNQFSLKLSSVTAADTAVYYCARDYDILTGDEDYWGQGTLVTVSS

SEQ ID NO: 279 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYYSYPRTFGQGTKVEIK

SEQ ID NO: 280 - protein, 5 aa, synthetic construct
NYGMH

SEQ ID NO: 281 - protein, 17 aa, synthetic construct
VIWYDGSNKYYADSVKG

SEQ ID NO: 282 - protein, 4 aa, synthetic construct
NDDY

SEQ ID NO: 283 - protein, 11 aa, synthetic construct
RASQSVSSYLA

SEQ ID NO: 284 - protein, 7 aa, synthetic construct
DASNRAT

SEQ ID NO: 285 - protein, 9 aa, synthetic construct
QQRSNWPLT

SEQ ID NO: 286 - protein, 5 aa, synthetic construct
NYGFH

SEQ ID NO: 287 - protein, 17 aa, synthetic construct
VIWYDGSKKYYADSVKG

SEQ ID NO: 288 - protein, 4 aa, synthetic construct
GDDY

SEQ ID NO: 289 - protein, 11 aa, synthetic construct
RASQSVSSYLA

SEQ ID NO: 290 - protein, 7 aa, synthetic construct
DTSNRAT

SEQ ID NO: 291 - protein, 9 aa, synthetic construct
QQRSNWPLT

SEQ ID NO: 292 - protein, 5 aa, synthetic construct
NYGMH

SEQ ID NO: 293 - protein, 17 aa, synthetic construct
LIWYDGSNKYYADSVKG

SEQ ID NO: 294 - protein, 4 aa, synthetic construct
NVDH

SEQ ID NO: 295 - protein, 7 aa, synthetic construct
RSSFFWG

SEQ ID NO: 296 - protein, 16 aa, synthetic construct
SIYYSGSTYYNPSLKS

SEQ ID NO: 297 - protein, 11 aa, synthetic construct
DYDILTGDEDY

SEQ ID NO: 298 - protein, 5 aa, synthetic construct
SYGFH

SEQ ID NO: 299 - protein, 17 aa, synthetic construct
VIWYDGSKKYYADSVKG

SEQ ID NO: 300 - protein, 4 aa, synthetic construct
GDDY

SEQ ID NO: 301 - protein, 11 aa, synthetic construct
RASQSVSSYLA

SEQ ID NO: 302 - protein, 7 aa, synthetic construct
DASNRAT

SEQ ID NO: 303 - protein, 9 aa, synthetic construct
QQRSNWPLT

SEQ ID NO: 304 - protein, 7 aa, synthetic construct
RSSYFWG

SEQ ID NO: 305 - protein, 16 aa, synthetic construct
SIFYSGETYFNPSLKS

SEQ ID NO: 306 - protein, 11 aa, synthetic construct
DYDILTGDEDY

SEQ ID NO: 307 - protein, 11 aa, synthetic construct
RASQGISSWLA

SEQ ID NO: 308 - protein, 7 aa, synthetic construct
AASSLQS

SEQ ID NO: 309 - protein, 9 aa, synthetic construct
QQYYSYPRT

SEQ ID NO: 310-350 - skipped (41 entries)

SEQ ID NO: 351 - protein, 20 aa, synthetic construct
  VARIANT 1..20: note = This sequence may encompass 1-10 GS repeating units
GSGSGSGSGSGSGSGSGSGS

SEQ ID NO: 352 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 353 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 354 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 355 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 356 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 357 - protein, 15 aa, synthetic construct
GGGGSGGGGSGGGGS

SEQ ID NO: 358 - protein, 20 aa, synthetic construct
GGGGSGGGGSGGGGSGGGGS

SEQ ID NO: 359 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 360 - protein, 230 aa, synthetic construct
  VARIANT 18: note = Any naturally occurring amino acid
  VARIANT 19: note = Any naturally occurring amino acid
  VARIANT 21: note = Any naturally occurring amino acid
  VARIANT 140: note = Any naturally occurring amino acid
  VARIANT 142: note = Any naturally occurring amino acid
  VARIANT 177: note = Any naturally occurring amino acid
PKSCDKTHTCPPCPAPEXXGXPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKXTPPVLDSDGSFFLYSKLTVDKSRWQQGNVFSCSVMHEALHNHYTQKSLSLSPG

SEQ ID NO: 361 - protein, 48 aa, synthetic construct
QETNPTENLYFQQKNMQCQRRFYEALHDPNLNEEQRNARIRSIRDDDC

SEQ ID NO: 362 - protein, 61 aa, synthetic construct
QTADNQKNMQCQRRFYEALHDPNLNEEQRNARIRSIRDDCSQSANLLAEAQQLNDAQAPQA

SEQ ID NO: 363 - protein, 38 aa, synthetic construct
QETKNMQCQRRFYEALHDPNLNEEQRNARIRSIRDDDC

SEQ ID NO: 364 - protein, 38 aa, synthetic construct
QETFNKQCQRRFYEALHDPNLNEEQRNARIRSIRDDDC

SEQ ID NO: 365 - protein, 38 aa, synthetic construct
QETFNMQCQRRFYEALHDPNLNKEQRNARIRSIRDDDC

SEQ ID NO: 366 - protein, 37 aa, synthetic construct
QETFNMQCQRRFYEALHDPNLNEEQRNARIRSIKDDC

SEQ ID NO: 367 - protein, 35 aa, synthetic construct
QETMQCQRRFYEALHDPNLNEEQRNARIRSIKDDC

SEQ ID NO: 368 - protein, 34 aa, synthetic construct
QETQCQRRFYEALHDPNLNEEQRNARIRSIKDDC

SEQ ID NO: 369 - protein, 33 aa, synthetic construct
QETCQRRFYEALHDPNLNEEQRNARIRSIKDDC

SEQ ID NO: 370 - protein, 20 aa, synthetic construct
QETRGNCAYHKGQLVWCTYH

SEQ ID NO: 371 - protein, 20 aa, synthetic construct
QETRGNCAYHKGQIIWCTYH

SEQ ID NO: 372 - protein, 6 aa, synthetic construct
  VARIANT 6: note = A or P
LLQGPX

SEQ ID NO: 373 - protein, 8 aa, synthetic construct
GGLLQGPP

SEQ ID NO: 374 - protein, 6 aa, synthetic construct
HHHHHH

SEQ ID NO: 375 - protein, 17 aa, synthetic construct
  SITE 1: note = Acetylated residue
  SITE 8: note = 3-((3-((2,5-dioxopyrrolidin-1-yl)oxy)-3-oxopropyl)d isulfaneyl)propanoate modified lysine
  SITE 17: note = Amidated residue
  DISULFID 4..14: note = Intrachain disulfide bond
RGNCAYHXGQLVWCTYH

SEQ ID NO: 376 - protein, 17 aa, synthetic construct
  SITE 1: note = Acetylated residue
  SITE 8: note = 4-oxo-4-((3-oxo-3-(phenylthio)propyl)thio)butanoate modified lysine
  SITE 17: note = Amidated residue
  DISULFID 4..14: note = Intrachain disulfide bond
RGNCAYHXGQLVWCTYH

SEQ ID NO: 377 - protein, 34 aa, synthetic construct
  SITE 1: note = Acetylated residue
  SITE 31: note = 3-(((3-oxo-3-(phenylthio)propyl)thio)carbonyl)benzo ate modified lysine
  SITE 34: note = Amidated residue
  DISULFID 5..34: note = Intrachain disulfide bond
FNMQCQRRFYEALHDPNLNEEQRNARIRSIXDDC

SEQ ID NO: 378 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNCLXNEFNFFKRHICDANKEGXFLFRAARKLRQFLKXNSTGDFXLHLLWVSEGTTILLNCTGQVKGRKPAALGEAQPTKSLXENKSLKEQKKLNDLCFLKRLLQEIKTCWNKILXGTKEH

SEQ ID NO: 379 - protein, 152 aa, synthetic construct; identical to SEQ ID NO: 6
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 81: note = N-adamantyl-L-lysine
  SITE 114: note = Homoserine
  SITE 147: note = Norleucine

SEQ ID NO: 380 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNALXNEFNFFKRHICDANKEGXFLFRAARKLRQFLKXNSTGDFXLHLLKVSEGTTILLNCTGQVKGRKPAALGEAQPTKSLXENKSLKEQKKLNDLAFLKRLLQEIKTCWNKILXGTKEH

SEQ ID NO: 381 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNCLXNEFNFFKRHIADANKEGXFLFRAARKLRQFLKXNSTGDFXLHLLKVSEGTTILLNCTGQVKGRKPAALGEAQPTKSLXENKSLKEQKKLNDLCFLKRLLQEIKTAWNKILXGTKEH

SEQ ID NO: 382 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DSDIEGKDGKQYESVLXVSIDQLLDSXKEIGSNCLXNEFNFFKRHICDANKEGXFLFRAARKLRQFLKXNSTGDFXLHLLKVSEGTTILLNSTGQVKGRKPAALGEAQPTKSLXENKSLKEQKKLNDLCFLKRLLQEIKTCWNKILXGTKEH

SEQ ID NO: 383 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNSLXNEFNFFKRHICDANKEGXFLFRAARKLRQFLKXNSTGDFXLHLLKVSEGTTILLNCTGQVKGRKPAALGEAQPTKSLXENKSLKEQKKLNDLSFLKRLLQEIKTCWNKILXGTKEH

SEQ ID NO: 384 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNCLXNEFNFFKRHISDANKEGXFLFRAARKLRQFLKXNSTGDFXLHLLKVSEGTTILLNCTGQVKGRKPAALGEAQPTKSLXENKSLKEQKKLNDLCFLKRLLQEIKTSWNKILXGTKEH

SEQ ID NO: 385 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNSLXNEFNFFKRHISDANKEGXFLFRAARKLRQFLKXNSTGDFXLHLLKVSEGTTILLNCTGQVKGRKPAALGEAQPTKSLXENKSLKEQKKLNDLSFLKRLLQEIKTSWNKILXGTKEH

SEQ ID NO: 386 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DSDIEGKDGKQYESVLXVSIDQLLDSXKEIGSNCLXNEFNFFKRHISDANKEGXFLFRAARKLRQFLKXNSTGDFXLHLLKVSEGTTILLNSTGQVKGRKPAALGEAQPTKSLXENKSLKEQKKLNDLCFLKRLLQEIKTSWNKILXGTKEH

SEQ ID NO: 387 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DSDIEGKDGKQYESVLXVSIDQLLDSXKEIGSNSLXNEFNFFKRHICDANKEGXFLFRAARKLRQFLKXNSTGDFXLHLLKVSEGTTILLNSTGQVKGRKPAALGEAQPTKSLXENKSLKEQKKLNDLSFLKRLLQEIKTCWNKILXGTKEH

SEQ ID NO: 388 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DSDIEGKDGKQYESVLXVSIDQLLDSXKEIGSNCLXNEFNFFKRHICDANKEGXFLFRAARKLRQFLKXNSTGDFXLHLLKVSENTTILLNSTGQVKGRKPAALGEAQPTKSLXENKSLKEQKKLNDLCFLKRLLQEIKTCWNKILXGTKEH

SEQ ID NO: 389 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 81: note = N-(7x Polyethylene glycol-amino)-L-lysine
  SITE 114: note = Homoserine
  SITE 147: note = Norleucine
DSDIEGKDGKQYESVLXVSIDQLLDSXKEIGSNCLXNEFNFFKRHICDANKEGXFLFRAARKLRQFLKXNSTGDFXLHLLXVSENTTILLNSTGQVKGRKPAALGEAQPTKSLXENKSLKEQKKLNDLCFLKRLLQEIKTCWNKILXGTKEH

SEQ ID NO: 390 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNCLXNEFNFFKRHISDANKEGXFLFRAARKLRQFLKXNSTGDFXLHLLKVSENTTILLNCTGQVKGRKPAALGEAQPTKSLXENKSLKEQKKLNDLCFLKRLLQEIKTSWNKILXGTKEH

SEQ ID NO: 391 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 81: note = N-(7x Polyethylene glycol-amino)-L-lysine
  SITE 114: note = Homoserine
  SITE 147: note = Norleucine
DCDIEGKDGKQYESVLXVSIDQLLDSXKEIGSNCLXNEFNFFKRHISDANKEGXFLFRAARKLRQFLKXNSTGDFXLHLLXVSENTTILLNCTGQVKGRKPAALGEAQPTKSLXENKSLKEQKKLNDLCFLKRLLQEIKTSWNKILXGTKEH